SEQUENCE LISTING (279 sequences)

SEQ ID NO: 1 - protein, 13 aa, Homo sapiens
SVPHFSDEDKDPE

SEQ ID NO: 2 - protein, 186 aa, Homo sapiens
YSPRTPDRVSEADIQRLLHGVMEQLGIARPRVEYPAHQAMNLVGPQSIEGGAHEGLQHLGPFGNIPNIVAELTGDNIPKDFSEDQGYPDPPNPCPVGKTADDGCLENTPDTAEFSREFQLHQHLFDPEHDYPGLGKWNKKLLYEKMKGGERRKRRSVNPYLQGQRLDNVVAKKSVPHFSDEDKDPE

SEQ ID NO: 3 - protein, 150 aa, Homo sapiens
YSPRTPDRVSEADIQRLLHGVMEQLGIARPRVEYPAHQAMNLVGPQSIEGGAHEGLQHLGPFGNIPNIVAELTGDNIPKDFSEDQGYPDPPNPCPVGKTADDGCLENTPDTAEFSREFQLHQHLFDPEHDYPGLGKWNKKLLYEKMKGGE

SEQ ID NO: 4 - protein, 88 aa, Homo sapiens
MALQADFDRAAEDVRKLKARPDDGELKELYGLYKQAIVGDINIACPGMLDLKGKAKWEAWNLKKGLSTEDATSAYISKAKELIEKYGI

SEQ ID NO: 5 - protein, 86 aa, Homo sapiens [sequence omitted]

SEQ ID NO: 6 - protein, 52 aa, Homo sapiens
YRQSMNNFQGLRSFGCRFGTCTVQKLAHQIYQFTDKDKDNVAPRSKISPQGY

SEQ ID NO: 7 - protein, 47 aa, Homo sapiens
TQAQLLRVGCVLGTCQVQNLSHRLWQLMGPAGRQDSAPVDPSSPHSY

SEQ ID NO: 8 - protein, 40 aa, Homo sapiens
VGCVLGTCQVQNLSHRLWQLMGPAGRQDSAPVDPSSPHSY

SEQ ID NO: 9 - protein, 20 aa, Homo sapiens
ARLDVASEFRKKWNKWALSR

SEQ ID NO: 10 - protein, 13 aa, Homo sapiens
QRPRLSHKGPMPF

SEQ ID NO: 11 - protein, 28 aa, Homo sapiens
NGPGPWQGGRRKFRRQRPRLSHKGPMPF

SEQ ID NO: 12 - protein, 31 aa, Homo sapiens
GSRNGPGPWQGGRRKFRRQRPRLSHKGPMPF

SEQ ID NO: 13 - protein, 36 aa, Homo sapiens
LVQPRGSRNGPGPWQGGRRKFRRQRPRLSHKGPMPF

SEQ ID NO: 14 - protein, 27 aa, Homo sapiens
VPLPAGGGTVLTKMYPRGNHWAVGHLM

SEQ ID NO: 15 - protein, 10 aa, Homo sapiens
GNLWATGHFM

SEQ ID NO: 16 - protein, 32 aa, Homo sapiens
APLSWDLPEPRSRASKIRVHSRGNLWATGHFM

SEQ ID NO: 17 - protein, 10 aa, Homo sapiens
GNHWAVGHLM

SEQ ID NO: 18 - protein, 9 aa, Homo sapiens
RPPGFSPFR

SEQ ID NO: 19 - protein, 10 aa, Homo sapiens
KRPPGFSPFR

SEQ ID NO: 20 - protein, 14 aa, Homo sapiens
ISLMKRPPGFSPFR

SEQ ID NO: 21 - protein, 32 aa, Homo sapiens
CGNLSTCMLGTYTQDFNKFHTFPQTAIGVGAP

SEQ ID NO: 22 - protein, 37 aa, Homo sapiens
ACDTATCVTHRLAGLLSRSGGVVKNNFVPTNVGSKAF

SEQ ID NO: 23 - protein, 37 aa, Homo sapiens
ACNTATCVTHRLAGLLSRSGGMVKSNFVPTNVGSKAF

SEQ ID NO: 24 - protein, 37 aa, Homo sapiens [sequence omitted]

SEQ ID NO: 25 - protein, 21 aa, Homo sapiens
DMSSDLERDHRPHVSMPQNAN

SEQ ID NO: 26 - protein, 39 aa, Homo sapiens
QEDAELQPRALDIYSAVDDASHEKELIEALQEVLKKLKS

SEQ ID NO: 27 - protein, 48 aa, Homo sapiens
VPIYEKKYGQVPMCDAGEQCAVRKGARIGKLCDCPRGTSCNSFLLKCL

SEQ ID NO: 28 - protein, 89 aa, Homo sapiens
QEDAELQPRALDIYSAVDDASHEKELIEALQEVLKKLKSKRVPIYEKKYGQVPMCDAGEQCAVRKGARIGKLCDCPRGTSCNSFLLKCL

SEQ ID NO: 29 - protein, 15 aa, Homo sapiens
GSAKVAFSAIRSTNH

SEQ ID NO: 30 - protein, 16 aa, Homo sapiens
SGSAKVAFSAIRSTNH

SEQ ID NO: 31 - protein, 172 aa, Homo sapiens
QNETEPIVLEGKCLVVCDSNPTSDPTGTALGISVRSGSAKVAFSAIRSTNHEPSEMSNRTMIIYFDQVLVNIGNNFDSERSTFIAPRKGIYSFNFHVVKVYNRQTIQVSLMLNGWPVISAFAGDQDVTREAASNGVLIQMEKGDRAYLKLERGNLMGGWKYSTFSGFLVFPL

SEQ ID NO: 32 - protein, 173 aa, Homo sapiens
QNDTEPIVLEGKCLVVCDSSPSADGAVTSSLGISVRSGSAKVAFSATRSTNHEPSEMSNRTMTIYFDQVLVNIGNHFDLASSIFVAPRKGIYSFSFHVVKVYNRQTIQVSLMQNGYPVISAFAGDQDVTREAASNGVLLLMEREDKVHLKLERGNLMGGWKYSTFSGFLVFPL

SEQ ID NO: 33 - protein, 173 aa, Homo sapiens
QEGSEPVLLEGECLVVCEPGRAAAGGPGGAALGEAPPGRVAFAAVRSHHHEPAGETGNGTSGAIYFDQVLVNEGGGFDRASGSFVAPVRGVYSFRFHVVKVYNRQTVQVSLMLNTWPVISAFANDPDVTREAATSSVLLPLDPGDRVSLRLRRGNLLGGWKYSSFSGFLIFPL

SEQ ID NO: 34 - protein, 174 aa, Homo sapiens
QNDTEPIVLEGKCLVVCDSNPATDSKGSSSSPLGISVRAANSKVAFSAVRSTNHEPSEMSNKTRIIYFDQILVNVGNFFTLESVFVAPRKGIYSFSFHVIKVYQSQTIQVNLMLNGKPVISAFAGDKDVTREAATNGVLLYLDKEDKVYLKLEKGNLVGGWQYSTFSGFLVFPL

SEQ ID NO: 35 - protein, 11 aa, Homo sapiens
AYGFRGPGPQL

SEQ ID NO: 36 - protein, 57 aa, Homo sapiens
SAEFPDFYDSEEPVSTHQEAENEKDRADQTVLTEDEKKELENLAAMDLELQKIAEKF

SEQ ID NO: 37 - protein, 439 aa, Homo sapiens
LPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 38 - protein, 92 aa, Homo sapiens
EDSKEAEKSGEATDGARPQALPEPMQESKAEGNNQAPGEEEEEEEEATNTHPPASLPSQKYPGPQAEGDSEGLSQGLVDREKGLSAEPGWQA

SEQ ID NO: 39 - protein, 37 aa, Homo sapiens
EEEGSANRRPEDQELESLSAIEAELEKVAHQLQALRR

SEQ ID NO: 40 - protein, 33 aa, Homo sapiens
EEEEEEEEEAEAGEEAVPEEEGPTVVLNPHPSL

SEQ ID NO: 41 - protein, 74 aa, Homo sapiens
FLGEGHHRVQENQMDKARRHPQGAWKELDRNYLNYGEEGAPGKWQQQGDLQDTKENREEARFQDKQYSSHHTAE

SEQ ID NO: 42 - protein, 44 aa, Homo sapiens
GYPEEKKEEEGSANRRPEDQELESLSAIEAELEKVAHQLQALRR

SEQ ID NO: 43 - protein, 19 aa, Homo sapiens
GWRPSSREDSLEAGLPLQV

SEQ ID NO: 44 - protein, 19 aa, Homo sapiens
LEGQEEEEDNRDSSMKLSF

SEQ ID NO: 45 - protein, 48 aa, Homo sapiens
SEALAVDGAGKPGAEEAQDPEGKGEQEHSQQKEEEEEMAVVPQGLFRG

SEQ ID NO: 46 - protein, 657 aa, Homo sapiens [sequence omitted]

SEQ ID NO: 47 - protein, 587 aa, Homo sapiens
QRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 48 - protein, 449 aa, Homo sapiens
FPKPGGSQDKSLHNRELSAERPLNEQIAEAEEDKIKKTYPPENKPGQSNYSFVDNLNLLKAITEKEKIEKERQSIRSSPLDNKLNVEDVDSTKNRKLIDDYDSTKSGLDHKFQDDPDGLHQLDGTPLTAEDIVHKIAARIYEENDRAVFDKIVSKLLNLGLITESQAHTLEDEVAEVLQKLISKEANNYEEDPNKPTSWTENQAGKIPEKVTPMAAIQDGLAKGENDETVSNTLTLTNGLERRTKTYSEDNFEELQYFPNFYALLKSIDSEKEAKEKETLITIMKTLIDFVKMMVKYGTISPEEGVSYLENLDEMIALQTKNKLEKNATDNISKLFPAPSEKSHEETDSTKEEAAKMEKEYGSLKDSTKDDNSNPGGKTDEPKGKTEAYLEAIRKNIEWLKKHDKKGNKEDYDLSKMRDFINKQADAYVEKGILDKEEAEAIKRIYSSL

SEQ ID NO: 49 - protein, 33 aa, Homo sapiens
TNEIVEEQYTPQSLATLESVFQELGKLTGPNNQ

SEQ ID NO: 50 - protein, 18 aa, Homo sapiens
SGELEQEEERLSKEWEDS

SEQ ID NO: 51 - protein, 76 aa, Homo sapiens
LPVNSPMNKGDTEVMKCIVEVISDTLSKPSPMPVSQECFETLRGDERILSILRHQNLLKELQDLALQGAKERAHQQ

SEQ ID NO: 52 - protein, 113 aa, Homo sapiens
LPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVME

SEQ ID NO: 53 - protein, 8 aa, Homo sapiens
WSKMDQLA

SEQ ID NO: 54 - protein, 14 aa, Homo sapiens
WSKMDQLAKELTAE

SEQ ID NO: 55 - protein, 626 aa, Homo sapiens
QESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 56 - protein, 362 aa, Homo sapiens
QESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMK

SEQ ID NO: 57 - protein, 255 aa, Homo sapiens
SSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQIDPNGLSFNPISDFPDTTSPKCPGRPWKSVSEINPTTQMKESYYFDLTDGLS

SEQ ID NO: 58 - protein, 4 aa, Homo sapiens
WGHE

SEQ ID NO: 59 - protein, 38 aa, Homo sapiens
CSCSSLMDKECVYFCHLDIIWVNTPEHVVPYGLGSPRS

SEQ ID NO: 60 - protein, 21 aa, Homo sapiens
CSCSSLMDKECVYFCHLDIIW

SEQ ID NO: 61 - protein, 21 aa, Homo sapiens
CSCSSWLDKECVYFCHLDIIW

SEQ ID NO: 62 - protein, 21 aa, Homo sapiens
CTCFTYKDKECVYYCHLDIIW

SEQ ID NO: 63 - protein, 18 aa, Homo sapiens
AGEGLNSQFWSLAAPQRF

SEQ ID NO: 64 - protein, 8 aa, Homo sapiens
FLFQPQRF

SEQ ID NO: 65 - protein, 37 aa, Homo sapiens
SLNFEELKDWGPKNVIKMSTPAVNKMPHSFANLPLRF

SEQ ID NO: 66 - protein, 8 aa, Homo sapiens
VPNLPQRF

SEQ ID NO: 67 - protein, 12 aa, Homo sapiens
MPHSFANLPLRF

SEQ ID NO: 68 - protein, 12 aa, Homo sapiens
SAGATANLPLRS

SEQ ID NO: 69 - protein, 11 aa, Homo sapiens
SQAFLFQPQRF

SEQ ID NO: 70 - protein, 20 aa, Homo sapiens
TPDINPAWYASRGIRPVGRF

SEQ ID NO: 71 - protein, 31 aa, Homo sapiens
SRTHRHSMEIRTPDINPAWYASRGIRPVGRF

SEQ ID NO: 72 - protein, 30 aa, Homo sapiens
GWTLNSAGYLLGPHAVGNHRSFSDKNGLTS

SEQ ID NO: 73 - protein, 59 aa, Homo sapiens
ELRPEDDMKPGSFDRSIPENNIMRTIIEFLSFLHLKEAGALDRLLDLPAAASSEDIERS

SEQ ID NO: 74 - protein, 60 aa, Homo sapiens
APAHRGRGGWTLNSAGYLLGPVLHLPQMGDQDGKRETALEILDLWKAIDGLPYSHPPQPS

SEQ ID NO: 75 - protein, 34 aa, Homo sapiens
QLGPQGPPHLVADPSKKQGPWLEEEEEAYGWMDF

SEQ ID NO: 76 - protein, 95 aa, Homo sapiens
QPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 77 - protein, 12 aa, Homo sapiens
ISDRDYMGWMDF

SEQ ID NO: 78 - protein, 18 aa, Homo sapiens
LDPSHRISDRDYMGWMDF

SEQ ID NO: 79 - protein, 25 aa, Homo sapiens
IVKNLQNLDPSHRISDRDYMGWMDF

SEQ ID NO: 80 - protein, 33 aa, Homo sapiens
KAPSGRMSIVKNLQNLDPSHRISDRDYMGWMDF

SEQ ID NO: 81 - protein, 39 aa, Homo sapiens
YIQQARKAPSGRMSIVKNLQNLDPSHRISDRDYMGWMDF

SEQ ID NO: 82 - protein, 5 aa, Homo sapiens
GWMDF

SEQ ID NO: 83 - protein, 58 aa, Homo sapiens
VSQRTDGESRAHLGALLARYIQQARKAPSGRMSIVKNLQNLDPSHRISDRDYMGWMDF

SEQ ID NO: 84 - protein, 49 aa, Homo sapiens
VSQRTDGESRAHLGALLARYIQQARKAPSGRMSIVKNLQNLDPSHRISD

SEQ ID NO: 85 - protein, 7 aa, Homo sapiens
YMGWMDF

SEQ ID NO: 86 - protein, 8 aa, Homo sapiens [sequence omitted]

SEQ ID NO: 87 - protein, 17 aa, Homo sapiens
QGPWLEEEEEAYGWMDF

SEQ ID NO: 88 - protein, 14 aa, Homo sapiens
WLEEEEEAYGWMDF

SEQ ID NO: 89 - protein, 52 aa, Homo sapiens
DLELPWLEQQGPASHHRRQLGPQGPPHLVADPSKKQGPWLEEEEEAYGWMDF

SEQ ID NO: 90 - protein, 6 aa, Homo sapiens
YGWMDF

SEQ ID NO: 91 - protein, 71 aa, Homo sapiens
SWKPRSQQPDAPLGTGANRDLELPWLEQQGPASHHRRQLGPQGPPHLVADPSKKQGPWLEEEEEAYGWMDF

SEQ ID NO: 92 - protein, 42 aa, Homo sapiens
YAEGTFISDYSIAMDKIHQQDFVNWLLAQKGKKNDWKHNITQ

SEQ ID NO: 93 - protein, 69 aa, Homo sapiens
RSLQDTEEKSRSFSASQADPLSDPDQMNEDKRHSQGTFTSDYSKYLDSRRAQDFVQWLMNTKRNRNNIA

SEQ ID NO: 94 - protein, 30 aa, Homo sapiens
RSLQDTEEKSRSFSASQADPLSDPDQMNED

SEQ ID NO: 95 - protein, 29 aa, Homo sapiens
HSQGTFTSDYSKYLDSRRAQDFVQWLMNT

SEQ ID NO: 96 - protein, 37 aa, Homo sapiens
HDEFERHAEGTFTSDVSSYLEGQAAKEFIAWLVKGRG

SEQ ID NO: 97 - protein, 30 aa, Homo sapiens
HAEGTFTSDVSSYLEGQAAKEFIAWLVKGR

SEQ ID NO: 98 - protein, 31 aa, Homo sapiens
HAEGTFTSDVSSYLEGQAAKEFIAWLVKGRG

SEQ ID NO: 99 - protein, 33 aa, Homo sapiens
HADGSFSDEMNTILDNLAARDFINWLIQTKITD

SEQ ID NO: 100 - protein, 27 aa, Homo sapiens
HADGVFTSDFSKLLGQLSAKKYLESLM

SEQ ID NO: 101 - protein, 42 aa, Homo sapiens
HADGVFTSDFSKLLGQLSAKKYLESLMGKRVSSNISEDPVPV

SEQ ID NO: 102 - protein, 37 aa, Homo sapiens
HSQGTFTSDYSKYLDSRRAQDFVQWLMNTKRNRNNIA

SEQ ID NO: 103 - protein, 48 aa, Homo sapiens
DVAHGILNEAYRKVLDQLSAGKHLQSLVARGVGGSLGGGAGDDAEPLS

SEQ ID NO: 104 - protein, 27 aa, Homo sapiens
HSDGIFTDSYSRYRKQMAVKKYLAAVL

SEQ ID NO: 105 - protein, 38 aa, Homo sapiens
HSDGIFTDSYSRYRKQMAVKKYLAAVLGKRYKQRVKNK

SEQ ID NO: 106 - protein, 27 aa, Homo sapiens
HSDGTFTSELSRLREGARLQRLLQGLV

SEQ ID NO: 107 - protein, 44 aa, Homo sapiens
YADAIFTNSYRKVLGQLSARKLLQDIMSRQQGESNQERGARARL

SEQ ID NO: 108 - protein, 28 aa, Homo sapiens
HSDAVFTDNYTRLRKQMAVKKYLNSILN

SEQ ID NO: 109 - protein, 56 aa, Homo sapiens
DAENLIDSFQEIVKEVGQLAETQRFECTTHQPRSPLRDLKGALESLIEEETGQKKI

SEQ ID NO: 110 - protein, 84 aa, Homo sapiens
ALSSAQDPQNALRPPGRALDTAAGSPVQTAHGLPSDALAPLDDSMPWEGRTTAQWSLHRKRHLARTLLTAAREPRPAPPSSNKV

SEQ ID NO: 111 - protein, 10 aa, Homo sapiens
QHWSYGLRPG

SEQ ID NO: 112 - protein, 10 aa, Homo sapiens
QHWSHGWYPG

SEQ ID NO: 113 - protein, 69 aa, Homo sapiens
QHWSYGLRPGGKRDAENLIDSFQEIVKEVGQLAETQRFECTTHQPRSPLRDLKGALESLIEEETGQKKI

SEQ ID NO: 114 - protein, 97 aa, Homo sapiens
QHWSHGWYPGGKRALSSAQDPQNALRPPGRALDTAAGSPVQTAHGLPSDALAPLDDSMPWEGRTTAQWSLHRKRHLARTLLTAAREPRPAPPSSNKV

SEQ ID NO: 115 - protein, 21 aa, Homo sapiens
GIVEQCCTSICSLYQLENYCN

SEQ ID NO: 116 - protein, 30 aa, Homo sapiens
FVNQHLCGSHLVEALYLVCGERGFFYTPKT

SEQ ID NO: 117 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 118 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 119 - protein, 66 aa, Homo sapiens
YRPSETLCGGELVDTLQFVCGDRGFYFSRPASRVSRRSRGIVEECCFRSCDLALLETYCATPAKSE

SEQ ID NO: 120 - protein, 34 aa, Homo sapiens
DVSTPPTVLPDNFPRYPVGKFFQYDTWKQSTQRL

SEQ ID NO: 121 - protein, 24 aa, Homo sapiens
QLYSALANKCCHVGCTKRSLARFC

SEQ ID NO: 122 - protein, 23 aa, Homo sapiens
PYVALFEKCCLIGCTKRSLAKYC

SEQ ID NO: 123 - protein, 29 aa, Homo sapiens
DSWMEEVIKLCGRELVRAQIAICGMSTWS

SEQ ID NO: 124 - protein, 31 aa, Homo sapiens
VAAKWKDDVIKLCGRELVRAQIAICGMSTWS

SEQ ID NO: 125 - protein, 24 aa, Homo sapiens
DVLAGLSSSCCKWGCSKSEISSLC

SEQ ID NO: 126 - protein, 27 aa, Homo sapiens
RAAPYGVRLCGREFIRAVIFTCGGSRW

SEQ ID NO: 127 - protein, 10 aa, Homo sapiens
YNWNSFGLRF

SEQ ID NO: 128 - protein, 13 aa, Homo sapiens
LPNYNWNSFGLRF

SEQ ID NO: 129 - protein, 14 aa, Homo sapiens
DLPNYNWNSFGLRF

SEQ ID NO: 130 - protein, 119 aa, Homo sapiens
EPLEKVASVGNSRPTGQQLESLGLLAPGEQSLPCTERKPAATARLSRRGTSLSPPPESSGSPQQPGLSAPHSRQIPAPQGAVLVQREKDLPNYNWNSFGLRFGKREAAPGNHGRSAGRG

SEQ ID NO: 131 - protein, 54 aa, Homo sapiens
GTSLSPPPESSGSPQQPGLSAPHSRQIPAPQGAVLVQREKDLPNYNWNSFGLRF

SEQ ID NO: 132 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 133 - protein, 19 aa, Homo sapiens
DFDMLRCMLGRVYRPCWQV

SEQ ID NO: 134 - protein, 13 aa, Homo sapiens
EIGDEENSAKFPI

SEQ ID NO: 135 - protein, 19 aa, Homo sapiens
GSVAFPAENGVQNTESTQE

SEQ ID NO: 136 - protein, 144 aa, Homo sapiens
ILLSASKSIRNLDDDMVFNTFRLGKGFQKEDTAEKSVIAPSLEQYKNDESSFMNEEENKVSKNTGSKHNFLNHGLPLNLAIKPYLALKGSVAFPAENGVQNTESTQEKREIGDEENSAKFPIGRRDFDMLRCMLGRVYRPCWQV

SEQ ID NO: 137 - protein, 27 aa, Homo sapiens
GSSFLSPEHQRVQQRKESKKPPAKLQP

SEQ ID NO: 138 - protein, 28 aa, Homo sapiens
GSSFLSPEHQRVQQRKESKKPPAKLQPR

SEQ ID NO: 139 - protein, 22 aa, Homo sapiens
FVPIFTYGELQRMQEKERNKGQ

SEQ ID NO: 140 - protein, 66 aa, Homo sapiens
SLSVWQRSGEEGPVDPAEPIREEENEMIKLTAPLEIGMRMNSRQLEKYPATLEGLLSEMLPQHAAK

SEQ ID NO: 141 - protein, 23 aa, Homo sapiens
FNAPFDVGIKLSGVQYQQHSQAL

SEQ ID NO: 142 - protein, 90 aa, Homo sapiens
FVPIFTYGELQRMQEKERNKGQKKSLSVWQRSGEEGPVDPAEPIREEENEMIKLTAPLEIGMRMNSRQLEKYPATLEGLLSEMLPQHAAK

SEQ ID NO: 143 - protein, 226 aa, Homo sapiens
ETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 144 - protein, 112 aa, Homo sapiens
AQMGLAPMEGIRRPDQALLPELPGLGLRAPLKKTTAEQAEEDLLQEAQALAEVLDLQDREPRSSRRCVRLHESCLGQQVPCCDPCATCYCRFFNAFCYCRKLGTAMNPCSRT

SEQ ID NO: 145 - protein, 73 aa, Homo sapiens
MIEVVCNDRLGKKVRVKCNTDDTIGDLKKLIAAQTGTRWNKIVLKKWYTIFKDHVSLGDYEIHDGMNLELYYQ

SEQ ID NO: 146 - protein, 491 aa, Homo sapiens [sequence omitted]

SEQ ID NO: 147 - protein, 28 aa, Homo sapiens
SLRRSSCFGGRMDRIGAQSGLGCNSFRY

SEQ ID NO: 148 - protein, 28 aa, Homo sapiens
SPKMVQGSGCFGRKMDRISSSSGLGCKV

SEQ ID NO: 149 - protein, 29 aa, Homo sapiens
SPKMVQGSGCFGRKMDRISSSSGLGCKVL

SEQ ID NO: 150 - protein, 30 aa, Homo sapiens
SPKMVQGSGCFGRKMDRISSSSGLGCKVLR

SEQ ID NO: 151 - protein, 30 aa, Homo sapiens
PKMVQGSGCFGRKMDRISSSSGLGCKVLRR

SEQ ID NO: 152 - protein, 27 aa, Homo sapiens
KMVQGSGCFGRKMDRISSSSGLGCKVL

SEQ ID NO: 153 - protein, 28 aa, Homo sapiens
KMVQGSGCFGRKMDRISSSSGLGCKVLR

SEQ ID NO: 154 - protein, 30 aa, Homo sapiens
KMVQGSGCFGRKMDRISSSSGLGCKVLRRH

SEQ ID NO: 155 - protein, 24 aa, Homo sapiens
MVQGSGCFGRKMDRISSSSGLGCK

SEQ ID NO: 156 - protein, 26 aa, Homo sapiens
MVQGSGCFGRKMDRISSSSGLGCKVL

SEQ ID NO: 157 - protein, 27 aa, Homo sapiens
MVQGSGCFGRKMDRISSSSGLGCKVLR

SEQ ID NO: 158 - protein, 28 aa, Homo sapiens
MVQGSGCFGRKMDRISSSSGLGCKVLRR

SEQ ID NO: 159 - protein, 29 aa, Homo sapiens
MVQGSGCFGRKMDRISSSSGLGCKVLRRH

SEQ ID NO: 160 - protein, 25 aa, Homo sapiens
VQGSGCFGRKMDRISSSSGLGCKVL

SEQ ID NO: 161 - protein, 27 aa, Homo sapiens
VQGSGCFGRKMDRISSSSGLGCKVLRR

SEQ ID NO: 162 - protein, 28 aa, Homo sapiens
VQGSGCFGRKMDRISSSSGLGCKVLRRH

SEQ ID NO: 163 - protein, 32 aa, Homo sapiens
SPKMVQGSGCFGRKMDRISSSSGLGCKVLRRH

SEQ ID NO: 164 - protein, 30 aa, Homo sapiens
NPMYNAVSNADLMDFKNLLDHLEEKMPLED

SEQ ID NO: 165 - protein, 22 aa, Homo sapiens
GLSKGCFGLKLDRIGSMSGLGC

SEQ ID NO: 166 - protein, 29 aa, Homo sapiens
YKGANKKGLSKGCFGLKLDRIGSMSGLGC

SEQ ID NO: 167 - protein, 53 aa, Homo sapiens
DLRVDTKSRAAWARLLQEHPNARKYKGANKKGLSKGCFGLKLDRIGSMSGLGC

SEQ ID NO: 168 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 169 - protein, 250 aa, Homo sapiens
ANLTNGGKSELLKSGSSKSTLKHIWTESSKDLSISRLLSQTFRGKENDTDLDLRYDTPEPYSEQDLWDWLRNSTDLQEPRPRAKRRPIVKTGKFKKMFGWGDFHSNIKTVKLNLLITGKIVDHGNGTFSVYFRHNSTGQGNVSVSLVPPTKIVEFDLAQQTVIDAKDSKSFNCRIEYEKVDKATKNTLCNYDPSKTCYQEQTQSHVSWLCSKPFKVICIYISFYSTDYKLVQKVCPDYNYHSDTPYFPSG

SEQ ID NO: 170 - protein, 242 aa, Homo sapiens
KEVVHATEGLDWEDKDAPGTLVGNVVHSRIISPLRLFVKQSPVPKPGPMAYADSMENFWDWLANITEIQEPLARTKRRPIVKTGKFKKMFGWGDFHSNIKTVKLNLLITGKIVDHGNGTFSVYFRHNSTGLGNVSVSLVPPSKVVEFEVSPQSTLETKESKSFNCRIEYEKTDRAKKTALCNFDPSKICYQEQTQSHVSWLCSKPFKVICIYIAFYSVDYKLVQKVCPDYNYHSETPYLSSG

SEQ ID NO: 171 - protein, 230 aa, Homo sapiens
QDDGPPGSEDPERDDHEGQPRPRVPRKRGHISPKSRPMANSTLLGLLAPPGEAWGILGQPPNRPNHSPPPSAKVKKIFGWGDFYSNIKTVALNLLVTGKIVDHGNGTFSVHFQHNATGQGNISISLVPPSKAVEFHQEQQIFIEAKASKIFNCRMEWEKVERGRRTSLCTHDPAKICSRDHAQSSATWSCSQPFKVVCVYIAFYSTDYRLVQKVCPDYNYHSDTPYYPSG

SEQ ID NO: 172 - protein, 285 aa, Homo sapiens
QIPESGRPQYLGLRPAAAGAGAPGQQLPEPRSSDGLGVGRAWSWAWPTNHTGALARAGAAGALPAQRTKRKPSIKAARAKKIFGWGDFYFRVHTLKFSLLVTGKIVDHVNGTFSVYFRHNSSSLGNLSVSIVPPSKRVEFGGVWLPGPVPHPLQSTLALEGVLPGLGPPLGMAAAAAGPGLGGSLGGALAGPLGGALGVPGAKESRAFNCHVEYEKTNRARKHRPCLYDPSQVCFTEHTQSQAAWLCAKPFKVICIFVSFLSFDYKLVQKVCPDYNFQSEHPYFG

SEQ ID NO: 173 - protein, 33 aa, Homo sapiens
ILQRGSGTAAVDFTKKDHTATWGRPFFLFRPRN

SEQ ID NO: 174 - protein, 25 aa, Homo sapiens
FRVDEEFQSPFASQSRGYFLFRPRN

SEQ ID NO: 175 - protein, 24 aa, Homo sapiens
WYKPAAGHSSYSVGRAAGLLSGLR

SEQ ID NO: 176 - protein, 29 aa, Homo sapiens
WYKPAAGHSSYSVGRAAGLLSGLRRSPYA

SEQ ID NO: 177 - protein, 20 aa, Homo sapiens
SFRNGVGTGMKKTSFQRAKS

SEQ ID NO: 178 - protein, 23 aa, Homo sapiens
WYKHVASPRYHTVGRAAGLLMGL

SEQ ID NO: 179 - protein, 30 aa, Homo sapiens
WYKHVASPRYHTVGRAAGLLMGLRRSPYLW

SEQ ID NO: 180 - protein, 125 aa, Homo sapiens
SDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYIL

SEQ ID NO: 181 - protein, 5 aa, Homo sapiens
IPYIL

SEQ ID NO: 182 - protein, 13 aa, Homo sapiens
QLYENKPRRPYIL

SEQ ID NO: 183 - protein, 5 aa, Homo sapiens
DSYYY

SEQ ID NO: 184 - protein, 30 aa, Homo sapiens
SSPETLISDLLMRESTENVPRTRLEDPAMW

SEQ ID NO: 185 - protein, 36 aa, Homo sapiens
YPSKPDNPGEDAPAEDMARYYSALRHYINLITRQRY

SEQ ID NO: 186 - protein, 36 aa, Homo sapiens
APLEPVYPGDNATPEQMAQYAADLRRYINMLTRPRY

SEQ ID NO: 187 - protein, 20 aa, Homo sapiens
HKEDTLAFSEWGSPHAAVPR

SEQ ID NO: 188 - protein, 36 aa, Homo sapiens
YPIKPEAPREDASPEELNRYYASLRHYLNLVTRQRY

SEQ ID NO: 189 - protein, 34 aa, Homo sapiens
IKPEAPREDASPEELNRYYASLRHYLNLVTRQRY

SEQ ID NO: 190 - protein, 82 aa, Homo sapiens
VPIDIDKTKVQNIHPVESAKIEPPDTGLYYDEYLKQVIDVLETDKHFREKLQKADIEEIKSGRLSKELDLVSHHVRTKLDEL

SEQ ID NO: 191 - protein, 435 aa, Homo sapiens
VPLERGAPNKEETPATESPDTGLYYHRYLQEVIDVLETDGHFREKLQAANAEDIKSGKLSRELDFVSHHVRTKLDELKRQEVSRLRMLLKAKMDAEQDPNVQVDHLNLLKQFEHLDPQNQHTFEARDLELLIQTATRDLAQYDAAHHEEFKRYEMLKEHERRRYLESLGEEQRKEAERKLEEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGDTGEGWETVEMHPAYTEEELRRFEEELAAREAELNAKAQRLSQETEALGRSQGRLEAQKRELQQAVLHMEQRKQQQQQQQGHKAPAAHPEGQLKFHPDTDDVPVPAPAGDQKEVDTSEKKLLERLPEVEVPQHL

SEQ ID NO: 192 - protein, 396 aa, Homo sapiens
VPIDIDKTKVQNIHPVESAKIEPPDTGLYYDEYLKQVIDVLETDKHFREKLQKADIEEIKSGRLSKELDLVSHHVRTKLDELKRQEVGRLRMLIKAKLDSLQDIGMDHQALLKQFDHLNHLNPDKFESTDLDMLIKAATSDLEHYDKTRHEEFKKYEMMKEHERREYLKTLNEEKRKEEESKFEEMKKKHENHPKVNHPGSKDQLKEVWEETDGLDPNDFDPKTFFKLHDVNSDGFLDEQELEALFTKELEKVYDPKNEEDDMVEMEEERLRMREHVMSEVDTNKDRLVTLEEFLKATEKKEFLEPDSWETLDQQQFFTEEELKEYENIIALQENELKKKADELQKQKEELQRQHDQLEAQKLEYHQVIQQMEQKKLQQGIPPSGPAGELKFEPHI

SEQ ID NO: 193 - protein, 10 aa, Homo sapiens
YGGFLRKYPK

SEQ ID NO: 194 - protein, 9 aa, Homo sapiens
YGGFLRKYP

SEQ ID NO: 195 - protein, 32 aa, Homo sapiens
YGGFLRRIRPKLKWDNQKRYGGFLRRQFKVVT

SEQ ID NO: 196 - protein, 13 aa, Homo sapiens
YGGFLRRIRPKLK

SEQ ID NO: 197 - protein, 17 aa, Homo sapiens
YGGFLRRIRPKLKWDNQ

SEQ ID NO: 198 - protein, 8 aa, Homo sapiens
YGGFLRRI

SEQ ID NO: 199 - protein, 5 aa, Homo sapiens
YGGFL

SEQ ID NO: 200 - protein, 29 aa, Homo sapiens
YGGFLRRQFKVVTRSQEDPNAYSGELFDA

SEQ ID NO: 201 - protein, 5 aa, Homo sapiens
YGGFM

SEQ ID NO: 202 - protein, 8 aa, Homo sapiens
YGGFMRGL

SEQ ID NO: 203 - protein, 7 aa, Homo sapiens
YGGFMRF

SEQ ID NO: 204 - protein, 30 aa, Homo sapiens
MPRVRSLFQEQEEPEPGMEEAGEMEQKQLQ

SEQ ID NO: 205 - protein, 17 aa, Homo sapiens
FSEFMRQYLVLSMQSSQ

SEQ ID NO: 206 - protein, 17 aa, Homo sapiens
FGGFTGARKSARKLANQ

SEQ ID NO: 207 - protein, 41 aa, Homo sapiens
DAEEDDSLANSSDLLKELLETGDNRERSHHQDGSDNEEEVS

SEQ ID NO: 208 - protein, 22 aa, Homo sapiens
FAEALPSDEEGESYSKEVPEME

SEQ ID NO: 209 - protein, 13 aa, Homo sapiens
YGGFLRRQFKVVT

SEQ ID NO: 210 - protein, 20 aa, Homo sapiens
MDELYPMEPEEEANGSEILA

SEQ ID NO: 211 - protein, 73 aa, Homo sapiens
ECSQDCATCSYRLVRPADINFLACVMECEGKLPSLKIWETCKELLQLSKPELPQDGTSTLRENSKPEESHLLA

SEQ ID NO: 212 - protein, 33 aa, Homo sapiens
QPLPDCCRQKTCSCRLYELLHGAGNHAAGILTL

SEQ ID NO: 213 - protein, 28 aa, Homo sapiens
RSGPPGLQGRLQRLLQASGNHAAGILTM

SEQ ID NO: 214 - protein, 33 aa, Homo sapiens
TRSAWLDSGVTGSGLEGDHLSDTSTTSLELDSR

SEQ ID NO: 215 - protein, 141 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 216 - protein, 36 aa, Homo sapiens
AVSEHQLLHDKGKSIQDLRRRFFLHHLIAEIHTAEI

SEQ ID NO: 217 - protein, 57 aa, Homo sapiens
ATSEVSPNSKPSPNTKNHPVRFGSDDEGRYLTQETNKVETYKEQPLKTPGKKKKGKP

SEQ ID NO: 218 - protein, 39 aa, Homo sapiens
SLALADDAAFRERARLLAALERRHWLNSYMHKLLVLDAP

SEQ ID NO: 219 - protein, 31 aa, Homo sapiens
YGGFMTSEKSQTPLVTLFKNAIIKNAYKKGE

SEQ ID NO: 220 - protein, 39 aa, Homo sapiens
SYSMEHFRWGKPVGKKRRPVKVYPNGAEDESAEAFPLEF

SEQ ID NO: 221 - protein, 21 aa, Homo sapiens
PVKVYPNGAEDESAEAFPLEF

SEQ ID NO: 222 - protein, 89 aa, Homo sapiens
ELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 223 - protein, 56 aa, Homo sapiens
ELTGQRLREGDGPDGPADDGAGAQADLEHSLLVAAEKKDEGPYRMEHFRWGSPPKD

SEQ ID NO: 224 - protein, 13 aa, Homo sapiens
SYSMEHFRWGKPV

SEQ ID NO: 225 - protein, 18 aa, Homo sapiens
DEGPYRMEHFRWGSPPKD

SEQ ID NO: 226 - protein, 11 aa, Homo sapiens
YVMGHFRWDRF

SEQ ID NO: 227 - protein, 76 aa, Homo sapiens
WCLESSQCQDLTTESNLLECIRACKPDLSAETPMFPGNGDEQPLTENPRKYVMGHFRWDRFGRRNSSSSGSSGAGQ

SEQ ID NO: 228 - protein, 30 aa, Homo sapiens
EDVSAGEDCGPLPEGGPEPRSDGAKPGPRE

SEQ ID NO: 229 - protein, 16 aa, Homo sapiens
LETPAPQVPARRLLPP

SEQ ID NO: 230 - protein, 40 aa, Homo sapiens
AADHDVGSELPPEGVLGALLRVKRLETPAPQVPARRLLPP

SEQ ID NO: 231 - protein, 26 aa, Homo sapiens
ARPVKEPRGLSAASPPLAETGAPRRF

SEQ ID NO: 232 - protein, 7 aa, Homo sapiens
ARPVKEP

SEQ ID NO: 233 - protein, 10 aa, Homo sapiens
LETPAPQVPA

SEQ ID NO: 234 - protein, 18 aa, Homo sapiens
GLSAASPPLAETGAPRRF

SEQ ID NO: 235 - protein, 22 aa, Homo sapiens
AADHDVGSELPPEGVLGALLRV

SEQ ID NO: 236 - protein, 227 aa, Homo sapiens
ARPVKEPRGLSAASPPLAETGAPRRFRRSVPRGEAAGAVQELARALAHLLEAERQERARAEAQEAEDQQARVLAQLLRVWGAPRNSDPALGLDDDPDAPAAQLARALLRARLDPAALAAQLVPAPVPAAALRPRPPVYDDGPAGPDAEEAGDETPDVDPELLRYLLGRILAGSADSEGVAAPRRLRRAADHDVGSELPPEGVLGALLRVKRLETPAPQVPARRLLPP

SEQ ID NO: 237 - protein, 90 aa, Homo sapiens
KTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 238 - protein, 88 aa, Homo sapiens
QCSLDSVMDKKIKDVLNSLEYSPSPISKKLSCASVKSQGRPSSCPAGMAVTGCACGYGCGSWDVQLETTCHCQCSVVDWTTARCCHLT

SEQ ID NO: 239 - protein, 43 aa, Homo sapiens
QDEGSEATGFLPAAGEKTSGPLGNLAEELNGYSRKKGGFSFRF

SEQ ID NO: 240 - protein, 41 aa, Homo sapiens
SEEPPISLDLTFHLLREVLEMARAEQLAQQAHSNRKLMEII

SEQ ID NO: 241 - protein, 40 aa, Homo sapiens
DNPSLSIDLTFHLLRTLLELARTQSQRERAEQNRIIFDSV

SEQ ID NO: 242 - protein, 41 aa, Homo sapiens
IVLSLDVPIGLLQILLEQARARAAREQATTNARILARVGHC

SEQ ID NO: 243 - protein, 38 aa, Homo sapiens
FTLSLDVPTNIMNLLFNIAKAKNLRAQAAANAHLMAQI

SEQ ID NO: 244 - protein, 4 aa, Homo sapiens
DRVY

SEQ ID NO: 245 - protein, 5 aa, Homo sapiens
DRVYI

SEQ ID NO: 246 - protein, 7 aa, Homo sapiens
DRVYIHP

SEQ ID NO: 247 - protein, 9 aa, Homo sapiens
DRVYIHPFH

SEQ ID NO: 248 - protein, 10 aa, Homo sapiens
DRVYIHPFHL

SEQ ID NO: 249 - protein, 8 aa, Homo sapiens
DRVYIHPF

SEQ ID NO: 250 - protein, 7 aa, Homo sapiens
RVYIHPF

SEQ ID NO: 251 - protein, 6 aa, Homo sapiens
VYIHPF

SEQ ID NO: 252 - protein, 452 aa, Homo sapiens
DRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 253 - protein, 383 aa, Homo sapiens
MDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDSELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPV

SEQ ID NO: 254 - protein, 403 aa, Homo sapiens
QPLLAHGDKSLQGPQPPRHQLSEPAPAYHRITPTITNFALRLYKELAADAPGNIFFSPVSISTTLALLSLGAQANTSALILEGLGFNLTETPEADIHQGFRSLLHTLALPSPKLELKVGNSLFLDKRLKPRQHYLDSIKELYGAFAFSANFTDSVTTGRQINDYLRRQTYGQVVDCLPEFSQDTFMVLANYIFFKAKWKHPFSRYQTQKQESFFVDERTSLQVPMMHQKEMHRFLYDQDLACTVLQIEYRGNALALLVLPDPGKMKQVEAALQPQTLRKWGQLLLPSLLDLHLPRFSISGTYNLEDILPQIGLTNILNLEADFSGVTGQLNKTISKVSHKAMVDMSEKGTEAGAASGLLSQPPSLNTMSDPHAHFNRPFLLLLWEVTTQSLLFLGKVVNPVAG

SEQ ID NO: 255 - protein, 394 aa, Homo sapiens
LKPSFSPRNYKALSEVQGWKQRMAAKELARQNMDLGFKLLKKLAFYNPGRNIFLSPLSISTAFSMLCLGAQDSTLDEIKQGFNFRKMPEKDLHEGFHYIIHELTQKTQDLKLSIGNTLFIDQRLQPQRKFLEDAKNFYSAETILTNFQNLEMAQKQINDFISQKTHGKINNLIENIDPGTVMLLANYIFFRARWKHEFDPNVTKEEDFFLEKNSSVKVPMMFRSGIYQVGYDDKLSCTILEIPYQKNITAIFILPDEGKLKHLEKGLQVDTFSRWKTLLSRRVVDVSVPRLHMTGTFDLKKTLSYIGVSKIFEEHGDLTKIAPHRSLKVGEAVHKAELKMDERGTEGAAGTGAQTLPMETPLVVKIDKPYLLLIYSEKIPSVLFLGKIVNPIGK

SEQ ID NO: 256 - protein, 387 aa, Homo sapiens
SRCSAQKNTEFAVDLYQEVSLSHKDNIIFSPLGITLVLEMVQLGAKGKAQQQIRQTLKQQETSAGEEFFVLKSFFSAISEKKQEFTFNLANALYLQEGFTVKEQYLHGNKEFFQSAIKLVDFQDAKACAEMISTWVERKTDGKIKDMFSGEEFGPLTRLVLVNAIYFKGDWKQKFRKEDTQLINFTKKNGSTVKIPMMKALLRTKYGYFSESSLNYQVLELSYKGDEFSLIIILPAEGMDIEEVEKLITAQQILKWLSEMQEEEVEISLPRFKVEQKVDFKDVLYSLNITEIFSGGCDLSGITDSSEVYVSQVTQKVFFEINEDGSEAATSTGIHIPVIMSLAQSQFIANHPFLFIMKHNPTESILFMGRVTNPDTQEIKGRDLDSL

SEQ ID NO: 257 - protein, 17 aa, Homo sapiens
DRMPCRNFFWKTFSSCK

SEQ ID NO: 258 - protein, 29 aa, Homo sapiens
QEGAPPQQSARRDRMPCRNFFWKTFSSCK

SEQ ID NO: 259 - protein, 14 aa, Homo sapiens
AGCKNFFWKTFTSC

SEQ ID NO: 260 - protein, 28 aa, Homo sapiens
SANSNPAMAPRERKAGCKNFFWKTFTSC

SEQ ID NO: 261 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 262 - protein, 16 aa, Homo sapiens
ALNSVAYERSAMQNYE

SEQ ID NO: 263 - protein, 10 aa, Homo sapiens
HKTDSFVGLM

SEQ ID NO: 264 - protein, 10 aa, Homo sapiens
DMHDFFVGLM

SEQ ID NO: 265 - protein, 36 aa, Homo sapiens
DADSSIEKQVALLKALYGHGQISHKRHKTDSFVGLM

SEQ ID NO: 266 - protein, 11 aa, Homo sapiens
RPKPQQFFGLM

SEQ ID NO: 267 - protein, 218 aa, Homo sapiens
QPEAAQQEAVTAAEHPGLDDFLRQVERLLFLRENIQRLQGDQGEHSASQIFQSDWLSKRQHPGKREEEEEEGVEEEEEEEGGAVGPHKRQHPGRREDEASWSVDVTQHKRQHPGRRSPWLAYAVPKRQHPGRRLADPKAQRSWEEEEEEEEREEDLMPEKRQHPGKRALGGPCGPQGAYGQAGLLLGLLDDLSRSQGAEEKRQHPGRRAAWVREPLEE

SEQ ID NO: 268 - protein, 3 aa, Homo sapiens
QHP

SEQ ID NO: 269 - protein, 11 aa, Homo sapiens
ETPDCFWKYCV

SEQ ID NO: 270 - protein, 8 aa, Homo sapiens
ACFWKYCV

SEQ ID NO: 271 - protein, 9 aa, Homo sapiens
CYFQNCPRG

SEQ ID NO: 272 - protein, 39 aa, Homo sapiens
ASDRSNATQLDGPAGALLLRLVQLAGAPEPFEPAQPDAY

SEQ ID NO: 273 - protein, 94 aa, Homo sapiens
AAPDLDVRKCLPCGPGGKGRCFGPNICCAEELGCFVGTAEALRCQEENYLPSPCQSGQKACGSGGRCAVLGLCCSPDGCHADPACDAEATFSQR

SEQ ID NO: 274 - protein, 93 aa, Homo sapiens
AMSDLELRQCLPCGPGGKGRCFGPSICCADELGCFVGTAEALRCQEENYLPSPCQSGQKACGSGGRCAAFGVCCNDESCVTEPECREGFHRRA

SEQ ID NO: 275 - protein, 9 aa, Homo sapiens
CYIQNCPLG

SEQ ID NO: 276 - protein, 24 aa, Homo sapiens
TLQPPSALRRRHYHHALPPSRHYP

SEQ ID NO: 277 - protein, 26 aa, Homo sapiens
RPESALLGGSEAGERLLQQGLAQVEA

SEQ ID NO: 278 - protein, 38 aa, Homo sapiens
QAEATRQAAAQEERLADLASDLLLQYLLQGGARQRGLG

SEQ ID NO: 279 - protein, 593 aa, Homo sapiens
APPGRPEAQPPPLSSEHKEPVAGDAVPGPKDGSAPEVRGARNSEPQDEGELFQGVDPRALAAVLLQALDRPASPPAPSGSQQGPEEEAAEALLTETVRSQTHSLPAPESPEPAAPPRPQTPENGPEASDPSEELEALASLLQELRDFSPSSAKRQQETAAAETETRTHTLTRVNLESPGPERVWRASWGEFQARVPERAPLPPPAPSQFQARMPDSGPLPETHKFGEGVSSPKTHLGEALAPLSKAYQGVAAPFPKARRPESALLGGSEAGERLLQQGLAQVEAGRRQAEATRQAAAQEERLADLASDLLLQYLLQGGARQRGLGGRGLQEAAEERESAREEEEAEQERRGGEERVGEEDEEAAEAEAEAEEAERARQNALLFAEEEDGEAGAEDKRSQEETPGHRRKEAEGTEEGGEEEDDEEMDPQTIDSLIELSTKLHLPADDVVSIIEEVEEKRKRKKNAPPEPVPPPRAAPAPTHVRSPQPPPPAPAPARDELPDWNEVLPPWDREEDEVYPPGPYHPFPNYIRPRTLQPPSALRRRHYHHALPPSRHYPGREAQARRAQEEAEAEERRLQEQEELENYIEHVLLRRP

(19) United States
(12) Patent Application Publication
(10) Pub. No.: US 2021/0171598 A1
(43) Pub. Date: Jun. 10, 2021

(54) PEPTIDE HORMONE WITH ONE OR MORE O-GLYCANS

(71) Applicant: UNIVERSITY OF COPENHAGEN, COPENHAGEN K. (DK)

(72) Inventors: Katrine TER-BORCH GRAM SCHJOLDAGER, Vedbaek (DK); Henrik CLAUSEN, Copenhagen Ø (DK); Sergey Y. VAKRUSHEV, Nyborg (DK); Christoffer Knak GOTH, Frederiksberg C (DK); Thomas Daugbjerg MADSEN, Copenhagen N. (DK); Lasse H. HANSEN, Glostrup (DK); Jens P. GØTZE, Virum (DK)

(73) Assignee: UNIVERSITY OF COPENHAGEN, COPENHAGEN K. (DK)

(21) Appl. No.: 16/768,584

(22) PCT Filed: Nov. 30, 2018
(86) PCT No.: PCT/EP2018/083235

§ 371 Date: May 29, 2020

(30) Foreign Application Priority Data
Dec. 1, 2017 (EP) 17204897.7

(51) Int. Cl. C07K 14/575; C07K 19/00; C07K 1/00

(52) U.S. Cl. CPC C07K 14/575; A61K 38/00; C07K 1/006; C07K 19/00

(57) ABSTRACT

The present invention relates to a peptide hormone with one or more O-glycans attached at specific amino acid residues as well as to formulations comprising the same.

### FIELD OF THE INVENTION

The present invention relates to a peptide hormone with one or more O-glycans attached at specific amino acid residues. The present invention also relates to formulations, in particular pharmaceutical formulations comprising these peptide hormones.

### BACKGROUND OF THE INVENTION

Peptide hormones, including neuropeptides and other biologically active peptides (here designated peptide hormones) are synthesized as precursor proteins that travel through the secretory pathway where they undergo limited proteolytic processing for activation (by e.g. proprotein convertases (PCs), carboxypeptidases, Corin)¹, C-terminal α-amidation² and a number of other PTMs like tyrosine sulfation³, N-terminal acetylation⁴ and serine phosphorylation⁵, during their biosynthesis and packaging into secretory vesicles, ready for secretion. Under appropriate physiological conditions, the mature peptide hormones are released from the cell, where they exert a multitude of functions regulating complex physiological processes. For this and other reasons, analogues of peptide hormones are emerging as major drug targets in neurological and metabolic disorders where they are tested as agonists or antagonists for their cognate receptors.

Once secreted most peptide hormones are prone to specific proteolytic degradation and have short half-lifes⁶⁻⁸, making them very difficult to isolate and characterize with respect to naturally occurring variants and PTMs. Recently, however, mass spectrometry based studies have identified peptide hormone PTMs like C-terminal amidation, N-terminal acetylation and serine phosphorylation.

Mucin-type (GalNAc-type) O-glycosylation (hereafter simply O-glycosylation) is an abundant PTM found on many proteins trafficking the secretory pathway, but the presence of O-glycans on peptide hormones have only been found in a few high-throughout mass spectrometry driven studies listed in large supplementary files⁹⁻¹². Just recently, glycosylation was described on insulin and calcitonin in more directed studies analysing pancreatic beta-cells and small cell lung cancer cell line, respectively^{13,14}. O-glycosylation of proteins is a non-template driven PTM initiated in the Golgi where up to 20 polypeptide GalNAc-transferase (GalNAc-T) isoenzymes initiate the transfer of α-GalNAc to the hydroxyl group of Ser and Thr (and possibly Tyr) residues¹⁵. The large number of 20 GalNAc-T isoenzymes have different albeit partly overlapping substrate specificities and the enzymes are differentially expressed in cells and tissues, which leaves this type of protein glycosylation the only one with high degree of differential and potentially dynamic regulation in eukaryotic cells compared to other PTMs.

Our understanding of the biosynthesis and genetic regulation of O-glycosylation is incomplete, partly because there are seemingly no simple primary peptide sequence motifs that guide us to the functions of GalNAc-T isoenzymes and their contributions to O-glycosylation, and partly because of overlap in functions as well as interdependent sequential functions among the many isoenzymes. The first congenital deficiencies in GALNT genes demonstrate that despite this, individual GalNAc-Ts serve highly specific regulatory roles of important body functions including phosphate homeostasis and lipoprotein metabolism. These fundamental functions are directed by non-redundant site-specific O-glycosylation^{11,16-18}.

Here, the present inventors used a novel strategy for exploring potential O-glycosylation of peptide hormones in mammalian neuronal and endocrine tissues as well as cerebrospinal fluid and plasma using sensitive mass spectrometry, and surprisingly identified wide occurrence of O-glycans on peptide hormones. The present inventors identified these O-glycans in the receptor ligand binding domains of mammalian native mature peptide hormones, and demonstrate that these O-glycans serve to modulate receptor signalling and peptide hormone stability.
U.S. Pat. No. 8,183,340 B2 relates to GLP-1 pegylated compounds
EP 1105409 B1 Protection of endogenous therapeutic peptides from peptidase activity through conjugation to blood components
WO 2006082517 A1 relates to Pyy agonists and uses thereof
WO 2015071355 A1 relates to selective pyy compounds and uses thereof
WO 2015177572 A1 relates to Peptide yy (pyy) analogues
WO 2006077035 A1 relates to Peptides with neuropeptide-2 receptor (y2r) agonist activity

### OBJECT OF THE INVENTION

An object of the present invention relates to a peptide hormone comprising one or more O-linked glycans at specific sites. The modified, that means the O-linked glycan bearing peptide hormone has different and/or improved stability and/or pharmacokinetic properties.

It is an object of embodiments of the invention to provide methods for preparing peptide hormones, wherein one or more O-linked glycan are placed at predetermined sites. Yet another object of embodiments of the invention is to provide formulations, in particular pharmaceutical formulations, which comprise said peptide hormones.

### SUMMARY OF THE INVENTION

Peptide hormones including neuropeptides and certain prohormones (here peptide hormones) encompass a large class of biologically active small peptides that have crucial biological functions. Peptide hormones are produced in a long preproform and undergo limited proteolytic cleavage to produce the final active peptides. Peptide hormones function as signaling molecules by binding to specific receptors and mediate intracellular signaling and stimuli. Peptide hormones can be classified into approximately 46 families where members undergo differential processing and give rise to approximately 279 known active peptide hormones. This invention relates to the identification of O-glycans in specific positions on proforms and mature active peptide hormones, and more specifically the presence of O-glycans in receptor-binding regions of peptide hormones that is demonstrated to modify the activity of such peptide hormones. The invention discloses multiple examples of such peptide hormones with O-glycans attached that have increased stability and lower bioactivity in receptor signaling and thus represent improved peptide hormone designs with altered drug effects.

This invention relates primarily to the Neuropeptide Y family (NPY, PPY and PYY), the Glucagon/Secretin family (GIP, Glucagon, GLP-1, GLP-2, PACAP, Secretin, Somatoliberin, PHM-27/PHV-42 and VIP), and the Natriuretic peptide family (ANP, BNP and CNP). Members of the neuropeptide Y family are well-known regulators of appetite and energy balance, and members of the Secretin family regulate glucose homeostasis (Glucagon, GLP-1, GLP-2, GIP), smooth muscle cell relaxation (VIP, PACAP), secretion of pancreatic juice (Secretin) and growth hormone release (Somatoliberin). Natriuretic peptides regulate the blood pressure through cardiorenal homeostasis.

It has been found by the present inventor(s) that 92 peptide hormones are O-glycosylated and it has been demonstrated for selected examples that the O-glycosylated proteoforms comprise a minor fraction of the total pool of the given peptide hormone in vivo.

As illustrative examples of this invention, the present inventors demonstrate that peptide hormones ANP, VIP, Secretin, GLP-1, Glucagon, NPY, PPY, PYY and Galanin with O-glycans in specific amino acid positions in the receptor-binding region require more than 28 fold higher concentrations in appropriate receptor stimulation assays to induce signaling compared to peptides without O-glycans. Furthermore, the present inventors demonstrate that the stability of peptide hormones ANP, VIP, Secretin, GLP-1, Glucagon, NPY, PYY and Galanin with O-glycans in specific amino acid positions is greater than the peptides without O-glycans in vitro using IDE/NEP/DPP-IV proteases as well as ex vivo using plasma and in vivo using rodent animal models.

Throughout the description, the term “peptide hormone species” is frequently used to refer to specific types of peptide hormones. For example, a peptide hormone with a given amino acid sequence may comprise more than one amino acid residue that serves as a site for O-linked glycan attachment. A peptide hormone with two such amino acid residues can have three different O-linked glycan patterns, because either one of the two or both amino acid residue may carry an O-linked glycan. As used herein, each pattern corresponds to one peptide hormone species.

It is generally challenging to use peptide hormones as therapeutic drugs due to their extremely short circulatory half-life and in some cases extreme potency.

So, in a first aspect the present invention relates to peptide hormones (or “peptide hormone species”) with different O-glycans attached at specific amino acids and the use of these to increase stability and circulatory half-life of drugs as well as to modulate the potency and receptor selectivity of peptide hormone drugs. These peptide hormones may have wide applications for the treatment of many common human diseases including hypertension, heart disease, metabolic syndromes and psychological disorders. In embodiments of the first aspect, the present invention relates to formulations, particularly pharmaceutical formulations, which comprise a peptide hormone, i.e. at least one molecule of a “peptide hormone species”, exhibiting a specific, determined glycosylation pattern of one or more O-linked glycan at a predetermined specific site of said peptide hormone, wherein specific, determined glycosylation pattern means that each molecule of said peptide hormone in said formulation, particularly in said pharmaceutical formulation, displays structural homogeneity with respect to the site of the glycan attachment and/or with respect to the glycan attachment. In further embodiments of the first aspect, the present invention relates also to mixtures of peptide hormones (“peptide hormone species”) as described above that are present in the formulations, particularly the pharmaceutical formulations according to the invention. A formulation mixture, particularly the pharmaceutical formulation mixture, comprises at least two or more, e.g., three, four, five, six, seven, eight, nine, ten, or even more peptide hormones (“peptide hormone species”) exhibiting each a specific, determined glycosylation pattern of one or more O-linked glycan at a predetermined specific site of said peptide hormone, wherein specific, determined glycosylation pattern means that each molecule of said peptide hormones in said formulation, particularly in said pharmaceutical formulation, displays structural homogeneity with respect to the site of the glycan attachment and/or with respect to the glycan attachment.

In a second aspect the present invention relates to an isolated peptide hormone, such as recombinant, such as a peptide hormone comprising one or more O-linked glycan at a predetermined specific site, such as in the receptor-binding region. In embodiments of the second aspect, the invention relates to the above mentioned formulations, particularly pharmaceutical formulations, and to mixtures of peptide hormones (“peptide hormone species”) as described above that are present in the formulations, particularly the pharmaceutical formulations according to the invention.

In a third aspect the present invention relates to a host cell comprising one or more glycosyltransferase genes that have been inactivated such that
a) Homogenous Tn (GalNAc) glycosylation is obtained (COSMC/C1GALT1);
b) Homogenous T (Gal/GalNAc) glycosylation is obtained (ST6GALNAC1-6/ST3GAL1/GCNT3/GCNT4/B3GNT6);
c) Homogenous ST or STn glycosylation is obtained (GCNT3/GCNT4/B3GNT6).

It is to be understood that specific, determined and/or homogenous Tn (GalNAc) glycosylation may be obtained by inactivation and/or downregulation of one or more genes selected from COSMC and C1GALT1; that homogenous T (Gal/GalNAc) glycosylation may be obtained by inactivation and/or downregulation of one or more genes selected from GCNT3, GCNT4, B3GNT6, and that homogenous ST or STn glycosylation may be obtained by inactivation and/or downregulation of one or more genes selected from ST6GALNAC1-6, ST3GAL1, GCNT3, GCNT4, B3GNT6.

In some embodiments, the host cell further comprising a gene encoding an exogenous peptide hormone, such as a peptide hormone according to the invention.

This combinatorial deconstruction of O-glycosylation pathways in cell lines is obtained using precise genetic engineering with Zinc Finger Nucleases or CRISPR/Cas9 to target specific glycosyl transferases in the mammalian O-glycan biosynthetic pathway (FIG. 1).

In a further aspect the present invention relates to a method for producing an isolated peptide hormone comprising one or more O-linked glycan(s) at a predetermined specific site(s), such as in the receptor-binding region, the method comprising; a) inactivation and/or downregulation of one or more glycosyltransferases, and/or endogenous activation or knock in of one or more glycosyltransferases, or any combination hereof in a host cell, and b) expression of said peptide hormone in said host cell. In some embodiments, one or more genes selected from COSMC, C1GALT1, GCNT3, GCNT4, B3GNT6, ST6GALNAC1-6, ST3GAL1 has been inactivated and/or downregulated.

In a further aspect the present invention relates to a method for the production of an isolated peptide hormone, such as a neuropeptide comprising one or more O-linked glycan at a predetermined specific site, such as in the receptor-binding region, said method comprising a) providing a non-O-glycosylated peptide hormone; and b) treating said non-O-glycosylated synthetic peptide hormone with one or more recombinant purified glycosyl transferase, such as a GalNAc-transferase, such as GalNAc-T1, T2, T3, T4, T5, T6, T7, T10, T11, T12, T13, T14, and/or T16, and/or a Galactosyl-transferases (C1GalT1) and/or a sialyl-transferases, such as ST6GalNAc1 and/or ST3Gall under conditions to add one or more specific O-linked glycan to said peptide hormone. In some embodiments, the non-O-glycosylated peptide hormone is provided as a chemically produced peptide hormone produced using solid phase peptide synthesis Fmoc SPPS. In some embodiments, the non-O-glycosylated peptide hormone is provided as a recombinantly produced peptide hormone, such as produced in a production cell line.

In a further aspect the present invention relates to a method for the production of an isolated peptide hormone, such as a neuropeptide comprising one or more O-linked glycan at a predetermined specific site, such as in the receptor-binding region, said method comprising the building of said peptide hormone using solid phase peptide synthesis Fmoc SPPS including the use of glycosylated amino acids building blocks at said predetermined specific site(s).

In a further aspect of the present invention, formulations comprising at least one of the herein disclosed peptide hormones (“peptide hormone species”) are provided. As known to a person skilled in the art, formulation is a mixture comprising an active principle (in the present case at least one of the herein disclosed peptide hormones/“peptide hormone species”) and excipients at specific, defined amounts. A formulation is different from a mere solution of an active principle in a solvent, i.e. without any further excipient. Further, a pharmaceutical formulation usually comprises the active principle, for example at least one of the herein described peptide hormones/“peptide hormone species” in admixture with pharmaceutical excipients, which are known to a person of skill in the art (reference can be made to the European Pharmacopoeia, which may be considered as a representation of the common knowledge of the person of skill in the art). Therefore, in specific aspects of embodiments relating to formulations of the herein disclosed peptide hormones, the formulations of pharmaceutical formulations. Also, encompassed by this disclosure are formulations, particularly pharmaceutical formulations, which comprise a mixture of at least two of the herein disclosed peptide hormones (“peptide hormone species”) in specific, defined (i.e. predetermined) quantities.

In a further aspect the present invention relates to a method for the production of formulations, particularly pharmaceutical compostions, comprising at least one of the herein disclosed peptide hormones (“peptide hormone species”), or mixtures thereof.

### LEGENDS TO THE FIGURES

FIG. 1 illustrates the biosynthetic pathways of Mucin-type O-glycosylation. The glycosylation process is initiated by the transfer of UDP-GalNAc to acceptor Ser/Thr/Tyr amino acid residues in the protein or peptide backbone by GalNAc-transferases (20 different isoforms). The O-glycan structure can be further elongated to up to 8 different core structures by a number of glycosyl-transferases. Only 4 core structures are illustrated here.

FIG. 2 illustrates the enrichment process and identification of glycosylated peptide hormones. A) Schematic workflow for analysis of O-glycosylated peptide hormones. The proteins of plasma, neuroendocrine cells (STC-1, N2a) and neuronal as well as endocrine tissues (Brain, Pancreas, Ileum, Heart, Prostate, Cerebellum) were extracted using up to three different extraction procedures pr. sample. Subsequently, The proteins were reduced, alkylated, digested with either trypsin, Glu-C or chymotrypsin followed by de-sialylation using neuraminidase. Glycopeptides were subjected to LWAC using either PNA, Jacalin or VVA lectins. Subsequently, fractionation using either isoelectric focusing or high pH-fractionation was performed before separation and sequencing of the glycopeptides on LC-MS/MS. The resulting O-glycoproteome was matched against the NeuroPeP database, and further realignment of the preproprotein glycopeptides was done against the homologous human proproteins annotated in the same database. B) Overlap of glycosylated peptide hormones or C) Proproteins identified and matched to the human preproproteins with previously published O-glycoproteins or O-glycopeptide hormones. D) The number of identified O-glycosylated peptide hormones in each sample analysed.

FIG. 3 illustrates the prevalence of O-glycosylation in peptide hormone families. Peptide hormones from NeuroPep database (279 peptide hormones) distributed across their respective gene families (46 peptide hormone families). Glycosylated peptide hormones identified in this study that have not previously been reported (red), glycosylated peptide hormones identified in this study and previously published (orange), peptide hormones not identified in this study but previously published (green), peptide hormones not identified in this study but predicted to be glycosylated by NetOGlyc 4.0 (yellow), peptide hormones not identified in this study and not predicted by NetOGlyc (grey).

FIG. 4 illustrates selected peptide hormone families and their identified O-glycosylation sites. Multiple sequence alignment analysis of the A) Secretin/Glucagon, B) Calcitonin, C) Insulin-like growth factor, D) Galanin, E) Neuropeptide Y family and F) Natriuretic peptides family with the identified, predicted and conserved O-glycosylation sites shown. Only the mature peptides are shown. Yellow boxes indicate identified glycosylation sites in this study, grey boxes indicate predicted glycosylation sites by NetOGlyc 4.0, and white boxes indicate conserved glycosylated residues. The sequence conservation of the mature peptides is shown below each peptide family. Dark grey: completely conserved sites; medium and light grey: less conserved sites; white: non-conserved sites. The peptide sequence shown in the alignments are A) secretin, B) calcitonin, C) insulin, D) galanin E) NPY and F) ANP.

FIG. 5 illustrates receptor activating capability of non-glycosylated and glycosylated peptide hormones. Secondary messenger accumulation assay for naked and Tn/T/ST-glycosylated peptide agonists for A) VPAC1, B) VPAC2, C) SCTR, D) GLP1R, E) GCGR, F & G) NPY1R, H-J) NPY2R, K&L) NPY4R, M&N) NPY5R. For VPAC1&2, GLP1R, GCGR and SCTR accumulation of cAMP was measured upon stimulation with increasing concentrations of the ligand whereas for NPY1R, NPY2R, NPY4R and NPY5R accumulation of IP-1 was measured upon stimulation with ligand after co-transfection with Gqo5. O) naked and ANP-19 and ANP-25 glycosylated variants receptor binding and activation measured as % cGMP generated.

FIG. 6 illustrates neprilysin (A) and insulin-degrading enzyme (B) proteolytic degradation of ANP and glycosylated proteoforms (monoglycosylated ANP-Ser19/Tn, ANP-Ser 25/Tn and double glycosylated ANP-Ser19+25/Tn in an time course from t=0 to t=24 hr monitored by MALDI-TOF analysis. Masses corresponding to intact peptide or glycopeptide are labelled “uncleaved” in green and indicated by arrows.

FIG. 7 illustrates Neprilysin, DPP-IV and IDE degradation pattern of non-glycosylated and glycosylated peptide hormones monitored by MALDI-TOF analysis. Peptides were incubated at 37 degrees in the presence of recombinant peptidase or 20% plasma. Aliquots were taken at 0, 15, 30, 60, 120 min for peptidase studies and 0 h, 1 h, 3 h, 6 h and 24 h for plasma studies and monitored by MALDI-TOF mass spectrometry. A) An illustrative example of peptidase digest with Neprilysin (NEP) on secretin and its glycoforms. The m/z of the degraded forms correlate with the N-terminal fragments shown in the sequence of secretin in panel B. B) Summary of cleavage sites and protection state of the glycans. a: DPPIV cleavage site, b: NEP cleavage site, c: plasma cleavage site. Nomenclature for protection: −: no protection, +: Partial protection, non-digested peak is present at least one timepoint after full degradation of the naked peptide has been observed. ++: full protection, no degradation products are observed within the timeframe of full degradation of the non-glycosylated peptide. In the example in panel A, partial protection is observed at the Tn form and the T form and full protection from the ST-form. *no protection in the N-terminus at the DPIV cleavage site, however, full protection was observed at C-terminal cleavage site already at the incorporation of Tn. C) summary of neprilysin, DPP-IV and plasma degradation assays. Nomenclature for protection: −: no protection, P: Partial protection, F: full protection, NT: Not tested, NC: No degradation of non-glycosylated peptide within timeframe. *Only the C-terminal inactivating cleavage of PYY is fully protected by glycans. The two N-terminal amino acids are removed and partial protected by only the ST-glycoform.

FIG. 8 illustrates a table summary of secondary messenger accumulation assay for naked and Tn/T/ST-glycosylated peptide for members of the glucagon- (VIP, GLP-1, Glucagon) and NPY (NPY, PYY)-families. For glucagon family members, cAMP accumulation was measured. For NPY family members, IP1-accumulation was measured. EC50-values are defined as concentration of peptide hormone needed to elicit 50% maximal response (Maximal achievable accumulation of either cAMP or IP1). The confidence interval is calculated from log-transformed data of at least 3 experiments.

FIG. 9 illustrates how natural o-glycans on ANP attenuate the acute renal and cardiovascular actions in vivo. A) schematic overview of the acute study protocol. B) Graph of changes in mean arterial pressure (MAP) overtime during the 60 minutes infusion period with ANP, ST-ANP19 and ST-ANP25 and after the 30 minutes clearance period. C) Urine flow (UV) was calculated as urine volume clearance per min. Values are plotted in a bar-graph displaying volume (uL) urine produced per minute in the 60-minutes infusion period and after the 30 minutes clearance period D) Urinary sodium excretion (UNaV) was calculated as urine sodium clearance per minute and values for ANP, ST-ANP19 and ST-ANP25 are plotted in a scatter-plot. E) Plasma ANP was measured after 90 minutes using a radioimmuneassay and values in pg/mL was plotted for ANP, ST-ANP19 and ST-ANP25. F) Urine ANP was measured after 90 minutes using a radioimmuneassay and values in pg/mL was plotted for ANP, ST-ANP19 and ST-ANP25.

### DETAILED DISCLOSURE OF THE INVENTION

O-glycosylation is emerging as an important regulator of protein stability and function. In this study, for the first time, the present inventors identify protein O-glycosylation as a common post translational modification of peptide hormones, present a detailed comprehensive map of O-glycosylation sites and suggest a biological function of site-specific O-linked glycosylation on peptide hormones.

Peptide hormones are produced by cells of the endocrine, neuronal or neuroendocrine tissues and are secreted in response to stimulus to bind to their cognate receptors and regulate complex physiological processes like appetite, blood pressure and anxiety. During biosynthesis peptide hormones undergo a range of PTMs. Besides a common complex proprotein convertase activation¹⁹, peptide hormones can undergo C-terminal amidation, N-terminal acetylation, tyrosine sulfation and serine phosphorylation²⁰ that may change the biochemical properties of the peptides. Furthermore, peptide hormones circulate in minute amounts and are inherently prone to proteolytic degradation. Thus, as a result of their instable nature, low abundance and complex post translational modifications peptide hormones have been difficult to isolate and characterize.

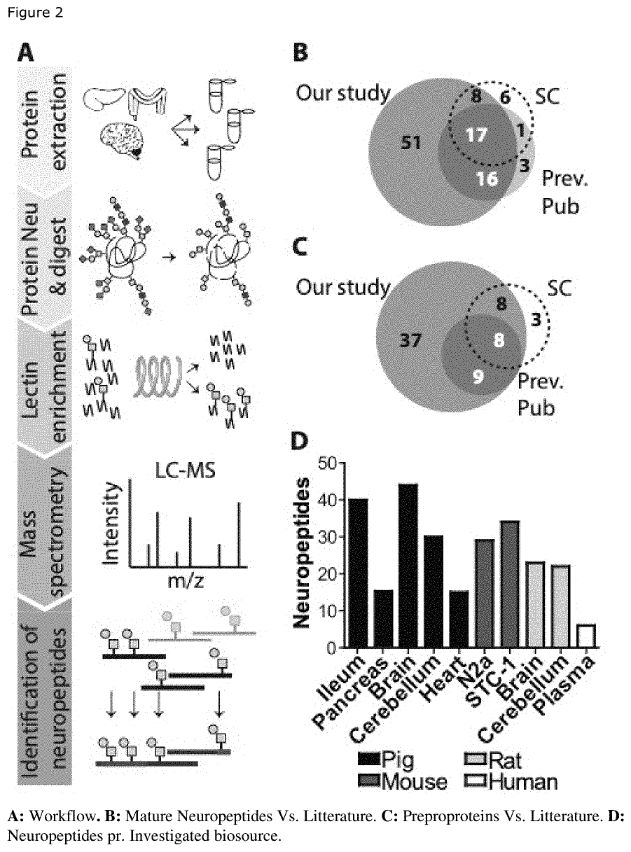

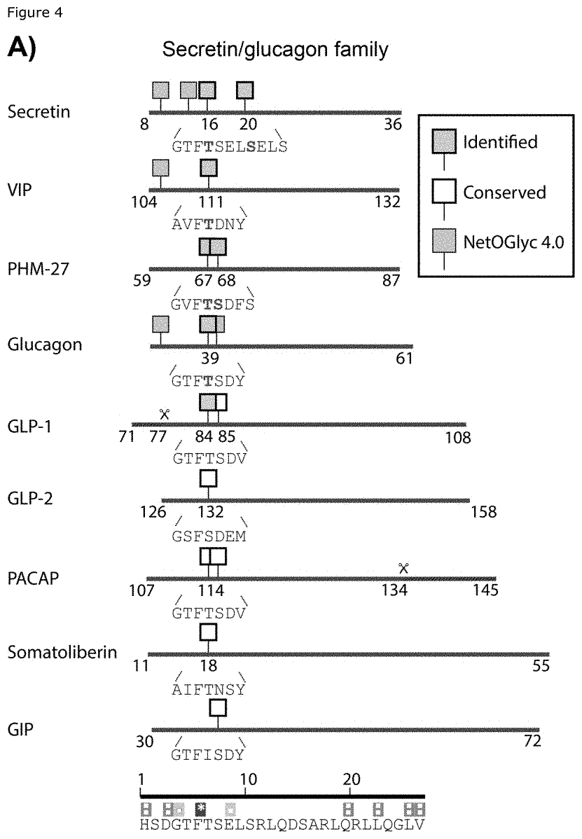

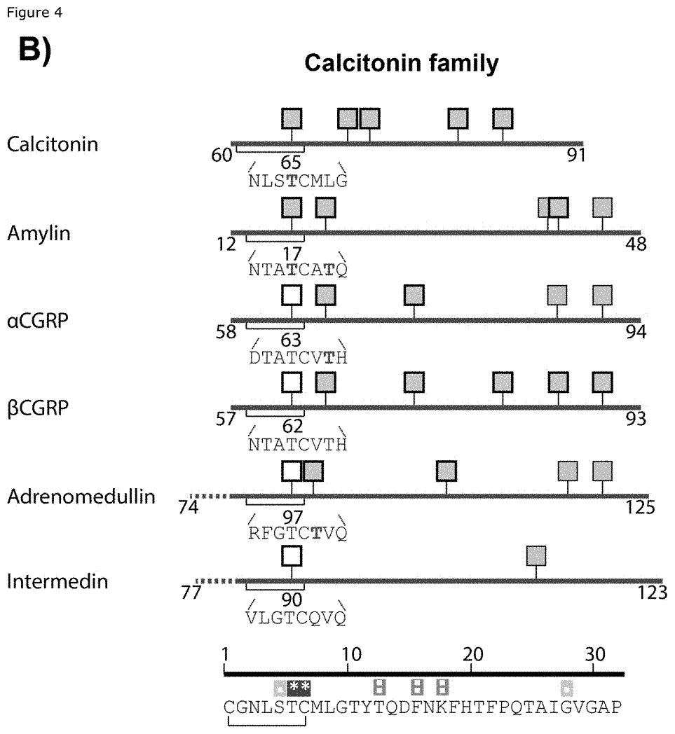

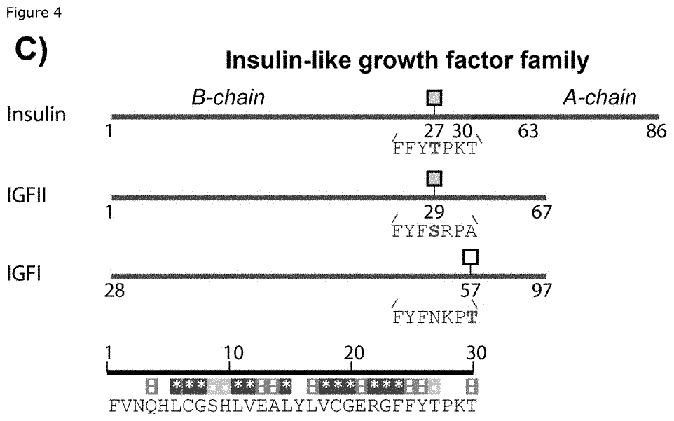

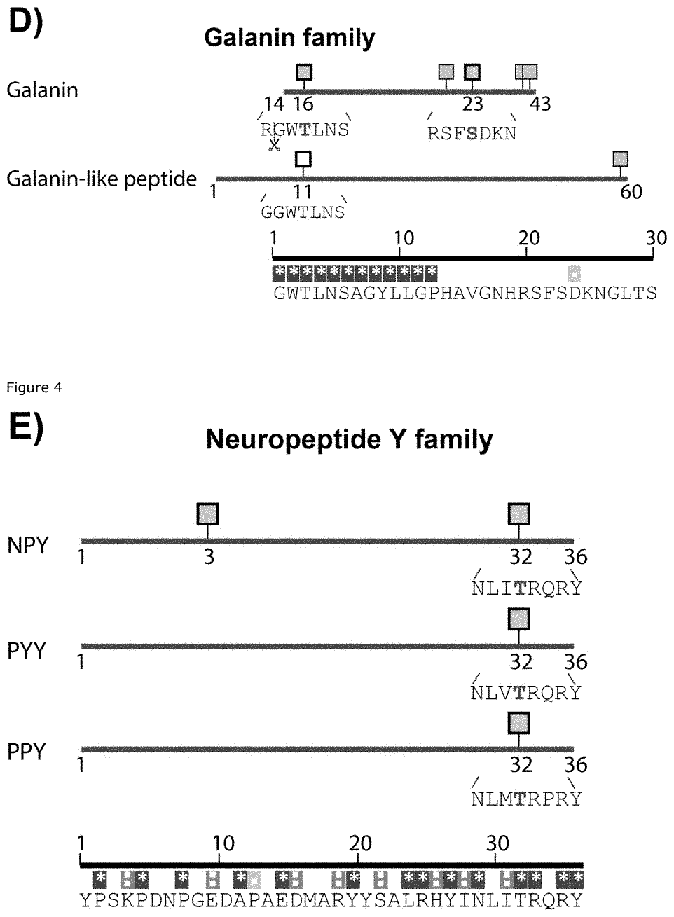

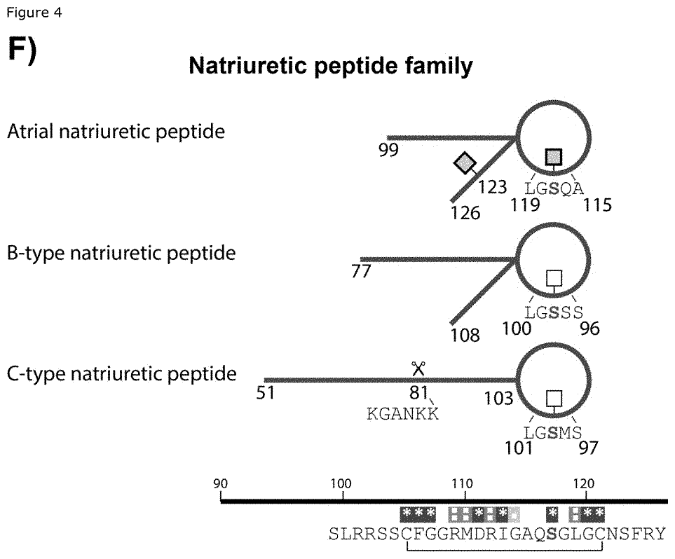

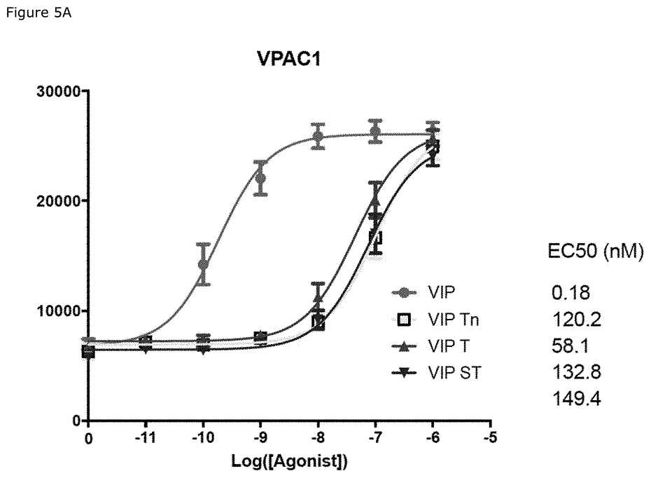

Now with the advantage of sensitive mass spectrometry the present inventors explore the occurrence of O-glycans on peptide hormones and show that approximately one third of all classified (NeuroPeP) peptide hormones are O-glycosylated (FIG. 2B and table 6), report the specific sites and demonstrate that the majority of sites resides within important structural or functional regions of the mature peptide hormones (FIG. 4 and table 6). Tables 6A to 6D disclose specific embodiments of the present invention, and table 6E discloses all of the herein disclosed peptide hormones with the respective sequence identity numbers (SEQ ID NOs).

GalNAc O-glycosylation is an exceptional PTM in that there are 20 differentially expressed isoforms with partly overlapping specificities conducting the addition of the initial GalNAc residue to the protein backbone Ser/Thr/Tyr residues. This leaves ample room for regulating the addition of site-specific O-glycosylation and the findings presented here may have revealed a novel regulatory level in peptide hormone biosynthesis and function.

It is becoming increasingly clear that site-specific O-glycosylation fine-tune the biological function of proteins²¹⁻³². Previously the present inventors have demonstrated that O-glycosylation protects proproteins from PC processing^{24,33,34}, that O-glycans increase the stability of GPCR N-termini³² and modulates ectodomain shedding²⁶ and most recently the present inventors have shown that loss of site-specific O-glycosylation impair ligand binding and uptake of the LDLR related receptor family^{35,36}.

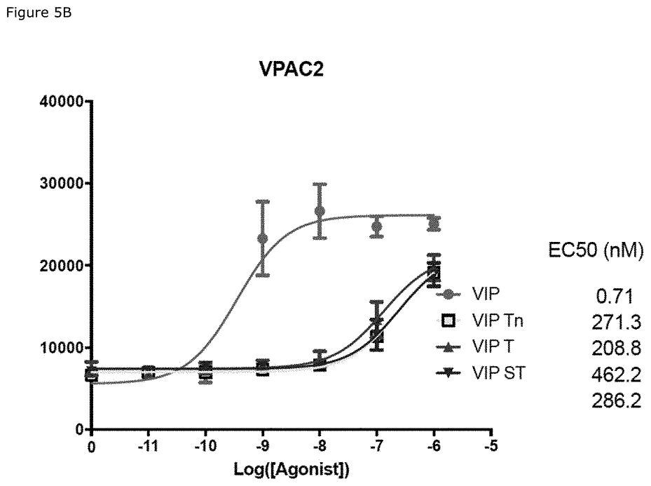

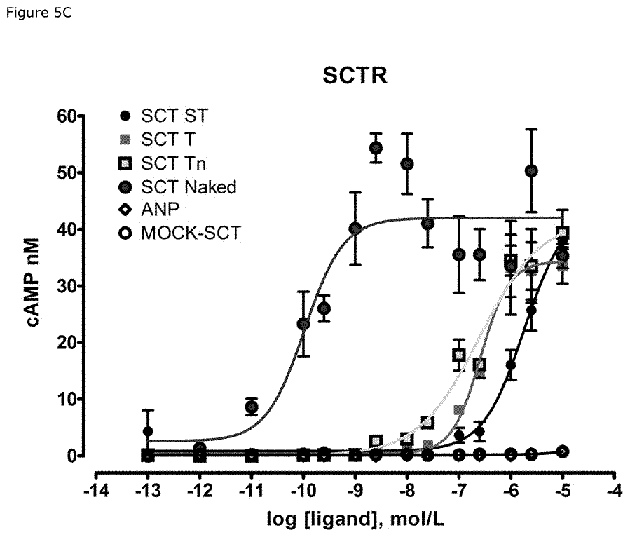

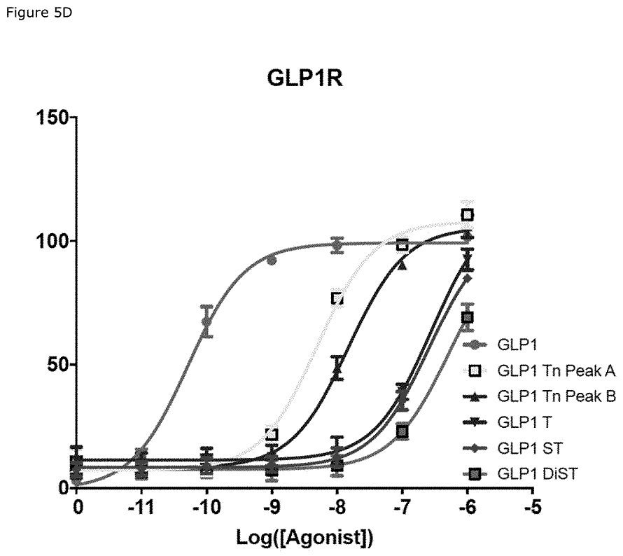

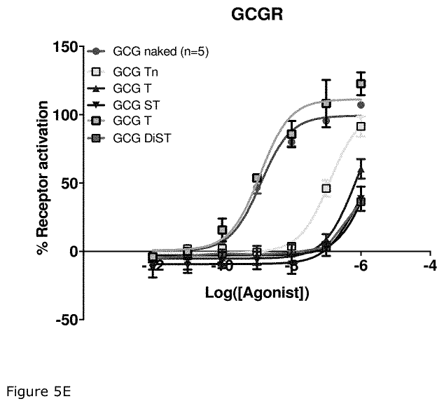

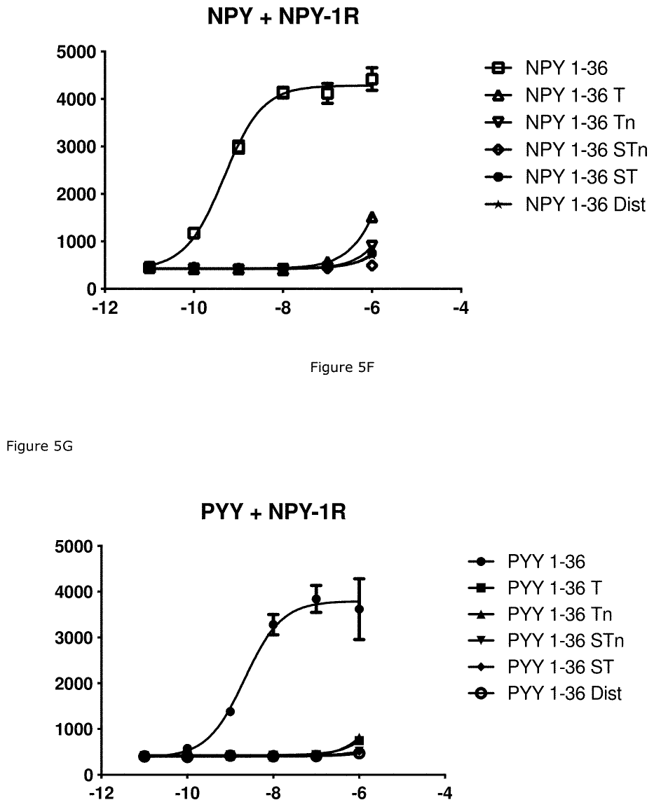

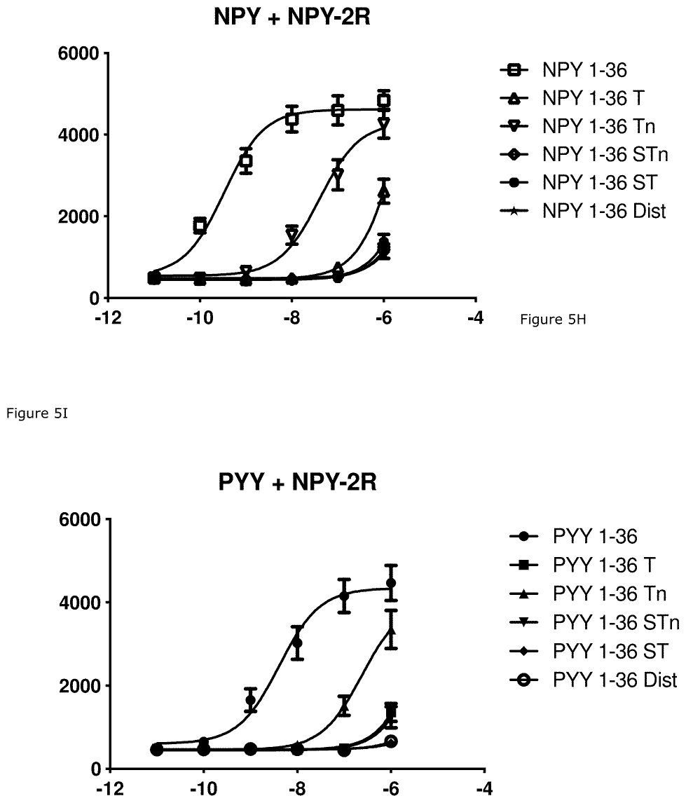

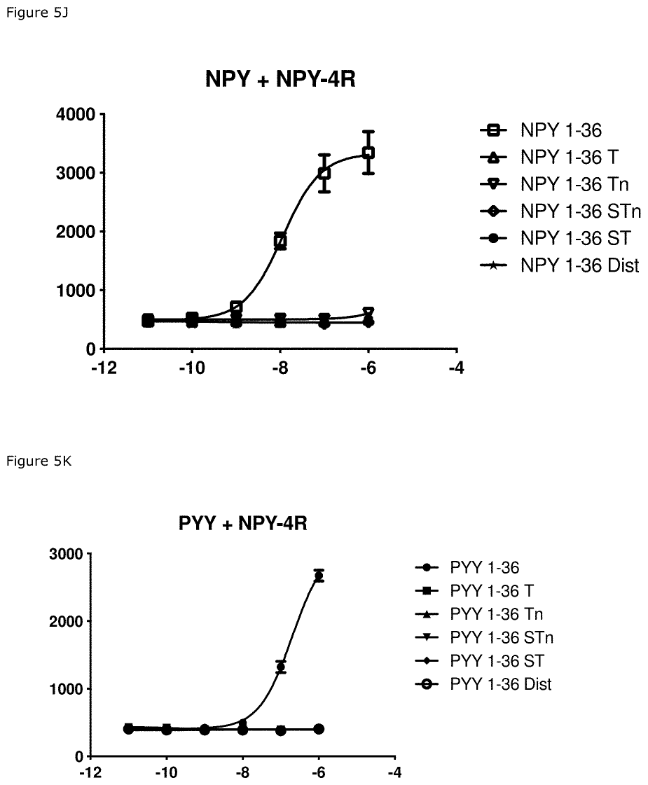

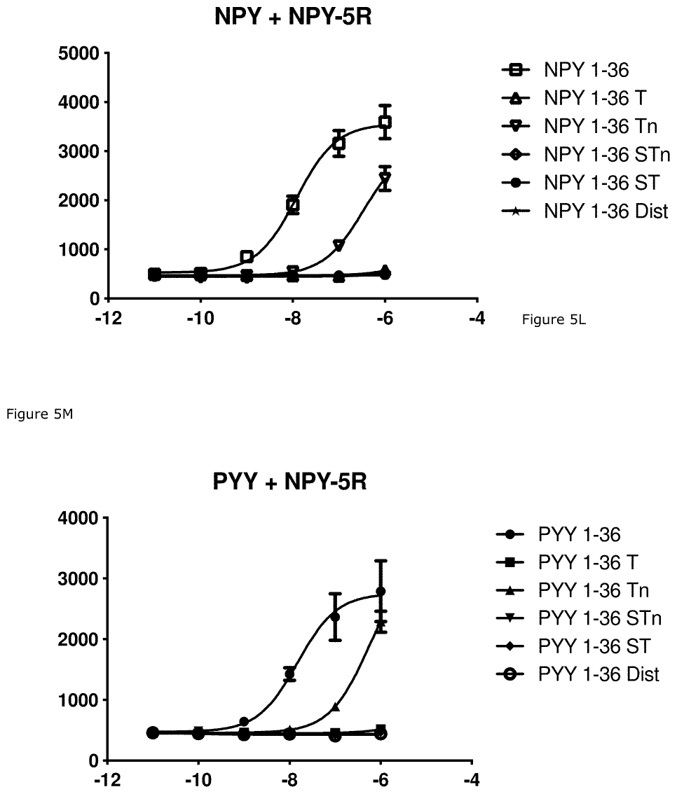

Here the present inventors demonstrate that O-glycosylation of VIP, Secretin, NPY, PYY and PPY in specific well conserved amino acid positions lowers receptor affinity and signalling, reducing EC50 more than 28-fold where the size of the glycan correlates with signal reduction (FIG. 5). Between the selected examples presented here, a similar effect was seen with ANP/NPRA, Secretin/SCTR, Glucagon/GCGR, GLP1/GLP1R, VIP/VPAC1, VIP/VPAC2, NPY/NPY1R, NPY/NPY2R, NPY/NPY4R, NPY/NPY5R, PYY/NPY1R, PYY/NPY2R, PYY/NPY4R and PYY/NPY5R.

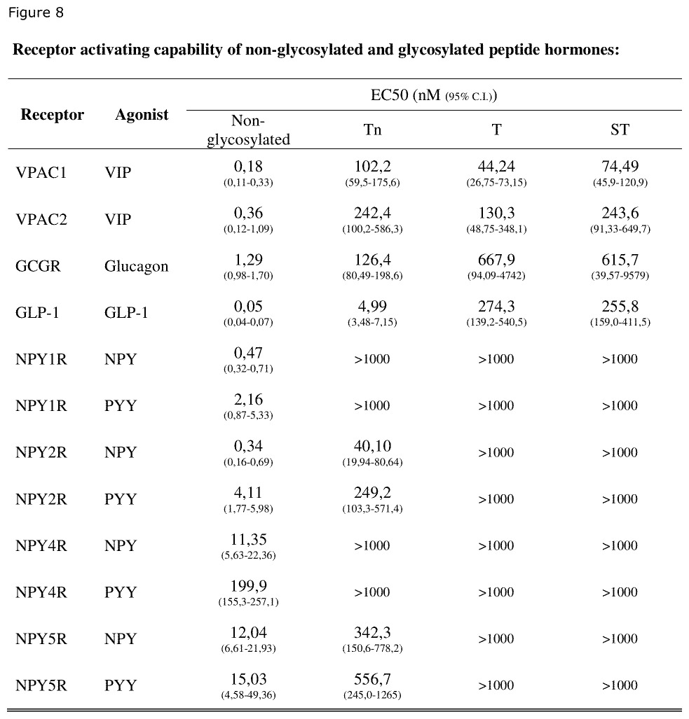

Our data indicates that the presence of glycan structures somehow alters the interaction between peptide hormone ligand and cognate receptor. Well in line with these data, especially well-studied in the Neuropeptide Y family, it has been demonstrated that other bulky chemical modifications of amino acids in the receptor binding domain alter receptor sub-class selectivity of the ligand³⁷. Such a sub-class receptor selectivity was observed for Thr32-Tn modified NPY that retained activity at the NPY2R and NPY5R receptors with a 118-fold and 37-fold decrease in potency (FIGS. 5H&J), respectively, whereas activity at the NPY1R and NPY4R receptors was almost abolished in the assayed range (FIGS. 5F&L). Thus glycosylation of NPY changes NPY's receptor sub-class selectivity from NPY2R>NPY1R>NPY5R>NPY4R to NPY2R>NPY5R>>NPY1R>NPY4R (FIG. 8). Thr32PYY-Tn showed same retained activity at NPY2R and NPY5R with 61-fold and 37-fold reduction in potency, respectively (FIGS. 5I&K), but minimal activity at the NPY1R and NPY4R in the assayed range (FIGS. 5G&K). Thus glycosylation of PYY changes NPY's receptor-subtype selectivity from NPY1R>NPY2R>NPY5R>NPY4R to NPY2R>NPY5R>>NPY1R>NPY4R (FIG. 8).

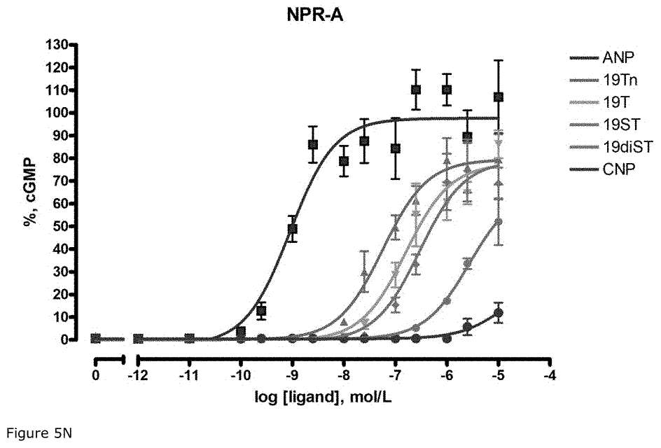

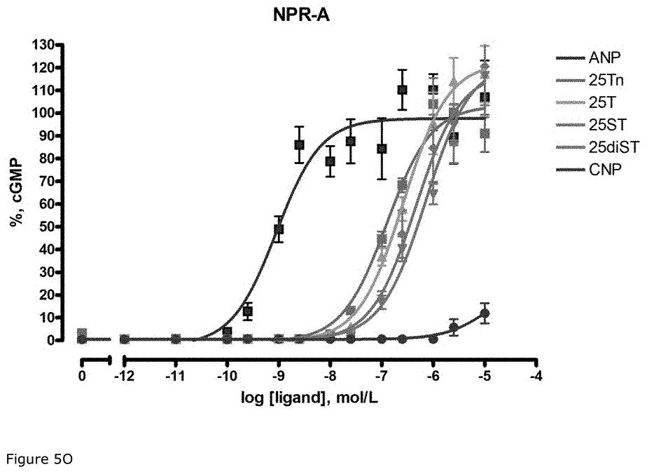

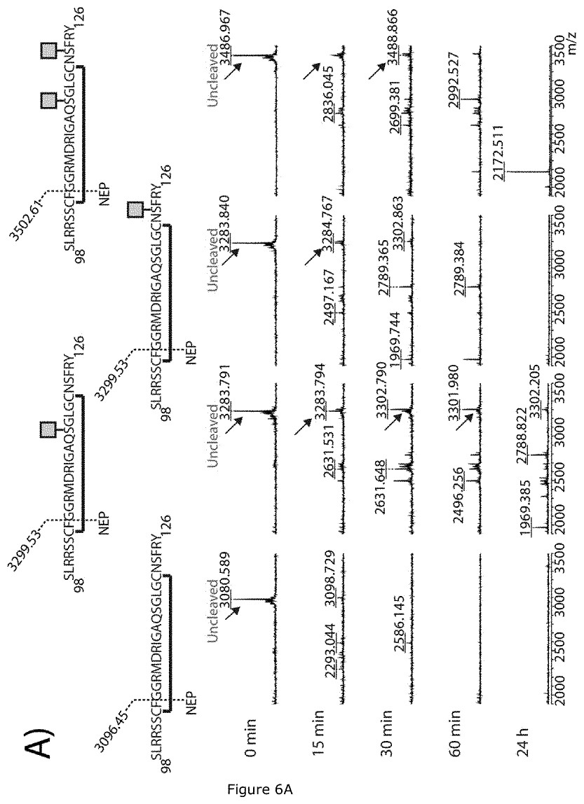

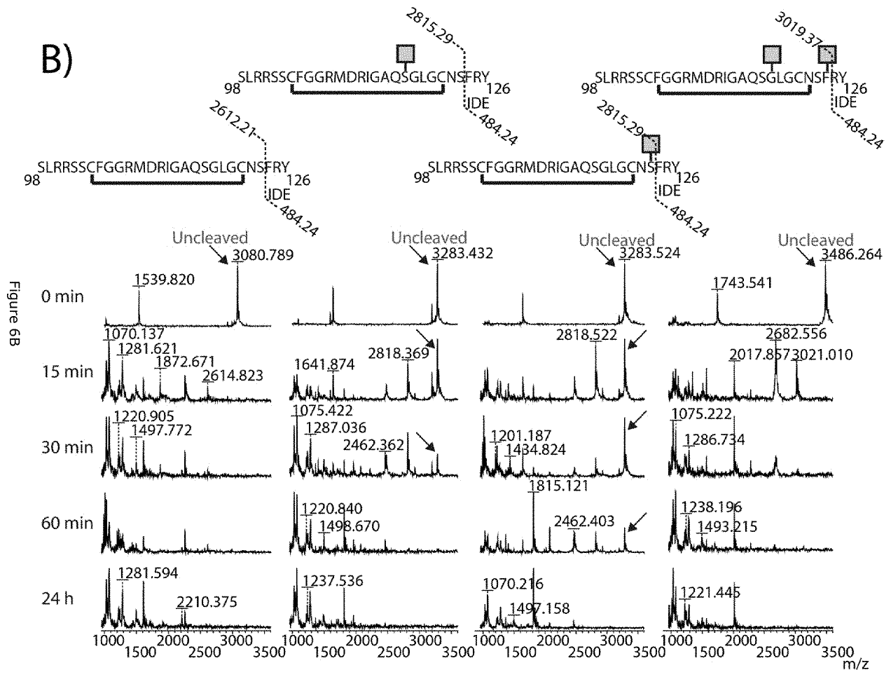

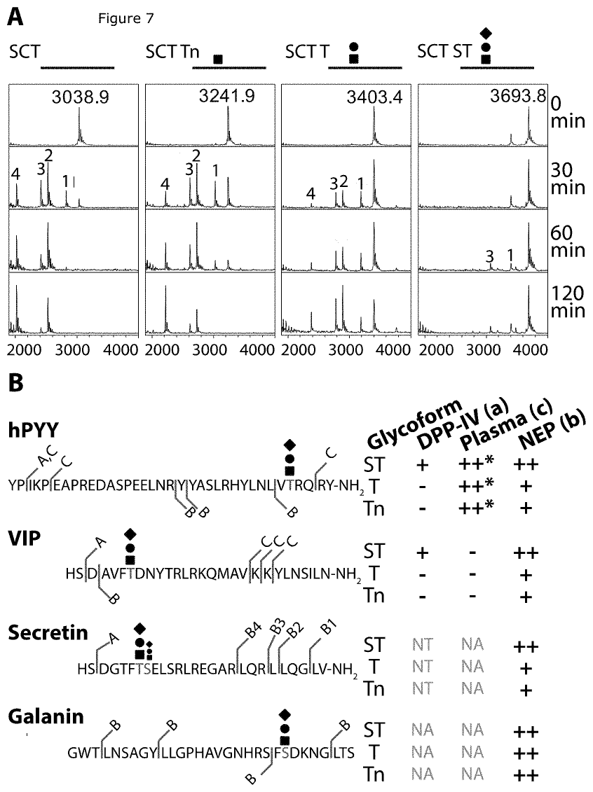

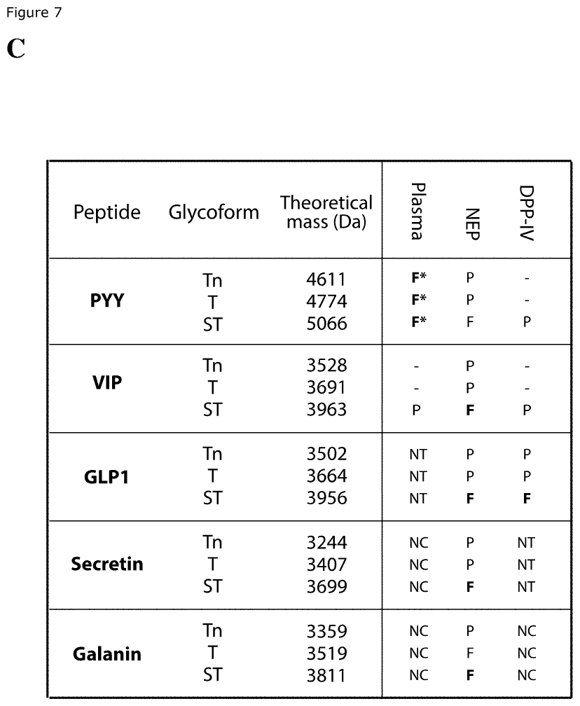

Peptide hormones are inherently unstable and circulate only in minute amounts. Here, it is demonstrated that site-specific O-glycosylation on Secretin, VIP, Galanin, PYY, GLP-1 and ANP protects the peptide hormones from proteolytic degradation in vivo using a rat model, ex vivo using plasma degradation assays and in vitro using recombinant proteases (FIG. 6 & FIG. 7). Most prominent is the stability of the naturally occurring sialylated structures where e.g. Thr-32 ST-PYY remained partially in its biologically active form even after prolonged incubation time with plasma and Ser-23 ST-Galanin remained partially intact after overnight incubation with neprilysin. Even though the sialylated structures of VIP and PYY were also protected from DPP-IV degradation, the Tn-glycosylated structures were in some cases somewhat faster degraded compared to non-glycosylated, perhaps related to the unphysiological nature of the Tn structure that is primarily observed in cancer cells.

The Neuropeptide Y family members are ubiquitously expressed in the body and act as neurotransmitters to regulate a vast array of physiological processes via binding and signalling through the Gi coupled NPY receptors (YR1, YR2, YR4 or YR5).

The main function of GLP-1 is to increase insulin secretion, i.e. to act as an “incretin”, but it also inhibits gastrointestinal motility and function as a physiological regulator of appetite. Recently it was demonstrated that GLP-1, Oxyntomodullin and PYY in combination injected subcutaneously using a pump device into obese volunteers reduced their mean caloric intake with 32%³⁸.

ANP is classically released from secretory granules from the atria in a regulated fashion which makes it able to rapidly regulate hemodynamics in response to increased pressure. Glycosylated ANP was protected from degradation in vitro by IDE and NEP which suggest that glycosylated ANP may have increased half-life. The combined effects of glycosylated ANP, i.e decreased potency and increased stability, could render a positive effect in the treatment of acute decompensated heart failure and hypertension, where ANP and BNP are already introduced as infusion therapy³⁹⁻⁴¹.

Thus, there is a need to identify selective peptide hormone receptor agonists or antagonists with good biostability to pursue as potential therapeutic candidates for treating e.g. cardiovascular diseases, anxiety, depression, obesity, epilepsy, alcoholism.

Peptide based design of therapeutics is attractive in many ways since biological active peptides have the potential to regulate specific functions of GPCRs and ion-channels and in general in vivo based drug design is favourable due to lower toxicity and immunogenicity and higher selectivity and predictable in vivo behaviour. However, probing the function and efficacy of what was thought of as “naturally occurring” unmodified peptides have demonstrated low biostability and circulation time and therefore low efficacy^{42,43} and a number of strategies have been taken to chemically alter the biochemical properties of peptide hormones or synthetic analogues and improve these parameters⁴⁴. It is generally found that large N-glycans on proteins may enhance circulatory half-life, although mainly by increase of the size and hydrodynamic size of proteins. However, introduction of O-glycans into smaller peptides have also been used to enhance circulatory half-life especially when combined with the GlycoPegylation strategy⁴⁵. In one relevant example an O-glycosylation sequon was introduced in the C-terminus of the GLP-1 receptor antagonist Exendin (9-39) increasing functional half-life⁴⁶. Other studies have explored the chemical synthesis of peptide hormones with glycans⁴⁶⁻⁵², e.g. O-linked galactose on vasopressin, PYY and VIP, glucose on Leu-enkephalin and PYY, N-linked GlcNAc on GLP-1 and N-terminal chemical glycation of GIP, GLP-1(7-36) and Insulin. Interestingly all studies, except one describing O-linked galactose on Vasopressin, find, very much in line with what the present inventors show here for the naturally occurring O-GalNAc glycans, that the chemically modified glycosylated analogues are less or equally potent in in vitro receptor activation assays, yet these other studies demonstrate higher stability and potency in vivo.

Naturally occurring O-glycans positioned within the receptor-interaction region of peptide hormones have not been demonstrated previously, and we hypothesize that such naturally occurring glycans selectively affect function and biostability as our preliminary studies suggest.

The inventors of the present invention also identified O-glycosylation on the pro-part of peptide hormones. Here, sequestered from the bioactive part, the sugars might regulate PC processing and activation and furthermore coincidentally mask antibody binding epitopes^{9,24,53-56}. One such well-studied example is proBNP which is synthesized in the ventricles of the failing heart and undergo limited proteolysis by PCs releasing the C-terminal peptide hormone BNP that regulate natriuresis and blood pressure. ProBNP is O-glycosylated in the N-terminal proprotein (NT-proBNP) close to the PC processing site and amino acid substitution experiments have validated that Thr71 protects proBNP from processing by Corin or Furin⁵³. The present inventors identified O-glycans on proBNP, POMC, Kininogen-1, Chromogranin A (Table 6). NT-proBNP is an important biomarker for heart failure and commercial immunoassays are being used in the clinic to quantify NT-proBNP in heart related diseases. However some variability among the assays have been noted and caution raised against O-glycans potentially masking antibody binding epitopes⁵⁷. As POMC⁵⁸, Kininogen-1⁵⁹ and chromogranin A⁶⁰ are also used as biomarkers it is particular important to note the degree of glycosylation identified in this study.

In summary the present inventors show that O-glycans in conserved residues in various peptide hormone families, are far more abundant than previously recognized, and that glycans change peptide hormone induced receptor activity and furthermore alter recognition by proteolytic enzymes that otherwise inactivate the peptide hormones.

### EXAMPLES

The purpose of the following examples are given as an illustration of various embodiments of the invention and are thus not meant to limit the present invention in any way. Along with the present examples the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

Biological samples were prepared as follows:

Tissue Extraction: Porcine brain, cerebellum, ileum and a pool of human prostate gland tissue⁶¹ was isolated according to standard protocols. Proteins were extracted by crushing the frozen tissue using a CryoPrep tissue extractor (Covaris, Woburn, Mass.), boiled in water for 20 minutes, and homogenized with an Ultra-Turrax (IKA, Staufen, Germany). For ileum tissue, instead of boiling in water, the tissue was homogenized and rotated at 4° C. for 4 hours in 0.18 M HCl/70% ethanol. After 30 minutes centrifugation at 13,000 g, the supernatants were collected and pooled, and protein concentration was determined by BCA assay (Pierce). Prostate gland samples were further processed as crude water extract, acetone precipitated extract and acetone precipitated extract after acidification. Brain and cerebellum extracts were either crude extracts or acetone precipitated extracts after acidification. For precipitation of insoluble proteins samples in water or adjusted to 0.5 M CH3COOH were added icecold acetone (67%), incubated for 1 h at −20° C., and centrifuged at 16.000 g for 30 min. Subsequently the supernatant was lyophilized and reconstituted in water.

Plasma extraction: Plasma for O-glycoproteomic analyses was collected and pooled from two healthy volunteers into EDTA-treated tubes (K2E K2EDTA Vacuette) followed by centrifugation at 5,000×g for 10 min and stored at −20° C. until use.

Both crude and low molecular weight fraction (LMWF) enriched plasma was subjected to the O-glycoproteome strategy. For LMWF enrichment a volume of plasma containing approximately 60 mg of protein (measured by BCA assay (Pierce)) was precipitated by adding two parts of 96% ethanol followed by incubation at RT for 30 min. Supernatant and pellet was separated by centrifugation at 10.000×g for 10 minutes, and the supernatant was lyophilized and resuspended in 0.05% rapigest. 50 mM sodium acetate buffer and desialylated with 0.1 U/mL neuraminidase (Clostridium perfringens neuramidase type VI, Sigma). The LMWF-enriched sample was further enriched for O-glycopeptides by capture on a short (300 ml contained in 1 ml syringe) PNA agarose column. Glycoproteins were eluted by heating the lectin (2×90° C., 10 min) with 0.05% RapiGest (as previous described¹¹). The LMWF-enriched plasma sample was 0.2 μm filtered prior to the glycoprotein enrichment. In parallel, 5 mg of total protein (as determined by BCA assay (Pierce)) from non-LMWF-enriched biofluid samples was desialylated by the same procedure as described above, before enzymatic degradation omitting the glycoprotein enrichment.

Cell protein extraction: Conditioned media cleared from dead cells and debris obtained from 2×T175 flasks cultured for 48-72 h were dialyzed, neuraminidase treated and enriched for glycoprotein as done for the the LMWF-enriched plasma. Total cell lysates (TCL) were obtained by washing a monolayer of cells in icecold PBS, scrabing off the cells and adding 2 ml 0.05% RapiGest to solubilize the cell pellet. The resulting homogenate was sonicated and cleared by centrifugation.

### Mass Spectrometry Workflow

Enzyme digestion and desialylation: The extracted samples from the various neuronal and endocrine sources were adjusted to 50 mM ammonium bicarbonate, heated for 10 min at 80° C., followed by reduction with 5 mM dithiothreitol (DTT) (60° C., 30 min) and alkylation with 10 mM iodoacetamide (30 min, room temperature, kept dark). Subsequently, the samples were incubated with trypsin, Glu-C or chymotrypsin (Roche) (37° C., overnight, 1 μg enzyme pr 100 μg protein). The following day, the enzyme reaction was quenched and RapiGest, if present in the sample, was precipitated by acidifying with trifluoroacetic acid (TFA). The solution was cleared by centrifugation (10,000×g, 10 min.) and peptides were purified on C18 Sep-Pak columns (Waters), and dried down using SpeedVac. If not already desialylated, the dried peptides were resuspended in 1 mL 50 mM Sodium acetate (pH 5.5) containing 0.1 U/mL Neuraminidase followed by incubation at 37° C. for 1 h, purified by Sep-Pak and dried down.

LWAC O-glycopeptide enrichment: Dried samples were reconstituted in 2 mL PNA/Jacalin/VVA buffer (PNA-binding buffer 10 mM HEPES (pH 7.4), 150 mM NaCl, 0.1 mM CaCl₂, and 0.01 mM MnCl₂; Jacalin-binding buffer 175 mM Tris (pH 7.5); VVA-binding buffer 20 mM Tris-HCl (pH 7.4), 150 mM NaCl, 1 mM CaCl₂/MgCl₂/MnCl₂/ZnCl₂, and 1 M urea), 0.45 μm filtered and injected onto a pre-equilibrated 2.6-m long column packed with lectin-bound (PNA, Jacalin or VVA, Vector Laboratories) agarose beads at a constant flow-rate of 0.1 mL/min. For VVA the column was first washed for 3×CV in 0.4 M glucose and then eluted with 2 CV 0.2 M GalNAc and 1×CV 0.4 M GalNAc. For PNA and Jacalin LWAC, the column was washed 2×CV in lectin-binding buffer, and then eluted with 2×1 column volume 0.5 M galactose and 1×1 column volume 1 M galactose, respectively. The elution fractions were concentrated and glycopeptides were purified using Stage tips and submitted for nLCMS/MS analysis.

nLC/MS/MS Analysis: Liquid chromatography-tandem mass spectrometry was performed on a system composed of an EASY-nLC 1000 (Thermo Fisher Scientific) interfaced via a nanoSpray Flex ion source to an LTQ-Orbitrap Velos pro hybrid spectrometer or Orbitrap Fusion Tribrid (Thermo Fisher Scientific), equipped for both higher energy collisional dissociation (HCD) and electron transfer dissociation (ETD) modes, enabling peptide sequence analysis without and with retention of glycan site-specific fragments, respectively. The nLC was operated in a one-column set up with an analytical column (20 cm length, 75 μm inner diameter) packed with C18 reverse phase material (1.9-μm particle size, ReproSil-Pur, Dr. Maisch). Each sample dissolved in 0.1% formic acid was injected onto the column and eluted in a gradient from 2 to 30% B in 105 min, from 30% to 100% B in 5 min and 100% B for 10 min at 200 nl min-1 (solvent A, 100% H₂O; solvent B, 100% acetonitrile; both containing 0.1% (v/v) formic acid). A data-dependent mass spectral acquisition routine, HCD triggering of subsequent ETD scan, was used for all runs. Briefly, a precursor MS1 scan (m/z 350-1,700) of intact peptides was acquired in the Orbitrap at a resolution setting of 30,000 (Velos Pro) or 120,000 (Fusion), followed by Orbitrap HCD-MS2 (m/z of 100-2,000) of the five most abundant multiply charged precursors in the MS1 spectrum; this event was followed up by an ETD-MS2 fragmentation for the same precursor ion. In cases where preliminary screening of fractions for glycopeptide enrichment was carried out prior to IEF, the ETD-MS2 step was omitted, and HCD-MS2 (m/z 100-2,000) of the five most abundant multiply charged precursors was acquired (“top five method”). These HCD-MS2 spectra were simply screened for the appearance of the HexNAc fragment at m/z 204.086.

Data analysis: The raw data were processed using Proteome Discoverer 1.4 software (Thermo Fisher Scientific) and searched against the human, porcine, mouse or rat-specific Uniprot database downloaded on January 2013. The Sequest HT search node was used for HCD and ETD data. In all cases the precursor mass tolerance was set to 15 p.p.m. and fragment ion mass tolerance to 20 millimass units (mmu). Carbamidomethylation on cysteine residues was used as a fixed modification. Methionine oxidation, C-terminal amidation (plasma and pancreas), and HexNAc or HexHexNAc attachment to serine, threonine or tyrosine were used as variable modifications. As an additional preprocessing procedure, all HCD data showing the presence of fragment ions at m/z 204.08 were extracted into a single .mgf file, and the exact mass of 1×, 2×, 3× and 4× HexNAc or HexHexNAc units was subtracted from the corresponding precursor ion mass, generating four distinct files. These preprocessed data files were submitted to a Sequest HT node under the same conditions mentioned above, except considering a HexNAc or HexHexNAc attachment. All spectra were searched against a decoy database using a target false discovery rate of 1%; unassigned spectra were submitted to a second Sequest HT node using the same parameters as above with the exception of performing the search using semi-specific trypsin cleavage. The final list was filtered to include only peptide hormones.

Multiple sequence alignment: All alignments were performed in ClustalW using the peptide sequences of H. sapiens, M. musculus, and R. Norvegicus.

In some aspects of the present application, mammalian host cells are modified to inactivate or downregulate certain glycosyltransferase genes. Details for for modifying or adding all subtypes of O-GalNAc linked mucin-type O-glycans are described in WO 2017/194699, which reference is hereby incorporated by reference.

In brief the present invention may incorporate the use of mammalian host cells with individual and combinatorial knock out of one or more of the GALNT1-T20 glycogenes (listed in Table 1 below). Determining changes in interactions with a plurality of mammalian cells with knock out of GALNT1 and/or GALNT2 and/or GALNT3 and/or GALNT4 and/or GALNT5 and/or GALNT6 and/or GALNT7 and/or GALNT9 and/or GALNT10 and/or GALNT11 and/or GALNT12 and/or GALNT13 and/or GALNT14 and/or GALNT16 and/or GALNT18 and/or GALNT19 is used to identify if said interaction occurs through subsets of O-GalNAc glycoproteins controlled by one or more of the 20 GALNTs, respectively, such that loss or reduction in measured interactions with mammalian cells with knock out of one or more of the named gene(s) confer that the O-glycoprotein(s) responsible for the interaction requires glycosylation by the corresponding GALNT(s).

TABLE 1

| | |
|---|---|
| O-Gly (Ser/Thr/Tyr) | GALNT1-T20 (O-GalNAc) |

### O-Glycan Branching

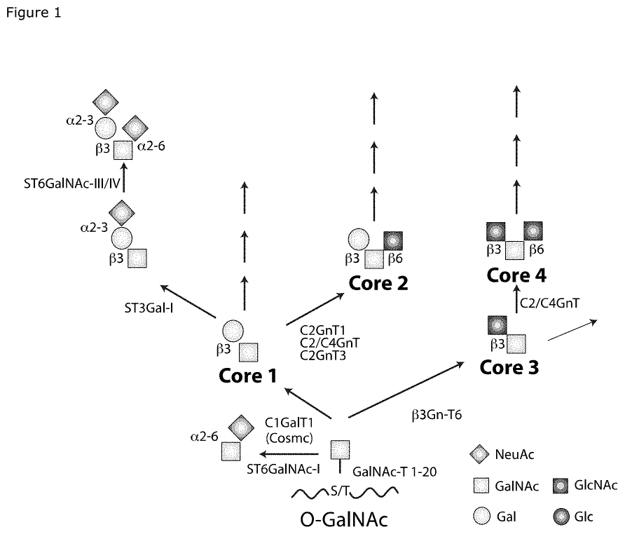

The present invention may incorporate the use of mammalian host cells with individual and combinatorial knock out of C1GalT1, GCNT1, GCNT2, GCNT3, GCNT4, GCNT6, GCNT7, B3GNT6 or B3GNT2 glycogenes (listed in Table 2 below) suitable for determining O-linked branching in Core2, Core3 and Core4 structures (FIG. 1), involved in observed interactions. Determining changes in interactions with a plurality of mammalian cells with knock out of GCNT1 and/or GCNT2 and/or GCNT3 and/or GCNT4 and/or GCNT6 and/or GCNT7 and/or B3GNT6 and/or B3GNT6 is used to identify if said interaction occurs through O-linked branched structures by one or a plurality of the branching enzymes, such that loss or reduction in measured interactions with mammalian cells with knock out of one or more of the named gene(s) confer that the O-linked branched structure is responsible for the interaction as indicated.

TABLE 2

| | |
|---|---|
| O-Gly | C1GALT1, B3GNT6 (O-GalNAc) |
| | GCNT1/T2/T3/T4/T6/T7 (O-GalNAc) |
| | B3GALNT1/T2 (O-Gly) |
| | B3GNT2/T3/T4/T7/T8/T9 (O-Gly) |
| | B4GALT1/T2/T3/T4 (O-Gly) |
| | B4GALNT3/T4 (O-Gly) |

The present invention may incorporate the use of mammalian host cells with individual and combinatorial knock out of genes involved in N and O-glycan and glycolipid capping (sialylation); ST3GAL1/2/3/4/5/6 (α2,3NeuAc capping/sialylation) and/or ST6GAL1/2 (α2,6NeuAc capping/sialylation) and/or ST8SIA1/2/3/4/5/6 (capping by poly-sialylation) and/or ST6GALNAC1/2/3/4/5/6 (α2,6NeuAc capping/sialylation) (glycogenes listed in table 3 below) suitable for determining the capped (sialylated or fucosylated) glycan structure involved in observed interactions. Determining changes in interactions with a plurality of mammalian cells with knock out of ST3GAL1/2/3/4/5/6 and/or ST6GAL1/2 and/or ST8SIA1/2/3/4/5/6 and/or ST6GALNAC1/2/3/4/5/6 is used to identify if said interaction occurs through the type of capping indicated in parenthesis, such that loss or reduction in measured interactions with mammalian cells with knock out of one or more of the named groups of genes confer that the type of capping is responsible for the interaction as indicated.

TABLE 3

| | |
|---|---|
| N-Gly, | FUT1/2/3/4/5/6/7/8/9/10/11 |
| O-Gly, | ST3GAL1/2/3/4/5/6 |
| Glycolipids | ST6GAL1/2 |
| | ST6GALNAC1/2/3/4/5/6 |
| | ST8SIA1/2/3/4/5/6 |
| | B3GAT1/T2 |
| | ABO |
| | A4GNT |

### Engineering GTfs in Cells

Only little information exists as to the effects of knock out of glycosyltransferase genes in mammalian cell lines. For human cell lines only a few spontaneous mutants of glycosyltransferase genes have been identified. For example the colon cancer cell line LSC derived from LS174T has a mutation in the COSMC chaperone that leads to misfolded and non-functional Core1 synthase C1GalT⁶². The COSMC gene is also mutated in the human lymphoblastoid Jurkat cell line^{62,63}.

### Knock Out of Glycosylation Genes in Cell Lines

The limited information of effects of knock out of glycosyltransferase genes in cell lines is partly due to past difficulties with making knock outs in cell lines before the recent advent of precise gene editing technologies⁶⁴. Thus, until recently essentially only one glycosyltransferase gene, FUT8, had been knocked out in a directed approach using two rounds of homologous recombination including massive clone screening efforts. The conventional gene disruption by homologous recombination is typically a very laborious process as evidenced by this knock out of Fut8 in CHO, as over 100,000 clonal cell lines were screened to identify a few growing Fut8−/− clones⁶⁵ (U.S. Pat. No. 7,214,775). With the advent of the Zinc finger nuclease (ZFN) gene targeting strategy it became less laborious to disrupt genes, which was first demonstrated by knock out of the Fut8 gene in a CHO cell line, where additional two other genes unrelated to glycosylation were also effectively targeted⁶⁶. More recently, TALENs and the CRISPR/Cas9 editing strategies have emerged, and the latter editing strategy was used to knock out the Fut8 gene⁶⁷.

It is thus clear that targeted genetic engineering is now a tool the skilled person may use but editing of the glycosylation genes in mammalian cells and animals are prone to substantial uncertainty, and thus identifying the optimal engineering targets for display of a given glycan structure will require extensive experimental efforts. Therefore a random type approach involving testing of a multiplicity of different glycogene and glycoform variations may be beneficial.

### Overexpression of Glycosylation Genes in Cell Lines

It is noteworthy that transient or stable overexpression of a glycosyltransferase gene in a cell most often result in only partial changes in the glycosylation pathways in which the encoded enzyme is involved. A number of studies have attempted to overexpress e.g. the core2 C2GnT1 enzyme in CHO to produce core2 branched O-glycans, the ST6GAL1 sialyltransferase to produce α2,6linked sialic acid capping on N-glycoproteins⁶⁸, and the ST6GALNAC1 sialyltransferase to produce α2,6linked sialic acid on O-glycoproteins forming the cancer-associated glycan STn⁶⁹. However, in all these studies heterogeneous and often unstable glycosylation characteristics in transfected cell lines have been obtained. This is presumably partly due to competing endogenous glycosyltransferase activities whether acting with the same substrates or diverging pathway substrates. Other factors may also explain the heterogeneous glycosylation characteristics.

### Definitions

Before disclosing the subject-matter in greater detail, definitions of terms/expressions used herein are provided.

Unless otherwise defined, all technical and scientific terms used herein have the same meaning as commonly understood by one of ordinary skill in the art to which this invention belongs. Although methods and materials similar to those described herein can be used in the practice or testing of the present invention, suitable methods and materials are described below. In addition, the materials, methods, and examples are illustrative only and not intended to be limiting.

Each of the patents, applications and articles cited herein, and each document cited or referenced therein, including during the prosecution of any of the patents and/or applications cited herein (“patent cited documents”), and any manufacturer's instructions or catalogues for any products cited herein or mentioned in any of the references and in any of the patent cited documents, are hereby incorporated herein by reference. Documents incorporated by reference into this text or any teachings therein may be used in the practice of this invention.

Documents incorporated by reference into this text are not admitted to be prior art. As used herein, the words “may” and “may be” are to be interpreted in an open-ended, non-restrictive manner. At minimum, “may” and “may be” are to be interpreted as definitively including structure or acts recited.

The articles “a” and “an” are used herein to refer to one or to more than one (i.e. to at least one) of the grammatical object of the article.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “therapeutically effective amount or dose” or “sufficient amount or dose” herein is meant a dose that produces effects for which it is administered. The exact dose will depend on the purpose of the treatment, and will be ascertainable by one skilled in the art using known techniques.

The terms “pharmaceutically effective”, “therapeutically effective”, “pharmaceutically active”, or “therapeutically active” means that a synthetic compound of the invention so described is determined to have activity that affects a medical parameter or disease state.

“Patient” as that term is used herein, refers to the recipient of the treatment. In a specific embodiment, the patient is a mammal, such as a human, canine, murine, feline, bovine, ovine, swine or caprine. In a particular embodiment, the patient is a human.

As used herein, the terms “function” or “functional activity” refer to a biological, e.g., enzymatic function.

By “isolated” is meant material that is substantially or essentially free or purified from components that normally accompany it in its native state. For example, the compound according to the invention may be modified subsequent to isolation from their natural or laboratory-produced environment, or they may be used in isolated form in vitro, or as components of devices, compositions, etc.

By “obtained from” is meant that a sample such as, for example, a polypeptide (peptide hormone) is isolated from, or derived from, a particular source of the host or cells cultured in vitro. For example, the extract can be obtained from a tissue or a biological fluid sample isolated directly from the host. Therefore, the compounds of the present invention may be recombinantly produced or obtained from biological sources and be purified before further use in vitro and/or in vivo.

By “pharmaceutically acceptable carrier” is meant a solid or liquid filler, stabilizer, diluent or encapsulating substance that can be safely used in administration routes when applied to an animal, e.g. a mammal, including humans.

“Therapeutic treatment”, and “treatment”, refers any type of therapy.

The terms “peptide hormone”, “polypeptide,” “peptide” and “protein” are used interchangeably herein to refer to a polymer of amino acid residues. The terms apply also to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” or “derivative” where the alteration results in the substitution of an amino acid, e.g., with a chemically similar amino acid. Conservative substitution tables providing functionally similar amino acids are well known in the art. According to the present invention, modified variants of the peptide hormones of the invention functionally retain their specific hormone activity when analyzed in a suitable model to determine said activity.

As used herein the term “peptide hormone” refers to any protein or peptide with hormonal activity, i.e. a peptide with signaling activity through the binding on its cognate receptor or to the class of peptide hormones involved in neuronal signaling, such as involved in a wide range of brain functions, including analgesia, reward, food intake, metabolism, reproduction, social behaviors, learning and memory. A peptide hormone may also be referred to as a neuropeptide.

The term “O-linked glycan” as used herein refers to the O-linked glycosylation with the addition of N-acetyl-galactosamine (GalNAc) to serine or threonine residues in the peptide hormones of the invention followed by other carbohydrates (such as galactose and sialic acid).

The phrase “at a predetermined specific site, such as in the receptor-binding region” as used herein refers to the addition or selection of peptides with an O-linked glycan at a site specifically selected.

The phrase “a truncated version or a variant as compared to the corresponding wild-type peptide hormone found in nature” is intended to refer to a peptide hormone which has been modified by either truncation or amino acid substitutions as compared with the same peptide hormone found in nature, such as found in vivo in the human body. Typically, the peptide hormone may be genetically engineered and/or chemically synthesized to include 1, 2, 3, 4, 5, 6, or 7 amino acids of the native peptide sequence being substituted with any other amino acid, such as conservative substitutions. Alternatively or in addition to this, 1, 2, 3, 4, 5, 6, or 7 amino acids may have been removed or added to the native peptide sequence. Accordingly, in some embodiments, the peptides hormone according to the present invention comprises 1, 2, 3, 4, 5, 6, or 7 substitutions, additions or deletions relative to the native wild-type peptide hormone. The amino acids used in the amino acid sequences according to the invention may be in both L- and/or D-form. It is to be understood that both L- and D-forms may be used for different amino acids within the same peptide sequence. In some embodiments the amino acids within the peptide sequence are in L-form, such as natural amino acids.

The term “improved stability” as used herein refers to peptides of the invention, which when tested e.g. in an in vitro assays as described in⁷⁰ exhibit improved stability as compared to the same peptide without this one or more O-linked glycan at a predetermined specific site, such as in the receptor-binding region.

The term “receptor sub-type selectivity switch” as used herein refers to peptides of the invention being receptor sub-type specific.

The term “O-linked glycan” or “O-glycosylation” refers to the attachment of a sugar molecule to an oxygen atom in an amino acid residue in a protein.

### Specific Embodiments of the Invention

1. A formulation comprising at least one molecule of a peptide hormone species exhibiting a specific glycosylation pattern of one or more O-linked glycan(s) at a predetermined specific site of said peptide hormone species, wherein specific, defined glycosylation pattern means that the each molecule of said peptide hormone in said pharmaceutical formulation displays structural homogeneity with respect to the site of glycan attachment and/or with respect to the glycan attachment.

2. A formulation comprising at least one molecule of a peptide hormone species according to embodiment 1, wherein said peptide hormone species is selected from the group of sequences comprising SEQ ID NOs: 1, 2, 3, 5, 6, 7, 14, 15, 16, 21, 22, 23, 24, 25, 36, 37, 38, 39, 40, 41, 42, 43, 45, 46, 47, 48, 49, 51, 52, 54, 55, 56, 57, 72, 73, 74, 76, 79, 8, 81, 83, 84, 91, 92, 93, 94, 95, 96, 97, 98, 99, 100, 101, 102, 106, 107, 108, 109, 113, 116, 117, 118, 119, 120, 130, 131, 135, 136, 143, 144, 147, 163, 164, 167, 168, 170, 184, 185, 186, 188, 189, 190, 191, 192, 204, 215, 217, 219, 222, 227, 229, 230, 231, 233, 234, 236, 252, 260, 262, 267, 272, and/or 279.

3. A formulation comprising at least one molecule of a peptide hormone species according to embodiment 1 or 2, wherein said peptide hormone species is selected from the group of sequences comprising SEQ ID NOs: 6, 7, 21, 22, 23, 24, 72, 74, 92, 95, 97, 99, 106, 107, 108, 116, 117, 118, 147, 163, 167, 185, 186, and/or 188.

4. A formulation comprising at least one molecule of a peptide hormone species according to any one of embodiments 1 to 3, wherein said peptide hormone species is selected from the group of sequences comprising SEQ ID NOs: 72, 95, 97, 106, 108, 147, 185, and/or 188.

5. A formulation comprising at least one molecule of a peptide hormone species according to any one of embodiments 1 to 4, wherein said peptide hormone species is SEQ ID NO: 147.

6. A formulation comprising at least one molecule of a peptide hormone species according to any one of embodiments 1 to 5, wherein said predetermined specific site of said peptide hormone is within the receptor-binding region.

7. A formulation comprising at least one molecule of a peptide hormone species according to any one of embodiments 1 to 6, wherein said peptide hormone exhibiting an specific glycosylation pattern of one or more O-linked glycan(s) at a predetermined specific site of said peptide hormone is obtainable by recombinant production using a host cell in which one or more glycosyltransferase gene(s) is/are inactivated or downregulated by inactivation and/or downregulation of one or more gene(s) selected from COSMC and C1GALT1; and/or by inactivation and/or downregulation of one or more gene(s) selected from GCNT3, GCNT4, B3GNT6, and/or by inactivation and/or downregulation and/or upregulation/activation of one or more gene(s) selected from ST6GALNAC1-6, ST3GAL1, GCNT3, GCNT4, and/or B3GNT6.

8. A formulation comprising comprising at least one molecule of a peptide hormone species according to any of embodiments 1 to 7, wherein said peptide hormone species exhibits a specific glycosylation pattern of one or more O-linked glycan(s) at a predetermined specific site of said peptide hormone and, wherein the one or more O-glycan structures include a glycan structure selected from a Core1, core2, core3, or core4 structure with optional elongation and sialic acid capping, wherein optionally the first monosaccharide attached in the synthesis of O-linked glycans is N-acetyl-galactosamine and wherein a core1 structure may be obtained by the addition of galactose, and wherein a core2 structure may be obtained by the addition of N-acetyl-glucosamine to the N-acetyl-galactosamine of the core1 structure and wherein the core3 structures may be obtained by the addition of a single N-acetyl-glucosamine to the first monosaccharide N-acetyl-galactosamin and core4 structures may be obtained by the addition of a second N-acetyl-glucosamine to the core3 structure.

9. A formulation comprising at least one molecule of a peptide hormone species according to any of embodiments 1 to 8, wherein the one or more O-glycan structures include a Tn (GalNAc) structure.

10. A formulation comprising at least one molecule of a peptide hormone species according to any of embodiments 1 to 9, wherein the one or more O-glycan structures include Tn (GalNAc) structure with one sialic acid capping (alpha2-6).

11. A formulation comprising at least one molecule of a peptide hormone species according to any of embodiments 1 to 10, wherein the one or more O-glycan structures include the core1 structures with one sialic acid capping (alpha2-6).

12. A formulation comprising at least one molecule of a peptide hormone species according to any of embodiments 1 to 11, wherein the one or more O-glycan structures include the core1 structures with one sialic acid capping (alpha 2-3)

13. A formulation comprising at least one molecule of a peptide hormone species according to any of embodiments 1 to 12, wherein the one or more O-glycan structures include the Core1 structures with two sialic acids capping (alpha 2-3 and alpha 2-6).

14. A formulation according to any one of embodiments 1 to 13, wherein said formulation is a pharmaceutical formulation.

15. A formulation comprising at least one molecule of a peptide hormone species as defined in any of the preceding embodiments 1 to 14, wherein said peptide hormone species comprises one or more O-linked glycan at a site indicated in and one of Tables 6A to 6E, particularly, wherein the site is a site in a human peptide hormone, more particularly, wherein the site is a conserved site in a human peptide hormone.

16. A formulation comprising at least one molecule of a peptide hormone species as defined in embodiment 15, wherein said peptide hormone species comprises one or more O-linked glycan at a site indicated in Table 6A or 6B, particularly, wherein the site is a site in a human peptide hormone, more particularly, wherein the site is a conserved site in a human peptide hormone.

17. A formulation comprising at least one molecule of a peptide hormone species as defined in embodiments 15 or 16, wherein said peptide hormone species comprises one or more O-linked glycan at a site of a peptide hormone indicated in Table 6B, particularly, wherein the site is a site in a human peptide hormone, more particularly, wherein the site is a conserved site in a human peptide hormone.

18. A formulation comprising at least one molecule of a peptide hormone species as defined in any one of embodiments 15 to 17, wherein said peptide hormone species comprises one or more O-linked glycan at a site of a peptide hormone indicated in Table 6C, particularly, wherein the site is a site in a human peptide hormone, more particularly, wherein the site is a conserved site in a human peptide hormone.

19. A formulation comprising at least one molecule of a peptide hormone species as defined in any one of embodiments 15 to 17, wherein said peptide hormone species comprises one or more O-linked glycan at a site of a peptide hormone indicated in Table 6C, particularly, wherein the site is a site in a human peptide hormone, more particularly, wherein the site is a conserved site in a human peptide hormone.

20. A formulation comprising at least one molecule of a peptide hormone species as defined in any one of embodiments 15 to 17, wherein said peptide hormone species comprises one or more O-linked glycan at a site of a peptide hormone indicated in Table 6D, particularly, wherein the site is a site in a human peptide hormone, more particularly, wherein the site is a conserved site in a human peptide hormone.

21. A modified peptide hormone comprising one or more O-linked glycan at a predetermined specific site selected from the group comprising the peptide hormones indicated in 6A, particularly, wherein the site is a site in a human peptide hormone, more particularly, wherein the site is a conserved site in a human peptide hormone, and wherein said peptide hormone is selected from the group comprising SEQ ID Nos: 1, 2, 3, 5, 6, 7, 14, 15, 16, 21, 22, 23, 24, 25, 36, 37, 38, 39, 40, 41, 42, 43, 45, 46, 47, 48, 49, 51, 52, 54, 55, 56, 57, 72, 73, 74, 76, 79, 8, 81, 83, 84, 91, 92, 93, 94, 95, 96, 97, 98, 99, 100, 101, 102, 106, 107, 108, 109, 113, 116, 117, 118, 119, 120, 130, 131, 135, 136, 143, 144, 147, 163, 164, 167, 168, 170, 184, 185, 186, 188, 189, 190, 191, 192, 204, 215, 217, 219, 222, 227, 229, 230, 231, 233, 234, 236, 252, 260, 262, 267, 272, and/or 279.

22. The modified peptide hormone according to embodiment 21 comprising one or more O-linked glycan at a predetermined specific site selected from the group comprising the peptide hormones indicated in 6B, particularly, wherein the site is a site in a human peptide hormone, more particularly, wherein the site is a conserved site in a human peptide hormone, and wherein said peptide hormone is selected from the group comprising SEQ ID Nos: 6, 7, 21, 22, 23, 24, 72, 74, 92, 95, 97, 99, 106, 107, 108, 116, 117, 118, 147, 163, 167, 185, 186, and/or 188.

23. The modified peptide hormone according to embodiments 21 or 22 comprising one or more O-linked glycan at a predetermined specific site selected from the group comprising the peptide hormones indicated in 6C, particularly, wherein the site is a site in a human peptide hormone, more particularly, wherein the site is a conserved site in a human peptide hormone, and wherein said peptide hormone is selected from the group comprising SEQ ID Nos: 72, 95, 97, 106, 108, 147, 185, and/or 188.

24. The modified peptide hormone according to any one of embodiments 20 to 23 comprising one or more O-linked glycan at a predetermined specific site selected from the group comprising the peptide hormones indicated in 6D, particularly, wherein the site is a site in a human peptide hormone, more particularly, wherein the site is a conserved site in a human peptide hormone, and wherein said peptide hormone is depicted in SEQ ID NO: 147.

25. The modified peptide hormone according to any one of embodiments 20 to 24, wherein the one or more O-glycan structures include a glycan structure selected from a Core1, core2, core3, or core4 structure with sialic acid capping, wherein the one or more O-glycan structures include a glycan structure selected from a core1, core2, core3, or core4 structure with optional elongation and sialic acid capping, wherein optionally the first monosaccharide attached in the synthesis of O-linked glycans is N-acetyl-galactosamine and wherein a core1 structure may be obtained by the addition of galactose, and wherein a core2 structure may be obtained by the addition of N-acetyl-glucosamine to the N-acetyl-galactosamine of the core1 structure and wherein the core3 structures may be obtained by the addition of a single N-acetyl-glucosamine to the first monosaccharide N-acetyl-galactosamin and core4 structures may be obtained by the addition of a second N-acetyl-glucosamine to the core3 structure.

26. The modified peptide hormone according to any of embodiments 21 to 25, wherein the one or more O-glycan structures include a Tn (GalNAc) structure.

27. The modified peptide hormone according to any of embodiments 21 to 26, wherein the one or more O-glycan structures include Tn (GalNAc) structure with one sialic acid capping (alpha2-6).

28. The modified peptide hormone according to any of embodiments 21 to 27, wherein the one or more O-glycan structures include the core1 structures with one sialic acid capping (alpha2-6).

29. The modified peptide hormone according to any of embodiments 21 to 28, wherein the one or more O-glycan structures include the core1 structures with one sialic acid capping (alpha 2-3)

30. The modified peptide hormone according to any of embodiments 21 to 29, wherein the one or more O-glycan structures include the core1 structures with two sialic acids capping (alpha 2-3 and alpha 2-6).

The present invention relates also to an isolated peptide hormone, such as recombinant, such as a neuropeptide comprising one or more O-linked glycan at a predetermined specific site, such as in the receptor-binding region.

In some embodiments according to the present invention, the one or more O-glycan structures include a glycan structure selected from a core1, core2, core3, or core4 structure with sialic acid capping, such as a structure as illustrated in FIG. 1.

In some embodiments according to the present invention, the one or more O-glycan structures include a Tn (GalNAc) structure.

In some embodiments according to the present invention, the one or more O-glycan structures include Tn (GalNAc) structure with one sialic acid capping (alpha2-6).

In some embodiments according to the present invention, the one or more O-glycan structures include the core1 structures with one sialic acid capping (alpha2-6).

In some embodiments according to the present invention, the one or more O-glycan structures include the core1 structures with one sialic acid capping (alpha 2-3)

In some embodiments according to the present invention, the one or more O-glycan structures include the core1 structures with two sialic acids capping (alpha 2-3 and alpha 2-6).

In some embodiments according to the present invention, the peptide hormone has improved, such as increased stability and/or circulatory half-life and/or other pharmacokinetic properties, such as improved stability in in vitro assays, plasma and/or bodyfluids.

In some embodiments according to the present invention, the peptide hormone has lower bioactivity in receptor signalling, such as decreased receptor stimulation in in vitro cell assays and/or in man.

In some embodiments according to the present invention, the peptide hormone exhibits improved receptor stimulation in in vitro cell assays and/or in animal models and/or in man.

In some embodiments according to the present invention, the peptide hormone exhibits altered blood-brain barrier uptake in animals or in man, such as increased blood-brain barrier uptake in animals or in man, or decreased blood-brain barrier uptake in animals or in human.

In some embodiments according to the present invention, the peptide hormone exhibits receptor sub-type selectivity switch.

In some embodiments according to the present invention, the peptide hormone is specific to one or more tissue in human, such as specific to tissue of the nervous system.

In some embodiments according to the present invention, the peptide hormone is selected from any one of tables 4, 5, or 6, such as selected from the list consisting of a peptide of the Neuropeptide Y family, such as NPY, PPY and PYY; a peptide of the Glucagon/Secretin family, such as GIP, Glucagon, GLP-1, GLP-2, PACAP, Secretin, PHM-27/PHV-42, Somatoliberin and VIP; a peptide of the Natriuretic peptide family, such as ANP, BNP and CNP, a peptide of the calcitonin family, such as calcitonin, and a peptide of the insulin family such as amylin.

In some embodiments according to the present invention, the peptide hormone is not found in nature. Accordingly, in some embodiments the peptide hormone according to the invention is not a wild-type hormone found in any species in nature. The peptide hormone according to the invention may be a peptide hormone that is a variant of a wild-type peptide hormone. Such peptide hormone variant may be a peptide that differ from the wild-type version by 1, 2, 3, 4, 5, 6, 7, 8, 9, 10, 11, 12, 13, 14, 15, 16, 17, 18, 19, or 20 amino acid differences, such as amino acid substitutions, additions or deletions. A peptide variant according to the invention may have more than 80%, such as more than 85, 90, 91, 92, 93, 94, 95, 96, 97, 98, or 99% sequence identity with a corresponding wild-type peptide hormone found in nature.

In some embodiments according to the present invention, the peptide hormone is a truncated version or a variant as compared to the corresponding wild-type peptide hormone found in nature.

In some embodiments according to the present invention, the peptide hormone is selected from any one of table 6 comprising one or more O-linked glycan at a site as indicated in table 6, such as at a bold underlined position and/or an italic underlined position.

In some embodiments according to the present invention, the peptide hormone is selected from any one of table 6 comprising at least, not more than, or the exact number of O-linked glycan sites as indicated in table 6.

In some embodiments according to the present invention, the peptide hormone is selected from any one of table 5.

Subject matter of the present invention is also a (pharmaceutical or diagnostic) composition or formulation comprising a compound as defined in any of the preceding embodiments.

Subject matter of the present invention is also a (pharmaceutical or diagnostic) composition or formulation comprising a synthetic compound as defined in any of the preceding embodiments, wherein said (pharmaceutical or diagnostic) composition or formulation is suitable for administration to a patient in need thereof.

Subject matter of the present invention is also a peptide hormone or a pharmaceutical composition or formulation according to any of the above embodiments, wherein said composition or formulation is suitable for the localized or systemic administration, wherein the localized administration is preferably selected from the group of topical administration, including transdermal, ophthalmic, nasal, otologic, enteral, pulmonal and urogenital administration or local or systemic injection, including subcutaneous, intra-articular, intravenous, intracardiac, intramuscular, intraosseous or intraperitoneal administration.

Subject matter of the present invention is also a device according to the previous embodiment, wherein the device is suitable as a delivery system for immediate and/or sustained release of a peptide hormone as defined in anyone of the preceding embodiments, e.g., they may be used as drug (peptide hormone) delivery system or controlled drug (peptide hormone) release systems.

Subject-matter of the invention is also a device comprising a pharmaceutical composition or a pharmaceutical formulation as defined in any of the foregoing embodiments. A device may take any form that is suitable to deliver the synthetic compounds or any one of the compositions or formulations of the present invention. It may comprise biological and/or synthetic materials and may take form of a patch, a stent, an implantable device, hydrogel, etc.

Subject-matter of the invention is also a device as defined in any of the foregoing embodiments, wherein the device is a delivery system for immediate and/or sustained release of the peptide hormone as defined in any of the foregoing embodiments.

Subject-matter of the invention is also a method of treatment of an individual in need thereof and/or the amelioration of and/or the prevention of deterioration of a disease in an individual in need thereof, by administration to said individual of a therapeutically efficient amount of any of the peptide hormones according to the present invention and/or pharmaceutical compositions as defined above.

The administration of the compounds (peptide hormones) according to this invention and pharmaceutical compositions according to the invention may be performed in any of the generally accepted modes of administration available in the art. Illustrative examples of suitable modes of administration include intravenous, oral, nasal, inhalable, parenteral, topical, transdermal and rectal delivery. Parenteral and intravenous delivery forms are preferred. In aspects of the invention injectable formulations comprising a therapeutically effective amount of the compounds (peptide hormones) of the invention are provided, including salts, esters, isomers, solvates, hydrates and polymorphs thereof, at least one vehicle comprising water, aqueous solvents, organic solvents, hydro-alcoholic solvents, oily substances, or mixtures thereof, and optionally one or more pharmaceutically acceptable excipients. Standard knowledge regarding these pharmaceutical ingredients and pharmaceutical formulations/compositions may be found, inter alia, in the ‘Handbook of Pharmaceutical Excipients’; Edited by Raymond C Rowe, Paul J Sheskey, Walter G Cook and Marian E Fenton; May 2012 and/or in Remington: The Science and Practice of Pharmacy, 19th edition. The pharmaceutical compositions/formulations may be formulated in the form of a dosage form fororal, intravenous, nasal, inhalable, parenteral, topical, transdermal and rectal and may thus comprise further pharmaceutically acceptable excipients, such as buffers, solvents, preservatives, disintegrants, stabilizers, carriers, diluents, fillers, binders, lubricants, glidants, colorants, pigments, taste masking agents, sweeteners, flavorants, plasticizers, and any acceptable auxiliary substances such as absorption enhancers, penetration enhancers, surfactants, co-surfactants, and specialized oils.

The proper excipient(s) is (are) selected based in part on the dosage form, the intended mode of administration, the intended release rate, and manufacturing reliability. Examples of common types of excipients include also various polymers, waxes, calcium phosphates, sugars, etc.

Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; polyalkylene glycols such as polyethylene glycol and polypropylene glycol; and polyacrylic acids including their copolymers and crosslinked polymers thereof, e.g., Eudragit® (Rohm), polycarbophil, and chitosan polymers. Waxes include white beeswax, microcrystalline wax, carnauba wax, hydrogenated castor oil, glyceryl behenate, glycerylpalmitol stearate, and saturated polyglycolyzed glycerate. Calcium phosphates include dibasic calcium phosphate, anhydrous dibasic calcium phosphate, and tribasic calcium phosphate. Sugars include simple sugars, such as lactose, maltose, mannitol, fructose, sorbitol, saccharose, xylitol, isomaltose, and glucose, as well as complex sugars (polysaccharides), such as maltodextrin, amylodextrin, starches, and modified starches.

The pharmaceutical compositions/formulations of the present invention may be formulated into various types of dosage forms, for instance as solutions or suspensions, or as tablets, capsules, granules, pellets or sachets for oral administration.

The pharmaceutical composition of the present invention can be manufactured according to standard methods known in the art. Granulates according to the invention can be obtained by dry compaction or wet granulation. These granulates can subsequently be mixed with e.g. suitable disintegrating agents, glidants and lubricants and the mixture can be compressed into tablets or filled into sachets or capsules of suitable size. Tablets can also be obtained by direct compression of a suitable powder mixture, i.e. without any preceding granulation of the excipients. Suitable powder or granulate mixtures according to the invention are also obtainable by spray drying, lyophilization, melt extrusion, pellet layering, coating of the active pharmaceutical ingredient or any other suitable method. The so obtained powders or granulates can be mixed with one or more suitable ingredients and the resulting mixtures can be delivered in sterile primary packaging devices for reconstitution before parenteral administration Injectable compositions of the present invention may contain a buffer (for example, sodium dihydrogen phosphate, disodium hydrogen phosphate and the like), an isotonizing agent (for example, glucose, sodium chloride and the like), a stabilizer (for example, sodium hydrogen sulfite and the like), a soothing agent (for example, glucose, benzyl alcohol, mepivacaine hydrochloride, xylocaine hydrochloride, procaine hydrochloride, carbocaine hydrochloride and the like), a preservative (for example, p-oxybenzoic acid ester such as methyl p-oxybenzoate and the like, thimerosal, chlorobutanol, benzyl alcohol and the like) and the like, if necessary. In addition, the injectable composition of the present invention may contain vitamins and the like. Further, injectable compositions of the present invention may contain an aqueous solvent, if necessary. Examples of the aqueous solvent include purified water for injection, physiological saline solution, and glucose solution. In injectable compositions of the present invention, the pharmaceutical compound (peptide hormone) may be solid. As used herein, the “solid” comprises crystals and amorphous substances which have conventional meanings. The form of the solid component is not particularly limited, but powder is preferred in view of dissolution rate.

### Pharmaceutical Formulations

Still another aspect of the present invention relates to the use of the peptide hormones according to the present invention, e.g. as shown in Tables 6A-E) as an active ingredient, together with at least one pharmaceutically acceptable carrier, excipient and/or diluents for the manufacture of a pharmaceutical composition for the treatment and/or prophylaxis of appropriate disorders or diseases.

Administration forms include, for example, pills, tablets, film tablets, coated tablets, capsules, liposomal formulations, micro- and nano-formulations, powders and deposits. Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain the compounds according to the present invention.

The compounds of the invention can also be administered in form of its pharmaceutically active salts. Suitable pharmaceutically active salts comprise acid addition salts and alkali or earth alkali salts. For instance, sodium, potassium, lithium, magnesium or calcium salts can be obtained.

The pharmaceutical compositions/formulations according to the present invention will typically be administered together with suitable carrier materials selected with respect to the intended form of administration, i.e. for oral administration in the form of tablets, capsules (either solid filled, semi-solid filled or liquid filled), powders for constitution, aerosol preparations consistent with conventional pharmaceutical practices. Other suitable formulations are hydrogels, elixirs, dispersible granules, syrups, suspensions, creams, lotions, solutions, emulsions, suspensions, dispersions, and the like. Suitable dosage forms for sustained release include tablets having layers of varying disintegration rates or controlled release polymeric matrices delivered with the active components. The pharmaceutical compositions may be comprised of 0.01 to 95% by weight of the peptide hormones of the invention.

As pharmaceutically acceptable carrier, excipient and/or diluents can be used HSA, lactose, sucrose, cellulose, mannitol.

Suitable binders include starch, gelatin, natural sugars, corn sweeteners, natural and synthetic gums such as acacia, sodium alginate, carboxymethyl-cellulose, polyethylene glycol and waxes. Among the lubricants that may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below. Additionally, the compositions of the present invention may be formulated in sustained release form to provide the rate controlled release of any one or more of the components or active ingredients to optimize the therapeutic effects. Suitable dosage forms for sustained release include controlled release polymeric matrices or hydrogels embedding the active components. Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for parenteral administration. Such liquid forms include solutions, suspensions and emulsions.

The compounds of the present invention may also be deliverable transdermally. The transdermal compositions may take the form of creams, lotions, aerosols and/or emulsions and can be included in a transdermal patch of the matrix or reservoir type as are conventional in the art for this purpose.

The transdermal formulation of the compounds of the invention is understood to increase the bioavailability of said compound into the circulating blood. One problem in the administration of peptidic drugs in general is the loss of bioactivity due to the formation of insolubles in aqueous environments or due to degradation. Therefore, stabilization of compounds for maintaining their fluidity and maintaining their biological activity upon administration to the patients in need thereof needs to be achieved. Prior efforts to provide active agents for medication include incorporating the medication in a polymeric matrix whereby the active ingredient is released into the systemic circulation. Known sustained-release delivery means of active agents are disclosed, for example, in U.S. Pat. Nos. 4,235,988, 4,188,373, 4,100,271, US447471, U.S. Pat. Nos. 4,474,752, 4,474,753, or U.S. Pat. No. 4,478,822 relating to polymeric pharmaceutical vehicles for delivery of pharmaceutically active chemical materials to mucous membranes. The pharmaceutical carriers are aqueous solutions of certain polyoxyethylene-polyoxypropylene condensates. These polymeric pharmaceutical vehicles are described as providing for increased drug absorption by the mucous membrane and prolonged drug action by a factor of two or more. The substituents are block copolymers of polyoxypropylene and polyoxyethylene used for stabilization of drugs such as insulin.

Aqueous solutions of polyoxyethylene-polyoxypropylene block copolymers (poloxamers) are useful as stabilizers for the compounds. Aside from serving as a stabilizer for the compound, poloxamers provide excellent vehicles for the delivery of the compound, and they are physiologically acceptable. Poloxamers, also known by the trade name Pluronics (e.g. Pluronic F127, Pluronic P85, Pluronic F68) have surfactant properties that make them useful in industrial applications. Among other things, they can be used to increase the water solubility of hydrophobic, oily substances or otherwise increase the miscibility of two substances with different hydrophobicities. For this reason, these polymers are commonly used in industrial applications, cosmetics, and pharmaceuticals. They have also been used as model systems for drug delivery applications. In situ gelation of pharmaceutical compositions based on poloxamer that are biologically triggered are known in the art (e.g. U.S. Pat. No. 5,256,396), describing compositions containing poloxamer 407 and water at specified concentrations.

Gels refer to the active ingredients dispersed or solubilized in a hydrophilic semi-solid matrix. Powders for constitution refer to powder blends containing the active ingredients and suitable diluents which can be suspended in water and may contain optionally buffer salts, lactose, amino acids, excipients, sugars and isotonisation reagents.

Recently, increasingly improved and potent protein-based and peptide-based drugs have been developed by the biotech industry. However, the prophylactic and/or therapeutic use of many other protein- or peptide-based compounds has been hampered because of their susceptibility to proteolytic breakdown, rapid plasma clearance, peculiar dose-response curves, immunogenicity, bioincompatibility, and/or the tendency of peptides and proteins to undergo aggregation, adsorption, and/or denaturation. These characteristics often render traditional methods of drug delivery ineffective or sub-optimal when applied to protein or peptide based drugs. Therefore, an immense amount of interest has been increasingly placed on controlled and/or sustained release drug delivery systems to maintain the therapeutic efficacy or diagnostic value of these important classes of biologically active agents. One of the primary goals of sustained delivery systems is to maintain the levels of an active agent within an effective range and ideally at a constant level. One approach for sustained delivery of an active agent is by microencapsulation, in which the active agent is enclosed within a polymeric matrix. The importance of biocompatible and/or biodegradable polymers as carriers for parenteral drug delivery systems is now well established. Biocompatible, biodegradable, and relatively inert substances such as poly(lactide) (PLA) or poly(lactide-co-glycolide) (PLG) structures such as microparticles or films containing the active agent to be administered are commonly employed sustained delivery devices (for review, see M. Chasin, Biodegradable polymers for controlled drug delivery. In: J.O. Hollinger Editor, Biomedical Applications of Synthetic Biodegradable Polymers CRC, Boca Raton, Fla. (1995), pp. 1-15; T. Hayashi, Biodegradable polymers for biomedical uses. Prog. Polym. Sci. 19 4 (1994), pp. 663-700; and Harjit Tamber, Pal Johansen, Hans P. Merkle and Bruno Gander, Formulation aspects of biodegradable polymeric microspheres for antigen delivery Advanced Drug Delivery Reviews, Volume 57, Issue 3, 10 Jan. 2005, Pages 357-376). A relatively steady release of one or more active agents incorporated within such polymers is possible because of the degradation profile of these polymers in an aqueous environment. By encapsulating active agents in a polymer matrix in various forms such as microparticles and/or films the active agent is released at a relatively slow rate over a prolonged time. Achieving sustained drug release in such a manner may afford less frequent administration, thereby increasing patient compliance and reducing discomfort; protection of the therapeutic compound within the body; potentially optimized prophylactic or therapeutic responses and prolonged efficacy; and avoidance of peak-related side-effects by maintaining more-constant blood levels of the active agent. Furthermore, these compositions can oftentimes be administered by injection, allowing for localized delivery and high local concentrations of the active agents.

With regard to highly active biologies, such as growth factors, local in the form of a bolus injection results in rapid diffusion from the region of interest and can cause severe side effects and limit efficacy. The oldest way is to use biophysical retention by changing the biophysical properties in form of viscosity, porosity, hydrophobicity or charge of the material to attain a purposeful delivery. This strategy often substantially modifies the properties of the tissue and conditions for cells, requiring more appropriate, biocompatible release mechanisms.

### Methods of Treatment

Treatment methods of the invention comprise the step of administering to a subject a therapeutically effective amount of at least one peptide hormone according to the invention or a pharmaceutical composition/formulation of the invention. The administration may be effected by any route, e.g., dermally, parenterally, topically, etc.

As indicated previously “therapeutically effective amount” of a at least one peptide hormone according to the invention preferably refers to the amount necessary to achieve the therapeutic outcome.

The choice of the optimal dosage regime and duration of medication, particularly the optimal dose and manner of administration of the active compounds necessary in each case can be determined by a person skilled in the art on the basis of his/her expert knowledge.

Subject matter of the present invention is also any of the above the above at least one peptide hormone in method of manufacturing a medicament for the treatment of an appropriate condition or diseases.

### EXAMPLES

### Example 1

### Development of a Sensitive O-Glycoproteomics Work Flow Enriching for Peptide Hormones

The present inventors originally developed the so-called SimpleCell O-glycoproteomics strategy^{18,71}, and provided a vast expansion of the knowledge of the human O-glycoproteome. While more than 3,000 O-glycosites were identified in almost 1,000 human proteins^{28,71}. In order to specifically explore potential O-glycans on peptide hormones the present inventors developed a novel proteomics based strategy selective for smaller peptides. The overall strategy for exploring O-glycosylation of peptide hormones is presented in FIG. 2A. The present inventors chose to use the strategy on cells and organs known to produce and secrete high levels of diverse peptide hormones. For tissues the present inventors selected the whole-brain and cerebellum from both rat and pig as neuronal sources, porcine pancreas, ileum and heart as endocrine sources. For human samples, prostate cancer was chosen as well as plasma since both endocrine and neuroendocrine tissues secrete peptide hormones into the blood stream. For cell lines, the present inventors chose the mouse neuroblastoma cell line N2A and the mouse enteroendocrine STC1 cell line that is a natural source of gut hormones.

Peptide hormones are typically short peptides of 30-50 amino acids, and to achieve optimal peptide hormone coverage in the mass spectrometry analysis, the present inventors used different pre-extraction methods for the tissue- and plasma samples (precipitation with organic solvents or acidic water extraction) to ensure enrichment of shorter peptides. For the cell lines, the present inventors furthermore used different proteolytic enzymes (trypsin, Glu-C and/or chymotrypsin) for protein digestion to facilitate better coverage of proteins in the LWAC-LC/MS workflow (FIG. 1N).

When studying protein O-glycosylation in complex mixtures enrichment of glycopeptides is essential for glycan detection due to suppression of glycopeptide ionization by the presence of large amounts of unglycosylated peptides⁷². In the case of N2A cells expressing truncated O-glycans due to a spontaneous mutation in the COSMC gene⁷³, the Tn (GalNAc) binding VVA (vicia villosa lectin) was used as previously described. Normal cells generally produce elongated O-glycans of core 1 or in some cases cores 2-4 structures with capping sialic acids (FIG. 1), and the majority of O-glycoproteins found in plasma carry the sialylated Core 1 structures (Core 1)^{12,74}. The present inventors therefore hypothesized that secreted and circulating peptide hormones would carry similar O-glycan structures if they were O-glycosylated, and based on this the present inventors designed an enrichment strategy that depended on initial neuraminidase treatment of plasma, tissue extracts and cell pellets to remove capping sialic acids followed by protease digestion and LWAC enrichment using the lectins PNA (peanut agglutinin) and/or Jacalin that recognize and bind Core 1 O-glycans as previously described^{11,12}. Following lectin enrichment, glycopeptides were fragmented and sequenced using HCD and ETD LC-MS/MS (FIG. 2A).

### Example 2

### Identification of O-Glycans on Peptide Hormones

In total the present inventors identified 2,327 O-glycoproteins (199 from human, 898 from pig, 509 from rat and 721 from mouse) with approximately 5,000 unambiguously assigned sites and another approximately 8,000 ambiguously assigned modification specific O-glycopeptides where site information was not obtained due to poor quality spectra or lack of ETD data from 10 different tissues and multiple species (FIG. 2A).

In order to extract information on glycosylated peptide hormones across several species (see FIG. 2A right panel for schematic summary), the sequenced glycopeptide fragments were aligned to the 104 human proproteins annotated in the most comprehensive available database of both neuropeptides and peptide hormones (NeuroPeP)⁷⁵. This analysis resulted in 6347 glycopeptide fragments from 135 orthologous proproteins.

To further explore the total number of O-glycosylated peptide hormones across the mammalian species analysed, the present inventors realigned the resulting peptide fragments to the corresponding human homolog resulting in 62 preproproteins (FIG. 2B). Subsequently, mapping the identified sites onto the mature peptide hormones demonstrated that 92 out of the 279 annotated mature human peptide hormones carried O-glycans at one of more sites (Table 6 and FIG. 2B). A minor fraction of these proteins and glycosites were reported previously, and the data thus confirmed the presence of known O-glycans on 17 preproproteins and 33 mature peptide hormones with the vast majority belonging to the well-described chromogranin and SAAS families^{9-14,54-56,76-79}. The majority of the identified O-glycoproteins were novel and included 45 preproproteins and 59 mature peptide hormones (FIG. 2B). Among these were well-characterized peptide hormones including GLP-1, insulin, cholecystokinin, PYY, galanin and secretin, which unexpectedly were found to carry O-glycans.

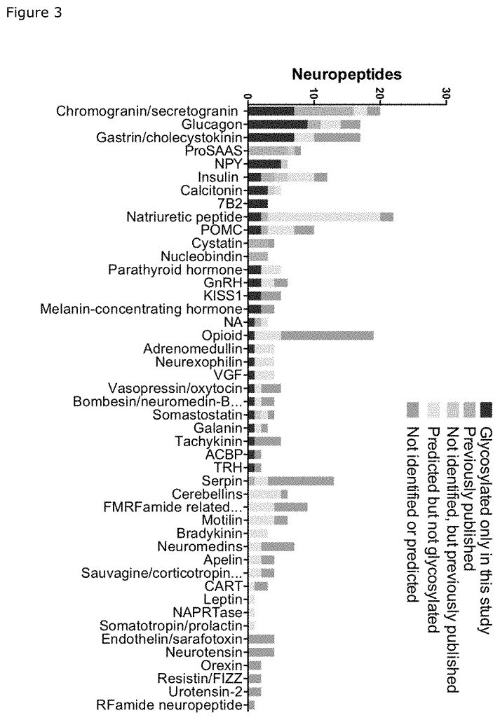

On average, our strategy resulted in the identification of 10-20 glycosylated peptide hormones per analyzed sample, with the exception of the plasma and brain samples that resulted in identification of 5 and 39 glycosylated peptide hormones, respectively (FIG. 2D). FIG. 3 presents a summary of glycosylated peptide hormones in the respective peptide hormone families. Out of 46 peptide hormone families (279 members) the present inventors found O-glycans in 29 families (92 members).

### Example 3

### O-Glycans Identified in Conserved Receptor Binding Domains of Peptide Hormone Families

PTMs are known to change the biochemical properties and diversify protein function. In particular O-glycosylation in close proximity to limited proteolytic cleavage sites has been demonstrated to e.g. co-regulate limited proteolytic processing. Therefore, the present inventors first explored if the identified sites were in close proximity (+/−3 a.a.) to physiological relevant cleavage sites in peptide hormones. However, mapping the identified sites relative to peptide hormone length revealed that this was not the case.

The major characterized structural or functional features of peptide hormones, besides limited proteolytic processing, are their ability to be recognized by and bind highly selective receptors. The present inventors therefore explored the positions of glycosites relative to characterized structural and/or functional receptor binding regions, and surprisingly, in six different peptide hormone families the majority of identified or predicted sites were indeed located in known receptor binding regions (FIG. 4A-F). Furthermore, evolutionary analysis by alignment of individual peptide hormones for each family revealed that clear conservation of the O-glycosites, both between members within families as well as through evolution of the individual peptide hormones (FIG. 4A-F), strongly suggesting that these O-glycosites have functionally and/or structurally importance.

The following details findings in each of the hormone families. Glycosylated residues are numbered according to the length of the mature processed bioactive peptide hormones:

### Example 4

### O-Glycans Identified in Conserved Receptor Binding Domains of the Secretin/Glucagon Family

The members of the Secretin/Glucagon family function in water homeostasis and regulation of feeding behavior and have remarkable sequence homology. Here the present inventors identified O-glycans on Secretin, Vasoactive Intestinal Peptide (VIP), Peptide Histidine Methionine/valine (PHM-27/PHV-42), Glucagon and Glucagon-like peptide 1(GLP-1), positioned in the N-terminal part of the peptide hormones, which has been shown to be important for receptor binding and activation⁸⁰ (FIG. 4A). While the present inventors did not identify glycans in all members of this family, the identified O-glycosite is fully conserved in Glucagon-like peptide 2 (GLP-2), Pituitary Adenylate Cyclase-activating Peptide (PACAP) and Somatoliberin, as well as partially in Gastric inhibitory peptide (GIP), where the predicted site is shifted 1 amino acid in the C-terminal direction. Furthermore, aligning all the members of the Secretin/Glucagon family demonstrated a highly conserved sequence motif Phe-Thr-Ser/Asp as a common denominator for glycosylation where both Thr and Ser are possible acceptor sites for glycosylation (FIG. 4A).

### Example 5

### O-Glycans Identified in Conserved Receptor Binding Domains of the Calcitonin Family

Members of the Calcitonin control a number of processes, including calcium/phosphate balance (Calcitonin), insulin dependent glucose metabolism (Amylin/IAPP) and vasodilation (Adrenomedullin and Intermedin)⁸¹. The present inventors identified O-glycans on all members of the Calcitonin family with the exception of Intermedin (FIG. 4B). Similar to the Secretin/Glucagon family, the present inventors identified a possible common sequence motif in the small conserved disulfide loop C(x)xxxTC for glycosylation of members of the Calcitonin family, where two cysteines are spaced by 4-5 amino acids including the Thr acceptor. This conserved Thr residue (Thr5 in Calcitonin) the present inventors found glycosylated in Amylin located in the disulfide ring structure, which is essential for receptor binding⁸². Moreover, in a study with artificial O-glycans it was shown that O-glycans may alter the alpha-helical structure of the Calcitonin peptide⁸³.

### Example 6

### O-Glycans Identified in Conserved Receptor Binding Domains of the Insulin-Like Growth Factor Family

In the IGF/Insulin subfamily of the Insulin gene superfamily, the present inventors identified an O-glycan on Insulin in the B-chain at Thr27 in a semi-conserved residue found as a serine in Insulin Growth Factor II (IGFII) and a threonine shifted a few positions C-terminally in Insulin Growth Factor I (IGFI) (FIG. 4C). This is surprising as Insulin is one of the most well-studied polypeptides, and the glycan identified in the sequence ⁴⁷GFFYTPKA⁵⁴ (HexHexNAc) was consistently found in 2 different species tested.

### Example 7

### O-Glycans Identified in Conserved Receptor Binding Domains of the Galanin Family

Galanin and Galanin-like peptide have multiple functions stimulating smooth muscle cell contraction and growth hormone and insulin release. On Galanin itself the present inventors identified an O-glycan on Thr3, which is an essential residue for receptor activation⁸⁴ and conserved in both members. In addition to this the present inventors found an O-glycan on Ser11, which is only present in Galanin and not the other family member Galanin-like peptide (FIG. 4D).

### Example 8

### O-Glycans Identified in Conserved Receptor Binding Domains of the Neuropeptide Y Family

The Neuropeptide Y family members, Neuropeptide Y (NPY), Peptide YY (PYY) and Pancreatic Polypeptide (PPY) share structural features and they all adopt a specific three-dimensional structure called the PP-fold. The peptides are involved in appetite regulation and anxious behavior, and the present inventors found all three members to carry O-glycans at the same conserved C-terminal Thr32 residue (FIG. 4E). The present inventors identified an additional N-terminal glycosylation site on NPY (Ser3) that was not conserved in the other members of the family. The C-terminal region of the NPY family members is essential for receptor binding, receptor selectivity and activation but also the mid-region and N-terminal has been shown to be important for receptor interaction⁸⁵.

### Example 9

### O-Glycans Identified in Conserved Receptor Binding Domains of the Natriuretic Peptides Family

The present inventors identified O-glycans on all three precursors (pro Atrial Natriuretic peptide (ANP), pro B-type Natriuretic Peptide (BNP), pro C-type Natriuretic Peptide (CNP)) as previously described for proBNP⁷⁹. However, the present inventors also identified O-glycans at Ser19 and Ser25 in the C-terminal cyclic receptor-binding region of mature ANP where Ser19 is highly conserved in all three members (FIG. 4F).

O-glycans on peptide hormones modulate receptor activation.

Considering the high degree of conservation and structural position of the identified O-glycans the present inventors next decided to explore the potential functional impact of site-specific O-glycosylation on mature peptide hormones. The present inventors selected Glucagon, GLP-1, Secretin, VIP, ANP, NPY, PYY and PPY as examples of peptide hormones where the present inventors identified O-glycosylation sites in known receptor activating regions for analysis in in vitro receptor binding assays. First, the present inventors used recombinant GalNAc-transferases for chemoenzymatic synthesis or chemically synthesized (SynPeptides, China) glycopeptide variants with Tn (GalNAcα1-O-Ser/Thr), elongated to T (Galβ1-3GalNAcα1-O-Ser/Thr) and sialylated ST (NeuAcα2-3Galβ1-3GalNAcα1-O-Ser/Thr) structures using recombinant purified glycosyl transferases. Secretin, Glucagon. GLP-1 and VIP were enzymatically GalNAc-glycosylated by GalNAc-T1 corresponding to position Thr7 (MS validated) in the mature peptide sequences and peptides from the NPY family were chemically synthesized with GalNAc at position Thr32. ANP was chemically synthesized with an O-glycan at position 19 and/or 25. Next HEK293 or COS-7 cells transiently expressing selected relevant cognate receptors were incubated with increasing concentrations of peptide or glycopeptide. Ligand/agonist efficacy and potency was measured by receptor activation as an increase in secondary messenger cAMP.

Cell Culture and Transfection: HEK293 and COS7-cells were cultured in DMEM containing 10% FBS in a humidified atmosphere at 37° C. with 5% C02 (Sigma-Aldritch, Germany). For experiments, cells were seeded onto 6- or 10-cm plates and cultured for 1-3 days to 60-80% confluency. Cells were transfected with 1-2.5 pg of receptor constructs for 24-48 h using Lipofectamine 2000 (Thermo Fisher Scientific) according to the manufacturer's instructions, or alternatively, using linear 25-kDa polyethyleneimine (Polysciences). Both reagents were used at 1:3 DNA to reagent ratio.

cAMP and cGMP Accumulation Measured by HTRF®: Intracellular cAMP/cGMP levels were measured using a homogeneous time-resolved fluorescence (HTRF®) cAMP/cGMP Gs dynamic assay kit (CisBio Bioassays). Forty-eight hours post-transfection cells were detached and seeded into white 384-well microplates with 1,000 cells/well in 5 μl of stimulation buffer (DMEM, 1 mM 3-isobutyl-1-methylxanthine (IBMX), 0.2% BSA). For their stimulation, μl/well of the stimulation buffer containing appropriate doses of either naked peptide hormone or glycosylated variants were added. Then, cells were incubated for 30 min at 37° C. followed by lysis by addition of 5 μl/well of each of the supplied conjugate-lysis buffer containing d2-labeled cAMP/cGMP and Europium cryptate-labeled anti-cAMP/cGMP antibody, both reconstituted according to the manufacturer's instructions. Plates were incubated for 1 h in the dark at room temperature and time-resolved fluorescence signals were excited at 340 nm and measured at 620 and 665 nm, respectively using the EnSpire Multilabel Reader (PerkinElmer Life and Analytical Sciences). The cAMP/cGMP generated was interpolated from a cAMP standard curve generated in parallel for each experiment.

IP-1 Accumulation Measured by HTRF®: Intracellular IP-1 levels were measured, similarly to cAMP, using a homogeneous time-resolved fluorescence (HTRF®) IP-1 Gq assay kit (CisBio Bioassays). This assay is dependent on co-transfection of the receptor with a chimeric G-protein to obtain sufficient signal of the Gq pathway. In this assay NPY2R was used together with Gqo5. Forty-eight hours post-transfection cells were detached and seeded into white 384-well microplates with 10,000 cells/well in 7 μl of supplied stimulation buffer supplemented with 0.1% BSA. For stimulation, 7 μl/well of the supplemented stimulation buffer containing appropriate doses of either naked peptide hormone or glycosylated variants were added. Then, cells were incubated for 2 h at 37° C. followed by lysis by addition of 3 μl/well of each of the supplied conjugate-lysis buffer containing d2-labeled IP-1 and Europium cryptate-labeled anti-IP-1 antibody, both reconstituted according to the manufacturer's instructions. Plates were incubated for 1 h in the dark at room temperature and time-resolved fluorescence signals were excited at 340 nm and measured at 620 and 665 nm, respectively using the EnSpire Multilabel Reader (PerkinElmer Life and Analytical Sciences). The IP-1 generated was interpolated from a IP-1 standard curve generated in parallel for each experiment.

### Example 10

### O-Glycans on VIP Modulate Receptor Activation.

The two VIP/PACAP receptors (VPAC1 and VPAC2) show comparable affinities for VIP^{52,86}, thus the present inventors selected VPAC1 for analysis of VIP binding and activation. In this assay VIP exhibited a potency of 0.2 nM and 0.4 for VPAC1 & 2 respectively, which is in good agreement with previous studies. VIP with one O-glycan (GalNAc/Tn) at residue 7 (VIP-Thr7/Tn) showed a 581-fold decrease in potency to 102 nM for VPAC1 (FIG. 5A) and a 681-fold decrease to 242 nM for VPAC2 (FIG. 5B). Elongation of the O-glycan on VIP to T and ST structures changed the potency to 44 nM and 74 nM, respectively for VPAC1 and 130 and 244 nM respectively for VPAC2 (FIG. 8).

### Example 11

### O-Glycans on Secretin Modulate Receptor Activation.

Secretin binds and signals exclusively through the secretin receptor (SCTR), and in our assay secretin had a potency of 0.1 nM for SCTR, comparable to values found in previous studies, whereas secretin with a single O-glycan (GalNAc/Tn) at residue 7 (Secretin-Thr7-Tn) decreased the potency 2200-fold to 205 nM. Elongation of the glycan on secretin to T and ST structures further reduced potency 1-7 fold for each elongation step to 292 nM and 1932 nM, respectively (FIG. 5C).

### Example 12

### O-Glycans on GLP-1 Modulates Receptor Activation.

GLP-1 binds and signals exclusively through the GLP-1 receptor (GLP-1R), and in our assay GLP-1 showed a potency of 0.04 nM for GLP-1R, comparable to values found in previous studies. Elongation of the glycan at position 7 on GLP-1 to T, ST and diST (GLP1-Thr7/Tn/T/diST) further reduced potency approximately 20-40 fold fold for each elongation step to 253 nM, 266 nM, and 461 nM, respectively (FIG. 5D and FIG. 8).

### Example 13

### O-Glycans on Glucagon Modulates Receptor Activation.

As with GLP-1 and the GLP-1R, Glucagon binds and signals exclusively through the glucagon receptor (GCGR). The non-glycosylated glucagon showed a potency of 1.29 nM in line with previous literature. Upon introducing a Tn-glycoform at Thr7 (Glucagon-Thr7/Tn), the potency is decreased almost a 100-fold to 126 nM. This potency is reduced 5 times further when elongating to T (667.9 nM). However, the introduction of sialic acid (ST) did not significantly influence the potency further compared to the non-sialylated T-structure (615.7 nM). Importantly, removal of the glycan from the T-glycosylated glucagon restored the potency to the non-glycosylated levels eliciting an EC50 of 1,235 nM (FIG. 5E and FIG. 8).

### Example 14

### O-Glycans on NPY, PYY and PPY Modulate Receptor Activation.

The NPY family peptide hormones activate members of the NPY receptor family (Y1, Y2, Y4, and Y5), where mature NPY (1-36) and PYY (1-36) preferentially binds Y1, Y2 and Y5, and PPY preferentially binds Y4. The Y1 receptor seem to have strict requirements for the N-terminal part of the peptides as N-terminal truncation gradually decreases affinity for NPY. In contrast the Y2 receptor is more sensitive to alterations in the C-terminus of NPY and PYY and single amino acid substitutions in the C-terminus can lower affinity for the Y2 receptor⁸⁷.

The present inventors developed a receptor assay for the binding study of the NPY family to the four known receptors. As expected according to reference values in the literature PYY, NPY, had respective potencies (EC50-values) of 0.47 nM and 2.16 nM at NPY1R, 0.34 nM and 4.11 nM at receptor NPY2R, 11.35 nM and 199.9 nM at receptor NPY4R, and 12.04 and 15.03 nM at receptor NPY5R (FIGS. 5F-M and FIG. 8). Introduction of a Tn-glycan on position Thr32 in NPY (NPY-Thr32/Tn) inferred a 118-fold and 37-fold decrease in potency at receptor NPY2R and NPY5R, respectively (FIGS. 5H&J) whereas receptor potencies at NPY4R and NPY5R was decreased to a level beyond the assayed range. Even though the receptor activation at these receptors did not did not reach Emax within the assayed range, activity was still observable which indicates potencies in terms of EC50-values that are above 1 pM. NPY-Thr32/T and NPY-Thr32-ST exhibited minimal activation and did not reach Emax within the assayed range at all receptors, thus indicating potencies in terms of EC50-values above 1 pM (FIGS. 5 F & H & J & L). As with NPY, Introduction of a Tn-glycan on position Thr32 in PYY inferred a 61-fold (249.2 nM) and 37-fold (556.7 nM) decrease in potency at receptors NPY2R and NPY5R, respectively (FIGS. 5I & K) whereas receptor activation at NPY4R and NPY5R was only retained to a minimal degree indicating potencies at levels above 1 μM. PYY-Thr32/T and PYY-Thr32/ST exhibited minimal activation and did not reach Emax in the assayed range at all receptors, thus indicating potencies in terms of EC50-values above 1 pM (FIGS. 5 G & I & K & M). Data is summarized in FIG. 8. A receptor preference shift was thus observed for both PYY and NPY when incorporating Tn at position 32, where the glycosylated peptide hormones activates the receptor in the following order NPY2R>NPY5R>>NPY1R>NPY4R upon increasing levels of agonist, whereas their non-glycosylated counterparts activates the receptors in a different order, namely: NPY2R>NPY1R>NPY5R>NPY4R for NPY and NPY1R>NPY2R>NPY5R>NPY4R for PYY.

### Example 15

### O-Glycans on ANP Modulate Receptor Activation.

ANP exerts its physiological effects mainly via the NPR-A receptor but binds also to NPR-C. NPR-C, however, is mainly regarded a clearance receptor thus the present inventors selected NPR-A for ANP binding and activation.

In this assay ANP exhibited a potency of 0.9 nM for NPR-A, which is in good agreement with previous studies⁸⁸. ANP with one O-glycan (GalNAc/Tn) at residue 19 or 25 showed a 63- and 139-fold decrease in potency to 57 and 125 nM, respectively, whereas simultaneous O-glycans at residue 19 and 25 completely dismiss receptor activation. Elongation of the O-glycan on ANP residue 19 to T, ST and diST further reduced the potency 3- to 50-fold to 160 nM and 296 nM and 2699 nM, respectively. Elongation of the O-glycan on ANP residue 25 to T, ST and diST further reduced the potency 2- to 3-fold to 265 nM and 717 nM and 460 nM respectively (FIGS. 5N and 5O). O-glycans on ANP residue 19 resulted in approximately 20% reduction in efficacy and O-glycans on ANP residue 25 resulted in approximately 20% increase in efficacy.

In summary, all peptide hormones with O-glycans attached at one or more specific sites elicited a right-shifted full, partial agonist or superagonist response positively correlated to glycan size.

### Example 16

### O-Glycans Modulate Peptide Hormone Stability In Vitro

Many peptide hormones are destined for endocrine circulation where they are rapidly degraded with half-lives reaching only a few minutes⁶⁻⁸. The present inventors have previously demonstrated that O-glycans in close proximity to proteolytic processing sites can modulate the rate of processing³³. To study if O-glycosylation of peptide hormones altered the inherent proteolytic instability of this class of biomolecules, the present inventors subjected selected glycopeptide hormones to ex vivo degradation assays using human plasma and in vitro degradation using neprilysin (NEP), insulin degrading enzyme (IDE) and dipeptidyl peptidase IV (DPP-IV) enzymes known to degrade peptide hormones and other bioactive peptides in vivo including ANP, GLP-1, PYY, VIP, Secretin and Galanin^{89,90} (FIGS. 6 and 7). The degradation pattern was monitored by MALDI-TOF analysis in a time-course assay with timepoints from 15 minutes to up to 24 hrs.

Degradation Assay:

For enzymatic degradation assays using either NEP or DPP-IV (R&D systems, UK), an enzyme titration was carried out to ensure full degradation within one hour of reaction time. 15 pM of non-glycosylated peptide substrate and either 50 mM Tris, pH 9, 0.05% Brij for NEP or 50 mM Tris, pH 8 for DPP-IV was treated with varying enzyme amounts in a total volume of 10 μL incubated at 37° C. The degradation assay of the three glycoforms (Tn, T and ST) was performed under same reaction-parameters along with the non-glycosylated peptide (four separate reactions). The following amounts of enzyme were used for NEP reactions: 150 pg/pL enzyme for VIP, Galanin, secretin and their glycoforms, 20 ng/pL for PYY and its glycoforms. 4 and 10 ng/pL DPIV was used for the degradation of VIP+glycoforms and PYY+glycoforms respectively. For ex vivo plasma degradation assays, plasma was diluted to a final concentration of 20% plasma, 50 mM Tris (pH 7.7) and degradation of 15 pM (glyco-) peptide substrate was investigated. Degradation was carried out at 37° C. and several aliquots were taken between 0 minutes and 24 hours and degradation was monitored by MALDI-TOF-MS.

MALDI-TOF-MS was performed on a Bruker Autoflex instrument (Bruker Daltonik GmbH, Bremen, Germany) by mixing the quenched aliquots with a saturated solution of α-Cyano-4-hydroxycinnamic acid in ACN/H₂O/TFA (70:30:0.1) at a ratio 1:1 on a target steel plate and mass-spectra were acquired in linear mode.

### Example 17

### O-Glycans on ANP Modulate Stability In Vitro

For the degradation assays with (glyco-)ANP, the amount of recombinant enzyme used was optimized to fully digest the naked peptide of interest within one hour of incubation at 37° C. In vitro cleavage activity was assayed by adding 2.5 ng Neprilysin (R&D Systems) or 125 ng Insulin-degrading enzyme (IDE, R&D Systems) to 813 pmol peptide or glycopeptide substrate in a total volume of 25 μL. Reactions were performed in 50 mM Tris, 0.05% Brij-35, pH 9 (Neprilysin), or 50 mM Tris, 20 mM NaCl, pH 7.5 (IDE) and incubated at 37° C. Product development was evaluated after 15 min, 30 min, 60 min, and 24 hours by MALDI-TOF and reverse-phase HPLC (C18). Neprilysin degraded ANP completely within 15 min, whereas and ANP-S25/Tn were degraded slower with residual full length ANP-S19/Tn detectable even after 60 min and residual ANP-S25/Tn detectable after 15 min. Residual ANP-S19/Tn, -S25/Tn was detectable after 30 min. After 1 hour, non-modified ANP was completely degraded whereas major degradation products of ANP-S19/Tn and ANP-S19/Tn, -S25/Tn remained detectable even after 24 hours incubation (FIG. 6A). In a similar time-course with insulin-degrading enzyme, ANP was completely degraded within 15 min, whereas ANP-S19/Tn and ANP-S25/Tn remained partly as full length glycopeptides after 30-60 minutes. Interestingly, ANP-S19/Tn, -S25/Tn was degraded within 15 minutes (FIG. 6B).

### Example 18

### O-Glycans on Secretin Modulate Stability In Vitro

In a similar manner, NEP completely degraded Secretin 1-27 to 1-22 within 60 minutes. Also here, glycosylation had a protective effect such that Thr34-Tn remained intact after 60 min and Thr34-T after 120 minutes. Again, sialylated Secretin Thr34-ST appeared resistant to NEP degradation even after 24 hrs (FIGS. 7A and 7C).

### Example 19

### O-Glycans on Galanin Modulate Stability In Vitro

Nonglycosylated Galanin 1-27 was degraded by NEP within 60 minutes whereas the Ser23-Tn and -T extended glycoforms were degraded after 120 min. The sialylated Galanin-Ser55/ST variant remained intact even after 24 hrs in solution suggesting that sialylation of Galanin is necessary for complete protection from NEP degradation, at least in vitro (FIGS. 7B and 7C).

### Example 20

### O-Glycans on VIP Increase Stability In Vitro

NEP cleaves VIP sequentially at Asp3/Ala4 then Phe6/Thr7, Lys21/Tyr22 and ultimately at Ala18/Val19 in vitro (FIG. 7 for a summary). Non-glycosylated VIP peptide was completely degraded after 15 minutes with NEP treatment whereas Thr7-Tn glycosylated VIP remained partially intact after 15 minutes, Thr7-T glycosylated VIP remained partially intact after 30 minutes and VIP-Thr7/ST remained completely intact after 60 minutes (FIGS. 7B and 7C).

### Example 21

### O-Glycans on VIP Increase Stability In Vitro

DPP-IV also degrades VIP both in vivo and in vitro initially cleaving off two N-terminal amino acids Ser2/Asp3, then cleaves at Ala4/Val5 and lastly C-terminally at Tyr22/Leu23. Where the non-glycosylated VIP, Thr7-Tn and Thr7-T glycosylated VIP were degraded equally fast within 60 minutes, the sialylated VIP-Thr7/ST remained fully intact after 30 minutes and partially intact after 60 minutes (FIGS. 7B and 7C).

### Example 22

### O-Glycans on PYY Increase Stability In Vitro

NEP cleaves PYY at 4 positions at Tyr20/Tyr21, Ser23/Leu24, His26/Tyr27 and Leu30/Val31 in a sequence the present inventors were not able to decide. However whereas non-glycosylated PYY was degraded after 15 minutes all three PYY-Thr32/Tn/T/ST glycosylated peptides remained intact up to 120 minutes (FIGS. 7B and 7C).

### Example 23

### O-Glycans on PYY Increase Stability In Vitro

DPP-IV cleaves PYY both in vivo and in vitro N-terminally at Pro2/Ile3. Subjecting the non-glycosylated and Thr32 glycopeptide variants to in vitro DPPIV degradation revealed that where Tn- and T-glycosylation had no effect on DPP-IV activity Thr32/ST weakly protected the peptide from N-terminal degradation (FIGS. 7B and 7C).

### Example 24

### O-Glycans on PYY Increase Stability Ex Vivo

PYY is quickly removed from circulation due to the action of a number of proteases. To approximate in vivo conditions the present inventors chose to analyse PYY degradation using human plasma ex vivo. In plasma PYY is degraded both N-terminally at Pro2/Ile3 and Pro5/Glu6 and C-terminally at Gln34/Arg35 and where O-glycans at residue Thr32 (Thr32/Tn/T/ST) had no effect on the N-terminal degradation, as seen for the in vitro DPP-IV degradation, the present inventors observed full protection from the C-terminal degradation up to 24 hours of the PYY-Thr32/Tn/T/ST glycosylated PYY peptides whereas the non-glycosylated peptide was C-terminally degraded only after 1 hour (FIGS. 7B and 7C).

### Example 25

### O-Glycans on GLP-1₇₋₃₆ Increase Stability In Vitro and Predicts Increased Stability in Vivo

DPP-IV is one of the primary enzymes degrading GLP-1₇₋₃₆ (from hereon GLP-1) in the circulation in vivo⁹¹. DPP-IV removes the two N-terminal amino acids by cleaving between Ala2/Ser3 both in vitro and in vivo thus inactivating GLP-1, and mutating this DPP-IV cleavage site greatly enhances GLP-1 half-life in vivo⁹². Due to this link, DPP-IV inhibitors have successfully been used therapeutically to enhance the effects of endogenous GLP-1⁹¹. To test the effect of glycosylation in close proximity to the DPP-IV cleavage site we incubated GLP-1 with and without glycans with DPP-IV in vitro. Where the non-glycosylated GLP-1 was fully degraded after 30 minutes incubation with equimolar amounts of monoglycosylated GLP-1-Thr5/Tn and GLP-1-Thr7/Tn and equimolar amounts of GLP-1-Thr5/T or GLP-1-Thr7/T were protected against degradation until 120 minutes incubation. Equimolar amounts of the sialylated monoglycosylated GLP-1-Thr5/ST or GLP-1-Thr7/ST remained fully intact until the 120 minute-timepoint and small amounts of intact silaylated GLP-1 was detectable even after 24 h of incubation. The large body of literature on DPP-IV resistant GLP-1 analogs⁹¹⁻⁹³ taken together with the presented data on Tn, T or ST glycosylated GLP-1, predict that the GLP-1 decorated with either GalNAc (Tn), Gal-GalNAc (T) or Sialyl-GalGalNAc on positions Thr5 or Thr7 has an extended half-life in vivo. Results are summarized in FIG. 7C.

NEP also play a role in the degradation of GLP-1 in the circulation, and it has been suggested that NEP is responsible for up to 50% of the degradation of GLP-1⁹³. In our in vitro assay, NEP cleaves GLP-1 initially at Trp25/Leu26 followed by a second cleavage at Glu21/Phe22 consistent with NEP cleavage sites on GLP-1 reported earlier⁹⁴ (FIG. 7C for a summary). Non-glycosylated GLP-1 peptide was completely degraded after 60-minutes whereas a equimolar amounts monoglycosylated GLP-₁-Thr5/Tn and GLP-₁-Thr7/Tn remained partially intact after 60 minutes when treated with same amount of NEP. When elongating the glycan structure, equimolar amounts of GLP-1-Thr5/T or GLP-1-Thr7/T remained partially intact after 60 minutes. Equimolar amounts of GLP-1-Thr5/ST or GLP1-Thr7/ST remained completely intact after 60 minutes incubation indicating that either glycosite provide protection of the peptide hormone from NEP-mediated degradation. Small amounts of sialylated GLP-1 was still detectable after 20 h of incubation. Results are summarized in FIG. 7C.

### Example 26

### O-Glycans on PYY and VIP are Present in Low Stoichiometry in Porcine Intestinal Extracts

Where the shotgun glycoproteomics strategy is designed to sequence and identify individual O-glycosylation sites it does not allow us to determine site occupancy in a given protein, i.e what is the proportion of glycosylated protein in the total pool of that protein in a given system (e.g. blood, lymph fluid, cell lysate, tissue etc.). To answer this question the present inventors developed a technique to quantify endogenous glycopeptides using either sensitive LC-MS or radioimmunaassay (RIA). Using extracted proteins from pig ileum the present inventors separated proteins from glycoproteins using Jacalin-LWAC and quantified non-glycosylated and glycosylated PYY using either a sensistive PYY-RIA⁹⁵ or in the case of VIP, sensitive LC-MS by comparing to isotope labelled standards in the form of in vitro synthesized (glyco-)VIP. In order to prepare the extract for mass spectrometry, the extracted proteins as well as the standards were digested with trypsin prior to Jacalin-LWAC in the case of LC-MS analysis of site occupancy on VIP.

The present inventors identified approximately 1% glycosylated PYY-Thr32/T and VIP-Thr7-T in porcine ileum tissue ethanol extracts confirming that (sialylated)-T-PYY and -VIP exist in the porcine intestine presumably at a concentration approximately two orders of magnitude below the non-glycosylated.

### Example 27

### O-Glycans on ANP Confer Retained and Prolonged Differential Agonist Effect In Vivo

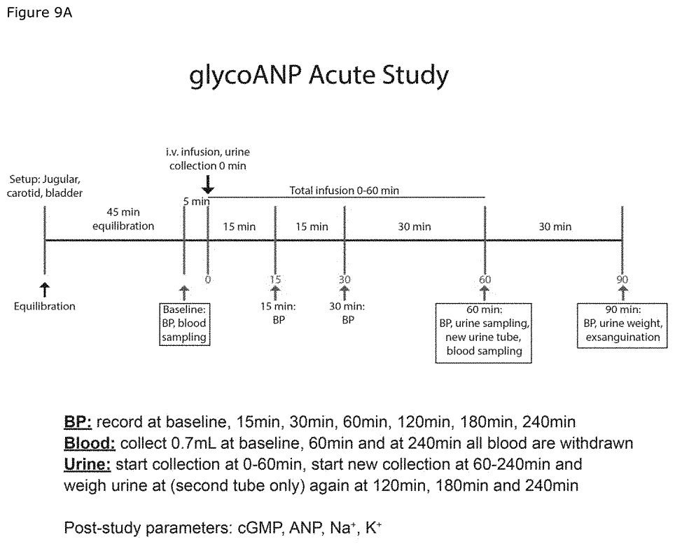

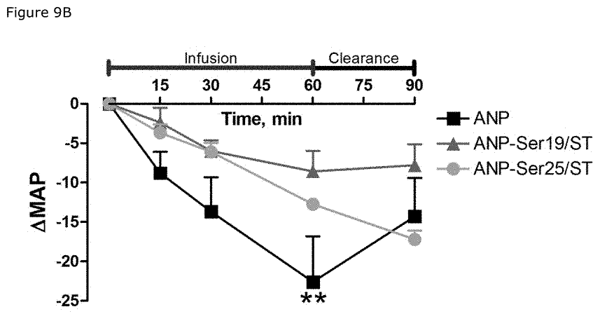

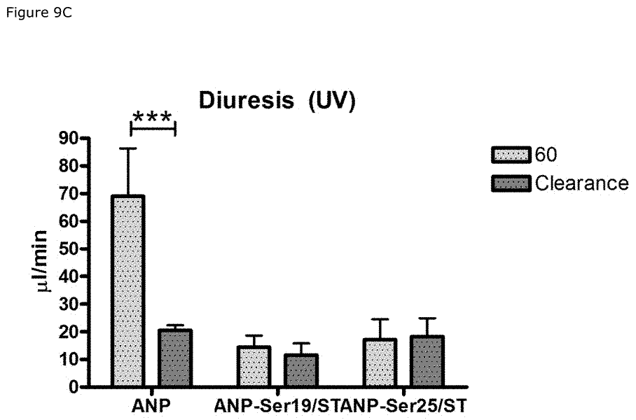

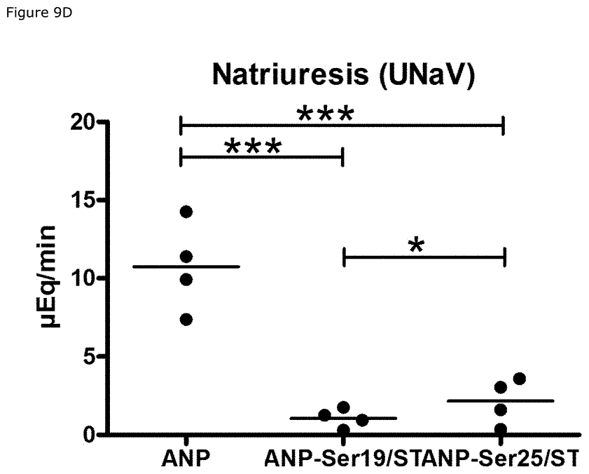

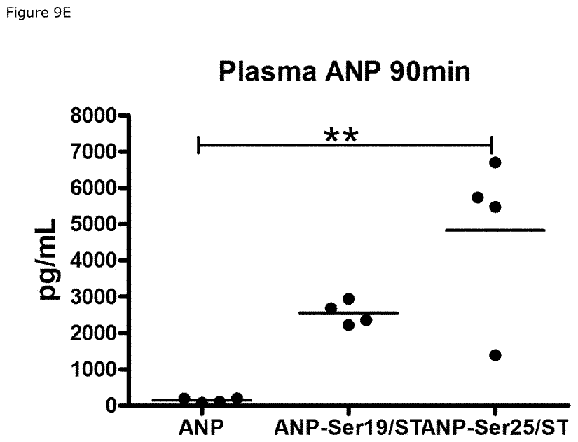

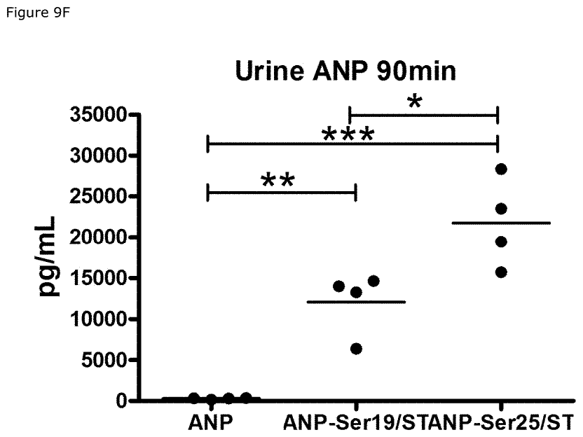

To evaluate the function of O-glycans on ANP in vivo we investigated the cGMP generating, renal and blood pressure actions of equimolar dose (600 pmol/kg/min) ANP-Ser19/ST or ANP-Ser25/ST [n=4/group] in male Sprague Dawley rats (250-350 grams; Charles River Laboratories, Wilmington, Mass.). The protocol is outlined in FIG. 9A.

Anesthesia in rats was induced with 133 mg/kg i.p. inactin (Sigma, St Louis, Mo.) and rats were maintained on a heating pad for 1 hour until completely anesthetized. Rats were then subjected to vessel and bladder cannulation for peptide infusion, BP measurement, blood sampling and urine collection. A polyethylene (PE)-5G tube catheter was placed into the jugular vein for inulin, peptide intravenous infusion. The carotid artery was cannulated with a PE-50 tube catheter for BP measurement (Sonometrics, London, Ontario, Canada) and blood sampling. The bladder was accessed and cannulated with a PE-50 tube catheter for passive urine collection. After completion of the above procedural set up, a 45 min equilibration period was performed that included continuous IV inulin and saline infusion. After the 45 min equilibration period, baseline (Time==Q min) parameters were recorded and one blood sampling (0.7 ml) was performed. The inulin and saline infusion was replaced by a continuous intravenously (i.v.) infusion of equimolar ANP-Ser19/ST or ANP-Ser25/ST for 60 min. The infusion rate was weight adjusted and equals weight*0.7/6000 ml/min. After 60 min infusion (Time=60 min), another blood sampling (0.7 ml) was conducted. A post-infusion clearance (Time=90 min) was performed for 30 min. At the end of the study, blood was collected to determine plasma ANP and cGMP levels and to calculate glomerular filtration rate (GFR). Urine was collected at the end of the infusion (Time=60 min) and the study (Time=90 min). Urinary sodium was measured with pHOx Ultra (Nova Biomedical, Waltham, Mass.). Urine flow (UV) and urinary sodium excretion (UNaV) were calculated as urine volume or sodium clearance per min, Inulin concentrations were measured with enthrone method and inulin clearance was used for GFR quantification. Urinary cGMP and ANP excretion rate was calculated based on raw values obtained in the urine and UV.

### In Vivo Cardiovascular and Renal Actions

Infusion with ANP, ANP-Ser19/ST and ANP-Ser25/ST resulted in decrease in mean arterial pressure (MAP) and increase in plasma cGMP over the 60 minutes infusion period (FIG. 9B). In the following 30 minutes clearance period, in rats infused with ANP, the MAP rebounded whereas the glycosylated ANP (ANP-Ser19/ST or ANP-Ser25/ST) variants produced a sustained or further reduced in MAP.

Infusion with ANP resulted in an 4-5 fold increase in urine volume (UV) measured after 60 minutes (FIG. 9C) and a 5-10 fold increase in urinary sodium excretion (UNaV) (FIG. 9D) after 90 minutes. After a clearance period of 30 minutes the UV was normalized. In striking contrast infusion with ANP-Ser19/ST and ANP-Ser25/ST did not result in increased diuresis nor natriuresis and remained constant at 11-18 uL/min. (FIG. 9D).

### Example 28

### O-Glycans on ANP Increase Stability In Vivo

The sustained cardiovascular effect seen with ANP-Ser19/ST and ANP-Ser25/ST suggested that the glycosylated peptides circulate longer compared to the non-glycosylated ANP. Measuring plasma ANP after 90 minutes demonstrated that the non-glycosylated ANP circulated at low concentrations (mean, 148 ng/mL) whereas ANP-Ser19/ST and ANP-Ser25/ST was still present at 17-33 fold higher concentrations (FIG. 9E). Further supporting an increased stability the present inventors could measure both ANP-Ser19/ST and ANP-Ser25/ST at concentrations 41-74 fold higher than ANP in the urine after 90 minutes (FIG. 9F).

### Comparison of Identified Glycosylated Peptide Hormones to Published Glycosylated Peptide Hormones

A number of pro-peptide hormones have been reported to be O-glycosylated on the pro-part (non-matured) (pro-brain natriuretic peptide (proBNP), POMC, proglucagon and kininogen). Very recently, two reports describing mature insulin, somatostatin and amylin as well as calcitonin O-glycosylation were released^{13,83}. Apart from these, only adiponectin has been described as being O-glycosylated before in mammalian studies.

### Definition of Peptide Hormones/Neuropeptides/Regulatory Peptides

Peptide hormones, regulatory peptides or neuropeptides are bioactive peptides that are approximately 3-100 amino acids long. They are involved in cell-cell signaling where they can bind and activate highly specific peptide hormone receptors upon binding.

TABLE 4
(Human peptide hormones the SEQ ID Nos. of which are disclosed in Tables 6A to 6E infra)

| ID | Sequence | Length | Family | Name |
|---|---|---|---|---|
| NP00002 | SVPHFSDEDKDPE | 13 | 7B2 | C-terminal peptide (By |
| | | | | similarity) |
| NP00012 | YSPRTPDRVSEADIQRLLHGVMEQLGIARPRVEYPAHQAMNLVGPQSIEGGAHEGL | 186 | 7B2 | Neuroendocrine protein 7B2 |
| | QHLGPFGNIPNIVAELTGDNIPKDFSEDQGYPDPPNPCPVGKTADDGCLENTPDTAE | | | |
| | FSREFQLHQHLFDPEHDYPGLGKWNKKLLYEKMKGGERRKRRSVNPYLQGQRLD | | | |
| | NVVAKKSVPHFSDEDKDPE | | | |
| NP00013 | YSPRTPDRVSEADIQRLLHGVMEQLGIARPRVEYPAHQAMNLVGPQSIEGGAHEGL | 150 | 7B2 | N-terminal peptide (By |
| | QHLGPFGNIPNIVAELTGDNIPKDFSEDQGYPDPPNPCPVGKTADDGCLENTPDTAE | | | similarity) |
| | FSREFQLHQHLFDPEHDYPGLGKWNKKLLYEKMKGGE | | | |
| NP00026 | MALQADFDRAAEDVRKLKARPDDGELKELYGLYKQAIVGDINIACPGMLDLKGK | 88 | ACBP | Acyl-CoA-binding domain- |
| | AKWEAWNLKKGLSTEDATSAYISKAKELIEKYGI | | | containing protein 7 |
| NP00034 | SQAEFEKAAEEVRHLKTKPSDEEMLFIYGHYKQATVGDINTERPGMLDFTGKAKW | 86 | ACBP | Acyl-CoA-binding protein |
| | DAWNELKGTSKEDAMKAYINKVEELKKKYGI | | | |
| NP00042 | TQAQLLRVGCVLGTCQVQNLSHRLWQLMGPAGRQDSAPVDPSSPHSY | 47 | Adrenomedullin | Adrenomedullin-2 (By |
| | | | | similarity) |
| NP00043 | VGCVLGTCQVQNLSHRLWQLMGPAGRQDSAPVDPSSPHSY | 40 | Adrenomedullin | Intermedin-short (Potential) |
| NP00044 | YRQSMNNFQGLRSFGCRFGTCTVQKLAHQIYQFTDKDKDNVAPRSKISPQGY | 52 | Adrenomedullin | Adrenomedullin |
| NP00045 | ARLDVASEFRKKWNKWALSR | 20 | Adrenomedullin | Proadrenomedullin N-20 |
| | | | | terminal peptide |
| NP00601 | QRPRLSHKGPMPF | 13 | Apelin | Apelin-13 (By similarity) |
| NP00602 | NGPGPWQGGRRKFRRQRPRLSHKGPMPF | 28 | Apelin | Apelin-28 (By similarity) |
| NP00603 | GSRNGPGPWQGGRRKFRRQRPRLSHKGPMPF | 31 | Apelin | Apelin-31 (By similarity) |
| NP00604 | LVQPRGSRNGPGPWQGGRRKFRRQRPRLSHKGPMPF | 36 | Apelin | Apelin-36 (By similarity) |
| NP00790 | VPLPAGGGTVLTKMYPRGNHWAVGHLM | 27 | Bombesin/neuromedin- | Gastrin-releasing peptide |
| | | | B/ranatensin | |
| NP00791 | GNHWAVGHLM | 10 | Bombesin/neuromedin- | Neuromedin-C |
| | | | B/ranatensin | |
| NP00792 | GNLWATGHFM | 10 | Bombesin/neuromedin- | Neuromedin-B |
| | | | B/ranatensin | |
| NP00793 | APLSWDLPEPRSRASKIRVHSRGNLWATGHFM | 32 | Bombesin/neuromedin- | Neuromedin-B-32 |
| | | | B/ranatensin | |
| NP00811 | RPPGFSPFR | 9 | Bradykinin | Bradykinin |
| NP00812 | KRPPGFSPFR | 10 | Bradykinin | Lysyl-bradykinin |
| NP00813 | ISLMKRPPGFSPFR | 14 | Bradykinin | T-kinin |
| NP00846 | CGNLSTCMLGTYTQDFNKFHTFPQTAIGVGAP | 32 | Calcitonin | Calcitonin |
| NP00847 | DMSSDLERDHRPHVSMPQNAN | 21 | Calcitonin | Katacalcin |
| NP00848 | ACDTATCVTHRLAGLLSRSGGVVKNNFVPTNVGSKAF | 37 | Calcitonin | Calcitonin gene-related |
| | | | | peptide 1 |
| NP00849 | ACNTATCVTHRLAGLLSRSGGMVKSNFVPTNVGSKAF | 37 | Calcitonin | Calcitonin gene-related |
| | | | | peptide 2 |
| NP00850 | KCNTATCATQRLANFLVHSSNNFGAILSSTNVGSNTY | 37 | Calcitonin | Islet amyloid polypeptide |
| NP00869 | QEDAELQPRALDIYSAVDDASHEKELIEALQEVLKKLKS | 39 | CART | CART(1-39) |
| NP00870 | VPIYEKKYGQVPMCDAGEQCAVRKGARIGKLCDCPRGTSCNSFLLKCL | 48 | CART | CART(42-89) |
| NP00881 | QEDAELQPRALDIYSAVDDASHEKELIEALQEVLKKLKSKRVPIYEKKYGQVPMCD | 89 | CART | Cocaine- and amphetamine- |
| | AGEQCAVRKGARIGKLCDCPRGTSCNSFLLKCL | | | regulated |
| NP00904 | QNDTEPIVLEGKCLVVCDSNPATDSKGSSSSPLGISVRAANSKVAFSAVRSTNHEPS | 174 | Cerebellins | Cerebellin-4 |
| | EMSNKTRIIYFDQILVNVGNFFTLESVFVAPRKGIYSFSFHVIKVYQSQTIQVNLMLN | | | |
| | GKPVISAFAGDKDVTREAATNGVLLYLDKEDKVYLKLEKGNLVGGWQYSTFSGFL | | | |
| | VFPL | | | |
| NP00909 | GSAKVAFSAIRSTNH | 15 | Cerebellins | [des-Ser1]-cerebellin |
| NP00910 | SGSAKVAFSAIRSTNH | 16 | Cerebellins | Cerebellin |
| NP00911 | QNETEPIVLEGKCLVVCDSNPTSDPTGTALGISVRSGSAKVAFSAIRSTNHEPSEMSN | 172 | Cerebellins | Cerebellin-1 |
| | RTMIIYFDQVLVNIGNNFDSERSTFIAPRKGIYSFNFHVVKVYNRQTIQVSLMLNGW | | | |
| | PVISAFAGDQDVTREAASNGVLIQMEKGDRAYLKLERGNLMGGWKYSTFSGFLVF | | | |
| | PL | | | |
| NP00912 | QNDTEPIVLEGKCLVVCDSSPSADGAVTSSLGISVRSGSAKVAFSATRSTNHEPSEM | 173 | Cerebellins | Cerebellin-2 |
| | SNRTMTIYFDQVLVNIGNHFDLASSIFVAPRKGIYSFSFHVVKVYNRQTIQVSLMQN | | | |
| | GYPVISAFAGDQDVTREAASNGVLLLMEREDKVHLKLERGNLMGGWKYSTFSGF | | | |
| | LVFPL | | | |
| NP00913 | QEGSEPVLLEGECLVVCEPGRAAAGGPGGAALGEAPPGRVAFAAVRSHHHEPAGE | 173 | Cerebellins | Cerebellin-3 |
| | TGNGTSGAIYFDQVLVNEGGGFDRASGSFVAPVRGVYSFRFHVVKVYNRQTVQVS | | | |
| | LMLNTWPVISAFANDPDVTREAATSSVLLPLDPGDRVSLRLRRGNLLGGWKYSSFS | | | |
| | GFLIFPL | | | |
| NP00983 | AYGFRGPGPQL | 11 | Chromogranin/ | AL-11 |
| | | | secretogranin | |
| NP00984 | LPVNSPMNKGDTEVMKCIVEVISDTLSKPSPMPVSQECFETLRGDERILSILRHQNLL | 439 | Chromogranin/ | Chromogranin-A |
| | KELQDLALQGAKERAHQQKKHSGFEDELSEVLENQSSQAELKEAVEEPSSKDVME | | secretogranin | |
| | KREDSKEAEKSGEATDGARPQALPEPMQESKAEGNNQAPGEEEEEEEEATNTHPPA | | | |
| | SLPSQKYPGPQAEGDSEGLSQGLVDREKGLSAEPGWQAKREEEEEEEEEAEAGEEA | | | |
| | VPEEEGPTVVLNPHPSLGYKEIRKGESRSEALAVDGAGKPGAEEAQDPEGKGEQEH | | | |
| | SQQKEEEEEMAVVPQGLFRGGKSGELEQEEERLSKEWEDSKRWSKMDQLAKELT | | | |
| | AEKRLEGQEEEEDNRDSSMKLSFRARAYGFRGPGPQLRRGWRPSSREDSLEAGLPL | | | |
| | QVRGYPEEKKEEEGSANRRPEDQELESLSAIEAELEKVAHQLQALRRG | | | |
| NP00985 | EDSKEAEKSGEATDGARPQALPEPMQESKAEGNNQAPGEEEEEEEEATNTHPPASL | 92 | Chromogranin/ | EA-92 |
| | PSQKYPGPQAEGDSEGLSQGLVDREKGLSAEPGWQA | | secretogranin | |
| NP00986 | EEEGSANRRPEDQELESLSAIEAELEKVAHQLQALRR | 37 | Chromogranin/ | ER-37 |
| | | | secretogranin | |
| NP00987 | EEEEEEEEEAEAGEEAVPEEEGPTVVLNPHPSL | 33 | Chromogranin/ | ES-43 |
| | | | secretogranin | |
| NP00988 | GYPEEKKEEEGSANRRPEDQELESLSAIEAELEKVAHQLQALRR | 44 | Chromogranin/ | GR-44 |
| | | | secretogranin | |
| NP00989 | GWRPSSREDSLEAGLPLQV | 19 | Chromogranin/ | GV-19 |
| | | | secretogranin | |
| NP00990 | LEGQEEEEDNRDSSMKLSF | 19 | Chromogranin/ | LF-19 |
| | | | secretogranin | |
| NP00991 | SEALAVDGAGKPGAEEAQDPEGKGEQEHSQQKEEEEEMAVVPQGLFRG | 48 | Chromogranin/ | Pancreastatin |
| | | | secretogranin | |
| NP00992 | SGELEQEEERLSKEWEDS | 18 | Chromogranin/ | SS-18 |
| | | | secretogranin | |
| NP00993 | LPVNSPMNKGDTEVMKCIVEVISDTLSKPSPMPVSQECFETLRGDERILSILRHQNLL | 76 | Chromogranin/ | Vasostatin-1 |
| | KELQDLALQGAKERAHQQ | | secretogranin | |
| NP00994 | LPVNSPMNKGDTEVMKCIVEVISDTLSKPSPMPVSQECFETLRGDERILSILRHQNLL | 113 | Chromogranin/ | Vasostatin-2 |
| | KELQDLALQGAKERAHQQKKHSGFEDELSEVLENQSSQAELKEAVEEPSSKDVME | | secretogranin | |
| NP00995 | WSKMDQLA | 8 | Chromogranin/ | WA-8 |
| | | | secretogranin | |
| NP00996 | WSKMDQLAKELTAE | 14 | Chromogranin/ | WE-14 |
| | | | secretogranin | |
| NP00997 | SAEFPDFYDSEEPVSTHQEAENEKDRADQTVLTEDEKKELENLAAMDLELQKIAEK | 57 | Chromogranin/ | CCB peptide |
| | F | | secretogranin | |
| NP00998 | FLGEGHHRVQENQMDKARRHPQGAWKELDRNYLNYGEEGAPGKWQQQGDLQD | 74 | Chromogranin/ | GAWK peptide |
| | TKENREEARFQDKQYSSHHTAE | | secretogranin | |
| NP00999 | MPVDNRNHNEGMVTRCIIEVLSNALSKSSAPPITPECRQVLKTSRKDVKDKETTEN | 657 | Chromogranin/ | Secretogranin-1 |
| | ENTKFEVRLLRDPADASEAHESSSRGEAGAPGEEDIQGPTKADTEKWAEGGGHSRE | | secretogranin | |
| | RADEPQWSLYPSDSQVSEEVKTRHSEKSQREDEEEEEGENYQKGERGEDSSEEKHL | | | |
| | EEPGETQNAFLNERKQASAIKKEELVARSETHAAGHSQEKTHSREKSSQESGEETG | | | |
| | SQENHPQESKGQPRSQEESEEGEEDATSEVDKRRTRPRHHHGRSRPDRSSQGGSLPS | | | |
| | EEKGHPQEESEESNVSMASLGEKRDHHSTHYRASEEEPEYGEEIKGYPGVQAPEDL | | | |
| | EWERYRGRGSEEYRAPRPQSEESWDEEDKRNYPSLELDKMAHGYGEESEEERGLE | | | |
| | PGKGRHHRGRGGEPRAYFMSDTREEKRFLGEGHHRVQENQMDKARRHPQGAWK | | | |
| | ELDRNYLNYGEEGAPGKWQQQGDLQDTKENREEARFQDKQYSSHHTAEKRKRLG | | | |
| | ELFNPYYDPLQWKSSHFERRDNMNDNFLEGEEENELTLNEKNFFPEYNYDWWEK | | | |
| | KPFSEDVNWGYEKRNLARVPKLDLKRQYDRVAQLDQLLHYRKKSAEFPDFYDSE | | | |
| | EPVSTHQEAENEKDRADQTVLTEDEKKELENLAAMDLELQKIAEKFSQRG | | | |
| NP01000 | QRNQLLQKEPDLRLENVQKFPSPEMIRALEYIENLRQQAHKEESSPDYNPYQGVSV | 587 | Chromogranin/ | Secretogranin-2 |
| | PLQQKENGDESHLPERDSLSEEDWMRIILEALRQAENEPQSAPKENKPYALNSEKN | | secretogranin | |
| | FPMDMSDDYETQQWPERKLKHMQFPPMYEENSRDNPFKRTNEIVEEQYTPQSLAT | | | |
| | LESVFQELGKLTGPNNQKRERMDEEQKLYTDDEDDIYKANNIAYEDVVGGEDWN | | | |
| | PVEEKIESQTQEEVRDSKENIEKNEQINDEMKRSGQLGIQEEDLRKESKDQLSDDVS | | | |
| | KVIAYLKRLVNAAGSGRLQNGQNGERATRLFEKPLDSQSIYQLIEISRNLQIPPEDLI | | | |
| | EMLKTGEKPNGSVEPERELDLPVDLDDISEADLDHPDLFQNRMLSKSGYPKTPGRA | | | |
| | GTEALPDGLSVEDILNLLGMESAANQKTSYFPNPYNQEKVLPRLPYGAGRSRSNQL | | | |
| | PKAAWIPHVENRQMAYENLNDKDQELGEYLARMLVKYPEIINSNQVKRVPGQGSS | | | |
| | EDDLQEEEQIEQAIKEHLNQGSSQETDKLAPVSKRFPVGPPKNDDTPNRQYWDEDL | | | |
| | LMKVLEYLNQEKAEKGREHIAKRAMENM | | | |
| NP01001 | TNEIVEEQYTPQSLATLESVFQELGKLTGPNNQ | 33 | Chromogranin/ | Secretoneurin |
| | | | secretogranin | |
| NP01002 | FPKPGGSQDKSLHNRELSAERPLNEQIAEAEEDKIKKTYPPENKPGQSNYSFVDNLN | 449 | Chromogranin/ | Secretogranin-3 |
| | LLKAITEKEKIEKERQSIRSSPLDNKLNVEDVDSTKNRKLIDDYDSTKSGLDHKFQD | | secretogranin | |
| | DPDGLHQLDGTPLTAEDIVHKIAARIYEENDRAVFDKIVSKLLNLGLITESQAHTLE | | | |
| | DEVAEVLQKLISKEANNYEEDPNKPTSWTENQAGKIPEKVTPMAAIQDGLAKGEN | | | |
| | DETVSNTLTLTNGLERRTKTYSEDNFEELQYFPNFYALLKSIDSEKEAKEKETLITIM | | | |
| | KTLIDFVKMMVKYGTISPEEGVSYLENLDEMIALQTKNKLEKNATDNISKLFPAPSE | | | |
| | KSHEETDSTKEEAAKMEKEYGSLKDSTKDDNSNPGGKTDEPKGKTEAYLEAIRKNI | | | |
| | EWLKKHDKKGNKEDYDLSKMRDFINKQADAYVEKGILDKEEAEAIKRIYSSL | | | |
| NP01097 | QESQSEEIDCNDKDLFKAVDAALKKYNSQNQSNNQFVLYRITEATKTVGSDTFYSF | 626 | Cystatin | Kininogen-1 |
| | KYEIKEGDCPVQSGKTWQDCEYKDAAKAATGECTATVGKRSSTKFSVATQTCQIT | | | |
| | PAEGPVVTAQYDCLGCVHPISTQSPDLEPILRHGIQYFNNNTQHSSLFMLNEVKRAQ | | | |
| | RQVVAGLNFRITYSIVQTNCSKENFLFLTPDCKSLWNGDTGECTDNAYIDIQLRIAS | | | |
| | FSQNCDIYPGKDFVQPPTKICVGCPRDIPTNSPELEETLTHTITKLNAENNATFYFKID | | | |
| | NVKKARVQVVAGKKYFIDFVARETTCSKESNEELTESCETKKLGQSLDCNAEVYV | | | |
| | VPWEKKIYPTVNCQPLGMISLMKRPPGFSPFRSSRIGEIKEETTVSPPHTSMAPAQDE | | | |
| | ERDSGKEQGHTRRHDWGHEKQRKHNLGHGHKHERDQGHGHQRGHGLGHGHEQ | | | |
| | QHGLGHGHKFKLDDDLEHQGGHVLDHGHKHKHGHGHGKHKNKGKKNGKHNG | | | |
| | WKTEHLASSSEDSTTPSAQTQEKTEGPTPIPSLAKPGVTVTFSDFQDSDLIATMMPPI | | | |
| | SPAPIQSDDDWIPDIQIDPNGLSFNPISDFPDTTSPKCPGRPWKSVSEINPTTQMKESY | | | |
| | YFDLTDGLS | | | |
| NP01098 | QESQSEEIDCNDKDLFKAVDAALKKYNSQNQSNNQFVLYRITEATKTVGSDTFYSF | 362 | Cystatin | Kininogen-1 heavy chain |
| | KYEIKEGDCPVQSGKTWQDCEYKDAAKAATGECTATVGKRSSTKFSVATQTCQIT | | | |
| | PAEGPVVTAQYDCLGCVHPISTQSPDLEPILRHGIQYFNNNTQHSSLFMLNEVKRAQ | | | |
| | RQVVAGLNFRITYSIVQTNCSKENFLFLTPDCKSLWNGDTGECTDNAYIDIQLRIAS | | | |
| | FSQNCDIYPGKDFVQPPTKICVGCPRDIPTNSPELEETLTHTITKLNAENNATFYFKID | | | |
| | NVKKARVQVVAGKKYFIDFVARETTCSKESNEELTESCETKKLGQSLDCNAEVYV | | | |
| | VPWEKKIYPTVNCQPLGMISLMK | | | |
| NP01099 | SSRIGEIKEETTVSPPHTSMAPAQDEERDSGKEQGHTRRHDWGHEKQRKHNLGHG | 255 | Cystatin | Kininogen-1 light chain |
| | HKHERDQGHGHQRGHGLGHGHEQQHGLGHGHKFKLDDDLEHQGGHVLDHGHK | | | |
| | HKHGHGHGKHKNKGKKNGKHNGWKTEHLASSSEDSTTPSAQTQEKTEGPTPIPSL | | | |
| | AKPGVTVTFSDFQDSDLIATMMPPISPAPIQSDDDWIPDIQIDPNGLSFNPISDFPDTT | | | |
| | SPKCPGRPWKSVSEINPTTQMKESYYFDLTDGLS | | | |
| NP01100 | WGHE | 4 | Cystatin | Low molecular weight |
| | | | | growth-promoting factor |
| NP01125 | CSCSSLMDKECVYFCHLDIIWVNTPEHVVPYGLGSPRS | 38 | Endothelin/sarafotoxin | Big endothelin-1 |
| NP01126 | CSCSSLMDKECVYFCHLDIIW | 21 | Endothelin/sarafotoxin | Endothelin-1 |
| NP01127 | CSCSSWLDKECVYFCHLDIIW | 21 | Endothelin/sarafotoxin | Endothelin-2 |
| NP01128 | CTCFTYKDKECVYYCHLDIIW | 21 | Endothelin/sarafotoxin | Endothelin-3 |
| NP01760 | AGEGLNSQFWSLAAPQRF | 18 | FMRFamide related | Neuropeptide AF |
| | | | peptide | |
| NP01761 | FLFQPQRF | 8 | FMRFamide related | Neuropeptide FF |
| | | | peptide | |
| NP01762 | SQAFLFQPQRF | 11 | FMRFamide related | Neuropeptide SF |
| | | | peptide | |
| NP01763 | SLNFEELKDWGPKNVIKMSTPAVNKMPHSFANLPLRF | 37 | FMRFamide related | Neuropeptide NPSF |
| | | | peptide | (Potential) |
| NP01764 | VPNLPQRF | 8 | FMRFamide related | Neuropeptide NPVF |
| | | | peptide | |
| NP01765 | MPHSFANLPLRF | 12 | FMRFamide related | Neuropeptide RFRP-1 |
| | | | peptide | |
| NP01766 | SAGATANLPLRS | 12 | FMRFamide related | Neuropeptide RFRP-2 |
| | | | peptide | (Potential) |
| NP02007 | TPDINPAWYASRGIRPVGRF | 20 | FMRFamide related | Prolactin-releasing peptide |
| | | | peptide | PrRP20 |
| NP02008 | SRTHRHSMEIRTPDINPAWYASRGIRPVGRF | 31 | FMRFamide related | Prolactin-releasing peptide |
| | | | peptide | PrRP31 |
| NP02025 | GWTLNSAGYLLGPHAVGNHRSFSDKNGLTS | 30 | Galanin | Galanin |
| NP02026 | ELRPEDDMKPGSFDRSIPENNIMRTIIEFLSFLHLKEAGALDRLLDLPAAASSEDIERS | 59 | Galanin | Galanin message-associated |
| | | | peptide | |
| NP02040 | APAHRGRGGWTLNSAGYLLGPVLHLPQMGDQDGKRETALEILDLWKAIDGLPYS | 60 | Galanin | Galanin-like peptide |
| | HPPQPS | | | |
| NP02242 | QPVPPADPAGSGLQRAEEAPRRQLRVSQRTDGESRAHLGALLARYIQQARKAPSGR | 95 | Gastrin/cholecystokinin | Cholecystokinin |
| | MSIVKNLQNLDPSHRISDRDYMGWMDFGRRSAEEYEYPS | | | |
| NP02243 | ISDRDYMGWMDF | 12 | Gastrin/cholecystokinin | Cholecystokinin-12 |
| NP02244 | LDPSHRISDRDYMGWMDF | 18 | Gastrin/cholecystokinin | Cholecystokinin-18 (By |
| | | | | similarity) |
| NP02245 | IVKNLQNLDPSHRISDRDYMGWMDF | 25 | Gastrin/cholecystokinin | Cholecystokinin-25 (By |
| | | | | similarity) |
| NP02246 | KAPSGRMSIVKNLQNLDPSHRISDRDYMGWMDF | 33 | Gastrin/cholecystokinin | Cholecystokinin-33 |
| NP02247 | YIQQARKAPSGRMSIVKNLQNLDPSHRISDRDYMGWMDF | 39 | Gastrin/cholecystokinin | Cholecystokinin-39 |
| NP02248 | GWMDF | 5 | Gastrin/cholecystokinin | Cholecystokinin-5 (By |
| | | | | similarity) |
| NP02249 | VSQRTDGESRAHLGALLARYIQQARKAPSGRMSIVKNLQNLDPSHRISDRDYMGW | 58 | Gastrin/cholecystokinin | Cholecystokinin-58 |
| | MDF | | | |
| NP02250 | VSQRTDGESRAHLGALLARYIQQARKAPSGRMSIVKNLQNLDPSHRISD | 49 | Gastrin/cholecystokinin | Cholecystokinin-58 |
| | | | | desnonopeptide (By |
| | | | | similarity) |
| NP02251 | YMGWMDF | 7 | Gastrin/cholecystokinin | Cholecystokinin-7 (By |
| | | | | similarity) |
| NP02252 | DYMGWMDF | 8 | Gastrin/cholecystokinin | Cholecystokinin-8 |
| NP02253 | QLGPQGPPHLVADPSKKQGPWLEEEEEAYGWMDF | 34 | Gastrin/cholecystokinin | Big gastrin |
| NP02254 | QGPWLEEEEEAYGWMDF | 17 | Gastrin/cholecystokinin | Gastrin |
| NP02255 | WLEEEEEAYGWMDF | 14 | Gastrin/cholecystokinin | Gastrin-14 |
| NP02256 | DLELPWLEQQGPASHHRRQLGPQGPPHLVADPSKKQGPWLEEEEEAYGWMDF | 52 | Gastrin/cholecystokinin | Gastrin-52 |
| NP02257 | YGWMDF | 6 | Gastrin/cholecystokinin | Gastrin-6 |
| NP02258 | SWKPRSQQPDAPLGTGANRDLELPWLEQQGPASHHRRQLGPQGPPHLVADPSKKQ | 71 | Gastrin/cholecystokinin | Gastrin-71 |
| | GPWLEEEEEAYGWMDF | | | |
| NP02464 | YAEGTFISDYSIAMDKIHQQDFVNWLLAQKGKKNDWKHNITQ | 42 | Glucagon | Gastric inhibitory polypeptide |
| NP02465 | RSLQDTEEKSRSFSASQADPLSDPDQMNEDKRHSQGTFTSDYSKYLDSRRAQDFVQ | 69 | Glucagon | Glicentin (By similarity) |
| | WLMNTKRNRNNIA | | | |
| NP02466 | RSLQDTEEKSRSFSASQADPLSDPDQMNED | 30 | Glucagon | Glicentin-related polypeptide |
| | | | | (By similarity) |
| NP02467 | HSQGTFTSDYSKYLDSRRAQDFVQWLMNT | 29 | Glucagon | Glucagon |
| NP02468 | HDEFERHAEGTFTSDVSSYLEGQAAKEFIAWLVKGRG | 37 | Glucagon | Glucagon-like peptide 1 |
| NP02469 | HAEGTFTSDVSSYLEGQAAKEFIAWLVKGR | 30 | Glucagon | Glucagon-like peptide 1(7-36) |
| NP02470 | HAEGTFTSDVSSYLEGQAAKEFIAWLVKGRG | 31 | Glucagon | Glucagon-like peptide 1(7-37) |
| NP02471 | HADGSFSDEMNTILDNLAARDFINWLIQTKITD | 33 | Glucagon | Glucagon-like peptide 2 (By |
| | | | | similarity) |
| NP02472 | HSQGTFTSDYSKYLDSRRAQDFVQWLMNTKRNRNNIA | 37 | Glucagon | Oxyntomodulin (By |
| | | | | similarity) |
| NP02473 | DVAHGILNEAYRKVLDQLSAGKHLQSLVARGVGGSLGGGAGDDAEPLS | 48 | Glucagon | PACAP-related peptide |
| NP02474 | HSDGIFTDSYSRYRKQMAVKKYLAAVL | 27 | Glucagon | Pituitary adenylate cyclase- |
| | | | | activating polypeptide 27 |
| NP02475 | HSDGIFTDSYSRYRKQMAVKKYLAAVLGKRYKQRVKNK | 38 | Glucagon | Pituitary adenylate cyclase- |
| | | | | activating polypeptide 38 |
| NP02476 | HSDGTFTSELSRLREGARLQRLLQGLV | 27 | Glucagon | Secretin |
| NP02477 | YADAIFTNSYRKVLGQLSARKLLQDIMSRQQGESNQERGARARL | 44 | Glucagon | Somatoliberin |
| NP02478 | HADGVFTSDFSKLLGQLSAKKYLESLM | 27 | Glucagon | Intestinal peptide PHM-27 |
| NP02479 | HADGVFTSDFSKLLGQLSAKKYLESLMGKRVSSNISEDPVPV | 42 | Glucagon | Intestinal peptide PHV-42 |
| NP02480 | HSDAVFTDNYTRLRKQMAVKKYLNSILN | 28 | Glucagon | Vasoactive intestinal peptide |
| NP02572 | DAENLIDSFQEIVKEVGQLAETQRFECTTHQPRSPLRDLKGALESLIEEETGQKKI | 56 | GnRH | GnRH-associated peptide 1 |
| NP02573 | QHWSYGLRPG | 10 | GnRH | Gonadoliberin-1 |
| NP02574 | QHWSYGLRPGGKRDAENLIDSFQEIVKEVGQLAETQRFECTTHQPRSPLRDLKGAL | 69 | GnRH | Progonadoliberin-1 |
| | ESLIEEETGQKKI | | | |
| NP02575 | ALSSAQDPQNALRPPGRALDTAAGSPVQTAHGLPSDALAPLDDSMPWEGRTTAQ | 84 | GnRH | GnRH-associated peptide 2 |
| | WSLHRKRHLARTLLTAAREPRPAPPSSNKV | | | |
| NP02576 | QHWSHGWYPG | 10 | GnRH | Gonadoliberin-2 |
| NP02577 | QHWSHGWYPGGKRALSSAQDPQNALRPPGRALDTAAGSPVQTAHGLPSDALAPL | 97 | GnRH | Progonadoliberin-2 |
| | DDSMPWEGRTTAQWSLHRKRHLARTLLTAAREPRPAPPSSNKV | | | |
| NP02765 | GPETLCGAELVDALQFVCGDRGFYFNKPTGYGSSSRRAPQTGIVDECCFRSCDLRR | 70 | Insulin | Insulin-like growth factor I |
| | LEMYCAPLKPAKSA | | | |
| NP02766 | AYRPSETLCGGELVDTLQFVCGDRGFYFSRPASRVSRRSRGIVEECCFRSCDLALLE | 67 | Insulin | Insulin-like growth factor II |
| | TYCATPAKSE | | | |
| NP02767 | YRPSETLCGGELVDTLQFVCGDRGFYFSRPASRVSRRSRGIVEECCFRSCDLALLET | 66 | Insulin | Insulin-like growth factor II |
| | YCATPAKSE | | | Ala-25 Del |
| NP02768 | DVSTPPTVLPDNFPRYPVGKFFQYDTWKQSTQRL | 34 | Insulin | Preptin |
| NP02769 | GIVEQCCTSICSLYQLENYCN | 21 | Insulin | Insulin A chain |
| NP02770 | FVNQHLCGSHLVEALYLVCGERGFFYTPKT | 30 | Insulin | Insulin B chain |
| NP02771 | PYVALFEKCCLIGCTKRSLAKYC | 23 | Insulin | Relaxin A chain (By |
| | | | | similarity) |
| NP02772 | VAAKWKDDVIKLCGRELVRAQIAICGMSTWS | 31 | Insulin | Relaxin B chain (By |
| | | | | similarity) |
| NP02773 | QLYSALANKCCHVGCTKRSLARFC | 24 | Insulin | Relaxin A chain |
| NP02774 | DSWMEEVIKLCGRELVRAQIAICGMSTWS | 29 | Insulin | Relaxin B chain |
| NP02775 | DVLAGLSSSCCKWGCSKSEISSLC | 24 | Insulin | Relaxin-3 A chain (By |
| | | | | similarity) |
| NP02776 | RAAPYGVRLCGREFIRAVIFTCGGSRW | 27 | Insulin | Relaxin-3 B chain (By |
| | | | | similarity) |
| NP02840 | YNWNSFGLRF | 10 | KISS1 | Kisspeptin-10 |
| NP02841 | LPNYNWNSFGLRF | 13 | KISS1 | Kisspeptin-13 |
| NP02842 | DLPNYNWNSFGLRF | 14 | KISS1 | Kisspeptin-14 |
| NP02843 | EPLEKVASVGNSRPTGQQLESLGLLAPGEQSLPCTERKPAATARLSRRGTSLSPPPES | 119 | KISS1 | Metastasis-suppressor KiSS-1 |
| | SGSPQQPGLSAPHSRQIPAPQGAVLVQREKDLPNYNWNSFGLRFGKREAAPGNHG | | | |
| | RSAGRG | | | |
| NP02844 | GTSLSPPPESSGSPQQPGLSAPHSRQIPAPQGAVLVQREKDLPNYNWNSFGLRF | 54 | KISS1 | Metastin |
| NP02870 | VPIQKVQDDTKTLIKTIVTRINDISHTQSVSSKQKVTGLDFIPGLHPILTLSKMDQTL | 146 | Leptin | Leptin |
| | AVYQQILTSMPSRNVIQISNDLENLRDLLHVLAFSKSCHLPWASGLETLDSLGGVLE | | | |
| | ASGYSTEVVALSRLQGSLQDMLWQLDLSPGC | | | |
| NP02908 | DFDMLRCMLGRVYRPCWQV | 19 | Melanin-concentrating | Melanin-concentrating |
| | | | hormone | hormone |
| NP02909 | EIGDEENSAKFPI | 13 | Melanin-concentrating | Neuropeptide-glutamic acid- |
| | | | hormone | isoleucine |
| NP02910 | GSVAFPAENGVQNTESTQE | 19 | Melanin-concentrating | Neuropeptide-glycine- |
| | | | hormone | glutamic acid(Potential) |
| NP02937 | ILLSASKSIRNLDDDMVFNTFRLGKGFQKEDTAEKSVIAPSLEQYKNDESSFMNEEE | 144 | Melanin-concentrating | Pro-MCH |
| | NKVSKNTGSKHNFLNHGLPLNLAIKPYLALKGSVAFPAENGVQNTESTQEKREIGD | | hormone | |
| | EENSAKFPIGRRDFDMLRCMLGRVYRPCWQV | | | |
| NP03000 | GSSFLSPEHQRVQQRKESKKPPAKLQP | 27 | Motilin | Ghrelins-27 |
| NP03001 | GSSFLSPEHQRVQQRKESKKPPAKLQPR | 28 | Motilin | Ghrelins-28 |
| NP03002 | FNAPFDVGIKLSGVQYQQHSQAL | 23 | Motilin | Obestatin |
| NP03003 | FVPIFTYGELQRMQEKERNKGQ | 22 | Motilin | Motilin |
| NP03004 | SLSVWQRSGEEGPVDPAEPIREEENEMIKLTAPLEIGMRMNSRQLEKYPATLEGLLS | 66 | Motilin | Motilin-associated peptide |
| | EMLPQHAAK | | | |
| NP03027 | FVPIFTYGELQRMQEKERNKGQKKSLSVWQRSGEEGPVDPAEPIREEENEMIKLTA | 90 | Motilin | Promotilin |
| | PLEIGMRMNSRQLEKYPATLEGLLSEMLPQHAAK | | | |
| NP03617 | ETTTQGPGVLLPLPKGACTGWMAGIPGHPGHNGAPGRDGRDGTPGEKGEKGDPGL | 226 | NA | Adiponectin |
| | IGPKGDIGETGVPGAEGPRGFPGIQGRKGEPGEGAYVYRSAFSVGLETYVTIPNMPI | | | |
| | RFTKIFYNQQNHYDGSTGKFHCNIPGLYYFAYHITVYMKDVKVSLFKKDKAMLFT | | | |
| | YDQYQENNVDQASGSVLLHLEVGDQVWLQVYGEGERNGLYADNDNDSTFTGFLL | | | |
| | YHDTN | | | |
| NP03618 | MIEVVCNDRLGKKVRVKCNTDDTIGDLKKLIAAQTGTRWNKIVLKKWYTIFKDHV | 73 | NA | Ubiquitin-like protein 5 |
| | SLGDYEIHDGMNLELYYQ | | | |
| NP03619 | AQMGLAPMEGIRRPDQALLPELPGLGLRAPLKKTTAEQAEEDLLQEAQALAEVLD | 112 | NA | Agouti-related protein |
| | LQDREPRSSRRCVRLHESCLGQQVPCCDPCATCYCRFFNAFCYCRKLGTAMNPCSR | | | |
| | T | | | |
| NP03624 | MNPAAEAEFNILLATDSYKVTHYKQYPPNTSKVYSYFECREKKTENSKLRKVKYE | 491 | NAPRTase | Nicotinamide |
| | ETVFYGLQYILNKYLKGKVVTKEKIQEAKDVYKEHFQDDVFNEKGWNYILEKYD | | | phosphoribosyltransferase |
| | GHLPIEIKAVPEGFVIPRGNVLFTVENTDPECYWLTNWIETILVQSWYPITVATNSRE | | | |
| | QKKILAKYLLETSGNLDGLEYKLHDFGYRGVSSQETAGIGASAHLVNFKGTDTVA | | | |
| | GLALIKKYYGTKDPVPGYSVPAAEHSTITAWGKDHEKDAFEHIVTQFSSVPVSVVS | | | |
| | DSYDIYNACEKIWGEDLRHLIVSRSTQAPLIIRPDSGNPLDTVLKVLEILGKKFPVTE | | | |
| | NSKGYKLLPPYLRVIQGDGVDINTLQEIVEGMKQKMWSIENIAFGSGGGLLQKLTR | | | |
| | DLLNCSFKCSYVVTNGLGINVFKDPVADPNKRSKKGRLSLHRTPAGNFVTLEEGKG | | | |
| | DLEEYGQDLLHTVFKNGKVTKSYSFDEIRKNAQLNIELEAAHH | | | |
| NP03673 | SLRRSSCFGGRMDRIGAQSGLGCNSFRY | 28 | Natriuretic peptide | Atrial natriuretic factor |
| NP03674 | NPMYNAVSNADLMDFKNLLDHLEEKMPLED | 30 | Natriuretic peptide | Cardiodilatin-related peptide |
| NP03675 | SPKMVQGSGCFGRKMDRISSSSGLGCKV | 28 | Natriuretic peptide | BNP(1-28) |
| NP03676 | SPKMVQGSGCFGRKMDRISSSSGLGCKVL | 29 | Natriuretic peptide | BNP(1-29) |
| NP03677 | SPKMVQGSGCFGRKMDRISSSSGLGCKVLR | 30 | Natriuretic peptide | BNP(1-30) |
| NP03678 | PKMVQGSGCFGRKMDRISSSSGLGCKVLRR | 30 | Natriuretic peptide | BNP(2-31) |
| NP03679 | KMVQGSGCFGRKMDRISSSSGLGCKVL | 27 | Natriuretic peptide | BNP(3-29) |
| NP03680 | KMVQGSGCFGRKMDRISSSSGLGCKVLR | 28 | Natriuretic peptide | BNP(3-30) |
| NP03681 | KMVQGSGCFGRKMDRISSSSGLGCKVLRRH | 30 | Natriuretic peptide | BNP(3-32) |
| NP03682 | MVQGSGCFGRKMDRISSSSGLGCK | 24 | Natriuretic peptide | BNP(4-27) |
| NP03683 | MVQGSGCFGRKMDRISSSSGLGCKVL | 26 | Natriuretic peptide | BNP(4-29) |
| NP03684 | MVQGSGCFGRKMDRISSSSGLGCKVLR | 27 | Natriuretic peptide | BNP(4-30) |
| NP03685 | MVQGSGCFGRKMDRISSSSGLGCKVLRR | 28 | Natriuretic peptide | BNP(4-31) |
| NP03686 | MVQGSGCFGRKMDRISSSSGLGCKVLRRH | 29 | Natriuretic peptide | BNP(4-32) |
| NP03687 | VQGSGCFGRKMDRISSSSGLGCKVL | 25 | Natriuretic peptide | BNP(5-29) |
| NP03688 | VQGSGCFGRKMDRISSSSGLGCKVLRR | 27 | Natriuretic peptide | BNP(5-31) |
| NP03689 | VQGSGCFGRKMDRISSSSGLGCKVLRRH | 28 | Natriuretic peptide | BNP(5-32) |
| NP03690 | SPKMVQGSGCFGRKMDRISSSSGLGCKVLRRH | 32 | Natriuretic peptide | Brain natriuretic peptide 32 |
| NP03691 | HPLGSPGSASDLETSGLQEQRNHLQGKLSELQVEQTSLEPLQESPRPTGVWKSREV | 108 | Natriuretic peptide | Natriuretic peptides B |
| | ATEGIRGHRKMVLYTLRAPRSPKMVQGSGCFGRKMDRISSSSGLGCKVLRRH | | | |
| NP03692 | GLSKGCFGLKLDRIGSMSGLGC | 22 | Natriuretic peptide | CNP-22 |
| NP03693 | YKGANKKGLSKGCFGLKLDRIGSMSGLGC | 29 | Natriuretic peptide | CNP-29 |
| NP03694 | DLRVDTKSRAAWARLLQEHPNARKYKGANKKGLSKGCFGLKLDRIGSMSGLGC | 53 | Natriuretic peptide | CNP-53 |
| NP03714 | ANLTNGGKSELLKSGSSKSTLKHIWTESSKDLSISRLLSQTFRGKENDTDLDLRYDT | 250 | Neurexophilin | Neurexophilin-1 |
| | PEPYSEQDLWDWLRNSTDLQEPRPRAKRRPIVKTGKFKKMFGWGDFHSNIKTVKL | | | |
| | NLLITGKIVDHGNGTFSVYFRHNSTGQGNVSVSLVPPTKIVEFDLAQQTVIDAKDSK | | | |
| | SFNCRIEYEKVDKATKNTLCNYDPSKTCYQEQTQSHVSWLCSKPFKVICIYISFYST | | | |
| | DYKLVQKVCPDYNYHSDTPYFPSG | | | |
| NP03715 | KEVVHATEGLDWEDKDAPGTLVGNVVHSRIISPLRLFVKQSPVPKPGPMAYADSM | 242 | Neurexophilin | Neurexophilin-2 |
| | ENFWDWLANITEIQEPLARTKRRPIVKTGKFKKMFGWGDFHSNIKTVKLNLLITGKI | | | |
| | VDHGNGTFSVYFRHNSTGLGNVSVSLVPPSKVVEFEVSPQSTLETKESKSFNCRIEY | | | |
| | EKTDRAKKTALCNFDPSKICYQEQTQSHVSWLCSKPFKVICIYIAFYSVDYKLVQK | | | |
| | VCPDYNYHSETPYLSSG | | | |
| NP03716 | QDDGPPGSEDPERDDHEGQPRPRVPRKRGHISPKSRPMANSTLLGLLAPPGEAWGI | 230 | Neurexophilin | Neurexophilin-3 |
| | LGQPPNRPNHSPPPSAKVKKIFGWGDFYSNIKTVALNLLVTGKIVDHGNGTFSVHF | | | |
| | QHNATGQGNISISLVPPSKAVEFHQEQQIFIEAKASKIFNCRMEWEKVERGRRTSLC | | | |
| | THDPAKICSRDHAQSSATWSCSQPFKVVCVYIAFYSTDYRLVQKVCPDYNYHSDTP | | | |
| | YYPSG | | | |
| NP03717 | QIPESGRPQYLGLRPAAAGAGAPGQQLPEPRSSDGLGVGRAWSWAWPTNHTGAL | 285 | Neurexophilin | Neurexophilin-4 |
| | ARAGAAGALPAQRTKRKPSIKAARAKKIFGWGDFYFRVHTLKFSLLVTGKIVDHV | | | |
| | NGTFSVYFRHNSSSLGNLSVSIVPPSKRVEFGGVWLPGPVPHPLQSTLALEGVLPGL | | | |
| | GPPLGMAAAAAGPGLGGSLGGALAGPLGGALGVPGAKESRAFNCHVEYEKTNRA | | | |
| | RKHRPCLYDPSQVCFTEHTQSQAAWLCAKPFKVICIFVSFLSFDYKLVQKVCPDYN | | | |
| | FQSEHPYFG | | | |
| NP03732 | ILQRGSGTAAVDFTKKDHTATWGRPFFLFRPRN | 33 | Neuromedins | Neuromedin-S |
| NP03733 | FRVDEEFQSPFASQSRGYFLFRPRN | 25 | Neuromedins | Neuromedin-U-25 |
| NP03744 | WYKPAAGHSSYSVGRAAGLLSGLR | 24 | Neuropeptide B/W | Neuropeptide B-23 |
| NP03745 | WYKPAAGHSSYSVGRAAGLLSGLRRSPYA | 29 | Neuropeptide B/W | Neuropeptide B-29 |
| NP03746 | WYKHVASPRYHTVGRAAGLLMGL | 23 | Neuropeptide B/W | Neuropeptide W-23 |
| NP03747 | WYKHVASPRYHTVGRAAGLLMGLRRSPYLW | 30 | Neuropeptide B/W | Neuropeptide W-30 |
| NP03755 | SFRNGVGTGMKKTSFQRAKS | 20 | Neuropeptide S | Neuropeptide S |
| NP03782 | SDSEEEMKALEADFLTNMHTSKISKAHVPSWKMTLLNVCSLVNNLNSPAEETGEV | 125 | Neurotensin | Large neuromedin N |
| | HEEELVARRKLPTALDGFSLEAMLTIYQLHKICHSRAFQHWELIQEDILDTGNDKN | | | |
| | GKEEVIKRKIPYIL | | | |
| NP03783 | IPYIL | 5 | Neurotensin | Neuromedin N |
| NP03784 | QLYENKPRRPYIL | 13 | Neurotensin | Neurotensin |
| NP03785 | DSYYY | 5 | Neurotensin | Tail peptide (Potential) |
| NP03886 | SSPETLISDLLMRESTENVPRTRLEDPAMW | 30 | NPY | C-flanking peptide of NPY |
| NP03887 | YPSKPDNPGEDAPAEDMARYYSALRHYINLITRQRY | 36 | NPY | Neuropeptide Y |
| NP03957 | APLEPVYPGDNATPEQMAQYAADLRRYINMLTRPRY | 36 | NPY | Pancreatic hormone |
| NP03958 | HKEDTLAFSEWGSPHAAVPR | 20 | NPY | Pancreatic icosapeptide |
| NP03959 | YPIKPEAPREDASPEELNRYYASLRHYLNLVTRQRY | 36 | NPY | Peptide YY |
| NP03960 | IKPEAPREDASPEELNRYYASLRHYLNLVTRQRY | 34 | NPY | Peptide YY(3-36) |
| NP04021 | VPLERGAPNKEETPATESPDTGLYYHRYLQEVIDVLETDGHFREKLQAANAEDIKS | 435 | Nucleobindin | Nucleobindin-1 |
| | GKLSRELDFVSHHVRTKLDELKRQEVSRLRMLLKAKMDAEQDPNVQVDHLNLLK | | | |
| | QFEHLDPQNQHTFEARDLELLIQTATRDLAQYDAAHHEEFKRYEMLKEHERRRYL | | | |
| | ESLGEEQRKEAERKLEEQQRRHREHPKVNVPGSQAQLKEVWEELDGLDPNRFNPK | | | |
| | TFFILHDINSDGVLDEQELEALFTKELEKVYDPKNEEDDMREMEEERLRMREHVM | | | |
| | KNVDTNQDRLVTLEEFLASTQRKEFGDTGEGWETVEMHPAYTEEELRRFEEELAA | | | |
| | REAELNAKAQRLSQETEALGRSQGRLEAQKRELQQAVLHMEQRKQQQQQQQGH | | | |
| | KAPAAHPEGQLKFHPDTDDVPVPAPAGDQKEVDTSEKKLLERLPEVEVPQHL | | | |
| NP04025 | VPIDIDKTKVQNIHPVESAKIEPPDTGLYYDEYLKQVIDVLETDKHFREKLQKADIE | 82 | Nucleobindin | Nesfatin-1 |
| | EIKSGRLSKELDLVSHHVRTKLDEL | | | |
| NP04026 | VPIDIDKTKVQNIHPVESAKIEPPDTGLYYDEYLKQVIDVLETDKHFREKLQKADIE | 396 | Nucleobindin | Nucleobindin-2 |
| | EIKSGRLSKELDLVSHHVRTKLDELKRQEVGRLRMLIKAKLDSLQDIGMDHQALLK | | | |
| | QFDHLNHLNPDKFESTDLDMLIKAATSDLEHYDKTRHEEFKKYEMMKEHERREYL | | | |
| | KTLNEEKRKEEESKFEEMKKKHENHPKVNHPGSKDQLKEVWEETDGLDPNDFDP | | | |
| | KTFFKLHDVNSDGFLDEQELEALFTKELEKVYDPKNEEDDMVEMEEERLRMREHV | | | |
| | MSEVDTNKDRLVTLEEFLKATEKKEFLEPDSWETLDQQQFFTEEELKEYENIIALQE | | | |
| | NELKKKADELQKQKEELQRQHDQLEAQKLEYHQVIQQMEQKKLQQGIPPSGPAGE | | | |
| | LKFEPHI | | | |
| NP04038 | YAFDVVG | 7 | Opioid | Deltorphin I |
| NP04039 | ELTGQRLRQGDGPNAGADDGPGAQADLEHSLLVAAEKKD | 57 | Opioid | Gamma-Lipotropin |
| | EGPYRMEHFRWGSPPKD | | | |
| NP04040 | YVMGHFRWDRFG | 12 | Opioid | □pmelanocyte-stimulating |
| | | | | hormone |
| NP04123 | YGGFLRKYPK | 10 | Opioid | Alpha-neoendorphin |
| NP04124 | YGGFLRKYP | 9 | Opioid | Beta-neoendorphin |
| NP04125 | YGGFLRRIRPKLKWDNQKRYGGFLRRQFKVVT | 32 | Opioid | Big dynorphin |
| NP04126 | YGGFLRRIRPKLK | 13 | Opioid | Dynorphin A(1-13) (By |
| | | | | similarity) |
| NP04127 | YGGFLRRIRPKLKWDNQ | 17 | Opioid | Dynorphin A(1-17) |
| NP04128 | YGGFLRRI | 8 | Opioid | Dynorphin A(1-8) (By |
| | | | | similarity) |
| NP04129 | YGGFL | 5 | Opioid | Leu-enkephalin |
| NP04130 | YGGFLRRQFKVVTRSQEDPNAYSGELFDA | 29 | Opioid | Leumorphin |
| NP04131 | YGGFLRRQFKVVT | 13 | Opioid | Rimorphin |
| NP04132 | YGGFM | 5 | Opioid | Met-enkephalin |
| NP04133 | YGGFMRGL | 8 | Opioid | Met-enkephalin-Arg-Gly-Leu |
| NP04134 | YGGFMRF | 7 | Opioid | Met-enkephalin-Arg-Phe |
| NP04135 | MDELYPMEPEEEANGSEILA | 20 | Opioid | PENK(114-133) (By |
| | | | | similarity) |
| NP04136 | DAEEDDSLANSSDLLKELLETGDNRERSHHQDGSDNEEEVS | 41 | Opioid | PENK(143-183) (By |
| | | | | similarity) |
| NP04137 | FAEALPSDEEGESYSKEVPEME | 22 | Opioid | PENK(237-258) (By |
| | | | | similarity) |
| NP04138 | ECSQDCATCSYRLVRPADINFLACVMECEGKLPSLKIWETCKELLQLSKPELPQDG | 73 | Opioid | Synenkephalin |
| | TSTLRENSKPEESHLLA | | | |
| NP04139 | MPRVRSLFQEQEEPEPGMEEAGEMEQKQLQ | 30 | Opioid | Neuropeptide 1 (Probable) |
| NP04140 | FSEFMRQYLVLSMQSSQ | 17 | Opioid | Neuropeptide 2 (Probable) |
| NP04141 | FGGFTGARKSARKLANQ | 17 | Opioid | Nociceptin |
| NP04400 | QPLPDCCRQKTCSCRLYELLHGAGNHAAGILTL | 33 | Orexin | Orexin-A |
| NP04401 | RSGPPGLQGRLQRLLQASGNHAAGILTM | 28 | Orexin | Orexin-B |
| NP04409 | SLALADDAAFRERARLLAALERRHWLNSYMHKLLVLDAP | 39 | Parathyroid hormone | Tuberoinfundibular peptide of |
| | | | | 39residues |
| NP04423 | TRSAWLDSGVTGSGLEGDHLSDTSTTSLELDSR | 33 | Parathyroid hormone | Osteostatin |
| NP04424 | AVSEHQLLHDKGKSIQDLRRRFFLHHLIAEIHTAEIRATSEVSPNSKPSPNTKNHPVR | 141 | Parathyroid hormone | Parathyroid hormone-related |
| | FGSDDEGRYLTQETNKVETYKEQPLKTPGKKKKGKPGKRKEQEKKKRRTRSAWL | | | protein |
| | DSGVTGSGLEGDHLSDTSTTSLELDSRRH | | | |
| NP04425 | AVSEHQLLHDKGKSIQDLRRRFFLHHLIAEIHTAEI | 36 | Parathyroid hormone | PTHrP[1-36] |
| NP04426 | ATSEVSPNSKPSPNTKNHPVRFGSDDEGRYLTQETNKVETYKEQPLKTPGKKKKGK | 57 | Parathyroid hormone | PTHrP[38-94] |
| | P | | | |
| NP04892 | YGGFMTSEKSQTPLVTLFKNAIIKNAYKKGE | 31 | POMC | Beta-endorphin |
| NP04893 | SYSMEHFRWGKPVGKKRRPVKVYPNGAEDESAEAFPLEF | 39 | POMC | Corticotropin |
| NP04894 | PVKVYPNGAEDESAEAFPLEF | 21 | POMC | Corticotropin-like |
| | | | | intermediary peptide |
| NP04895 | ELTGQRLREGDGPDGPADDGAGAQADLEHSLLVAAEKKDEGPYRMEHFRWGSPP | 89 | POMC | Lipotropin beta |
| | KDKRYGGFMTSEKSQTPLVTLFKNAIIKNAYKKGE | | | |
| NP04896 | ELTGQRLREGDGPDGPADDGAGAQADLEHSLLVAAEKKDEGPYRMEHFRWGSPP | 56 | POMC | Lipotropin gamma |
| | KD | | | |
| NP04897 | SYSMEHFRWGKPV | 13 | POMC | Melanotropin alpha |
| NP04898 | DEGPYRMEHFRWGSPPKD | 18 | POMC | Melanotropin beta |
| NP04899 | YVMGHFRWDRF | 11 | POMC | Melanotropin gamma |
| NP04900 | WCLESSQCQDLTTESNLLECIRACKPDLSAETPMFPGNGDEQPLTENPRKYVMGHF | 76 | POMC | NPP |
| | RWDRFGRRNSSSSGSSGAGQ | | | |
| NP04901 | EDVSAGEDCGPLPEGGPEPRSDGAKPGPRE | 30 | POMC | Potential peptide |
| NP04921 | ARPVKEPRGLSAASPPLAETGAPRRFRRSVPRGEAAGAVQELARALAHLLEAERQE | 227 | ProSAAS | ProSAAS |
| | RARAEAQEAEDQQARVLAQLLRVWGAPRNSDPALGLDDDPDAPAAQLARALLRA | | | |
| | RLDPAALAAQLVPAPVPAAALRPRPPVYDDGPAGPDAEEAGDETPDVDPELLRYL | | | |
| | LGRILAGSADSEGVAAPRRLRRAADHDVGSELPPEGVLGALLRVKRLETPAPQVPA | | | |
| | RRLLPP | | | |
| NP04922 | LETPAPQVPARRLLPP | 16 | ProSAAS | Big LEN (By similarity) |
| NP04923 | AADHDVGSELPPEGVLGALLRVKRLETPAPQVPARRLLPP | 40 | ProSAAS | Big PEN-LEN (By similarity) |
| NP04924 | ARPVKEPRGLSAASPPLAETGAPRRF | 26 | ProSAAS | Big SAAS (By similarity) |
| NP04925 | ARPVKEP | 7 | ProSAAS | KEP (By similarity) |
| NP04926 | LETPAPQVPA | 10 | ProSAAS | Little LEN (By similarity) |
| NP04927 | GLSAASPPLAETGAPRRF | 18 | ProSAAS | Little SAAS (By similarity) |
| NP04928 | AADHDVGSELPPEGVLGALLRV | 22 | ProSAAS | PEN (By similarity) |
| NP05218 | KTLCSMEEAINERIQEVAGSLIFRAISSIGLECQSVTSRGDLATCPRGFAVTGCTCGS | 90 | Resistin/FIZZ | Resistin |
| | ACGSWDVRAETTCHCQCAGMDWTGARCCRVQP | | | |
| NP05219 | QCSLDSVMDKKIKDVLNSLEYSPSPISKKLSCASVKSQGRPSSCPAGMAVTGCACG | 88 | Resistin/FIZZ | Resistin-like beta |
| | YGCGSWDVQLETTCHCQCSVVDWTTARCCHLT | | | |
| NP05222 | QDEGSEATGFLPAAGEKTSGPLGNLAEELNGYSRKKGGFSFRF | 43 | RFamide neuropeptide | QRF-amide |
| NP05241 | SEEPPISLDLTFHLLREVLEMARAEQLAQQAHSNRKLMEII | 41 | Sauvagine/corticotropin- | Corticoliberin |
| | | | releasing | |
| | | | factor/urotensin I | |
| NP05242 | DNPSLSIDLTFHLLRTLLELARTQSQRERAEQNRIIFDSV | 40 | Sauvagine/corticotropin- | Urocortin |
| | | | releasing | |
| | | | factor/urotensin I | |
| NP05243 | IVLSLDVPIGLLQILLEQARARAAREQATTNARILARVGHC | 41 | Sauvagine/corticotropin- | Urocortin-2 |
| | | | releasing | |
| | | | factor/urotensin I | |
| NP05244 | FTLSLDVPTNIMNLLFNIAKAKNLRAQAAANAHLMAQI | 38 | Sauvagine/corticotropin- | Urocortin-3 |
| | | | releasing | |
| | | | factor/urotensin I | |
| NP05299 | MDPNAAYVNMSNHHRGLASANVDFAFSLYKHLVALSPKKNIFISPVSISMALAMLS | 383 | Serpin | Corticosteroid-binding |
| | LGTCGHTRAQLLQGLGFNLTERSETEIHQGFQHLHQLFAKSDTSLEMTMGNALFLD | | | globulin |
| | GSLELLESFSADIKHYYESEVLAMNFQDWATASRQINSYVKNKTQGKIVDLFSGLD | | | |
| | SPAILVLVNYIFFKGTWTQPFDLASTREENFYVDETTVVKVPMMLQSSTISYLHDSE | | | |
| | LPCQLVQMNYVGNGTVFFILPDKGKMNTVIAALSRDTINRWSAGLTSSQVDLYIPK | | | |
| | VTISGVYDLGDVLEEMGIADLFTNQANFSRITQDAQLKSSKVVHKAVLQLNEEGV | | | |
| | DTAGSTGVTLNLTSKPIILRFNQPFIIMIFDHFTWSSLFLARVMNPV | | | |
| NP05300 | QPLLAHGDKSLQGPQPPRHQLSEPAPAYHRITPTITNFALRLYKELAADAPGNIFFSP | 403 | Serpin | Serpin A11 |
| | VSISTTLALLSLGAQANTSALILEGLGFNLTETPEADIHQGFRSLLHTLALPSPKLELK | | | |
| | VGNSLFLDKRLKPRQHYLDSIKELYGAFAFSANFTDSVTTGRQINDYLRRQTYGQV | | | |
| | VDCLPEFSQDTFMVLANYIFFKAKWKHPFSRYQTQKQESFFVDERTSLQVPMMHQ | | | |
| | KEMHRFLYDQDLACTVLQIEYRGNALALLVLPDPGKMKQVEAALQPQTLRKWGQ | | | |
| | LLLPSLLDLHLPRFSISGTYNLEDILPQIGLTNILNLEADFSGVTGQLNKTISKVSHKA | | | |
| | MVDMSEKGTEAGAASGLLSQPPSLNTMSDPHAHFNRPFLLLLWEVTTQSLLFLGK | | | |
| | VVNPVAG | | | |
| NP05301 | LKPSFSPRNYKALSEVQGWKQRMAAKELARQNMDLGFKLLKKLAFYNPGRNIFLS | 394 | Serpin | Serpin A12 |
| | PLSISTAFSMLCLGAQDSTLDEIKQGFNFRKMPEKDLHEGFHYIIHELTQKTQDLKL | | | |
| | SIGNTLFIDQRLQPQRKFLEDAKNFYSAETILTNFQNLEMAQKQINDFISQKTHGKIN | | | |
| | NLIENIDPGTVMLLANYIFFRARWKHEFDPNVTKEEDFFLEKNSSVKVPMMFRSGI | | | |
| | YQVGYDDKLSCTILEIPYQKNITAIFILPDEGKLKHLEKGLQVDTFSRWKTLLSRRV | | | |
| | VDVSVPRLHMTGTFDLKKTLSYIGVSKIFEEHGDLTKIAPHRSLKVGEAVHKAELK | | | |
| | MDERGTEGAAGTGAQTLPMETPLVVKIDKPYLLLIYSEKIPSVLFLGKIVNPIGK | | | |
| NP05302 | SRCSAQKNTEFAVDLYQEVSLSHKDNIIFSPLGITLVLEMVQLGAKGKAQQQIRQTL | 387 | Serpin | Serpin I2 |
| | KQQETSAGEEFFVLKSFFSAISEKKQEFTFNLANALYLQEGFTVKEQYLHGNKEFFQ | | | |
| | SAIKLVDFQDAKACAEMISTWVERKTDGKIKDMFSGEEFGPLTRLVLVNAIYFKGD | | | |
| | WKQKFRKEDTQLINFTKKNGSTVKIPMMKALLRTKYGYFSESSLNYQVLELSYKG | | | |
| | DEFSLIIILPAEGMDIEEVEKLITAQQILKWLSEMQEEEVEISLPRFKVEQKVDFKDV | | | |
| | LYSLNITEIFSGGCDLSGITDSSEVYVSQVTQKVFFEINEDGSEAATSTGIHIPVIMSL | | | |
| | AQSQFIANHPFLFIMKHNPTESILFMGRVTNPDTQEIKGRDLDSL | | | |
| NP05326 | DRVY | 4 | Serpin | Angiotensin 1-4 |
| NP05327 | DRVYI | 5 | Serpin | Angiotensin 1-5 |
| NP05328 | DRVYIHP | 7 | Serpin | Angiotensin 1-7 |
| NP05329 | DRVYIHPFH | 9 | Serpin | Angiotensin 1-9 |
| NP05330 | DRVYIHPFHL | 10 | Serpin | Angiotensin-1 |
| NP05331 | DRVYIHPF | 8 | Serpin | Angiotensin-2 |
| NP05332 | RVYIHPF | 7 | Serpin | Angiotensin-3 |
| NP05333 | VYIHPF | 6 | Serpin | Angiotensin-4 |
| NP05334 | DRVYIHPFHLVIHNESTCEQLAKANAGKPKDPTFIPAPIQAKTSPVDEKALQDQLVL | 452 | Serpin | Angiotensinogen |
| | VAAKLDTEDKLRAAMVGMLANFLGFRIYGMHSELWGVVHGATVLSPTAVFGTLA | | | |
| | SLYLGALDHTADRLQAILGVPWKDKNCTSRLDAHKVLSALQAVQGLLVAQGRAD | | | |
| | SQAQLLLSTVVGVFTAPGLHLKQPFVQGLALYTPVVLPRSLDFTELDVAAEKIDRF | | | |
| | MQAVTGWKTGCSLMGASVDSTLAFNTYVHFQGKMKGFSLLAEPQEFWVDNSTSV | | | |
| | SVPMLSGMGTFQHWSDIQDNFSVTQVPFTESACLLLIQPHYASDLDKVEGLTFQQN | | | |
| | SLNWMKKLSPRTIHLTMPQLVLQGSYDLQDLLAQAELPAILHTELNLQKLSNDRIR | | | |
| | VGEVLNSIFFELEADEREPTESTQQLNKPEVLEVTLNRPFLFAVYDQSATALHFLGR | | | |
| | VANPLSTA | | | |
| NP05404 | DRMPCRNFFWKTFSSCK | 17 | Somastostatin | Cortistatin-17 |
| NP05405 | QEGAPPQQSARRDRMPCRNFFWKTFSSCK | 29 | Somastostatin | Cortistatin-29 (Potential) |
| NP05406 | AGCKNFFWKTFTSC | 14 | Somastostatin | Somatostatin-14 |
| NP05407 | SANSNPAMAPRERKAGCKNFFWKTFTSC | 28 | Somastostatin | Somatostatin-28 |
| NP05514 | LPICPGGAARCQVTLRDLFDRAVVLSHYIHNLSSEMFSEFDKRYTHGRGFITKAINS | 199 | Somatotropin/prolactin | Prolactin |
| | CHTSSLATPEDKEQAQQMNQKDFLSLIVSILRSWNEPLYHLVTEVRGMQEAPEAIL | | | |
| | SKAVEIEEQTKRLLEGMELIVSQVHPETKENEIYPVWSGLPSLQMADEESRLSAYY | | | |
| | NLLHCLRRDSHKIDNYLKLLKCRIIHNNNC | | | |
| NP05529 | RPKPQFFGLM | 10 | Tachykinin | Substance P |
| NP05682 | ALNSVAYERSAMQNYE | 16 | Tachykinin | C-terminal-flanking peptide |
| NP05683 | HKTDSFVGLM | 10 | Tachykinin | Neurokinin A |
| NP05684 | DADSSIEKQVALLKALYGHGQISHKRHKTDSFVGLM | 36 | Tachykinin | Neuropeptide K |
| NP05685 | RPKPQQFFGLM | 11 | Tachykinin | Substance P |
| NP05686 | DMHDFFVGLM | 10 | Tachykinin | Neurokinin-B |
| NP05799 | QPEAAQQEAVTAAEHPGLDDFLRQVERLLFLRENIQRLQGDQGEHSASQIFQSDWL | 218 | TRH | Pro-thyrotropin-releasing |
| | SKRQHPGKREEEEEEGVEEEEEEEGGAVGPHKRQHPGRREDEASWSVDVTQHKRQ | | | hormone |
| | HPGRRSPWLAYAVPKRQHPGRRLADPKAQRSWEEEEEEEEREEDLMPEKRQHPGK | | | |
| | RALGGPCGPQGAYGQAGLLLGLLDDLSRSQGAEEKRQHPGRRAAWVREPLEE | | | |
| NP05800 | QHP | 3 | TRH | Thyrotropin-releasing |
| | | | | hormone |
| NP05810 | ETPDCFWKYCV | 11 | Urotensin-2 | Urotensin-2 |
| NP05811 | ACFWKYCV | 8 | Urotensin-2 | Urotensin-2B |
| NP05890 | AAPDLDVRKCLPCGPGGKGRCFGPNICCAEELGCFVGTAEALRCQEENYLPSPCQS | 94 | Vasopressin/oxytocin | Neurophysin 1 |
| | GQKACGSGGRCAVLGLCCSPDGCHADPACDAEATFSQR | | | |
| NP05891 | CYIQNCPLG | 9 | Vasopressin/oxytocin | Oxytocin |
| NP05892 | CYFQNCPRG | 9 | Vasopressin/oxytocin | Arg-vasopressin |
| NP05893 | ASDRSNATQLDGPAGALLLRLVQLAGAPEPFEPAQPDAY | 39 | Vasopressin/oxytocin | Copeptin |
| NP05894 | AMSDLELRQCLPCGPGGKGRCFGPSICCADELGCFVGTAEALRCQEENYLPSPCQS | 93 | Vasopressin/oxytocin | Neurophysin 2 |
| | GQKACGSGGRCAAFGVCCNDESCVTEPECREGFHRRA | | | |
| NP05912 | TLQPPSALRRRHYHHALPPSRHYP | 24 | VGF | Antimicrobial peptide |
| | | | | VGF[554-577] |
| NP05913 | RPESALLGGSEAGERLLQQGLAQVEA | 26 | VGF | Neuroendocrine regulatory |
| | | | peptide-1 | |
| NP05914 | QAEATRQAAAQEERLADLASDLLLQYLLQGGARQRGLG | 38 | VGF | Neuroendocrine regulatory |
| | | | peptide-2 | |
| NP05915 | APPGRPEAQPPPLSSEHKEPVAGDAVPGPKDGSAPEVRGARNSEPQDEGELFQGVD | 593 | VGF | Neurosecretory protein VGF |
| | PRALAAVLLQALDRPASPPAPSGSQQGPEEEAAEALLTETVRSQTHSLPAPESPEPA | | | |
| | APPRPQTPENGPEASDPSEELEALASLLQELRDFSPSSAKRQQETAAAETETRTHTLT | | | |
| | RVNLESPGPERVWRASWGEFQARVPERAPLPPPAPSQFQARMPDSGPLPETHKFGE | | | |
| | GVSSPKTHLGEALAPLSKAYQGVAAPFPKARRPESALLGGSEAGERLLQQGLAQVE | | | |
| | AGRRQAEATRQAAAQEERLADLASDLLLQYLLQGGARQRGLGGRGLQEAAEERE | | | |
| | SAREEEEAEQERRGGEERVGEEDEEAAEAEAEAEEAERARQNALLFAEEEDGEAG | | | |
| | AEDKRSQEETPGHRRKEAEGTEEGGEEEDDEEMDPQTIDSLIELSTKLHLPADDVVS | | | |
| | IIEEVEEKRKRKKNAPPEPVPPPRAAPAPTHVRSPQPPPPAPAPARDELPDWNEVLPP | | | |
| | WDREEDEVYPPGPYHPFPNYIRPRTLQPPSALRRRHYHHALPPSRHYPGREAQARR | | | |
| | AQEEAEAEERRLQEQEELENYIEHVLLRRP | | | |

TABLE 5

| | Peptide | | | Analogue suquence (potential glycan highlighted |
|---|---|---|---|---|
| Peptide hormone name | analogue name | Trade name | Producer | in bold/underscore) (SEQ ID NO) |
| Amylin | NN9838/AM833 | NA | Novo Nordisk | ? |
| Amylin | Pramlintide | Symlin | Bristol-Myers | KCNTATCATQRLANFLVHSSNNFGPILPPTNVGSNTY- |
| | | | Squibb | NH2 (SEQ ID NO: 280) |
| ANP 32 | Urodilatin | Ularitide | ? | TAPRSLRRSSCFGGRMDRIGAQSGLGCNSFRY (SEQ ID |
| | | | | NO: 281) |
| ANP 28 | Carperitide | HANP | Daiichi Sankyo | SLRRSSCFGGRMDRIGAQSGLGCNSFRY (SEQ ID NO: |
| | | | | 282) |
| ANP 40 | mutant ANP | ? | ? | SLRRSSCFGGRMDRIGAQSGLGCNSFRYRITAREDKQ |
| | | | | GWA (SEQ ID NO: 283) |
| BNP 32 | Nesiritide | Natrecor | Scios, Inc. | SPKMVQGSGCFGRKMDRISSSSGLGCKVLRRH (SEQ |
| | | | | ID NO: 284) |
| Bradykinin | Icatibant | Firazyr | Shire | RRPXGXSXXR (SEQ ID NO: 285) |
| Calcitonin | Salmon Calcitonin | Fortical/ | Several | CSNLSTCVLGKLSQELHKLQTYPRTNTGSGTP (SEQ ID |
| | | Miacalcin... | | NO: 286) |
| DNP/CNP | cdNP/cenderitide | ? | ? | GLSKGCFGLKLDRIGSMSGLGCPSLRDPRPNAPSTSA |
| (snake DNP CNP combination) | | | | (SEQ ID NO: 287) |
| GIP/GLP-1 | NN9277 GG dual | NA | Novo Nordisk | ? |
| | agonist | | | |
| GIP/GLP-1/Glucagon | NN9423 Triagonist | NA | Novo Nordisk | ? |
| | 1706 | | | |
| GLP-1 | Exenatide | BYETTA/ | Bristol-Myers | HGEGTFTSDLSKQMEEEAVRLFIEWLKNGGPSSGAPPP |
| | | BYDUREON | Squibb | S (SEQ ID NO: 288) |
| GLP-1 | Lixisenatide | Lyxumia/Adlyxin | Sanofi | HGEGTFTSDLSKQMEEEAVRLFIEWLKNGGPSSGAPPS |
| | | | | KKKKKK (SEQ ID NO: 289) |
| GLP-1 GLP-1(7-37) linked to Albumin) | Albiglutide | Tanzeum | GlaxoSmithKline | ? |
| GLP-1 GLP-1(7-37) linked to an Fc | Dulaglutide | Trulicity | Eli Lilly | ? |
| fragment of human IgG4 | | | | |
| GLP-1 | LIRAGLUTIDE | Victoza/Saxenda | Novo Nordisk | HAEGTFTSDVSSYLEGQAAK-E-EFIAWLVRGRG (SEQ |
| | | | | ID NO: 290) |
| GLP-1 (37 aa acylated peptide) | Semaglutide | NA | Novo Nordisk | HAEGTFTSDVSSYLEGQAAKEEFIIAWLVKGRG (SEQ |
| | | | | ID NO: 291) |
| GLP-2 | Teduglutide | GATTEX | NPS | HGDGSFSDEMNTILDNLAARDFINWLIQTKITD (SEQ |
| | | | Pharmaceuticals | ID NO: 292) |
| Glucagon | NN9030/G530S | NA | Novo Nordisk | ? |
| Glucagon (produced in Saccharomyces) | Glucagon | GlucaGen | Novo Nordisk | HSQGTFTSDYSKYLDSRRAQDFVQWLMNT (SEQ ID |
| | | | | NO: 293) |
| Glucagon (produced in Escherichia coli) | Glucagon | Glucagon | Eli Lilly | HSQGTFTSDYSKYLDSRRAQDFVQWLMNT (SEQ ID |
| | | | | NO: 293; same as previous line) |
| PTHR | Abaloparatide/ | Tymlos | Radiuspharm | AVSEHQLLHDKGKSIQDLRRRELLEKLLXKLHTA |
| | BA058 | | | (SEQ ID NO: 294) |
| PYY | NN9747/NN9748 | NA | Novo Nordisk | |
| Secretin | Secretin | Secremax/ | Repligen Corp | HSDGTFTSELSRLRDSARLQRLLQGLV (SEQ ID NO: |
| | | Secreflow | | 295) |
| Somatoliberin | Sermorelin | Sermorelin | Emd serono inc | YADAIFTNSYRKVLGQLSARKLLQDIMSRQ (SEQ ID |
| | | acetate | | NO: 296) |
| Somatoliberin | Tesamorelin | Egrifta | Thera- | YADAIFTNSYRKVLGQLSARKLLQDIMSRQQGESNQE |
| | | | technologies | RGARARL (SEQ ID NO: 297) |

TABLE 6
*(Bold; identified or conserved in human, Italic; identified in non-human and not conserved; the SEQ ID Nos. are found in the tables infra)

| | | | | | | *Site position | |
|---|---|---|---|---|---|---|---|
| | | | | | | (Bold; identified | |
| | | | | | | or conserved in | |
| | | | | | | human, Italic; | |
| | | | | | Peptide | identified in non- | No. |
| Entry_ | Peptide | | Peptide | Peptide | Hormone | human and not | of |
| Human | Start | Peptide_Sequence | end | Name | Family | conserved) | sites |
| P05408 | 200 | SVPHFSDEDKDPE | 212 | C-terminal | 7B2 | S200 | 1 |
| | | | | peptide (By | | | |
| | | | | similarity) | | | |
| P05408 | 27 | YSPRTPDRVSEADIQRLLHGVMEQLGIARPRVEYPAHQA | 212 | Neuroendocrine | 7B2 | S28, T31, S36, | 4 |
| | | MNLVGPQSIEGGAHEGLQHLGPFGNIPNIVAELTGDNIPK | | protein 7B2 | | T134 S200 | |
| | | DFSEDQGYPDPPNPCPVGKTADDGCLENTPDTAEFSREFQ | | | | | |
| | | LHQHLFDPEHDYPGLGKWNKKLLYEKMKGGERRKRRSV | | | | | |
| | | NPYLQGQRLDNVVAKKSVPHFSDEDKDPE | | | | | |
| P05408 | 27 | YSPRTPDRVSEADIQRLLHGVMEQLGIARPRVEYPAHQA | 176 | N-terminal | 7B2 | S28, T31, S36, | 3 |
| | | MNLVGPQSIEGGAHEGLQHLGPFGNIPNIVAELTGDNIPK | | peptide (By | | T134 | |
| | | DFSEDQGYPDPPNPCPVGKTADDGCLENTPDTAEFSREFQ | | similarity) | | | |
| | | LHQHLFDPEHDYPGLGKWNKKLLYEKMKGGE | | | | | |
| Q8N6N7 | 1 | MALQADFDRAAEDVRKLKARPDDGELKELYGLYKQAIV | 88 | Acyl-CoA- | ACBP | | |
| | | GDINIACPGMLDLKGKAKWEAWNLKKGLSTEDATSAYIS | | binding domain- | | | |
| | | KAKELIEKYGI | | containing | | | |
| | | | | protein 7 | | | |
| P07108 | 2 | SQAEFEKAAEEVRHLKTKPSDEEMLFIYGHYKQATVGDI | 87 | Acyl-CoA- | ACBP | T36 or T42 | 1 |
| | | NTERPGMLDFTGKAKWDAWNELKGTSKEDAMKAYINK | | binding protein | | (ambiguous) | |
| | | VEELKKKYGI | | | | | |
| P35318 | 95 | YRQSMNNFQGLRSEGCREGTCTVQKLAHQIYQFTDKDKD | 146 | Adrenomedullin | Adrenomedullin | | |
| | | NVAPRSKISPQGY | | | | | |
| Q7Z4H4 | 101 | TQAQLLRVGCVLGTCQVQNLSHRLWQLMGPAGRQDSAP | 147 | Adrenomedullin- | Adrenomedullin | | |
| | | VDPSSPHSY | | 2 (By similarity) | | | |
| Q7Z4H4 | 108 | VGCVLGTCQVQNLSHRLWQLMGPAGRQDSAPVDPSSPH | 147 | Intermedin-short | Adrenomedullin | | |
| | | SY | | (Potential) | | | |
| P35318 | 22 | ARLDVASEFRKKWNKWALSR | 41 | Proadrenomedull | Adrenomedullin | | |
| | | | | in N-20 terminal | | | |
| | | | | peptide | | | |
| Q9ULZ1 | 65 | QRPRLSHKGPMPF | 77 | Apelin-13 (By | Apelin | | |
| | | | | similarity) | | | |
| Q9ULZ1 | 50 | NGPGPWQGGRRKFRRQRPRLSHKGPMPF | 77 | Apelin-28 (By | Apelin | | |
| | | | | similarity) | | | |
| Q9ULZ1 | 47 | GSRNGPGPWQGGRRKFRRQRPRLSHKGPMPF | 77 | Apelin-31 (By | Apelin | | |
| | | | | similarity) | | | |
| Q9ULZ1 | 42 | LVQPRGSRNGPGPWQGGRRKFRRQRPRLSHKGPMPF | 77 | Apelin-36 (By | Apelin | | |
| | | | | similarity) | | | |
| P07492 | 24 | VPLPAGGGTVLTKMYPRGNHWAVGHLM | 50 | Gastrin-releasing | Bombesin/neurom | P27, T32 | 2 |
| | | | | peptide | edin-B/ranatensin | | |
| P08949 | 47 | GNLWATGHFM | 56 | Neuromedin-B | Bombesin/neurom | T52 | 0 |
| | | | | | edin-B/ranatensin | | |
| P08949 | 25 | APLSWDLPEPRSRASKIRVHSRGNLWATGHFM | 56 | Neuromedin-B- | Bombesin/neurom | T52 | 0 |
| | | | | 32 | edin-B/ranatensin | | |
| P07492 | 41 | GNHWAVGHLM | 50 | Neuromedin-C | Bombesin/neurom | | |
| | | | | | edin-B/ranatensin | | |
| P01042 | 381 | RPPGFSPFR | 389 | Bradykinin | Bradykinin | | |
| P01042 | 380 | KRPPGFSPFR | 389 | Lysyl-bradykinin | Bradykinin | | |
| P01042 | 376 | ISLMKRPPGFSPFR | 389 | T-kinin | Bradykinin | | |
| P01258 | 85 | CGNLSTCMLGTYTQDFNKFHTFPQTAIGVGAP | 116 | Calcitonin | Calcitonin | | |
| P06881 | 83 | ACDTATCVTHRLAGLLSRSGGVVKNNFVPTNVGSKAF | 119 | Calcitonin gene- | Calcitonin | T91 | 1 |
| | | | | related peptide 1 | | | |
| P10092 | 82 | ACNTATCVTHRLAGLLSRSGGMVKSNFVPTNVGSKAF | 118 | Calcitonin gene- | Calcitonin | T90 | 1 |
| | | | | related peptide 2 | | | |
| P10997 | 34 | KCNTATCATQRLANFLVHSSNNFGAILSSTNVGSNTY | 70 | Islet amyloid | Calcitonin | T38, T42, T63 | 3 |
| | | | | polypeptide | | | |
| P01258 | 121 | DMSSDLERDHRPHVSMPQNAN | 141 | Katacalcin | Calcitonin | S135 | 0 |
| Q16568 | 28 | QEDAELQPRALDIYSAVDDASHEKELIEALQEVLKKLKS | 66 | CART(1-39) | CART | | |
| Q16568 | 69 | VPIYEKKYGQVPMCDAGEQCAVRKGARIGKLCDCPRGTS | 116 | CART(42-89) | CART | | |
| | | CNSFLLKCL | | | | | |
| Q16568 | 28 | QEDAELQPRALDIYSAVDDASHEKELIEALQEVLKKLKSK | 116 | Cocaine- and | CART | | |
| | | RVPIYEKKYGQVPMCDAGEQCAVRKGARIGKLCDCPRGT | | amphetamine- | | | |
| | | SCNSFLLKCL | | regulated | | | |
| P23435 | 58 | GSAKVAFSAIRSTNH | 72 | [des-Ser1] | Cerebellins | | |
| | | | | cerebellin | | | |
| P23435 | 57 | SGSAKVAFSAIRSTNH | 72 | Cerebellin | Cerebellins | | |
| P23435 | 22 | QNEIEPIVLEGKCLVVCDSNPTSDPTGTALGISVRSGSAKV | 193 | Cerebellin-1 | Cerebellins | | |
| | | AFSAIRSTNHEPSEMSNRTMIIYEDQVLVNIGNNEDSERST | | | | | |
| | | FIAPRKGIYSENFHVVKVYNRQTIQVSLMLNGWPVISAFA | | | | | |
| | | GDQDVTREAASNGVLIQMEKGDRAYLKLERGNLMGGW | | | | | |
| | | KYSTFSGFLVFPL | | | | | |
| Q8IUK8 | 52 | QNDIEPIVLEGKCLVVCDSSPSADGAVTSSLGISVRSGSAK | 224 | Cerebellin-2 | Cerebellins | | |
| | | VAFSATRSTNHEPSEMSNRTMTIYEDQVLVNIGNHFDLAS | | | | | |
| | | SIFVAPRKGIYSFSFHVVKVYNRQTIQVSLMQNGYPVISAF | | | | | |
| | | AGDQDVTREAASNGVLLLMEREDKVHLKLERGNLMGG | | | | | |
| | | WKYSTFSGFLVFPL | | | | | |
| Q6UW01 | 33 | QEGSEPVLLEGECLVVCEPGRAAAGGPGGAALGEAPPGR | 205 | Cerebellin-3 | Cerebellins | | |
| | | VAFAAVRSHHHEPAGETGNGTSGAIYEDQVLVNEGGGED | | | | | |
| | | RASGSFVAPVRGVYSFRFHVVKVYNRQTVQVSLMLNTW | | | | | |
| | | PVISAFANDPDVTREAATSSVLLPLDPGDRVSLRLRRGNL | | | | | |
| | | LGGWKYSSFSGFLIFPL | | | | | |
| Q9NTU7 | 28 | QNDIEPIVLEGKCLVVCDSNPATDSKGSSSSPLGISVRAAN | 201 | Cerebellin-4 | Cerebellins | | |
| | | SKVAFSAVRSTNHEPSEMSNKTRIIYEDQILVNVGNEFTLE | | | | | |
| | | SVEVAPRKGIYSFSFHVIKVYQSQTIQVNLMLNGKPVISAF | | | | | |
| | | AGDKDVTREAATNGVLLYLDKEDKVYLKLEKGNLVGG | | | | | |
| | | WQYSTFSGFLVFPL | | | | | |
| P10645 | 380 | AYGFRGPGPQL | 390 | AL-11 | Chromogranin/ | | |
| | | | | | secretogranin | | |
| P05060 | 617 | SAEFPDFYDSEEPSVTHQEAENEKDRADQTVLTEDEKKEL | 673 | CCB peptide | Chromogranin/ | S631 | 1 |
| | | ENLAAMDLELQKIAEKF | | | secretogranin | | |
| P10645 | 19 | LPVNSPMNKGDIEVMKCIVEVISDTLSKPSPMPVSQECFE | 457 | Chromogranin-A | Chromogranin/ | S45, S48, S54, | 39 |
| | | TLRGDERILSILRHQNLLKELQDLALQGAKERAHQQKKHS | | | secretogranin | S98, E122, S126, | |
| | | GFEDELSEVLENQSSQAELKEAVEEPSSKDVMEKREDSKE | | | | A145, T146, | |
| | | AEKSGEATDGARPQALPEPMQESKAEGNNQAPGEEEEEEE | | | | A153, L154, | |
| | | EATNTHPPASLPSQKYPGPQAEGDSEGLSQGLVDREKGLS | | | | S161, A169, | |
| | | AEPGWQAKREEEEEEEEEAEAGEEAVPEEEGPTVVLNPHP | | | | N182, T183, | |
| | | SLGYKEIRKGESRSEALAVDGAGKPGAEEAQDPEGKGEQ | | | | P185, A187, | |
| | | EHSQQKEEEEEMAVVPQGLFRGGKSGELEQEEERLSKEW | | | | S188, S191, | |
| | | EDSKRWSKMDQLAKELTAEKRLEGQEEEEDNRDSSMKL | | | | E200, L206, | |
| | | SFRARAYGFRGPGPQLRRGWRPSSREDSLEAGLPLQVRGY | | | | S207, V211, | |
| | | PEEKKEEEGSANRRPEDQELESLSAIEAELEKVAHQLQAL | | | | S218, T251 | |
| | | RRG | | | | L254, N255, | |
| | | | | | | P256, S259, | |
| | | | | | | Y262, P283, | |
| | | | | | | E286, P291, | |
| | | | | | | S300, M309, | |
| | | | | | | V312, T353, | |
| | | | | | | A378, S398, | |
| | | | | | | G413 | |
| P10645 | 134 | EDSKEAEKSGEATDGARPQALPEPMQESKAEGNNQAPGE | 225 | EA-92 | Chromogranin/ | A145, T146, | 17 |
| | | EEEEEEEATNTHPPASLPSQKYPGPQAEGDSEGLSQGLVD | | | secretogranin | A153, L154, | |
| | | REKGLSAEPGWQA | | | | S161, A169, | |
| | | | | | | N182, T183, | |
| | | | | | | P185, A187, | |
| | | | | | | S188, S191, | |
| | | | | | | E200, L206, | |
| | | | | | | S207, V211, | |
| | | | | | | S218 | |
| P10645 | 420 | EEEGSANRRPEDQELESLSAIEAELEKVAHQLQALRR | 456 | ER-37 | Chromogranin/ | S436 or | 1 |
| | | | | | secretogranin | S438(Ambiguous) | |
| P10645 | 228 | EEEEEEEEEAEAGEEAVPEEEGPTVVLNPHPSL | 260 | ES-43 | Chromogranin/ | T251, L254, | 5 |
| | | | | | secretogranin | N255, P256, | |
| | | | | | | S259 | |
| P05060 | 440 | FLGEGHHRVQENQMDKARRHPQGAWKELDRNYLNYGE | 513 | GAWK peptide | Chromogranin/ | H446, N451, | 5 |
| | | EGAPGKWQQQGDLQDTKENREEARFQDKQYSSHHTAE | | | secretogranin | Y474, T511, | |
| | | | | | | A512 | |
| P10645 | 413 | GYPEEKKEEEGSANRRPEDQELESLSAIEAELEKVAHQLQ | 456 | GR-44 | Chromogranin/ | G413 | 1 |
| | | ALRR | | | secretogranin | | |
| P10645 | 393 | GWRPSSREDSLEAGLPLQV | 411 | GV-19 | Chromogranin/ | 5398 | 1 |
| | | | | | secretogranin | | |
| P10645 | 358 | LEGQEEEEDNRDSSMKLSF | 376 | LF-19 | Chromogranin/ | | |
| | | | | | secretogranin | | |
| P10645 | 272 | SEALAVDGAGKPGALEAQDPEGKGEQEHSQQKEEEEEM | 319 | Pancreastatin | Chromogranin/ | P283, E286, | 6 |
| | | AVVPQGLFRG | | | secretogranin | P291, S300, | |
| | | | | | | M309, V312 | |
| P05060 | 21 | MPVDNRNHNEGMVTRCIIEVLSNALSKSSAPPITPECRQV | 677 | Secretogranin-1 | Chromogranin/ | T34, S46, S48, | 40 |
| | | LKTSRKDVKDKETFENENTKFEVRLLRDPADASEAHESSS | | | secretogranin | S49, P52, T79, | |
| | | RGEAGAPGEEDIQGPTKADILKWAEGGGHSRERADEPQ | | | | S93, A95, S100, | |
| | | WSLYPSDSQVSEEVKTRHSEKSQREDEEEEEGENYQKGE | | | | A104, E109, | |
| | | RGEDSSEEKHLEEPGETQNAFLNERKQASAIKKEELVARS | | | | T116, S140, | |
| | | ETHAAGHSQEKTHSREKSSQESGEETGSQENHPQESKGQP | | | | S144, S149, | |
| | | RSQEESEEGEEDATSEVDKRRTRPRHHHGRSRPDRSSQGG | | | | T194, N196 | , |
| | | SLPSEEKGHPQEESEESNVSMASLGEKRDHHSTHYRASEE | | | | S206, S293, | |
| | | EPEYGEEIKGYPGVQAPEDLEWERYRGRGSEEYRAPRPQS | | | | S294, S298, | |
| | | EESWDEEDKRNYPSLELDKMAHGYGEESEEERGLEPGKG | | | | P307, S317, | |
| | | RHHRGRGGEPRAYFMSDTREEKRFLGEGHHRVQENQMD | | | | M318, S320, | |
| | | KARRHPQGAWKELDRNYLNYGEEGAPGKWQQQGDLQD | | | | D326, T330, | |
| | | TKENREEARFQDKQYSSHHTAEKRKRLGELFNPYYDPLQ | | | | Y348, P349, | |
| | | WKSSHFERRDNMNDNFLEGEEENELTLNEKNFFPEYNYD | | | | G350, K396, | |
| | | WVVEKKPFSEDVNWGYEKRNLARVPKLDLKRQYDRVAQ | | | | M397, H446, | |
| | | LDQLLHYRKKSAEFPDFYDSEEPVSTHQEAENEKDRADQ | | | | Y474, T511, | |
| | | TVLTEDEKKELENLAAMDLELQKIAEKFSQRG | | | | A512, T556 | |
| | | | | | | S577, N588, | |
| | | | | | | S631 | |
| P13521 | 31 | QRNQLLQKEPDLRLENVQKFPSPEMIRALEYIENLRQQAH | 617 | Secretogranin-2 | Chromogranin/ | S52, S75, A136, | 16 |
| | | KEESSPDYNPYQGVSVPLQQKENGDESHLPERDSLSEED | | | secretogranin | Y190, T191, | |
| | | WMRIILEALRQAENEPQSAPKENKPYALNSEKNFPMDMS | | | | S194, T197, | |
| | | DDYETQQWPERKLKHMQFPPMYEENSRDNPFKRTNEIVE | | | | S259, T261, | |
| | | EQYTPQSLATLESVFQELGKLTGPNNQKRERMDEEQKLY | | | | S378, T450, | |
| | | TDDEDDIYKANNIAYEDVVGGEDWNPVEEKIESQTQEEV | | | | N489, G529, | |
| | | RDSKENIEKNEQINDEMKRSGQLGIQEEDLRKESKDQLSD | | | | S556, S566, | |
| | | DVSKVIAYLKRLVNAAGSGRLQNGQNGERATRLFEKPLD | | | | P573 | |
| | | SQSIYQLIEISRNLQIPPEDLIEMLKTGEKPNGSVEPERELD | | | | | |
| | | LPVDLDDISEADLDHPDLFQNRMLSKSGYPKTPGRAGILA | | | | | |
| | | LPDGLSVEDILNLLGMESAANQKTSYFPNPYNQEKVLPRL | | | | | |
| | | PYGAGRSRSNQLPKAAWIPHVENRQMAYENLNDKDQEL | | | | | |
| | | GEYLARMLVKYPEIINSNQVKRVPGQGSSEDDLQEEEQIE | | | | | |
| | | QAIKEHLNQGSSQETDKLAPVSKRFPVGPPKNDDTPNRQ | | | | | |
| | | YVVDEDLLMKVLEYLNQEKAEKGREHIAKRAMENM | | | | | |
| Q8WXD2 | 20 | FPKPGGSQDKSLHNRELSAERPLNEQIAEAEEDKIKKTYPP | 468 | Secretogranin-3 | Chromogranin/ | P60, T82, S122, | 15 |
| | | ENKPGQSNYSFVDNLNLLKAITEKEKIEKERQSIRSSPLDN | | | secretogranin | T123, T144, | |
| | | KLNVEDVDSTKNRKLIDDYDSTKSGLDHKFQDDPDGLHQ | | | | A157, D211, | |
| | | LDGTPLTAEDIVHKIAARIYEENDRAVFDKIVSKLLNLGLI | | | | P212, T216, | |
| | | FESQAHTLEDEVAEVLQKLISKEANNYEEDPNKPTSWTE | | | | S217, T219, | |
| | | NQAGKIPEKVTPMAAIQDGLAKGENDETVSNTLTLTNGL | | | | T231, I236, | |
| | | ERRTKTYSEDNFLELQYFPNFYALLKSIDSEKEAKEKETLI | | | | A241, S359 | |
| | | TIMKTLIDFVKMMVKYGTISPEEGVSYLENLDEMIALQTK | | | | | |
| | | NKLEKNATDNISKLFPAPSEKSHEETDSTKEEAAKMEKEY | | | | | |
| | | GSLKDSTKDDNSNPGGKTDEPKGKTEAYLEAIRKNIEWL | | | | | |
| | | KKHDKKGNKEDYDLSKMRDFINKQADAYVEKGILDKEE | | | | | |
| | | AEAIKRIYSSL | | | | | |
| P13521 | 182 | TNEIVEEQYTPQSLATLESVFQELGKLTGPNNQ | 214 | Secretoneurin | Chromogranin/ | Y190, T191, | 4 |
| | | | | | secretogranin | S194, T197 | |
| P10645 | 322 | SGELEQEEERLSKEWEDS | 339 | SS-18 | Chromogranin/ | | |
| | | | | | secretogranin | | |
| P10645 | 19 | LPVNSPMNKGDIEVMKCIVEVISDTLSKPSPMPVSQECFE | 94 | Vasostatin-1 | Chromogranin/ | S45, S48, S54 | 3 |
| | | TLRGDERILSILRHQNLLKELQDLALQGAKERAHQQ | | | secretogranin | | |
| P10645 | 19 | LPVNSPMNKGDIEVMKCIVEVISDTLSKPSPMPVSQECFE | 131 | Vasostatin-2 | Chromogranin/ | S45, S48, S54, | 6 |
| | | TLRGDERILSILRHQNLLKELQDLALQGAKERAHQQKKHS | | | secretogranin | S98, E122, S126 | |
| | | GFEDELSEVLENQSSQAELKEAVEEPSSKDVME | | | | | |
| P10645 | 342 | WSKMDQLA | 349 | WA-8 | Chromogranin/ | | |
| | | | | | secretogranin | | |
| P10645 | 342 | WSKMDQLAKELTAE | 355 | WE-14 | Chromogranin/ | T353 | 1 |
| | | | | | secretogranin | | |
| P01042 | 19 | QESQSEEIDCNDKDLFKAVDAALKKYNSQNQSNNQFVLY | 644 | Kininogen-1 | Cystatin | T137, T151, | 15 |
| | | RITEATKTVGSDTFYSFKYEIKEGDCPVQSGKTWQDCEYK | | | | T261, T273, | |
| | | DAAKAATGECTATVGKRSSTKFSVATQTCQITPAEGPVV | | | | E399, T400, | |
| | | TAQYDCLGCVHPISTQSPDLEPILRHGIQYFNNNTQHSSLF | | | | T401, S403, | |
| | | MLNEVKRAQRQVVAGLNFRITYSIVQTNCSKENFLFLTPD | | | | T407, S408, | |
| | | CKSLWNGDTGECTDNAYIDIQLRIASFSQNCDIYPGKDEV | | | | T546, S604, | |
| | | QPPTKICVGCPRDIPTNSPELEETLTHTITKLNAENNATFYF | | | | T610, S611, | |
| | | KIDNVKKARVQVVAGKKYFIDFVARETTCSKESNEELTES | | | | T628 | |
| | | CETKKLGQSLDCNAEVYVVPWEKKIYPTVNCQPLGMISL | | | | | |
| | | MKRPPGESPERSSRIGEIKEETTVSPPHTSMAPAQDEERDS | | | | | |
| | | GKEQGHTRRHDWGHEKQRKHNLGHGHKHERDQGHGHQ | | | | | |
| | | RGHGLGHGHEQQHGLGHGHKFKLDDDLEHQGGHVLDH | | | | | |
| | | GHKHKHGHGHGKHKNKGKKNGKHNGWKTEHLASSSED | | | | | |
| | | STTPSAQTQEKTEGPTPIPSLAKPGVTVTFSDFQDSDLIAT | | | | | |
| | | MMPPISPAPIQSDDDWIPDIQIDPNGLSFNPISDFPDTTSPK | | | | | |
| | | CPGRPWKSVSEINPTTQMKESYYFDLTDGLS | | | | | |
| P01042 | 19 | QESQSEEIDCNDKDLFKAVDAALKKYNSQNQSNNQFVLY | 380 | Kininogen-1 | Cystatin | T137, | 4 |
| | | RITEATKTVGSDTFYSFKYEIKEGDCPVQSGKTWQDCEYK | | heavy chain | | T151, T261, | |
| | | DAAKAATGECTATVGKRSSTKFSVATQTCQITPAEGPVV | | | | T273 | |
| | | TAQYDCLGCVHPISTQSPDLEPILRHGIQYFNNNTQHSSLF | | | | | |
| | | MLNEVKRAQRQVVAGLNFRITYSIVQTNCSKENFLFLTPD | | | | | |
| | | CKSLWNGDTGECTDNAYIDIQLRIASFSQNCDIYPGKDEV | | | | | |
| | | QPPTKICVGCPRDIPTNSPELEETLTHTITKLNAENNATFYF | | | | | |
| | | KIDNVKKARVQVVAGKKYFIDFVARETTCSKESNEELTES | | | | | |
| | | CETKKLGQSLDCNAEVYVVPWEKKIYPTVNCQPLGMISL | | | | | |
| | | MK | | | | | |
| P01042 | 390 | SSRIGEIKEETTVSPPHTSMAPAQDEERDSGKEQGHTRRH | 644 | Kininogen-1 | Cystatin | E399, T400, | 11 |
| | | DWGHEKQRKHNLGHGHKHERDQGHGHQRGHGLGHGHE | | light chain | | T401, S403, | |
| | | QQHGLGHGHKFKLDDDLEHQGGHVLDHGHKHKHGHGH | | | | T407, S408, | |
| | | GKHKNKGKKNGKHNGWKTEHLASSSEDSTTPSAQTQEK | | | | T546, S604, | |
| | | FEGPTPIPSLAKPGVTVTFSDFQDSDLIATMMPPISPAPIQS | | | | T610, S611, | |
| | | DDDWIPDIQIDPNGLSFNPISDFPDTTSPKCPGRPWKSVSEI | | | | T628 | |
| | | NPTTQMKESYYFDLTDGLS | | | | | |
| P01042 | 431 | WGHE | 434 | Low molecular | Cystatin | | |
| | | | | weight growth- | | | |
| | | | | promoting factor | | | |
| P05305 | 53 | CSCSSLMDKECVYFCHLDIIWVNTPEHVVPYGLGSPRS | 90 | Big endothelin-1 | Endothelin/ | | |
| | | | | | sarafotoxin | | |
| P05305 | 53 | CSCSSLMDKECVYFCHLDIIW | 73 | Endothelin-1 | Endothelin/ | | |
| | | | | | sarafotoxin | | |
| P20800 | 49 | CSCSSWLDKECVYFCHLDIIW | 69 | Endothelin-2 | Endothelin/ | | |
| | | | | | sarafotoxin | | |
| P14138 | 97 | CTCFTYKDKECVYYCHLDIIW | 117 | Endothelin-3 | Endothelin/ | | |
| | | | | | sarafotoxin | | |
| O15130 | 93 | AGEGLNSQFWSLAAPQRF | 110 | Neuropeptide AF | FMRFamide | | |
| | | | | | related peptide | | |
| O15130 | 69 | FLFQPQRF | 76 | Neuropeptide FF | FMRFamide | | |
| | | | | | related peptide | | |
| Q9HCQ7 | 56 | SLNFEELKDWGPKNVIKMSTPAVNKMPHSFANLPLRF | 92 | Neuropeptide | FMRFamide | | |
| | | | | NPSF (Potential) | related peptide | | |
| Q9HCQ7 | 124 | VPNLPQRF | 131 | Neuropeptide | FMRFamide | | |
| | | | | NPVF | related peptide | | |
| Q9HCQ7 | 81 | MPHSFANLPLRF | 92 | Neuropeptide | FMRFamide | | |
| | | | | RFRP-1 | related peptide | | |
| Q9HCQ7 | 101 | SAGATANLPLRS | 112 | Neuropeptide | FMRFamide | | |
| | | | | RFRP-2 | related peptide | | |
| | | | | (Potential) | | | |
| O15130 | 66 | SQAFLFQPQRF | 76 | Neuropeptide SF | FMRFamide | | |
| | | | | related peptide | | | |
| P81277 | 34 | TPDINPAWYASRGIRPVGRF | 53 | Prolactin- | FMRFamide | | |
| | | | | releasing peptide | related peptide | | |
| | | | | PrRP20 | | | |
| P81277 | 23 | SRTHRHSMEIRTPDINPAWYASRGIRPVGRF | 53 | Prolactin- | FMRFamide | | |
| | | | | releasing peptide | related peptide | | |
| | | | | PrRP31 | | | |
| P22466 | 33 | GWTLNSAGYLLGPHAVGNHRSFSDKNGLTS | 62 | Galanin | Galanin | S55 | 0 |
| P22466 | 65 | ELRPEDDMKPGSFDRSIPENNIMRTHEFLSELHLKEAGALD | 123 | Galanin | Galanin | A113 | 1 |
| | | RLLDLPAAASSEDIERS | | message- | | | |
| | | | | associated | | | |
| | | | | peptide | | | |
| Q9UBC7 | 25 | APAHRGRGGWTLNSAGYLLGPVLHLPQMGDQDGKRETA | 84 | Galanin-like | Galanin | | |
| | | LEILDLWKAIDGLPYSHPPQPS | | peptide | | | |
| P01350 | 59 | QLGPQGPPHLVADPSKKQGPWLEEEEEAYGWMDF | 92 | Big gastrin | Gastrin/ | | |
| | | | | | cholecystokinin | | |
| P06307 | 21 | QPVPPADPAGSGLQRAEEAPRRQLRVSQRTDGESRAHLG | 115 | Cholecystokinin | Gastrin/ | A28, T50, N85 | 3 |
| | | ALLARYIQQARKAPSGRMSIVKNLQNLDPSHRISDRDYM | | | cholecystokinin | | |
| | | GWMDFGRRSAEEYEYPS | | | | | |
| P06307 | 92 | ISDRDYMGWMDF | 103 | Cholecystokinin- | Gastrin/ | | |
| | | | | 12 | cholecystokinin | | |
| P06307 | 86 | LDPSHRISDRDYMGWMDF | 103 | Cholecystokinin- | Gastrin/ | | |
| | | | | 18 (By | cholecystokinin | | |
| | | | | similarity) | | | |
| P06307 | 79 | IVKNLQNLDPSHRISDRDYMGWMDF | 103 | Cholecystokinin- | Gastrin/ | N85 | 1 |
| | | | | 25 (By | cholecystokinin | | |
| | | | | similarity) | | | |
| P06307 | 71 | KAPSGRMSIVKNLQNLDPSHRISDRDYMGWMDF | 103 | Cholecystokinin- | Gastrin/ | N85 | 1 |
| | | | | 33 | cholecystokinin | | |
| P06307 | 65 | YIQQARKAPSGRMSIVKNLQNLDPSHRISDRDYMGWMDF | 103 | Cholecystokinin- | Gastrin/ | N85 | 1 |
| | | | | 39 | cholecystokinin | | |
| P06307 | 99 | GWMDF | 103 | Cholecystokinin- | Gastrin/ | | |
| | | | | 5 (By similarity) | cholecystokinin | | |
| P06307 | 46 | VSQRTDGESRAHLGALLARYIQQARKAPSGRMSIVKNLQ | 103 | Cholecystokinin- | Gastrin/ | T50, N85 | 2 |
| | | NLDPSHRISDRDYMGWMDF | | 58 | cholecystokinin | | |
| P06307 | 46 | VSQRTDGESRAHLGALLARYIQQARKAPSGRMSIVKNLQ | 94 | Cholecystokinin- | Gastrin/ | T50, N85 | 2 |
| | | NLDPSHRISD | | 58 | cholecystokinin | | |
| | | | | desnonopeptide | | | |
| | | | | (By similarity) | | | |
| P06307 | 97 | YMGWMDF | 103 | Cholecystokinin- | Gastrin/ | | |
| | | | | 7 (By similarity) | cholecystokinin | | |
| P06307 | 96 | DYMGWMDF | 103 | Cholecystokinin- | Gastrin/ | | |
| | | | | 8 | cholecystokinin | | |
| P01350 | 76 | QGPWLEEEEEAYGWMDF | 92 | Gastrin | Gastrin/ | | |
| | | | | | cholecystokinin | | |
| P01350 | 79 | WLEEEEEAYGWMDF | 92 | Gastrin-14 | Gastrin/ | | |
| | | | | | cholecystokinin | | |
| P01350 | 41 | DLELPWLEQQGPASHHRRQLGPQGPPHLVADPSKKQGPW | 92 | Gastrin-52 | Gastrin/ | | |
| | | LEEEEEAYGWMDF | | | cholecystokinin | | |
| P01350 | 87 | YGWMDF | 92 | Gastrin-6 | Gastrin/ | | |
| | | | | | cholecystokinin | | |
| P01350 | 22 | SWKPRSQQPDAPLGTGANRDLELPWLEQQGPASHHRRQL | 92 | Gastrin-71 | Gastrin/ | L33 or G34 | 1 |
| | | GPQGPPHLVADPSKKQGPWLEEEEEAYGWMDF | | | cholecystokinin | (Ambiguous) | |
| P09681 | 52 | YAEGTFISDYSIAMDKIHQQDFVNWLLAQKGKKNDWKH | 93 | Gastric inhibitory | Glucagon | | |
| | | NITQ | | polypeptide | | | |
| P01275 | 21 | RSLQDTEEKSRSFSASQADPLSDPDQMNEDKRHSQGTFTS | 89 | Glicentin (By | Glucagon | S32, T59 | 2 |
| | | DYSKYLDSRRAQDFVQWLMNTKRNRNNIA | | similarity) | | | |
| P01275 | 21 | RSLQDTEEKSRSFSASQADPLSDPDQMNED | 50 | Glicentin-related | Glucagon | S32 | 1 |
| | | | | polypeptide (By | | | |
| | | | | similarity) | | | |
| P01275 | 53 | HSQGTFTSDYSKYLDSRRAQDFVQWLMNT | 81 | Glucagon | Glucagon | T59 | 1 |
| P01275 | 92 | HDEFERHAEGTFTSDVSSYLEGQAAKEFIAWLVKGRG | 128 | Glucagon-like | Glucagon | T102 or T104 or | 1 |
| | | | | peptide 1 | | S105 | |
| | | | | | | (Ambiguous) | |
| P01275 | 98 | HAEGTFTSDVSSYLEGQAAKEFIAWLVKGR | 128 | Glucagon-like | Glucagon | T102 or T104 or | 1 |
| | | | | peptide 1(7-36) | | S105 | |
| | | | | | | (Ambiguous) | |
| P01275 | 98 | HAEGTFTSDVSSYLEGQAAKEFIAWLVKGRG | 127 | Glucagon-like | Glucagon | T102 or T104 or | 1 |
| | | | | peptide 1(7-37) | | S105 | |
| | | | | | | (Ambiguous) | |
| P01275 | 146 | HADGSFSDEMNTILDNLAARDFINVVLIQTKITD | 178 | Glucagon-like | Glucagon | | |
| | | | | peptide 2 (By | | | |
| | | | | similarity) | | | |
| P01282 | 81 | HADGVFTSDFSKLLGQLSAKKYLESLM | 107 | Intestinal peptide | Glucagon | T87, S88 | 2 |
| | | | | PHM-27 | | | |
| P01282 | 81 | HADGVFTSDFSKLLGQLSAKKYLESLMGKRVSSNISEDPV | 122 | Intestinal peptide | Glucagon | T87, S88 | 2 |
| | | PV | | PHV-42 | | | |
| P01275 | 53 | HSQGTFTSDYSKYLDSRRAQDFVQWLMNTKRNRNNIA | 89 | Oxyntomodulin | Glucagon | T59 | 1 |
| | | | | (By similarity) | | | |
| P18509 | 82 | DVAHGILNEAYRKVLDQLSAGKHLQSLVARGVGGSLGG | 129 | PACAP-related | Glucagon | | |
| | | GAGDDAEPLS | | peptide | | | |
| P18509 | 132 | HSDGIFTDSYSRYRKQMAVKKYLAAVL | 158 | Pituitary | Glucagon | | |
| | | | | adenylate | | | |
| | | | | cyclase- | | | |
| | | | | activating | | | |
| | | | | polypeptide 27 | | | |
| P18509 | 132 | HSDGIFTDSYSRYRKQMAVKKYLAAVLGKRYKQRVKNK | 169 | Pituitary | Glucagon | | |
| | | | | adenylate | | | |
| | | | | cyclase- | | | |
| | | | | activating | | | |
| | | | | polypeptide 38 | | | |
| P09683 | 28 | HSDGTFTSELSRLREGARLQRLLQGLV | 54 | Secretin | Glucagon | T34, S35, S38 | 3 |
| P01286 | 32 | YADAIFTNSYRKVLGQLSARKLLQDIMSRQQGESNQERG | 75 | Somatoliberin | Glucagon | | |
| | | ARARL | | | | | |
| P01282 | 125 | HSDAVFTDNYTRLRKQMAVKKYLNSILN | 152 | Vasoactive | Glucagon | T131 | 1 |
| | | | | intestinal peptide | | | |
| P01148 | 37 | DAENLIDSFQEIVKEVGQLAETQRFECTTHQPRSPLRDLK | 92 | GnRH-associated | GnRH | T64 | 1 |
| | | GALESLIEEETGQKKI | | peptide 1 | | | |
| O43555 | 37 | ALS SAQDPQNALRPPGRALDTAAGSPVQTAHGLPSDALA | 120 | GnRH-associated | GnRH | | |
| | | PLDDSMPWEGRTTAQWSLHRKRHLARTLLTAAREPRPAP | | peptide 2 | | | |
| | | PSSNKV | | | | | |
| P01148 | 24 | QHWSYGLRPG | 33 | Gonadoliberin-1 | GnRH | | |
| O43555 | 24 | QHWSHGWYPG | 33 | Gonadoliberin-2 | GnRH | | |
| P01148 | 24 | QHWSYGLRPGGKRDAENLIDSFQEIVKEVGQLAETQRFE | 92 | Progonadoliberin- | GnRH | T64 | 1 |
| | | CTTHQPRSPLRDLKGALESLIEEETGQKKI | | 1 | | | |
| O43555 | 24 | QHWSHGWYPGGKRALSSAQDPQNALRPPGRALDTAAGS | 120 | Progonadoliberin- | GnRH | | |
| | | PVQTAHGLPSDALAPLDDSMPWEGRTTAQWSLHRKRHL | | 2 | | | |
| | | ARTLLTAAREPRPAPPSSNKV | | | | | |
| P01308 | 90 | GIVEQCCTSICSLYQLENYCN | 110 | Insulin A chain | Insulin | | |
| P01308 | 25 | FVNQHLCGSHLVEALYLVCGERGFFYTPKT | 54 | Insulin B chain | Insulin | T51 | 1 |
| | | | | | | (Ambiguous) | |
| P05019 | 49 | GPETLCGAELVDALQFVCGDRGFYFNKPTGYGSSSRRAP | 118 | Insulin-like | Insulin | | |
| | | QTGIVDECCFRSCDLRRLEMYCAPLKPAKSA | | growth factor I | | | |
| P01344 | 25 | AYRPSETLCGGELVDTLQFVCGDRGFYFSRPASRVSRRSR | 91 | Insulin-like | Insulin | S74, T86 | 0 |
| | | GIVEECCFRSCDLALLETYCATPAKSE | | growth factor II | | | |
| P01344 | 26 | YRPSETLCGGELVDTLQFVCGDRGFYFSRPASRVSRRSRG | 91 | Insulin-like | Insulin | S74, T86 | 0 |
| | | IVEECCFRSCDLALLETYCATPAKSE | | growth factor II | | | |
| | | | | Ala-25 Del | | | |
| P01344 | 93 | DVSTPPTVLPDNFPRYPVGKFFQYDTVVKQSTQRL | 126 | Preptin | Insulin | S95, T96, T99 | 3 |
| P04090 | 162 | QLYSALANKCCHVGCTKRSLARFC | 185 | Relaxin A chain | Insulin | | |
| P04808 | 163 | PYVALFEKCCLIGCTKRSLAKYC | 185 | Relaxin A chain | Insulin | | |
| | | | | (By similarity) | | | |
| P04090 | 25 | DSWMEEVIKLCGRELVRAQIAICGMSTWS | 53 | Relaxin B chain | Insulin | | |
| P04808 | 23 | VAAKWKDDVIKLCGRELVRAQIAICGMSTWS | 53 | Relaxin B chain | Insulin | | |
| | | | | (By similarity) | | | |
| Q8WXF3 | 119 | DVLAGLSSSCCKWGCSKSEISSLC | 142 | Relaxin-3 A | Insulin | | |
| | | | | chain (By | | | |
| | | | | similarity) | | | |
| Q8WXF3 | 26 | RAAPYGVRLCGREFIRAVIFTCGGSRW | 52 | Relaxin-3 B | Insulin | | |
| | | | | chain (By | | | |
| | | | | similarity) | | | |
| Q15726 | 112 | YNWNSFGLRF | 121 | Kisspeptin-10 | KISS1 | | |
| Q15726 | 109 | LPNYNVVNSFGLRF | 121 | Kisspeptin-13 | KISS1 | | |
| Q15726 | 108 | DLPNYNWNSFGLRF | 121 | Kisspeptin-14 | KISS1 | | |
| Q15726 | 20 | EPLEKVASVGNSRPTGQQLESLGLLAPGEQSLPCTERKPA | 138 | Metastasis- | KISS1 | A88 | 1 |
| | | ATARLSRRGTSLSPPPESSGSPQQPGLSAPHSRQIPAPQGA | | suppressor KiSS- | | | |
| | | VLVQREKDLPNYNWNSFGLRFGKREAAPGNHGRSAGRG | | 1 | | | |
| Q15726 | 68 | GTSLSPPPESSGSPQQPGLSAPHSRQIPAPQGAVLVQREKD | 121 | Metastin | KISS1 | A88 | 1 |
| | | LPNYNVVNSFGLRF | | | | | |
| P41159 | 22 | VPIQKVQDDTKTLIKTIVTRINDISHTQSVSSKQKVTGLDFI | 167 | Leptin | Leptin | | |
| | | PGLHPILTLSKMDQTLAVYQQILTSMPSRNVIQISNDLENL | | | | | |
| | | RDLLHVLAFSKSCHLPWASGLETLDSLGGVLEASGYSFEV | | | | | |
| | | VALSRLQGSLQDMLWQLDLSPGC | | | | | |
| P20382 | 147 | DFDMLRCMLGRVYRPCWQV | 165 | Melanin- | Melanin- | | |
| | | | | concentrating | concentrating | | |
| | | | | hormone | hormone | | |
| P20382 | 131 | EIGDEENSAKFPI | 143 | Neuropeptide- | Melanin- | | |
| | | | | glutamic acid- | concentrating | | |
| | | | | isoleucine | hormone | | |
| P20382 | 110 | GSVAFPAENGVQNTESTQE | 128 | Neuropeptide- | Melanin- | T123, T125, | 3 |
| | | | | glycine-glutamic | concentrating | T126 | |
| | | | | acid(Potential) | hormone | | |
| P20382 | 22 | ILLSASKSIRNLDDDMVFNTFRLGKGFQKEDTAEKSVIAPS | 165 | Pro-MCH | Melanin- | S57, S62, N93, | 6 |
| | | LEQYKNDESSFMNEEENKVSKNTGSKHNFLNHGLPLNLAI | | | concentrating | T123, T125 | |
| | | KPYLALKGSVAFPAENGVQNTESTQEKREIGDEENSAKFP | | | hormone | T126 | |
| | | IGRRDFDMLRCMLGRVYRPCWQV | | | | | |
| Q9UBU3 | 24 | GSSFLSPEHQRVQQRKESKKPPAKLQP | 50 | Ghrelins-27 | Motilin | | |
| Q9UBU3 | 24 | GSSFLSPEHQRVQQRKESKKPPAKLQPR | 51 | Ghrelins-28 | Motilin | | |
| P12872 | 26 | FVPIFTYGELQRMQEKERNKGQ | 47 | Motilin | Motilin | | |
| P12872 | 50 | SLSVWQRSGEEGPVDPAEPIREEENEMIKLTAPLEIGMRM | 115 | Motilin- | Motilin | | |
| | | NSRQLEKYPATLEGLLSEMLPQHAAK | | associated | | | |
| | | | | peptide | | | |
| Q9UBU3 | 76 | FNAPFDVGIKLSGVQYQQHSQAL | 98 | Obestatin | Motilin | | |
| P12872 | 26 | FVPIFTYGELQRMQEKERNKGQKKSLSVWQRSGEEGPVD | 115 | Promotilin | Motilin | | |
| | | PAEPIREEENEMIKLTAPLEIGMRMNSRQLEKYPATLEGLL | | | | | |
| | | SEMLPQHAAK | | | | | |
| Q15848 | 19 | ETTTQGPGVLLPLPKGACTGWMAGIPGHPGHNGAPGRDG | 244 | Adiponectin | NA | T22 | 1 |
| | | RDGTPGEKGEKGDPGLIGPKGDIGETGVPGAEGPRGFPGI | | | | | |
| | | QGRKGEPGEGAYVYRSAFSVGLETYVTIPNMPIRFTKIFY | | | | | |
| | | NQQNHYDGSTGKFHCNIPGLYYFAYHITVYMKDVKVSLF | | | | | |
| | | KKDKAMLFTYDQYQENNVDQASGSVLLHLEVGDQVWL | | | | | |
| | | QVYGEGERNGLYADNDNDSTFTGFLLYHDTN | | | | | |
| O00253 | 21 | AQMGLAPMEGIRRPDQALLPELPGLGLRAPLKKTTAEQA | 131 | Agouti-related | NA | T55 | 1 |
| | | EEDLLQEAQALAEVLDLQDREPRSSRRCVRLHESCLGQQ | | protein | | | |
| | | VPCCDPCATCYCRFFNAFCYCRKLGTAMNPCSRT | | | | | |
| Q9BZL1 | 1 | MIEVVCNDRLGKKVRVKCNTDDTIGDLKKLIAAQTGTRW | 73 | Ubiquitin-like | NA | | |
| | | NKIVLKKWYTIFKDHVSLGDYEIHDGMNLELYYQ | | protein 5 | | | |
| P43490 | 1 | MNPAAEAEFNILLATDSYKVTHYKQYPPNTSKVYSYFEC | 491 | Nicotinamide | NAPRTase | | |
| | | REKKFENSKLRKVKYEETVEYGLQVILNKYLKGKVVTKE | | phosphoribosyltr | | | |
| | | KIQEAKDVYKEHFQDDVFNEKGWNYILEKYDGHLPIEIK | | ansferase | | | |
| | | AVPEGFVIPRGNVLFTVENTDPECYWLTNVVIETILVQSWY | | | | | |
| | | PITVATNSREQKKILAKYLLETSGNLDGLEYKLHDFGYRG | | | | | |
| | | VSSQETAGIGASAHLVNFKGTDTVAGLALIKKYYGTKDP | | | | | |
| | | VPGYSVPAAEHSTITAWGKDHEKDAFEHIVTQFSSVPVSV | | | | | |
| | | VSDSYDIYNACEKIWGEDLRHLIVSRSTQAPLIIRPDSGNP | | | | | |
| | | LDTVLKVLEILGKKFPVTENSKGYKLLPPYLRVIQGDGVD | | | | | |
| | | INTLQEIVEGMKQKMVVSIENIAFGSGGGLLQKLTRDLLNC | | | | | |
| | | SFKCSYVVTNGLGINVFKDPVADPNKRSKKGRLSLHRTPA | | | | | |
| | | GNFVTLEEGKGDLEEYGQDLLHTVFKNGKVTKSYSFDEI | | | | | |
| | | RKNAQLNIELEAAHH | | | | | |
| P01160 | 124 | SLRRSSCFGGRMDRIGAQSGLGCNSFRY | 151 | Atrial natriuretic | Natriuretic peptide | S142, S148 | 2 |
| | | | | factor | | | |
| P16860 | 103 | SPKMVQGSGCFGRKMDRISSSSGLGCKV | 130 | BNP(1-28) | Natriuretic peptide | | |
| P16860 | 103 | SPKMVQGSGCFGRKMDRISSSSGLGCKVL | 131 | BNP(1-29) | Natriuretic peptide | | |
| P16860 | 103 | SPKMVQGSGCFGRKMDRISSSSGLGCKVLR | 132 | BNP(1-30) | Natriuretic peptide | | |
| P16860 | 104 | PKMVQGSGCFGRKMDRISSSSGLGCKVLRR | 133 | BNP(2-31) | Natriuretic peptide | | |
| P16860 | 105 | KMVQGSGCFGRKMDRISSSSGLGCKVL | 131 | BNP(3-29) | Natriuretic peptide | | |
| P16860 | 105 | KMVQGSGCFGRKMDRISSSSGLGCKVLR | 132 | BNP(3-30) | Natriuretic peptide | | |
| P16860 | 105 | KMVQGSGCFGRKMDRISSSSGLGCKVLRRH | 134 | BNP(3-32) | Natriuretic peptide | | |
| P16860 | 106 | MVQGSGCFGRKMDRISSSSGLGCK | 129 | BNP(4-27) | Natriuretic peptide | | |
| P16860 | 106 | MVQGSGCFGRKMDRISSSSGLGCKVL | 131 | BNP(4-29) | Natriuretic peptide | | |
| P16860 | 106 | MVQGSGCFGRKMDRISSSSGLGCKVLR | 132 | BNP(4-30) | Natriuretic peptide | | |
| P16860 | 106 | MVQGSGCFGRKMDRISSSSGLGCKVLRR | 133 | BNP(4-31) | Natriuretic peptide | | |
| P16860 | 106 | MVQGSGCFGRKMDRISSSSGLGCKVLRRH | 134 | BNP(4-32) | Natriuretic peptide | | |
| P16860 | 107 | VQGSGCFGRKMDRISSSSGLGCKVL | 131 | BNP(5-29) | Natriuretic peptide | | |
| P16860 | 107 | VQGSGCFGRKMDRISSSSGLGCKVLRR | 133 | BNP(5-31) | Natriuretic peptide | | |
| P16860 | 107 | VQGSGCFGRKMDRISSSSGLGCKVLRRH | 134 | BNP(5-32) | Natriuretic peptide | | |
| P16860 | 103 | SPKMVQGSGCFGRKMDRISSSSGLGCKVLRRH | 134 | Brain natriuretic | Natriuretic peptide | | |
| | | | | peptide 32 | | | |
| P01160 | 26 | NPMYNAVSNADLMDFKNLLDHLEEKMPLED | 55 | Cardiodilatin- | Natriuretic peptide | Y29, A31, S33 | 1 |
| | | | | related peptide | | (Ambiguous) | |
| P23582 | 105 | GLSKGCFGLKLDRIGSMSGLGC | 126 | CNP-22 | Natriuretic peptide | | |
| P23582 | 98 | YKGANKKGLSKGCFGLKLDRIGSMSGLGC | 126 | CNP-29 | Natriuretic peptide | | |
| P23582 | 74 | DLRVDTKSRAAWARLLQEHPNARKYKGANKKGLSKGCF | 126 | CNP-53 | Natriuretic peptide | | |
| | | GLKLDRIGSMSGLGC | | | | | |
| P16860 | 27 | HPLGSPGSASDLETSGLQEQRNHLQGKLSELQVEQTSLEP | 134 | Natriuretic | Natriuretic peptide | T74, E85 | 2 |
| | | LQESPRPTGVWKSREVATEGIRGHRKMVLYTLRAPRSPK | | peptides B | | | |
| | | MVQGSGCFGRKMDRISSSSGLGCKVLRRH | | | | | |
| P58417 | 22 | ANLTNGGKSELLKSGSSKSTLKHIW1ESSKDLSISRLLSQT | 271 | Neurexophilin-1 | Neurexophilin | | |
| | | FRGKENDTDLDLRYDTPEPYSEQDLWDWLRNSTDLQEPR | | | | | |
| | | PRAKRRPIVKTGKFKKMFGWGDFHSNIKTVKLNLLITGKI | | | | | |
| | | VDHGNGTFSVYFRHNSTGQGNVSVSLVPPTKIVEFDLAQ | | | | | |
| | | QTVIDAKDSKSFNCRIEYEKVDKATKNTLCNYDPSKTCY | | | | | |
| | | QEQTQSHVSWLCSKPFKVICIYISFYSTDYKLVQKVCPDY | | | | | |
| | | NYHSDTPYFPSG | | | | | |
| O95156 | 23 | KEVVHATEGLDWEDKDAPGTLVGNVVHSRIISPLRLFVK | 264 | Neurexophilin-2 | Neurexophilin | T29 | 1 |
| | | QSPVPKPGPMAYADSMENFWDWLANIFEIQEPLARTKRR | | | | | |
| | | PIVKTGKFKKMFGWGDFHSNIKTVKLNLLITGKIVDHGNG | | | | | |
| | | TFSVYFRHNSTGLGNVSVSLVPPSKVVEFEVSPQSTLETKE | | | | | |
| | | SKSFNCRIEYEKTDRAKKTALCNFDPSKICYQEQTQSHVS | | | | | |
| | | WLCSKPFKVICIYIAFYSVDYKLVQKVCPDYNYHSETPYL | | | | | |
| | | SSG | | | | | |
| O95157 | 23 | QDDGPPGSEDPERDDHEGQPRPRVPRKRGHISPKSRPMAN | 252 | Neurexophilin-3 | Neurexophilin | | |
| | | STLLGLLAPPGEAWGILGQPPNRPNHSPPPSAKVKKIFGW | | | | | |
| | | GDFYSNIKTVALNLLVTGKIVDHGNGTFSVHFQHNATGQ | | | | | |
| | | GNISISLVPPSKAVEFHQEQQIFIEAKASKIFNCRMEWEKV | | | | | |
| | | ERGRRTSLCTHDPAKICSRDHAQSSATVVSCSQPFKVVCVY | | | | | |
| | | IAFYSTDYRLVQKVCPDYNYHSDTPYYPSG | | | | | |
| O95158 | 24 | QIPESGRPQYLGLRPAAAGAGAPGQQLPEPRSSDGLGVGR | 308 | Neurexophilin-4 | Neurexophilin | | |
| | | AWSWAWPTNHTGALARAGAAGALPAQRTKRKPSIKAAR | | | | | |
| | | AKKIFGWGDFYFRVHTLKFSLLVTGKIVDHVNGTFSVYFR | | | | | |
| | | HNSSSLGNLSVSIVPPSKRVEFGGVWLPGPVPHPLQSTLAL | | | | | |
| | | EGVLPGLGPPLGMAAAAAGPGLGGSLGGALAGPLGGAL | | | | | |
| | | GVPGAKESRAFNCHVEYEKTNRARKHRPCLYDPSQVCFT | | | | | |
| | | EHTQSQAAWLCAKPFKVICIFVSFLSFDYKLVQKVCPDYN | | | | | |
| | | FQSEHPYFG | | | | | |
| Q5H8A3 | 109 | ILQRGSGTAAVDFTKKDHTATWGRPFFLFRPRN | 141 | Neuromedin-S | Neuromedins | | |
| P48645 | 142 | FRVDEEFQSPFASQSRGYFLFRPRN | 166 | Neuromedin-U- | Neuromedins | | |
| | | | | 25 | | | |
| Q8NG41 | 25 | WYKPAAGHSSYSVGRAAGLLSGLR | 48 | Neuropeptide B- | Neuromedins | | |
| | | | | 23 | | | |
| Q8NG41 | 25 | WYKPAAGHSSYSVGRAAGLLSGLRRSPYA | 53 | Neuropeptide B- | Neuromedins | | |
| | | | | 29 | | | |
| P0C0P6 | 70 | SFRNGVGTGMKKTSFQRAKS | 89 | Neuropeptide S | Neuromedins | | |
| Q8N729 | 33 | WYKHVASPRYHTVGRAAGLLMGL | 55 | Neuropeptide W- | Neuromedins | | |
| | | | | 23 | | | |
| Q8N729 | 33 | WYKHVASPRYHTVGRAAGLLMGLRRSPYLW | 62 | Neuropeptide W- | Neuromedins | | |
| | | | | 30 | | | |
| P30990 | 24 | SDSEEEMKALEADFLTNMHTSKISKAHVPSWKMTLLNVC | 148 | Large | Neurotensin | | |
| | | SLVNNLNSPAEETGEVHEEELVARRKLPTALDGFSLEAML | | neuromedin N | | | |
| | | TIYQLHKICHSRAFQHWELIQEDILDTGNDKNGKEEVIKR | | | | | |
| | | KIPYIL | | | | | |
| P30990 | 144 | IPYIL | 148 | Neuromedin N | Neurotensin | | |
| P30990 | 151 | QLYENKPRRPYIL | 163 | Neurotensin | Neurotensin | | |
| P30990 | 166 | DSYYY | 170 | Tail peptide | Neurotensin | | |
| | | | | (Potential) | | | |
| P01303 | 68 | SSPETLISDLLMRESTENVPRTRLEDPAMW | 97 | C-flanking | NPY | S68, S69, T83 | 3 |
| | | | | peptide of NPY | | T88, A95 | |
| P01303 | 29 | YPSKPDNPGEDAPAEDMARYYSALRHYINLITRQRY | 64 | Neuropeptide Y | NPY | S31, | 2 |
| | | | | | | (Ambiguous | |
| | | | | | | Y55 or T60 | |
| P01298 | 30 | APLEPVYPGDNATPEQMAQYAADLRRYINMLTRPRY | 65 | Pancreatic | NPY | T61 | 1 |
| | | | | hormone | | | |
| P01298 | 69 | HKEDTLAFSEWGSPHAAVPR | 88 | Pancreatic | NPY | | |
| | | | | icosapeptide | | | |
| P10082 | 29 | YPIKPEAPREDASPEELNRYYASLRHYLNLVTRQRY | 64 | Peptide YY | NPY | T60 | 1 |
| P10082 | 31 | IKPEAPREDASPEELNRYYASLRHYLNLVTRQRY | 64 | Peptide YY(3- | NPY | T60 | 1 |
| | | | | 36) | | | |
| P80303 | 25 | VPIDIDKTKVQNIHPVESAKIEPPDTGLYYDEYLKQVIDVL | 106 | Nesfatin-1 | Nucleobindin | T32, I37, S42, | 8 |
| | | ETDKHFREKLQKADIEEIKSGRLSKELDLVSHHVRTKLDE | | | | T50, Y53, Y54, | |
| | | L | | | | T67, S96 | |
| Q02818 | 27 | VPLERGAPNKEETPATESPDTGLYYHRYLQEVIDVLETDG | 461 | Nucleobindin-1 | Nucleobindin | P34, T39, T42, | 22 |
| | | HFREKLQAANAEDIKSGKLSRELDFVSHHVRTKLDELKR | | | | S44, T47, Y50, | |
| | | QEVSRLRMLLKAKMDAEQDPNVQVDHLNLLKQFEHLDP | | | | S93, T148, | |
| | | QNQHTFEARDLELLIQTATRDLAQYDAAHHEEFKRYEML | | | | T162, S224, | |
| | | KEHERRRYLESLGEEQRKEAERKLEEQQRRHREHPKVNV | | | | S320, S321, | |
| | | PGSQAQLKEVWEELDGLDPNRFNPKTFFILHDINSDGVLD | | | | T335, H339, | |
| | | EQELEALFTKELEKVYDPKNEEDDMREMEEERLRMREHV | | | | S369, T372, | |
| | | MKNVDTNQDRLVTLEEFLASTQRKEFGDTGEGWETVEM | | | | S378, Q407, | |
| | | HPAYFEEELRRFEEELAAREAELNAKAQRLSQETEALGRS | | | | A414, T426, | |
| | | QGRLEAQKRELQQAVLHMEQRKQQQQQQQGHKAPAAH | | | | L452 | |
| | | PEGQLKFHPDTDDVPVPAPAGDQKEVDTSEKKLLERLPE | | | | x459(deleterious | |
| | | VEVPQHL | | | | mutation) | |
| P80303 | 25 | VPIDIDKTKVQNIHPVESAKIEPPDTGLYYDEYLKQVIDVL | 420 | Nucleobindin-2 | Nucleobindin | T32, | 16 |
| | | ETDKHFREKLQKADIEEIKSGRLSKELDLVSHHVRTKLDE | | | | I37, | |
| | | LKRQEVGRLRMLIKAKLDSLQDIGMDHQALLKQFDHLNH | | | | S42, T50, Y53, | |
| | | LNPDKFESTDLDMLIKAATSDLEHYDKTRHEEFKKYEMM | | | | Y54, T67, S96, | |
| | | KEHERREYLKTLNEEKRKEEESKFEEMKKKHENHPKVNH | | | | S152, T163, | |
| | | PGSKDQLKEVWEETDGLDPNDFDPKTFFKLHDVNSDGFL | | | | T172, A355, | |
| | | DEQELEALFTKELEKVYDPKNEEDDMVEMEEERLRMRE | | | | Y389, P406, | |
| | | HVMSEVDTNKDRLVTLEEFLKATEKKEFLEPDSWETLDQ | | | | S408, L414 | |
| | | QQFFTEEELKEYENIIALQENELKKKADELQKQKEELQRQ | | | | | |
| | | HDQLEAQKLEYHQVIQQMEQKKLQQGIPPSGPAGELKFE | | | | | |
| | | PHI | | | | | |
| P01213 | 175 | YGGFLRKYPK | 184 | Alpha- | Opioid | | |
| | | | | neoendorphin | | | |
| P01213 | 175 | YGGFLRKYP | 183 | Beta- | Opioid | | |
| | | | | neoendorphin | | | |
| P01213 | 207 | YGGFLRRIRPKLKWDNQKRYGGFLRRQFKVVT | 238 | Big dynorphin | Opioid | | |
| P01213 | 207 | YGGFLRRIRPKLK | 219 | Dynorphin A(1- | Opioid | | |
| | | | | 13) (By | | | |
| | | | | similarity) | | | |
| P01213 | 207 | YGGFLRRIRPKLKWDNQ | 223 | Dynorphin A(1- | Opioid | | |
| | | | | 17) | | | |
| P01213 | 207 | YGGFLRRI | 214 | Dynorphin A(1- | Opioid | | |
| | | | | 8) (By similarity) | | | |
| P01213 | 175 | YGGFL | 179 | Leu-enkephalin | Opioid | | |
| P01213 | 226 | YGGFLRRQFKVVTRSQEDPNAYSGELFDA | 254 | Leumorphin | Opioid | | |
| P01210 | 100 | YGGFM | 104 | Met-enkephalin | Opioid | | |
| P01210 | 186 | YGGFMRGL | 193 | Met-enkephalin- | Opioid | | |
| | | | | Arg-Gly-Leu | | | |
| P01210 | 261 | YGGFMRF | 267 | Met-enkephalin- | Opioid | | |
| | | | | Arg-Phe | | | |
| Q13519 | 98 | MPRVRSLFQEQEEPEPGMEEAGEMEQKQLQ | 127 | Neuropeptide 1 | Opioid | P111 | 1 |
| | | | | (Probable) | | | |
| Q13519 | 149 | FSEFMRQYLVLSMQSSQ | 165 | Neuropeptide 2 | Opioid | | |
| | | | | (Probable) | | | |
| Q13519 | 130 | FGGFTGARKSARKLANQ | 146 | Nociceptin | Opioid | | |
| P01210 | 143 | DAEEDDSLANSSDLLKELLETGDNRERSHHQDGSDNEEE | 183 | PENK(143-183) | Opioid | | |
| | | VS | | (By similarity) | | | |
| P01210 | 237 | FAEALPSDEEGESYSKEVPEME | 258 | PENK(237-258) | Opioid | | |
| | | | | (By similarity) | | | |
| P01213 | 226 | YGGFLRRQFKVVT | 238 | Rimorphin | Opioid | | |
| P01210 | 114 | MDELYPMEPEEEANGSEILA | 133 | rimorphin | Opioid | | |
| P01210 | 25 | ECSQDCATCSYRLVRPADINFLACVMECEGKLPSLKIWET | 97 | Synenkephalin | Opioid | | |
| | | CKELLQLSKPELPQDGTSTLRENSKPEESHLLA | | | | | |
| O43612 | 34 | QPLPDCCRQKTCSCRLYELLHGAGNHAAGILTL | 66 | Orexin-A | Orexin | | |
| O43612 | 70 | RSGPPGLQGRLQRLLQASGNHAAGILTM | 97 | Orexin-B | Orexin | | |
| P12272 | 143 | TRSAWLDSGVTGSGLEGDHLSDTSTTSLELDSR | 175 | Osteostatin | Parathyroid | | |
| | | | | | hormone | | |
| P12272 | 37 | AVSEHQLLHDKGKSIQDLRRREFLHHLIAEIHTAEIRATSE | 177 | Parathyroid | Parathyroid | T75 | 1 |
| | | VSPNSKPSPNTKNHPVRFGSDDEGRYLTQETNKVETYKE | | hormone-related | hormone | | |
| | | QPLKTPGKKKKGKPGKRKEQEKKKRRTRSAWLDSGVTG | | protein | | | |
| | | SGLEGDHLSDTSTTSLELDSRRH | | | | | |
| P12272 | 37 | AVSEHQLLHDKGKSIQDLRRREFLHHLIAEIHTAEI | 72 | PTHrP[1-36] | Parathyroid | | |
| | | | | | hormone | | |
| P12272 | 74 | ATSEVSPNSKPSPNTKNHPVRFGSDDEGRYLTQETNKVET | 130 | PTHrP[38-94[ | Parathyroid | T75 | 1 |
| | | YKEQPLKTPGKKKKGKP | | | hormone | | |
| Q96A98 | 62 | SLALADDAAFRERARLLAALERRHWLNSYMHKLLVLDA | 100 | Tuberoinfundibular | Parathyroid | | |
| | | P | | peptide of | hormone | | |
| | | | | 39residues | | | |
| P01189 | 237 | YGGFMTSEKSQTPLVTLEKNAIIKNAYKKGE | 267 | Beta-endorphin | POMC | S246, T248, | 3 |
| | | | | | | T252 | |
| P01189 | 138 | SYSMEHFRWGKPVGKKRRPVKVYPNGAEDESAEAFPLEF | 176 | Corticotropin | POMC | | |
| P01189 | 156 | PVKVYPNGAEDESAEAFPLEF | 176 | Corticotropin- | POMC | | |
| | | | | like intermediary | | | |
| | | | | peptide | | | |
| P01189 | 179 | ELTGQRLREGDGPDGPADDGAGAQADLEHSLLVAAEKK | 267 | Lipotropin beta | POMC | S246, T248, | 3 |
| | | DEGPYRMEHFRWGSPPKDKRYGGFMTSEKSQTPLVTLFK | | | | T252 | |
| | | NAIIKNAYKKGE | | | | | |
| P01189 | 179 | ELTGQRLREGDGPDGPADDGAGAQADLEHSLLVAAEKK | 234 | Lipotropin | POMC | | |
| | | DEGPYRMEHFRWGSPPKD | | gamma | | | |
| P01189 | 138 | SYSMEHFRWGKPV | 150 | Melanotropin | POMC | | |
| | | | | alpha | | | |
| P01189 | 217 | DEGPYRMEHFRWGSPPKD | 234 | Melanotropin | POMC | | |
| | | | | beta | | | |
| P01189 | 77 | YVMGHFRWDRF | 87 | Melanotropin | POMC | | |
| | | | | gamma | | | |
| P01189 | 27 | WCLESSQCQDLTTESNLLECIRACKPDLSAETPMFPGNGD | 102 | NPP | POMC | T71 | 1 |
| | | EQPLTENPRKYVMGHFRWDRFGRRNSSSSGSSGAGQ | | | | | |
| P01189 | 105 | EDVSAGEDCGPLPEGGPEPRSDGAKPGPRE | 134 | Potential peptide | POMC | | |
| Q9UHG2 | 245 | LETPAPQVPARRLLPP | 260 | Big LEN (By | ProSAAS | T247, P248, | 2 |
| | | | | similarity) | | A249 | |
| Q9UHG2 | 221 | AADHDVGSELPPEGVLGALLRVKRLETPAPQVPARRLLPP | 260 | Big PEN-LEN | ProSAAS | T247, P248, | 2 |
| | | | | (By similarity) | | A249 | |
| Q9UHG2 | 34 | ARPVKEPRGLSAASPPLAETGAPRRF | 59 | Big SAAS (By | ProSAAS | G42, S47, T53, | 5 |
| | | | | similarity) | | G54, A55 | |
| Q9UHG2 | 34 | ARPVKEP | 40 | KEP (By | ProSAAS | | |
| | | | | similarity) | | | |
| Q9UHG2 | 245 | LETPAPQVPA | 254 | Little LEN (By | ProSAAS | T247, P248, | 2 |
| | | | | similarity) | | A249 | |
| Q9UHG2 | 42 | GLSAASPPLAETGAPRRF | 59 | Little SAAS (By | ProSAAS | G42, S47, T53, | 5 |
| | | | | similarity) | | G54, A55 | |
| Q9UHG2 | 221 | AADHDVGSELPPEGVLGALLRV | 242 | PEN (By | ProSAAS | | |
| | | | | similarity) | | | |
| Q9UHG2 | 34 | ARPVKEPRGLSAASPPLAETGAPRRFRRSVPRGEAAGAVQ | 260 | ProSAAS | ProSAAS | G42, S47, T53, | 13 |
| | | ELARALAHLLEAERQERARAEAQEAEDQQARVLAQLLR | | | | G54, A55, A115, | |
| | | VWGAPRNSDPALGLDDDPDAPAAQLARALLRARLDPAAL | | | | N118, S119, | |
| | | AAQLVPAPVPAAALRPRPPVYDDGPAGPDAEEAGDETPD | | | | A176, S206, | |
| | | VDPELLRYLLGRILAGSADSEGVAAPRRLRRAADHDVGSE | | | | A207, T247, | |
| | | LPPEGVLGALLRVKRLETPAPQVPARRLLPP | | | | P248, A249 | |
| Q9HD89 | 19 | KTLCSMEEAINERIQEVAGSLIFRAISSIGLECQSVTSRGDL | 108 | Resistin | Resistin/FIZZ | | |
| | | ATCPRGFAVTGCTCGSACGSWDVRAETTCHCQCAGMDW | | | | | |
| | | TGARCCRVQP | | | | | |
| Q9BQ08 | 24 | QCSLDSVMDKKIKDVLNSLEYSPSPISKKLSCASVKSQGR | 111 | Resistin-like beta | Resistin/FIZZ | | |
| | | PSSCPAGMAVTGCACGYGCGSWDVQLETTCHCQCSVVD | | | | | |
| | | WTTARCCHLT | | | | | |
| P83859 | 91 | QDEGSEATGFLPAAGEKTSGPLGNLAEELNGYSRKKGGF | 133 | QRF-amide | RFamide | | |
| | | SFRF | | | neuropeptide | | |
| P06850 | 154 | SEEPPISLDLTFHLLREVLEMARAEQLAQQAHSNRKLMEII | 194 | Corticoliberin | Sauvagine/ | | |
| | | | | | corticotropin- | | |
| | | | | | releasing | | |
| | | | | | factor/urotensin I | | |
| P55089 | 83 | DNPSLSIDLTFHLLRTLLELARTQSQRERAEQNRIIFDSV | 122 | Urocortin | Sauvagine/ | | |
| | | | | | corticotropin- | | |
| | | | | | releasing | | |
| | | | | | factor/urotensin I | | |
| Q96RP3 | 72 | IVLSLDVPIGLLQILLEQARARAAREQATTNARILARVGHC | 112 | Urocortin-2 | Sauvagine/ | | |
| | | | | | corticotropin- | | |
| | | | | releasing | | | |
| | | | | | factor/urotensin I | | |
| Q969E3 | 120 | FTLSLDVPTNIMNLLFNIAKAKNLRAQAAANAHLMAQI | 157 | Urocortin-3 | Sauvagine/ | | |
| | | | | | corticotropin- | | |
| | | | | | releasing | | |
| | | | | | factor/urotensin I | | |
| P01019 | 34 | DRVY | 37 | Angiotensin 1-4 | Serpin | | |
| P01019 | 34 | DRVYI | 38 | Angiotensin 1-5 | Serpin | | |
| P01019 | 34 | DRVYIHP | 40 | Angiotensin 1-7 | Serpin | | |
| P01019 | 34 | DRVYIHPFH | 42 | Angiotensin 1-9 | Serpin | | |
| P01019 | 34 | DRVYIHPFHL | 43 | Angiotensin-1 | Serpin | | |
| P01019 | 34 | DRVYIHPF | 41 | Angiotensin-2 | Serpin | | |
| P01019 | 35 | RVYIHPF | 41 | Angiotensin-3 | Serpin | | |
| P01019 | 36 | VYIHPF | 41 | Angiotensin-4 | Serpin | | |
| P01019 | 34 | DRVYIHPFHLVIHNESTCEQLAKANAGKPKDPTFIPAPIQA | 485 | Angiotensinogen | Serpin | T440 or S442 or | 1 |
| | | KTSPVDEKALQDQLVLVAAKLDFEDKLRAAMVGMLANF | | | | T443 or T455 | |
| | | LGFRIYGMHSELWGVVHGATVLSPTAVFGTLASLYLGAL | | | | (Ambiguous) | |
| | | DHTADRLQAILGVPWKDKNCTSRLDAHKVLSALQAVQG | | | | | |
| | | LLVAQGRADSQAQLLLSTVVGVFTAPGLHLKQPFVQGLA | | | | | |
| | | LYTPVVLPRSLDFTELDVAAEKIDRFMQAVTGWKTGCSL | | | | | |
| | | MGASVDSTLAFNTYVHFQGKMKGFSLLAEPQEFWVDNS | | | | | |
| | | TSVSVPMLSGMGTFQHWSDIQDNFSVTQVPFTESACLLLI | | | | | |
| | | QPHYASDLDKVEGLTFQQNSLNVVMKKLSPRTIHLTMPQL | | | | | |
| | | VLQGSYDLQDLLAQAELPAILHFELNLQKLSNDRIRVGEV | | | | | |
| | | LNSIFFELEADEREPTESTQQLNKPEVLEVTLNRPFLFAVY | | | | | |
| | | DQSATALHFLGRVANPLSTA | | | | | |
| P08185 | 23 | MDPNAAYVNMSNHHRGLASANVDFAFSLYKHLVALSPK | 405 | Corticosteroid- | Serpin | | |
| | | KNIFISPVSISMALAMLSLGTCGHTRAQLLQGLGFNLTERS | | binding globulin | | | |
| | | ETEIHQGFQHLHQLFAKSDTSLEMTMGNALFLDGSLELLE | | | | | |
| | | SFSADIKHYYESEVLAMNFQDWATASRQINSYVKNKTQG | | | | | |
| | | KIVDLFSGLDSPAILVLVNYIFFKGTWTQPFDLASTREENF | | | | | |
| | | YVDETTVVKVPMMLQSSTISYLHDSELPCQLVQMNYVGN | | | | | |
| | | GTVFFILPDKGKMNTVIAALSRDTINRWSAGLTSSQVDLY | | | | | |
| | | IPKVTISGVYDLGDVLEEMGIADLFTNQANFSRITQDAQL | | | | | |
| | | KSSKVVHKAVLQLNEEGVDTAGSTGVTLNLTSKPIILRFN | | | | | |
| | | QPFIIMIFDHFTWSSLFLARVMNPV | | | | | |
| Q86U17 | 20 | QPLLAHGDKSLQGPQPPRHQLSEPAPAYHRITPTITNFALR | 422 | Serpin A11 | Serpin | | |
| | | LYKELAADAPGNIFFSPVSISTTLALLSLGAQANTSALILEG | | | | | |
| | | LGFNLTETPEADIHQGFRSLLHTLALPSPKLELKVGNSLFL | | | | | |
| | | DKRLKPRQHYLDSIKELYGAFAFSANFTDSVTTGRQINDY | | | | | |
| | | LRRQTYGQVVDCLPEFSQDTFMVLANYIFFKAKWKHPFS | | | | | |
| | | RYQTQKQESFFVDERTSLQVPMMHQKEMHRFLYDQDLA | | | | | |
| | | CTVLQIEYRGNALALLVLPDPGKMKQVEAALQPQTLRK | | | | | |
| | | WGQLLLPSLLDLHLPRFSISGTYNLEDILPQIGLTNILNLEA | | | | | |
| | | DFSGVTGQLNKTISKVSHKAMVDMSEKGFLAGAASGLLS | | | | | |
| | | QPPSLNTMSDPHAHFNRPFLLLLWEVTTQSLLFLGKVVNP | | | | | |
| | | VAG | | | | | |
| Q8IW75 | 21 | LKPSFSPRNYKALSEVQGWKQRMAAKELARQNMDLGFK | 414 | Serpin A12 | Serpin | | |
| | | LLKKLAFYNPGRNIFLSPLSISTAFSMLCLGAQDSTLDEIK | | | | | |
| | | QGFNFRKMPEKDLHEGFHYIIHELTQKTQDLKLSIGNTLFI | | | | | |
| | | DQRLQPQRKFLEDAKNFYSAETILTNFQNLEMAQKQINDF | | | | | |
| | | ISQKTHGKINNLIENIDPGTVMLLANYIFFRARWKHEFDPN | | | | | |
| | | VTKEEDFFLEKNSSVKVPMMFRSGIYQVGYDDKLSCTILE | | | | | |
| | | IPYQKNITAIFILPDEGKLKHLEKGLQVDTFSRWKTLLSRR | | | | | |
| | | VVDVSVPRLHMTGTFDLKKTLSYIGVSKIFLEHGDLTKIA | | | | | |
| | | PHRSLKVGEAVHKAELKMDERGFEGAAGTGAQTLPMET | | | | | |
| | | PLVVKIDKPYLLLIYSEKIPSVLFLGKIVNPIGK | | | | | |
| O75830 | 19 | SRCSAQKNTEFAVDLYQEVSLSHKDNIIFSPLGITLVLEMV | 405 | Serpin 12 | Serpin | | |
| | | QLGAKGKAQQQIRQTLKQQETSAGEEFFVLKSFFSAISEK | | | | | |
| | | KQEFTFNLANALYLQEGFTVKEQYLHGNKEFFQSAIKLV | | | | | |
| | | DEQDAKACAEMISTWVERKTDGKIKDMFSGEEFGPLTRL | | | | | |
| | | VLVNAIYFKGDWKQKFRKEDTQLINFTKKNGSTVKIPMM | | | | | |
| | | KALLRTKYGYFSESSLNYQVLELSYKGDEFSLIIILPAEGM | | | | | |
| | | DIEEVEKLITAQQILKWLSEMQEEEVEISLPREKVEQKVDF | | | | | |
| | | KDVLYSLNITEIFSGGCDLSGITDSSEVYVSQVTQKVFFEIN | | | | | |
| | | EDGSEAATSTGIHIPVIMSLAQSQFIANHPFLFIMKHNPTES | | | | | |
| | | ILFMGRVTNPDTQEIKGRDLDSL | | | | | |
| O00230 | 89 | DRMPCRNFFVVICITSSCK | 105 | Cortistatin-17 | Somastostatin | | |
| O00230 | 77 | QEGAPPQQSARRDRMPCRNFFWKTFSSCK | 105 | Cortistatin-29 | Somastostatin | | |
| | | | | (Potential) | | | |
| P61278 | 103 | AGCKNFFWKTFTSC | 116 | Somatostatin-14 | Somastostatin | | |
| P61278 | 89 | SANSNPAMAPRERKAGCKNFFWKTFTSC | 116 | Somatostatin-28 | Somastostatin | S89 or S92 | 1 |
| | | | | | | (Ambiguous) | |
| P01236 | 29 | LPICPGGAARCQVTLRDLFDRAVVLSHYIHNLSSEMFSEF | 227 | Prolactin | Somatotropin/ | | |
| | | DKRYTHGRGFITKAINSCHTSSLATPEDKEQAQQMNQKD | | | prolactin | | |
| | | FLSLIVSILRSWNEPLYHLVTEVRGMQEAPEAILSKAVEIE | | | | | |
| | | EQTKRLLEGMELIVSQVHPETKENEIYPVWSGLPSLQMAD | | | | | |
| | | EESRLSAYYNLLHCLRRDSHKIDNYLKLLKCRIIHNNNC | | | | | |
| P20366 | 111 | ALNSVAYERSAMQNYE | 125 | C-terminal- | Tachykinin | A111, S114 | 2 |
| | | | | flanking peptide | | | |
| P20366 | 98 | HKTDSFVGLM | 107 | Neurokinin A | Tachykinin | | |
| Q9UHF0 | 81 | DMHDFFVGLM | 90 | Neurokinin-B | Tachykinin | | |
| P20366 | 72 | DADSSIEKQVALLKALYGHGQISHKRHKTDSFVGLM | 107 | Neuropeptide K | Tachykinin | | |
| P20366 | 58 | RPKPQQFFGLM | 68 | Substance P | Tachykinin | | |
| P20396 | 25 | QPEAAQQEAVTAAEHPGLDDFLRQVERLLFLRENIQRLQ | 242 | Pro-thyrotropin- | TRH | 174 | 1 |
| | | GDQGEHSASQIFQSDWLSKRQHPGKREEEEEEGVEEEEEE | | releasing | | | |
| | | EGGAVGPHKRQHPGRREDEASWSVDVTQHKRQHPGRRS | | hormone | | | |
| | | PWLAYAVPKRQHPGRRLADPKAQRSWEEEEEEEEREEDL | | | | | |
| | | MPEKRQHPGKRALGGPCGPQGAYGQAGLLLGLLDDLSRS | | | | | |
| | | QGAEEKRQHPGRRAAWVREPLEE | | | | | |
| P20396 | 84 | QHP | 86 | Thyrotropin- | TRH | | |
| | | | | releasing | | | |
| | | | | hormone | | | |
| O95399 | 114 | ETPDCFWKYCV | 124 | Urotensin-2 | Urotensin-2 | | |
| Q76510 | 112 | ACFVVKYCV | 119 | Urotensin-2B | Urotensin-2 | | |
| P01185 | 20 | CYFQNCPRG | 28 | Arg-vasopressin | Vasopressin/ | | |
| | | | | | oxytocin | | |
| P01185 | 126 | ASDRSNATQLDGPAGALLLRLVQLAGAPEPFEPAQPDAY | 164 | Copeptin | Vasopressin/ | A152 | 1 |
| | | | | | oxytocin | | |
| P01178 | 32 | AAPDLDVRKCLPCGPGGKGRCFGPNICCAEELGCFVGTA | 125 | Neurophysin 1 | Vasopressin/ | | |
| | | EALRCQEENYLPSPCQSGQKACGSGGRCAVLGLCCSPDG | | | oxytocin | | |
| | | CHADPACDAEATFSQR | | | | | |
| P01185 | 32 | AMSDLELRQCLPCGPGGKGRCFGPSICCADELGCFVGTAE | 124 | Neurophysin 2 | Vasopressin/ | | |
| | | ALRCQEENYLPSPCQSGQKACGSGGRCAAFGVCCNDESC | | | oxytocin | | |
| | | VTEPECREGFHRRA | | | | | |
| P01178 | 20 | CYIQNCPLG | 28 | Oxytocin | Vasopressin/ | | |
| | | | | | oxytocin | | |
| O15240 | 554 | TLQPPSALRRRHYHHALPPSRHYP | 577 | Antimicrobial | VGF | | |
| | | | | peptide | | | |
| | | | | VGF[554-577] | | | |
| O15240 | 281 | RPESALLGGSEAGERLLQQGLAQVEA | 306 | Neuroendocrine | VGF | | |
| | | | | regulatory | | | |
| | | | | peptide-1 | | | |
| O15240 | 310 | QAEATRQAAAQEERLADLASDLLLQYLLQGGARQRGLG | 347 | Neuroendocrine | VGF | | |
| | | | | regulatory | | | |
| | | | | peptide-2 | | | |
| O15240 | 25 | APPGRPEAQPPPLSSEHKEPVAGDAVPGPKDGSAPEVRGA | 615 | Neurosecretory | VGF | P25, S36, P226, | 5 |
| | | RNSEPQDEGELFQGVDPRALAAVLLQALDRPASPPAPSGS | | protein VGF | | S229, T501 | |
| | | QQGPEEEAAEALLTETVRSQTHSLPAPESPEPAAPPRPQTP | | | | | |
| | | ENGPEASDPSEELEALASLLQELRDFSPSSAKRQQETAAA | | | | | |
| | | ETETRTHTLTRVNLESPGPERVWRASWGEFQARVPERAP | | | | | |
| | | LPPPAPSQFQARMPDSGPLPETHKFGEGVSSPKTHLGEAL | | | | | |
| | | APLSKAYQGVAAPFPKARRPESALLGGSEAGERLLQQGL | | | | | |
| | | AQVEAGRRQAEATRQAAAQEERLADLASDLLLQYLLQG | | | | | |
| | | GARQRGLGGRGLQEAAEERESAREEEEAEQERRGGEERV | | | | | |
| | | GEEDEEAAEAEAEAEEAERARQNALLFAEEEDGEAGAED | | | | | |
| | | KRSQEETPGHRRKEAEGTEEGGEEEDDEEMDPQTIDSLIE | | | | | |
| | | LSTKLHLPADDVVSIIEEVEEKRKRKKNAPPEPVPPPRAAP | | | | | |
| | | APTHVRSPQPPPPAPAPARDELPDWNEVLPPWDREEDEV | | | | | |
| | | YPPGPYHPFPNYIRPRTLQPPSALRRRHYHHALPPSRHYPG | | | | | |
| | | REAQARRAQEEAEAEERRLQEQEELENYIEHVLLRRP | | | | | |

Specific embodiments of the invention are disclosed below with indication of the respective SEQ ID NO.

TABLE 6A

| | | | | | | | *Site position (Bold; identified | |
|---|---|---|---|---|---|---|---|---|
| SEQ | | | | | | Peptide | or conserved in human, Italic: | No. |
| ID | Entry_ | Peptide | | Peptide | Peptide | Hormone | identified in non-human and not | of |
| NO | Human | Start | Peptide_Sequence | end | Name | Family | conserved | sites |
| 147 | P01160 | 1 | SLRRSSCFGGRMDRIGAQSGLGCNSFRY | 28 | Atrial | Natriuretic | S19N, S25 | 2 |
| | | | | | natriuretic | peptide | | |
| | | | | | factor | | | |

TABLE 6B

| | | | | | | | *Site position (Bold; identified | |
|---|---|---|---|---|---|---|---|---|
| SEQ | | | | | | Peptide | or conserved in human, Italic: | No. |
| ID | Entry_ | Peptide | | Peptide | Peptide | Hormone | identified in non-human and not | of |
| NO | Human | Start | Peptide_Sequence | end | Name | Family | conserved | sites |
| 72 | P22466 | 1 | GWTLNSAGYLLGPHAVGNHRSFSDKNGLTS | 30 | Galanin | Galanin | T3, S23 | 2 |
| 95 | P01275 | 1 | HSQGTFTSDYSKYLDSRRAQDFVQWLMNT | 29 | Glucagon | Glucagon | T7 | 1 |
| 97 | P01275 | 1 | HAEGTFTSDVSSYLEGQAAKEFIAWLVKGR | 31 | Glucago-like | Glucagon | T5 or T7 or S8 (Ambiguous) | 1 |
| | | | | | peptide 1 | | | |
| | | | | | (7-36) | | | |
| 106 | P09683 | 1 | HSDGTFTSELSRLREGARLQRLLQGLV | 27 | Secretin | Glucagon | T7, S8, S11 | 3 |
| 108 | P01282 | 1 | HSDAVFTDNYTRLRKQMAVKKYLNSILN | 28 | Vasoactive | Glucagon | T7 | 1 |
| | | | | | intestinal | | | |
| | | | | | peptide | | | |
| 147 | P01160 | 1 | SLRRSSCFGGRMDRIGAQSGLGCNSFRY | 28 | Atrial | Natriuretic | S19, S25 | 2 |
| | | | | | natriuretic | peptide | | |
| | | | | | factor | | | |
| 185 | P01303 | 1 | YPSKPDNPGEDAPAEDMARYYSALRHYINLITRQRY | 36 | Neuropeptide Y | NPY | S3, T32 | 2 |
| 188 | P10082 | 1 | YPIKPAPREDASPEELNRYYASLRHYLNLVTRQRY | 36 | Peptide YY | NPY | T32 | 1 |

TABLE 6C

| SEQ | | | | | | Peptide | | No. |
|---|---|---|---|---|---|---|---|---|
| ID | Entry_ | Peptide | | Peptide | Peptide | Hormone | *Site position (identified | of |
| NO | Human | Start | Peptide_Sequence | end | Name | Family | or conserved in humans) | sites |
| 92 | P09681 | 1 | YAEGTFISDYSIAMDKIHQQDFVNWLLAQKGKKNDW | 42 | Gastric | Glucagon | S8 | 1 |
| | | | KHNITQ | | inhibitory | | | |
| | | | | | polypeptide | | | |
| 95 | P01275 | 1 | HSQGTFTSDYSKYLDSRRAQDFVQWLMNT | 29 | Glucagon | Glucagon | T7 | 1 |
| 97 | P01275 | 1 | HAEGTFTSDVSSYLEGQAAKEFIAWLVKGR | 31 | Glucago-like | Glucagon | T5 or T7 or S8 (Ambiguous) | 1 |
| | | | | | peptide 1 | | | |
| | | | | | (7-36) | | | |
| 99 | P01275 | 1 | HADGSFSDEMNTILDNLAARDFINWLIQTKITD | 33 | Glucagon-like | Glucagon | S7 | 1 |
| | | | | | peptide 2 | | | |
| | | | | | (By similarity) | | | |
| 100 | P01282 | 1 | HADGVFTSDFSKLLGQLSAKKYLESLM | 27 | Intestinal | Glucagon | T7, S8 | 2 |
| | | | | | peptide | | | |
| | | | | | PHM-27 | | | |
| 106 | P09683 | 1 | HSDGTFTSELSRLREGARLQRLLQGLV | 27 | Secretin | Glucagon | T7, S8, S11 | 3 |
| 107 | P01286 | 1 | YADAIFTNSYRKVLGQLSARKLLQDIMSRQQGESNQ | 44 | Somatoliberin | Blucagon | T7 | 1 |
| | | | ERGARARL | | | | | |
| 108 | P01282 | 1 | HSDAVFTDNYTRLRKQMAVKKYLNSILN | 28 | Vasoactive | Glucagon | T7 | 1 |
| | | | | | intestinal | | | |
| | | | | | peptide | | | |
| 21 | P01258 | 1 | CGNLSTCMLGTYTQDFNKFHTFPQTAIGVGAP | 32 | Calcitonin | Calcitonin | T6, T11. T13, T21, T25 | 5 |
| 22 | P06881 | 1 | ACDTATCVTHRLAGLLSRSGGVVKNNFVPTNVGSK | 37 | Calcitonin | Calcitonin | T9, S17 | 2 |
| | | | AF | | gene- | | | |
| | | | | | related | | | |
| | | | | | peptide 1 | | | |
| 23 | P10092 | 1 | ACNTATCVTHRLAGLLSRSGGMVKSNFVPTNVGSK | 37 | Calcitonin | Calcitonin | T9, S17, S25, T30, S34 | 5 |
| | | | AG | | gene- | | | |
| | | | | | related | | | |
| | | | | | peptide 2 | | | |
| 24 | P10997 | 1 | KCNTATCATQRLANFLVHSSNNFGAILSSTNVGSN | 37 | Islet amyloid | Calcitonin | T6, T9 | 3 |
| | | | TY | | polypeptide | | | |
| 6 | P35318 | 1 | YRQSMNNFQGLRSFGCRFGTCTVQKLAHQIYQFTD | 52 | Adrenomedullin | Adrenomedullin | T22, Y31, T34 | 3 |
| | | | KDKDNVAPRSKISPQGY | | | | | |
| 7 | Q7Z4H4 | 1 | TQAQLLRVGCVLGTCQVQNLSHRLWQLMGPAGRQDS | 47 | Adrenomedullin- | Adrenomedullin | T14 | 1 |
| | | | APVDPSSPHSY | | 2 (By | | | |
| | | | | | similarity) | | | |
| 116 | P01308 | 1 | FVNQHLCGSHLVEALYLVCGERGFFYTPKT | 30 | Insulin B | Insulin | T27 (Ambiguous) | 1 |
| | | | | | chain | | | |
| 117 | P05019 | 1 | GPETLCGAELVDALQFVCGDRGFYFNKPTGYGSSSS | 70 | Inslulin-like | Insulin | T29 | 1 |
| | | | RAPQTGIVDECCFRSCDLRRLEMYCAPLKPAKSA | | growth factor I | | | |
| 118 | P01344 | 1 | AYRPSETLCGGELVDTLQFVCGDRGFYFSRPASRVS | 67 | Inslulin-like | Insulin | S29, S50, T62 | 2 |
| | | | RRSRGIVEECCFRSCDLALLETYCATPAKSE | | growth factor | | | |
| | | | | | II | | | |
| 72 | P22466 | 1 | GWTLNSAGYLLGPHAVGNHRSFSDKNGLTS | 30 | Galanin | Galanin | T3, S23 | 2 |
| 74 | Q9UBC7 | 1 | APAHRGRGGWTLNSAGYLLGPVLHLPQMGDQDGKRE | 60 | Galanin-like | Galanin | T11 | 1 |
| | | | TALEILDLWKAIDGLPYSHPPQPS | | peptide | | | |
| 185 | P01303 | 1 | YPSKPDNPGEDAPAEDMARYYSALRHYINLITRQRY | 36 | Neuropeptide Y | NPY | S3, T32 | 2 |
| 186 | P01298 | 1 | APLEPVYPGDNATPEQMAQYAADLRRYINMLTRPRY | 36 | Pancreatic | NPY | T32 | 1 |
| | | | | | hormone | | | |
| 188 | P10082 | 1 | YPIKPAPREDASPEELNRYYASLRHYLNLVTRQRY | 36 | Peptide YY | NPY | T32 | 1 |
| 147 | P01160 | 1 | SLRRSSCFGGRMDRIGAQSGLGCNSFRY | 28 | Atrial | Natriuretic | S19, S25 | 2 |
| | | | | | natriuretic | peptide | | |
| | | | | | factor | | | |
| 163 | P16860 | 1 | SPKMVQGSGCFGRKMDRISSSSGLGCKVLRRH | 32 | Brain | Natriuretic | S22 | 1 |
| | | | | | natriuretic | peptide | | |
| 167 | P23582 | 1 | DLRVDTKSRAAWARLLQEHPNARKYKGANKKGLSKG | 53 | CNP-53 | Natriuretic | S47 | 1 |
| | | | CFGLKLDRIGSMSGLGC | | | peptide | | |

TABLE 6D

| | | | | | | | *Site position (Bold: | |
|---|---|---|---|---|---|---|---|---|
| | | | | | | | identified or conserved | |
| SEQ | | Pep- | | Pep- | | Peptide | in human, Italic; | No. |
| ID | Entry_ | tide | | tide | Peptide | Hormone | identified in non-human | of |
| NO. | Human | Start | Peptide_Sequence | end | Name | Family | and not conserved) | sites |
| 1 | P05408 | 1 | SVPHFSDEDKDPE | 13 | C-terminal | 7B2 | S1 | 1 |
| | | | | | peptide (By | | | |
| | | | | | similarity) | | | |
| 2 | P05408 | 1 | YSPRTPDRVSEADIQRLLHGVMEQLGIARPRVEYPAHQ | 186 | Neuroendocrine | 7B2 | S2, T5, S10, T108, S174 | 5 |
| | | | AMNLVGPQSIEGGAHEGLQHLGPFGNIPNIVAELTGDN | | protein 7B2 | | | |
| | | | IPKDFSEDQGYPDPPNPCPVGKTADDGCLENTPDTAEF | | | | | |
| | | | SREFQLHQHLFDPEHDYPGLGKWNKKLLYEKMKGGE | | | | | |
| | | | RRKRRSVNPYLQGQRLDNVVAKKSVPHFSDEDKDPE | | | | | |
| 3 | P05408 | 1 | YSPRTPDRVSEADIQRLLHGVMEQLGIARPRVEYPAHQ | 150 | N-terminal | 7B2 | S2, T5, S10, T108 | 4 |
| | | | AMNLVGPQSIEGGAHEGLQHLGPFGNIPNIVAELTGDN | | peptide (By | | | |
| | | | IPKDFSEDQGYPDPPNPCPVGKTADDGCLENTPDTAEF | | similarity) | | | |
| | | | SREFQLHQHLFDPEHDYPGLGKWNKKLLYEKMKGGE | | | | | |
| 5 | P07108 | 1 | SQAEFEKAAEEVRHLKTKPSDEEMLFIYGHYKQATVG | 86 | Acyl-CoA- | ACBP | T35 or T41 (ambiguous) | 1 |
| | | | DINTERPGMLDFTGKAKWDAWNELKGTSKEDAMKA | | binding protein | | | |
| | | | YINKVEELKKKYGI | | | | | |
| 6 | P35318 | 1 | YRQSMNNFQGLRSFGCRFGTCTVQKLAHQIYQFTDKD | 52 | Adrenomedullin | Adrenomedullin | T22, Y31, T34 | 3 |
| | | | KDNVAPRSKISPQGY | | | | | |
| 7 | Q7Z4H4 | 1 | TQAQLLRVGCVLGTCQVQNLSHRLWQLMGPAGRQDS | 47 | Adrenomedullin-2 | Adrenomedullin | T14 | 1 |
| | | | APVDPSSPHSY | | (By similarity) | | | |
| 14 | P07492 | 1 | VPLPAGGGTVLTKMYPRGNHWAVGHLM | 27 | Gastrin-releasing | Bombesin/neuromedin- | P4, T9 | 2 |
| | | | | | peptide | B/ranatensin | | |
| 15 | P08949 | 1 | GNLWATGHFM | 10 | Neuromedin-B | Bombesin/neuromedin- | T6 | 1 |
| | | | | | | B/ranatensin | | |
| 16 | P08949 | 1 | APLSWDLPEPRSRASKIRVHSRGNLWATGHFM | 32 | Neuromedin-B-32 | Bombesin/neuromedin- | T28 | 1 |
| | | | | | | B/ranatensin | | |
| 21 | P01258 | 1 | CGNLSTCMLGTYTQDFNKFHTFPQTAIGVGAP | 32 | Calcitonin | Calcitonin | T6, T11, T13, T21, T25 | 5 |
| 22 | P06881 | 1 | ACDTATCVTHRLAGLLSRSGGVVKNNFVPTNVGSKAF | 37 | Calcitonin gene- | Calcitonin | T9, S17 | 2 |
| | | | | | related peptide 1 | | | |
| 23 | P10092 | 1 | ACNTATCVTHRLAGLLSRSGGMVKSNFVPTNVGSKAF | 37 | Calcitonin gene- | Calcitonin | T9, S17, S25, T30, S34 | 5 |
| | | | | | related peptide 2 | | | |
| 24 | P10997 | 1 | KCNTATCATQRLANFLVHSSNNFGAILSSTNVGSNTY | 37 | Islet amyloid | Calcitonin | T5, T9, T30 | 3 |
| | | | | | polypeptide | | | |
| 25 | P01258 | 1 | DMSSDLERDHRPHVSMPQNAN | 21 | Katacalcin | Calcitonin | S15 | 1 |
| 36 | P05060 | 1 | SAEFPDFYDSEEPVSTHQEAENEKDRADQTVLTEDEKK | 57 | CCB peptide | Chromogranin/ | S15 | 1 |
| | | | ELENLAAMDLELQKIAEKF | | | secretogranin | | |
| 37 | P10645 | 1 | LPVNSPMNKGDTEVMKCIVEVISDTLSKPSPMPVSQEC | 439 | Chromogranin-A | Chromogranin/ | S27, S30, S36, S80, E104, S108, | 39 |
| | | | FETLRGDERILSILRHQNLLKELQDLALQGAKERAHQQ | | | secretogranin | A127, T128, A135, L136, S143 | |
| | | | KKHSGFEDELSEVLENQSSQAELKEAVEEPSSKDVME | | | | A151, N164, T165 P167, A169, | |
| | | | KREDSKEAEKSGEATDGARPQALPEPMQESKAEGNNQ | | | | S170, S173, E182, L188 S189, | |
| | | | APGEEEEEEEEATNTHPPASLPSQKYPGPQAEGDSEGLS | | | | V193, S200, T233, L236, N237, | |
| | | | QGLVDREKGLSAEPGWQAKREEEEEEEEEAEAGEEAV | | | | P238, S241, Y244, P265, E268, | |
| | | | PEEEGPTVVLNPHPSLGYKEIRKGESRSEALAVDGAGK | | | | P273, S282, M291, V294, T335, | |
| | | | PGAEEAQDPEGKGEQEHSQQKEEEEEMAVVPQGLFRG | | | | A360, S380, G395 | |
| | | | GKSGELEQEEERLSKEWEDSKRWSKMDQLAKELTAE | | | | | |
| | | | KRLEGQEEEEDNRDSSMKLSFRARAYGFRGPGPQLRR | | | | | |
| | | | GWRPSSREDSLEAGLPLQVRGYPEEKKEEEGSANRRPE | | | | | |
| | | | DQELESLSAIEAELEKVAHQLQALRRG | | | | | |
| 38 | P10645 | 1 | EDSKEAEKSGEATDGARPQALPEPMQESKAEGNNQAP | 92 | EA-92 | Chromogranin/ | A12, T13, A20, L21, S28, A36, | 17 |
| | | | GEEEEEEEEATNTHPPASLPSQKYPGPQAEGDSEGLSQ | | | secretogranin | N49, T50, P52, A54, S55, S58, | |
| | | | GLVDREKGLSAEPGWQA | | | | E67, L73, S74, V78, S85 | |
| 39 | P10645 | 1 | EEEGSANRRPEDQELESLSAIEAELEKVAHQLQALRR | 37 | ER-37 | Chromogranin/ | S17 or S19 (Ambiguous) | 1 |
| | | | | | | secretogranin | | |
| 40 | P10645 | 1 | EEEEEEEEEAEAGEEAVPEEEGPTVVLNPHPSL | 33 | ES-43 | Chromogranin/ | T24, L27, N28, P29, S32 | 5 |
| | | | | | | secretogranin | | |
| 41 | P05060 | 1 | FLGEGHHRVQENQMDKARRHPQGAWKELDRNYLNY | 74 | GAWK peptide | Chromogranin/ | H7, N12, Y35, T72, A73 | 5 |
| | | | GEEGAPGKWQQQGDLQDTKENREEARFQDKQYSSHH | | | secretogranin | | |
| | | | TAE | | | | | |
| 42 | P10645 | 1 | GYPEEKKEEEGSANRRPEDQELESLSAIEAELEKVAHQ | 44 | GR-44 | Chromogranin/ | G1 | 1 |
| | | | LQALRR | | | secretogranin | | |
| 43 | P10645 | 1 | GWRPSSREDSLEAGLPLQV | 19 | GV-19 | Chromogranin/ | S6 | 1 |
| | | | | | | secretogranin | | |
| 45 | P10645 | 1 | SEALAVDGAGKPGAEEAQDPEGKGEQEHSQQKEEEEE | 48 | Pancreastatin | Chromogranin/ | P12, E15, P20, S29, M38, V41 | 6 |
| | | | MAVVPQGLFRG | | | secretogranin | | |
| 46 | P05060 | 1 | MPVDNRNHNEGMVTRCIIEVLSNALSKSSAPPITPECR | 657 | Secretogranin-1 | Chromogranin/ | T14, S26, S28, S29, P32, T59, | 40 |
| | | | QVLKTSRKDVKDKETTENENTKFEVRLLRDPADASEA | | | secretogranin | S73, A75, S80, A84, E89, T96, | |
| | | | HESSSRGEAGAPGEEDIQGPTKADTEKWAEGGGHSRE | | | | S120, S124, S129, T174, N176, | |
| | | | RADEPQWSLYPSDSQVSEEVKTRHSEKSQREDEEEEEG | | | | S186, S273, S274, S278, P287, | |
| | | | ENYQKGERGEDSSEEKHLEEPGETQNAFLNERKQASAI | | | | S22L, M298, S300, D306, T310, | |
| | | | KKEELVARSETHAAGHSQEKTHSREKSSQESGEETGSQ | | | | Y328, P329, G330, K376, M377, | |
| | | | ENHPQESKGQPRSQEESEEGEEDATSEVDKRRTRPRHH | | | | H426, Y454, T491, A492, T536, | |
| | | | HGRSRPDRSSQGGSLPSEEKGHPQEESEESNVSMASLG | | | | S557, N568, S611 | |
| | | | EKRDHHSTHYRASEEEPEYGEEIKGYPGVQAPEDLEW | | | | | |
| | | | ERYRGRGSEEYRAPRPQSEESWDEEDKRNYPSLELDK | | | | | |
| | | | MAHGYGEESEEERGLEPGKGRHHRGRGGEPRAYFMS | | | | | |
| | | | DTREEKRFLGEGHHRVQENQMDKARRHPQGAWKELD | | | | | |
| | | | RNYLNYGEEGAPGKWQQQGDLQDTKENREEARFQDK | | | | | |
| | | | QYSSHHTAEKRKRLGELFNPYYDPLQWKSSHFERRDN | | | | | |
| | | | MNDNFLEGEEENELTLNEKNFFPEYNYDWWEKKPFSE | | | | | |
| | | | DVNWGYEKRNLARVPKLDLKRQYDRVAQLDQLLHY | | | | | |
| | | | RKKSAEFPDFYDSEEPVSTHQEAENEKDRADQTVLTE | | | | | |
| | | | DEKKELENLAAMDLELQKIAEKFSQRG | | | | | |
| 47 | P13521 | 1 | QRNQLLQKEPDLRLENVQKFPSPEMIRALEYIENLRQQ | 587 | Secretogranin-2 | Chromogranin/ | S22, S45, A106, Y160, T161, | 16 |
| | | | AHKEESSPDYNPYQGVSVPLQQKENGDESHLPERDSLS | | | secretogranin | S164, T167, S229, T231, S348, | |
| | | | EEDWMRIILEALRQAENEPQSAPKENKPYALNSEKNFP | | | | T420, N459, G499, S526, S536, | |
| | | | MDMSDDYETQQWPERKLKHMQFPPMYEENSRDNPFK | | | | P543 | |
| | | | RTNEIVEEQYTPQSLATLESVFQELGKLTGPNNQKRER | | | | | |
| | | | MDEEQKLYTDDEDDIYKANNIAYEDVVGGEDWNPVE | | | | | |
| | | | EKIESQTQEEVRDSKENIEKNEQINDEMKRSGQLGIQEE | | | | | |
| | | | DLRKESKDQLSDDVSKVIAYLKRLVNAAGSGRLQNGQ | | | | | |
| | | | NGERATRLFEKPLDSQSIYQLIEISRNLQIPPEDLIEMLK | | | | | |
| | | | TGEKPNGSVEPERELDLPVDLDDISEADLDHPDLFQNR | | | | | |
| | | | MLSKSGYPKTPGRAGTEALPDGLSVEDILNLLGMESA | | | | | |
| | | | ANQKTSYFPNPYNQEKVLPRLPYGAGRSRSNQLPKAA | | | | | |
| | | | WIPHVENRQMAYENLNDKDQELGEYLARMLVKYPEII | | | | | |
| | | | NSNQVKRVPGQGSSEDDLQEEEQIEQAIKEHLNQGSSQ | | | | | |
| | | | ETDKLAPVSKRFPVGPPKNDDTPNRQYWDEDLLMKV | | | | | |
| | | | LEYLNQEKAEKGREHIAKRAMENM | | | | | |
| 48 | Q8WXD2 | 1 | FPKPGGSQDKSLHNRELSAERPLNEQIAEAEEDKIKKT | 449 | Secretogranin-3 | Chromogranin/ | P41, T63, S103, T104, T125, | 15 |
| | | | YPPENKPGQSNYSFVDNLNLLKAITEKEKIEKERQSIRS | | | secretogranin | A138, D192, P193, T197, S198, | |
| | | | SPLDNKLNVEDVDSTKNRKLIDDYDSTKSGLDHKFQD | | | | T200, T212, I217, A222, S340 | |
| | | | DPDGLHQLDGTPLTAEDIVHKIAARIYEENDRAVFDKI | | | | | |
| | | | VSKLLNLGLITESQAHTLEDEVAEVLQKLISKEANNYE | | | | | |
| | | | EDPNKPTSWTENQAGKIPEKVTPMAAIQDGLAKGEND | | | | | |
| | | | ETVSNTLTLTNGLERRTKTYSEDNFEELQYFPNFYALL | | | | | |
| | | | KSIDSEKEAKEKETLITIMKTLIDFVKMMVKYGTISPEE | | | | | |
| | | | GVSYLENLDEMIALQTKNKLEKNATDNISKLFPAPSEK | | | | | |
| | | | SHEETDSTKEEAAKMEKEYGSLKDSTKDDNSNPGGKT | | | | | |
| | | | DEPKGKTEAYLEAIRKNIEWLKKHDKKGNKEDYDLSK | | | | | |
| | | | MRDFINKQADAYVEKGILDKEEAEAIKRIYSSL | | | | | |
| 49 | P13521 | 1 | TNEIVEEQYTPQSLATLESVFQELGKLTGPNNQ | 33 | Secretoneurin | Chromogranin/ | Y9, T10, S13, T16, | 4 |
| | | | | | | secretogranin | | |
| 51 | P10645 | 1 | LPVNSPMNKGDTEVMKCIVEVISDTLSKPSPMPVSQEC | 76 | Vasostatin-1 | Chromogranin/ | S27, S30, S36 | 3 |
| | | | FETLRGDERILSILRHQNLLKELQDLALQGAKERAHQQ | | | secretogranin | | |
| 52 | P10645 | 1 | LPVNSPMNKGDTEVMKCIVEVISDTLSKPSPMPVSQEC | 113 | Vasostatin-2 | Chromogranin/ | S27, S30, S36, S80, E104, | 6 |
| | | | FETLRGDERILSILRHQNLLKELQDLALQGAKERAHQQ | | | secretogranin | S108 | |
| | | | KKHSGFEDELSEVLENQSSQAELKEAVEEPSSKDVME | | | | | |
| 54 | P10645 | 1 | WSKMDQLAKELTAE | 14 | WE-14 | Chromogranin/ | T12 | 1 |
| | | | | | | secretogranin | | |
| 55 | P01042 | 1 | QESQSEEIDCNDKDLFKAVDAALKKYNSQNQSNNQFV | 626 | Kininogen-1 | Cystatin | T119, T133, T243, T255, E381, | 15 |
| | | | LYRITEATKTVGSDTFYSFKYEIKEGDCPVQSGKTWQD | | | | T382, T383, S385, T389, S390, | |
| | | | CEYKDAAKAATGECTATVGKRSSTKFSVATQTCQITP | | | | T528, S586, T592, S593, T610 | |
| | | | AEGPVVTAQYDCLGCVHPISTQSPDLEPILRHGIQYFN | | | | | |
| | | | NNTQHSSLFMLNEVKRAQRQVVAGLNFRITYSIVQTN | | | | | |
| | | | CSKENFLFLTPDCKSLWNGDTGECTDNAYIDIQLRIASF | | | | | |
| | | | SQNCDIYPGKDFVQPPTKICVGCPRDIPTNSPELEETLT | | | | | |
| | | | HTITKLNAENNATFYFKIDNVKKARVQVVAGKKYFID | | | | | |
| | | | FVARETTCSKESNEELTESCETKKLGQSLDCNAEVYVV | | | | | |
| | | | PWEKKIYPTVNCQPLGMISLMKRPPGFSPFRSSRIGEIK | | | | | |
| | | | EETTVSPPHTSMAPAQDEERDSGKEQGHTRRHDWGH | | | | | |
| | | | EKQRKHNLGHGHKHERDQGHGHQRGHGLGHGHEQQ | | | | | |
| | | | HGLGHGHKFKLDDDLEHQGGHVLDHGHKHKHGHGH | | | | | |
| | | | GKHKNKGKKNGKHNGWKTEHLASSSEDSTTPSAQTQ | | | | | |
| | | | EKTEGPTPIPSLAKPGVTVTFSDFQDSDLIATMMPPISP | | | | | |
| | | | APIQSDDDWIPDIQIDPNGLSFNPISDFPDTTSPKCPGRP | | | | | |
| | | | WKSVSEINPTTQMKESYYFDLTDGLS | | | | | |
| 56 | P01042 | 1 | QESQSEEIDCNDKDLFKAVDAALKKYNSQNQSNNQFV | 362 | Kininogen-1 | Cystatin | T119, T133, T243, T255 | 4 |
| | | | LYRITEATKTVGSDTFYSFKYEIKEGDCPVQSGKTWQD | | heavy chain | | | |
| | | | CEYKDAAKAATGECTATVGKRSSTKFSVATQTCQITP | | | | | |
| | | | AEGPVVTAQYDCLGCVHPISTQSPDLEPILRHGIQYFN | | | | | |
| | | | NNTQHSSLFMLNEVKRAQRQVVAGLNFRITYSIVQTN | | | | | |
| | | | CSKENFLFLTPDCKSLWNGDTGECTDNAYIDIQLRIASF | | | | | |
| | | | SQNCDIYPGKDFVQPPTKICVGCPRDIPTNSPELEETLT | | | | | |
| | | | HTITKLNAENNATFYFKIDNVKKARVQVVAGKKYFID | | | | | |
| | | | FVARETTCSKESNEELTESCETKKLGQSLDCNAEVYVV | | | | | |
| | | | PWEKKIYPTVNCQPLGMISLMK | | | | | |
| 57 | P01042 | 1 | SSRIGEIKEETTVSPPHTSMAPAQDEERDSGKEQGHTR | 255 | Kininogen-1 light | Cystatin | E10, T11, T12, S14, T18, S19, | 11 |
| | | | RHDWGHEKQRKHNLGHGHKHERDQGHGHQRGHGLG | | chain | | T157, S215, T221, S222, T239 | |
| | | | HGHEQQHGLGHGHKFKLDDDLEHQGGHVLDHGHKH | | | | | |
| | | | KHGHGHGKHKNKGKKNGKHNGWKTEHLASSSEDSTT | | | | | |
| | | | PSAQTQEKTEGPTPIPSLAKPGVTVTFSDFQDSDLIATM | | | | | |
| | | | MPPISPAPIQSDDDWIPDIQIDPNGLSFNPISDFPDTT | | | | | |
| | | | SPKCPGRPWKSVSEINPTTQMKESYYFDLTDGLS | | | | | |
| 72 | P22466 | 1 | GWTLNSAGYLLGPHAVGNHRSFSDKNGLTS | 30 | Galanin | Galanin | T3, S23 | 2 |
| 73 | P22466 | 1 | ELRPEDDMKPGSFDRSIPENNIMRTIIEFLSFLHLKEA | 59 | Galanin message- | Galanin | A49 | 1 |
| | | | GALDRLLDLPAAASSEDIERS | | associated peptide | | | |
| 74 | Q9UBC7 | 1 | APAHRGRGGWTLNSAGYLLGPVLHLPQMGDQDGKRE | 60 | Galanin-like | Galanin | T11 | 1 |
| | | | TALEILDLWKAIDGLPYSHPPQPS | | peptide | | | |
| 76 | P06307 | 1 | QPVPPADPAGSGLQRAEEAPRRQLRVSQRTDGESRAH | 95 | Cholecystokinin | Gastrin/ | A8, T30, N65 | 3 |
| | | | LGALLARYIQQARKAPSGRMSIVKNLQNLDPSHRISDR | | | cholecystokinin | | |
| | | | DYMGWMDFGRRSAEEYEYPS | | | | | |
| 79 | P06307 | 1 | IVKNLQNLDPSHRISDRDYMGWMDF | 25 | Cholecystokinin- | Gastrin/ | N7 | 1 |
| | | | | | 25 (By | cholecystokinin | | |
| | | | | | similarity) | | | |
| 80 | P06307 | 1 | KAPSGRMSIVKNLQNLDPSHRISDRDYMGWMDF | 33 | Cholecystokinin- | Gastrin/ | N15 | 1 |
| | | | | | 33 | cholecystokinin | | |
| 81 | P06307 | 1 | YIQQARKAPSGRMSIVKNLQNLDPSHRISDRDYMGWM | 39 | Cholecystokinin- | Gastrin/ | N21 | 1 |
| | | | DF | | 39 | cholecystokinin | | |
| 83 | P06307 | 1 | VSQRTDGESRAHLGALLARYIQQARKAPSGRMSIVKN | 58 | Cholecystokinin- | Gastrin/ | T5, N40 | 2 |
| | | | LQNLDPSHRISDRDYMGWMDF | | 58 | cholecystokinin | | |
| 84 | P06307 | 1 | VSQRTDGESRAHLGALLARYIQQARKAPSGRMSIVKN | 49 | Cholecystokinin- | Gastrin/ | T5, N40 | 2 |
| | | | LQNLDPSHRISD | | 58 | cholecystokinin | | |
| | | | | | desnonopeptide | | | |
| | | | | | (By similarity) | | | |
| 91 | P01350 | 1 | SWKPRSQQPDAPLGTGANRDLELPWLEQQGPASHHRR | 71 | Gastrin-71 | Gastrin/ | L12 or G13 (Ambiguous) | 1 |
| | | | QLGPQGPPHLVADPSKKQGPWLEEEEEAYGWMDF | | | cholecystokinin | | |
| 92 | P09681 | 1 | YAEGTFISDYSIAMDKIHQQDFVNWLLAQKGKKNDW | 42 | Gastric | Glucagon | S8 | 1 |
| | | | KHNITQ | | inhibitory | | | |
| | | | | | polypeptide | | | |
| 93 | P01275 | 1 | RSLQDTEEKSRSFSASQADPLSDPDQMNEDKRHSQGTF | 69 | Glicentin (By | Glucagon | S12, T39 | 2 |
| | | | TSDYSKYLDSRRAQDFVQWLMNTKRNRNNIA | | similarity) | | | |
| 94 | P01275 | 1 | RSLQDTEEKSRSFSASQADPLSDPDQMNED | 30 | Glicentin-related | Glucagon | S12 | 1 |
| | | | | | polypeptide (By | | | |
| | | | | | similarity) | | | |
| 95 | P01275 | 1 | HSQGTFTSDYSKYLDSRRAQDFVQWLMNT | 29 | Glucagon | Glucagon | T7 | 1 |
| 96 | P01275 | 1 | HDEFERHAEGTFTSDVSSYLEGQAAKEFIAWLVKGRG | 37 | Glucagon-like | Glucagon | T11 or T13 or S14 (Ambiguous) | 1 |
| | | | | | peptide 1 | | | |
| 97 | P01275 | 1 | HAEGTFTSDVSSYLEGQAAKEFIAWLVKGR | 31 | Glucagon-like | Glucagon | T5 or T7 or S8 (Ambiguous) | 1 |
| | | | | | peptide 1(7-36) | | | |
| 98 | P01275 | 1 | HAEGTFTSDVSSYLEGQAAKEFIAWLVKGRG | 30 | Glucagon-like | Glucagon | T5 or T7 or S8 (Ambiguous) | 1 |
| | | | | | peptide 1(7-37) | | | |
| 99 | P01275 | 1 | HADGSFSDEMNTILDNLAARDFINWLIQTKITD | 33 | Glucagon-like | Glucagon | S7 | 1 |
| | | | | | peptide 2 (By | | | |
| | | | | | similarity) | | | |
| 100 | P01282 | 1 | HADGVFTSDFSKLLGQLSAKKYLESLM | 27 | Intestinal | Glucagon | T7, S8 | 2 |
| | | | | | peptide PHM-27 | | | |
| 101 | P01282 | 1 | HADGVFTSDFSKLLGQLSAKKYLESLMGKRVSSNISED | 42 | Intestinal | Glucagon | T7, S8 | 2 |
| | | | PVPV | | peptide PHV-42 | | | |
| 102 | P01275 | 1 | HSQGTFTSDYSKYLDSRRAQDFVQWLMNTKRNRNNI | 3 7 | Oxyntomodulin | Glucagon | T7 | 1 |
| | | | A | | (By similarity) | | | |
| 106 | P09683 | 1 | HSDGTFTSELSRLREGARLQRLLQGLV | 27 | Secretin | Glucagon | T7, S8, S11 | 3 |
| 107 | P01286 | 1 | YADAIFTNSYRKVLGQLSARKLLQDIMSRQQGESNQE | 44 | Somatoliberin | Glucagon | T7 | 1 |
| | | | RGARARL | | | | | |
| 108 | P01282 | 1 | HSDAVFTDNYTRLRKQMAVKKYLNSILN | 28 | Vasoactive | Glucagon | T7 | 1 |
| | | | | | intestinal peptide | | | |
| 109 | P01148 | 1 | DAENLIDSFQEIVKEVGQLAETQRFECTTHQPRSPLRDL | 56 | GnRH-associated | GnRH | T28 | 1 |
| | | | KGALESLIEEETGQKKI | | peptide 1 | | | |
| 113 | P01148 | 1 | QHWSYGLRPGGKRDAENLIDSFQEIVKEVGQLAETQR | 69 | Progonadoliberin- | GnRH | T41 | 1 |
| | | | FECTTHQPRSPLRDLKGALESLIEEETGQKKI | | 1 | | | |
| 116 | P01308 | 1 | FVNQHLCGSHLVEALYLVCGERGFFYTPKT | 30 | Insulin B chain | Insulin | T27 (Ambiguous) | 1 |
| 117 | P05019 | 1 | GPETLCGAELVDALQFVCGDRGFYFNKPTGYGSSSRR | 70 | Insulin-like | Insulin | T29 | 1 |
| | | | APQTGIVDECCFRSCDLRRLEMYCATPLKPAKSA | | growth factor I | | | |
| 118 | P01344 | 1 | AYRPSETLCGGELVDTLQFVCGDRGFYFSRPASRVSRR | 67 | Insulin-like | Insulin | S50, T62 | 2 |
| | | | SRGIVEECCFRSCDLALLETYCATPAKSE | | growth factor II | | | |
| 119 | P01344 | 1 | YRPSETLCGGELVDTLQFVCGDRGFYFSRPASRVSRRS | 66 | Insulin-like | Insulin | S49, T61 | 2 |
| | | | RGIVEECCFRSCDLALLETYCATPAKSE | | growth factor II | | | |
| | | | | | Ala-25 Del | | | |
| 120 | P01344 | 1 | DVSTPPTVLPDNFPRYPVGKFFQYDTWKQSTQRL | 34 | Preptin | Insulin | S3, T4, T7 | 3 |
| 130 | Q15726 | 1 | EPLEKVASVGNSRPTGQQLESLGLLAPGEQSLPCTERK | 119 | Metastasis- | KISS1 | A69 | 1 |
| | | | PAATARLSRRGTSLSPPPESSGSPQQPGLSAPHSRQIPA | | suppressor KiSS-1 | | | |
| | | | PQGAVLVQREKDLPNYNWNSFGLRFGKREAAPGNHGR | | | | | |
| | | | SAGRG | | | | | |
| 131 | Q15726 | 1 | GTSLSPPPESSGSPQQPGLSAPHSRQIPAPQGAVLVQRE | 54 | Metastin | KISS1 | A21 | 1 |
| | | | KDLPNYNWNSFGLRF | | | | | |
| 135 | P20382 | 1 | GSVAFPAENGVQNTESTQE | 19 | Neuropeptide- | Melanin- | T14, T16, T17 | 3 |
| | | | | | glycine-glutamic | concentrating | | |
| | | | | | acid (Potential) | hormone | | |
| 136 | P20382 | 1 | ILLSASKSIRNLDDDMVFNTFRLGKGFQKEDTAEKSVI | 144 | Pro-MCH | Melanin- | S36, S41, N72, T102, T104, | 6 |
| | | | APSLEQYKNDESSFMNEEENKVSKNTGSKHNFLNHGL | | | concentrating | T105 | |
| | | | PLNLAIKPYLALKGSVAFPAENGVQNTESTQEKREIGD | | | hormone | | |
| | | | EENSAKFPIGRRDFDMLRCMLGRVYRPCWQV | | | | | |
| 143 | Q15848 | 1 | ETTTQGPGVLLPLPKGACTGWMAGIPGHPGHNGAPGR | 226 | Adiponectin | NA | T4 | 1 |
| | | | DGRDGTPGEKGEKGDPGLIGPKGDIGETGVPGAEGPR | | | | | |
| | | | GFPGIQGRKGEPGEGAYVYRSAFSVGLETYVTIPNMPI | | | | | |
| | | | RFTKIFYNQQNHYDGSTGKFHCNIPGLYYFAYHITVYM | | | | | |
| | | | KDVKVSLFKKDKAMLFTYDQYQENNVDQASGSVLLH | | | | | |
| | | | LEVGDQVWLQVYGEGERNGLYADNDNDSTFTGFLLY | | | | | |
| | | | HDTN | | | | | |
| 144 | O00253 | 1 | AQMGLAPMEGIRRPDQALLPELPGLGLRAPLKKTTAE | 111 | Agouti-related | NA | T35 | 1 |
| | | | QAEEDLLQEAQALAEVLDLQDREPRSSRRCVRLHESC | | protein | | | |
| | | | LGQQVPCCDPCATCYCRFFNAFCYCRKLGTAMNPCSR | | | | | |
| | | | T | | | | | |
| 147 | P01160 | 1 | SLRRSSCFGGRMDRIGAQSGLGCNSFRY | 28 | Atrial | Natriuretic peptide | S19, S25 | 2 |
| | | | | | natriuretic | | | |
| | | | | | factor | | | |
| 163 | P16860 | 1 | SPKMVQGSGCFGRKMDRISSSSGLGCKVLRRH | 32 | Brain natriuretic | Natriuretic peptide | S22 | 1 |
| | | | | | peptide 32 | | | |
| 164 | P01160 | 1 | NPMYNAVSNADLMDFKNLLDHLEEKMPLED | 30 | Cardiodilatin- | Natriuretic peptide | Y4, A6, S8 (Ambiguous) | 1 |
| | | | | | related peptide | | | |
| 167 | P23582 | 1 | DLRVDTKSRAAWARLLQEHPNARKYKGANKKGLSKG | 53 | CNP-53 | Natriuretic peptide | S47 | 1 |
| | | | CFGLKLDRIGSMSGLGC | | | | | |
| 168 | P16860 | 1 | HPLGSPGSASDLETSGLQEQRNHLQGKLSELQVEQTSL | 108 | Natriuretic | Natriuretic peptide | T48, E59 | 2 |
| | | | EPLQESPRPTGVWKSREVATEGIRGHRKMVLYTLRAP | | peptides B | | | |
| | | | RSPKMVQGSGCFGRKMDRISSSSGLGCKVLRRH | | | | | |
| 170 | O95156 | 1 | KEVVHATEGLDWEDKDAPGTLVGNVVHSRIISPLRLF | 242 | Neurexophilin-2 | Neurexophilin | T7 | 1 |
| | | | VKQSPVPKPGPMAYADSMENFWDWLANITEIQEPLAR | | | | | |
| | | | TKRRPIVKTGKFKKMFGWGDFHSNIKTVKLNLLITGKI | | | | | |
| | | | VDHGNGTFSVYFRHNSTGLGNVSVSLVPPSKVVEFEVS | | | | | |
| | | | PQSTLETKESKSFNCRIEYEKTDRAKKTALCNFDPSKIC | | | | | |
| | | | YQEQTQSHVSWLCSKPFKVICIYIAFYSVDYKLVQKVC | | | | | |
| | | | PDYNYHSETPYLSSG | | | | | |
| 184 | P01303 | 1 | SSPETLISDLLMRESTENVPRTRLEDPAMW | 30 | C-flanking | NPY | S1, S2, T16, T21, A28 | 3 |
| | | | | | peptide of NPY | | | |
| 185 | P01303 | 1 | YPSKPDNPGEDAPAEDMARYYSALRHYINLITRQRY | 36 | Neuropeptide Y | NPY | S3, T32 | 2 |
| 186 | P01298 | 1 | APLEPVYPGDNATPEQMAQYAADLRRYINMLTRPRY | 36 | Pancreatic | NPY | T32 | 1 |
| | | | | | hormone | | | |
| 188 | P10082 | 1 | YPIKPEAPREDASPEELNRYYASLRHYLNLVTRQRY | 36 | Peptide YY | NPY | T32 | 1 |
| 189 | P10082 | 1 | IKPEAPREDASPEELNRYYASLRHYLNLVTRQRY | 34 | Peptide YY(3-36) | NPY | T30 | 1 |
| 190 | P80303 | 1 | VPIDIDKTKVQNIHPVESAKIEPPDTGLYYDEYLKQVID | 82 | Nesfatin-1 | Nucleobindin | T8, I13, S18, T26, Y29, Y30, | 8 |
| | | | VLETDKHFREKLQKADIEEIKSGRLSKELDLVSHHVRT | | | | T43, S72 | |
| | | | KLDEL | | | | | |
| 191 | Q02818 | 1 | VPLERGAPNKEETPATESPDTGLYYHRYLQEVIDVLET | 435 | Nucleobindin-1 | Nucleobindin | P8, T13, T16, S18, T21, Y24, | 22 |
| | | | DGHFREKLQAANAEDIKSGKLSRELDFVSHHVRTKLD | | | | S67, T122, T136, S198, S294, | |
| | | | ELKRQEVSRLRMLLKAKMDAEQDPNVQVDHLNLLKQ | | | | S295, T309, H313, S343, T346, | |
| | | | FEHLDPQNQHTFEARDLELLIQTATRDLAQYDAAHHE | | | | S352, Q381, A388, T400, L1126, | |
| | | | EFKRYEMLKEHERRRYLESLGEEQRKEAERKLEEQQR | | | | x433 (deleterious mutation) | |
| | | | RHREHPKVNVPGSQAQLKEVWEELDGLDPNRFNPKTF | | | | | |
| | | | FILHDINSDGVLDEQELEALFTKELEKVYDPKNEEDDM | | | | | |
| | | | REMEEERLRMREHVMKNVDTNQDRLVTLEEFLASTQ | | | | | |
| | | | RKEFGDTGEGWETVEMHPAYTEEELRRFEEELAAREA | | | | | |
| | | | ELNAKAQRLSQETEALGRSQGRLEAQKRELQQAVLH | | | | | |
| | | | MEQRKQQQQQQQGHKAPAAHPEGQLKFHPDTDDVPV | | | | | |
| | | | PAPAGDQKEVDTSEKKLLERLPEVEVPQHL | | | | | |
| 192 | P80303 | 1 | VPIDIDKTKVQNIHPVESAKIEPPDTGLYYDEYLKQVID | 396 | Nucleobindin-2 | Nucleobindin | T8, I13, S18, T26, Y29, Y30, | 16 |
| | | | VLETDKHFREKLQKADIEEIKSGRLSKELDLVSHHVRT | | | | T43, S72, S128, T139, T148, | |
| | | | KLDELKRQEVGRLRMLIKAKLDSLQDIGMDHQALLKQ | | | | A331, Y365, P382, S384, L390 | |
| | | | FDHLNHLNPDKFESTDLDMLIKAATSDLEHYDKTRHE | | | | | |
| | | | EFKKYEMMKEHERREYLKTLNEEKRKEEESKFEEMKK | | | | | |
| | | | KHENHPKVNHPGSKDQLKEVWEETDGLDPNDFDPKTF | | | | | |
| | | | FKLHDVNSDGFLDEQELEALFTKELEKVYDPKNEEDD | | | | | |
| | | | MVEMEEERLRMREHVMSEVDTNKDRLVTLEEFLKAT | | | | | |
| | | | EKKEFLEPDSWETLDQQQFFTEEELKEYENIIALQENEL | | | | | |
| | | | KKKADELQKQKEELQRQHDQLEAQKLEYHQVIQQME | | | | | |
| | | | QKKLQQGIPPSGPAGELKFEPHI | | | | | |
| 204 | Q13519 | 1 | MPRVRSLFQEQEEPEPGMEEAGEMEQKQLQ | 30 | Neuropeptide 1 | Opioid | P14 | 1 |
| | | | | | (Probable) | | | |
| 215 | P12272 | 1 | AVSEHQLLHDKGKSIQDLRRRFFLHHLIAEIHTAEIRAT | 141 | Parathyroid | Parathyroid hormone | T39 | 1 |
| | | | SEVSPNSKPSPNTKNHPVRFGSDDEGRYLTQETNKVET | | | hormone-related | | |
| | | | YKEQPLKTPGKKKKGKPGKRKEQEKKKRRTRSAWLD | | | protein | | |
| | | | SGVTGSGLEGDHLSDTSTTSLELDSRRH | | | | | |
| 217 | P12272 | 1 | ATSEVSPNSKPSPNTKNHPVRFGSDDEGRYLTQETNKV | 57 | PTHrP[38-94] | Parathyroid hormone | T2 | 1 |
| | | | ETYKEQPLKTPGKKKKGKP | | | | | |
| 219 | P01189 | 1 | YGGFMTSEKSQTPLVTLFKNAIIKNAYKKGE | 31 | Beta-endorphin | POMC | S10, T12, T16 | 3 |
| 222 | P01189 | 1 | ELTGQRLREGDGPDGPADDGAGAQADLEHSLLVAAE | 89 | Lipotropin beta | POMC | S68, T70, T74 | 3 |
| | | | KKDEGPYRMEHFRWGSPPKDKRYGGFMTSEKSQTPL | | | | | |
| | | | VTLFKNAIIKNAYKKGE | | | | | |
| 227 | P01189 | 1 | WCLESSQCQDLTTESNLLECIRACKPDLSAETPMFPGN | 76 | NPP | POMC | T45 | 1 |
| | | | GDEQPLTENPRKYVMGHFRWDRFGRRNSSSSGSSGAG | | | | | |
| | | | Q | | | | | |
| 229 | Q9UHG2 | 1 | LETPAPQVPARRLLPP | 16 | Big LEN (By | ProSAAS | T3, P4, A5 | 2 |
| | | | | | similarity) | | | |
| 230 | Q9UHG2 | 1 | AADHDVGSELPPEGVLGALLRVKRLETPAPQVPARRL | 40 | Big PEN-LEN | ProSAAS | T27, P28, A29 | 2 |
| | | | LPP | | (By similarity) | | | |
| 231 | Q9UHG2 | 1 | ARPVKEPRGLSAASPPLAETGAPRRF | 26 | Big SAAS (By | ProSAAS | G9, S14, T20, G21, A22 | 5 |
| | | | | | similarity) | | | |
| 233 | Q9UHG2 | 1 | LETPAPQVPA | 10 | Little LEN (By | ProSAAS | T3, P4, A5 | 2 |
| | | | | | similarity) | | | |
| 234 | Q9UHG2 | 1 | GLSAASPPLAETGAPRRF | 18 | Little SAAS (By | ProSAAS | G1, S6, T12, G13, A14 | 5 |
| | | | | | similarity) | | | |
| 236 | Q9UHG2 | 1 | ARPVKEPRGLSAASPPLAETGAPRRFRRSVPRGEAAGA | 227 | ProSAAS | ProSAAS | 13 | |
| | | | VQELARALAHLLEAERQERARAEAQEAEDQQARVLA | | | | | |
| | | | QLLRVWGAPRNSDPALGLDDDPDAPAAQLARALLRAR | | | | G9, S14, T20, G21, A22, A82, | |
| | | | LDPAALAAQLVPAPVPAAALRPRPPVYDDGPAGPDAE | | | | N85, S86, A143, S173, A174, | |
| | | | EAGDETPDVDPELLRYLLGRILAGSADSEGVAAPRRLR | | | | T214, P215, A216 | |
| | | | RAADHDVGSELPPEGVLGALLRVKRLETPAPQVPARR | | | | | |
| | | | LLPP | | | | | |
| 252 | P01019 | 1 | DRVYIHPFHLVIHNESTCEQLAKANAGKPKDPTFIPAPI | 452 | Angiotensinogen | Serpin | T407 or S409 or T410 or T422 | 1 |
| | | | QAKTSPVDEKALQDQLVLVAAKLDTEDKLRAAMVG | | | | (Ambiguous) | |
| | | | MLANFLGFRIYGMHSELWGVVHGATVLSPTAVFGTLA | | | | | |
| | | | SLYLGALDHTADRLQAILGVPWKDKNCTSRLDAHKVL | | | | | |
| | | | SALQAVQGLLVAQGRADSQAQLLLSTVVGVFTAPGLH | | | | | |
| | | | LKQPFVQGLALYTPVVLPRSLDFTELDVAAEKIDRFMQ | | | | | |
| | | | AVTGWKTGCSLMGASVDSTLAFNTYVHFQGKMKGFS | | | | | |
| | | | LLAEPQEFWVDNSTSVSVPMLSGMGTFQHWSDIQDNF | | | | | |
| | | | SVTQVPFTESACLLLIQPHYASDLDKVEGLTFQQNSLN | | | | | |
| | | | WMKKLSPRTIHLTMPQLVLQGSYDLQDLLAQAELPAI | | | | | |
| | | | LHTELNLQKLSNDRIRVGEVLNSIFFELEADEREPTEST | | | | | |
| | | | QQLNKPEVLEVTLNRPFLFAVYDQSATALHFLGRVAN | | | | | |
| | | | PLSTA | | | | | |
| 260 | P61278 | 1 | SANSNPAMAPRERKAGCKNFFWKTFTSC | 28 | Somatostatin-28 | Somastostatin | S1 or S4 (Ambiguous) | 1 |
| 262 | P20366 | 1 | ALNSVAYERSAMQNYE | 15 | C-terminal- | Tachykinin | A1, S4 | 2 |
| | | | | | flanking peptide | | | |
| 267 | P20396 | 1 | QPEAAQQEAVTAAEHPGLDDFLRQVERLLFLRENIQRL | 218 | Pro-thyrotropin- | TRH | I50 | 1 |
| | | | QGDQGEHSASQIFQSDWLSKRQHPGKREEEEEEGVEE | | releasing hormone | | | |
| | | | EEEEEGGAVGPHKRQHPGRREDEASWSVDVTQHKRQ | | | | | |
| | | | HPGRRSPWLAYAVPKRQHPGRRLADPKAQRSWEEEEE | | | | | |
| | | | EEEREEDLMPEKRQHPGKRALGGPCGPQGAYGQAGLL | | | | | |
| | | | LGLLDDLSRSQGAEEKRQHPGRRAAWVREPLEE | | | | | |
| 272 | P01185 | 1 | ASDRSNATQLDGPAGALLLRLVQLAGAPEPFEPAQPDA | 39 | Copeptin | Vasopressin/oxytocin | A27 | 1 |
| | | | Y | | | | | |
| 279 | O15240 | 1 | APPGRPEAQPPPLSSEHKEPVAGDAVPGPKDGSAPEVR | 591 | Neurosecretory | VGF | P1, S12, P202, S205, | 5 |
| | | | GARNSEPQDEGELFQGVDPRALAAVLLQALDRPASPP | | protein VGF | T477 | | |
| | | | APSGSQQGPEEEAAEALLTETVRSQTHSLPAPESPEPAA | | | | | |
| | | | PPRPQTPENGPEASDPSEELEALASLLQELRDFSPSSAK | | | | | |
| | | | RQQETAAAETETRTHTLTRVNLESPGPERVWRASWGE | | | | | |
| | | | FQARVPERAPLPPPAPSQFQARMPDSGPLPETHKFGEG | | | | | |
| | | | VSSPKTHLGEALAPLSKAYQGVAAPFPKARRPESALLG | | | | | |
| | | | GSEAGERLLQQGLAQVEAGRRQAEATRQAAAQEERL | | | | | |
| | | | ADLASDLLLQYLLQGGARQRGLGGRGLQEAAEERESA | | | | | |
| | | | REEEEAEQERRGGEERVGEEDEEAAEAEAEAEEAERA | | | | | |
| | | | RQNALLFAEEEDGEAGAEDKRSQEETPGHRRKEAEGT | | | | | |
| | | | EEGGEEEDDEEMDPQTIDSLIELSTKLHLPADDVVSIIEE | | | | | |
| | | | VEEKRKRKKNAPPEPVPPPRAAPAPTHVRSPQPPPPAP | | | | | |
| | | | APARDELPDWNEVLPPWDREEDEVYPPGPYHPFPNYIR | | | | | |
| | | | PRTLQPPSALRRRHYHHALPPSRHYPGREAQARRAQEE | | | | | |
| | | | AEAEERRLQEQEELENYIEHVLLRRP | | | | | |

TABLE 6E

| | | | | | | | *Site position (Bold: | |
|---|---|---|---|---|---|---|---|---|
| | | | | | | | identified or conserved | |
| SEQ | | Pep- | | Pep- | | Peptide | in human, Italic; | No. |
| ID | Entry_ | tide | | tide | Peptide | Hormone | identified in non-human | of |
| NO: | Human | Start | Peptide_Sequence | end | Name | Family | and not conserved) | sites |
| 1 | P05408 | 1 | SVPHFSDEDKDPE | 13 | C-terminal | 7B2 | S1 | 1 |
| | | | | | peptide (By | | | |
| | | | | | similarity) | | | |
| 2 | P05408 | 1 | YSPRTPDRVSEADIQRLLHGVMEQLGIARPRVEYPA | 186 | Neuroendocrine | 7B2 | S2, T5, S10, T108, S174 | 5 |
| | | | HQAMNLVGPQSIEGGAHEGLQHLGPFGNIPNIVAEL | | protein 7B2 | | | |
| | | | TGDNIPKDFSEDQGYPDPPNPCPVGKTADDGCLENT | | | | | |
| | | | PDTAEFSREFQLHQHLFDPEHDYPGLGKWNKKLLY | | | | | |
| | | | EKMKGGERRKRRSVNPYLQGQRLDNVVAKKSVPH | | | | | |
| | | | FSDEDKDPE | | | | | |
| 3 | P05408 | 1 | YSPRTPDRVSEADIQRLLHGVMEQLGIARPRVEYPA | 150 | N-terminal | 7B2 | S2, T5, S10, T108 | 4 |
| | | | HQAMNLVGPQSIEGGAHEGLQHLGPFGNIPNIVAEL | | peptide (By | | | |
| | | | TGDNIPKDFSEDQGYPDPPNPCPVGKTADDGCLENT | | similarity) | | | |
| | | | PDTAEFSREFQLHQHLFDPEHDYPGLGKWNKKLLY | | | | | |
| | | | EKMKGGE | | | | | |
| 4 | Q8N6N7 | 1 | MALQADFDRAAEDVRKLKARPDDGELKELYGLYK | 88 | Acyl-CoA- | ACBP | | |
| | | | QAIVGDINIACPGMLDLKGKAKWEAWNLKKGLSTE | | binding domain- | | | |
| | | | DATSAYISKAKELIEKYGI | | containing | | | |
| | | | | | protein 7 | | | |
| 5 | P07108 | 1 | SQAEFEKAAEEVRHLKTKPSDEEMLFIYGHYKQAT | 86 | Acyl-CoA- | ACBP | T35 or T41 (ambiguous) | 1 |
| | | | VGDINTERPGMLDFTGKAKWDAWNELKGTSKEDA | | binding protein | | | |
| | | | MKAYINKVEELKKKYGI | | | | | |
| 6 | P35318 | 1 | YRQSMNNFQGLRSFGCRFGTCTVQKLAHQIYQFTD | 52 | Adrenomedullin | Adrenomedullin | T22, Y31, T34 | 3 |
| | | | KDKDNVAPRSKISPQGY | | | | | |
| 7 | Q7Z4H4 | 1 | TQAQLLRVGCVLGTCQVQNLSHRLWQLMGPAGRQ | 47 | Adrenomedullin- | Adrenomedullin | | |
| | | | DSAPVDPSSPHSY | | 2 (By similarity) | | | |
| 8 | Q7Z4H4 | 1 | VGCVLGTCQVQNLSHRLWQLMGPAGRQDSAPVDP | 40 | Intermedin-short | Adrenomedullin | | |
| | | | SSPHSY | | (Potential) | | | |
| 9 | P35318 | 1 | ARLDVASEFRKKWNKWALSR | 20 | Proadreno- | Adrenomedullin | | |
| | | | | | medullin N-20 | | | |
| | | | | | terminal peptide | | | |
| 10 | Q9ULZ1 | 1 | QRPRLSHKGPMPF | 13 | Apelin-13 (By | Apelin | | |
| | | | | | similarity) | | | |
| 11 | Q9ULZ1 | 1 | NGPGPWQGGRRKFRRQRPRLSHKGPMPF | 28 | Apelin-28 (By | Apelin | | |
| | | | | | similarity) | | | |
| 12 | Q9ULZ1 | 1 | GSRNGPGPWQGGRRKFRRQRPRLSHKGPMPF | 31 | Apelin-31 (By | Apelin | | |
| | | | | | similarity) | | | |
| 13 | Q9ULZ1 | 1 | LVQPRGSRNGPGPWQGGRRKFRRQRPRLSHKGPMP | 36 | Apelin-36 (By | Apelin | | |
| | | | F | | similarity) | | | |
| 14 | P07492 | 1 | VPLPAGGGTVLTKMYPRGNHWAVGHLM | 27 | Gastrin-releasing | Bombesin/ | P4, T9 | 2 |
| | | | | | peptide | neuromedin- | | |
| | | | | | | B/ranatensin | | |
| 15 | P08949 | 1 | GNLWATGHFM | 10 | Neuromedin-B | Bombesin/ | T6 | 1 |
| | | | | | | neuromedin- | | |
| | | | | | | B/ranatensin | | |
| 16 | P08949 | 1 | APLSWDLPEPRSRASKIRVHSRGNLWATGHFM | 32 | Neuromedin-B- | Bombesin/ | T28 | 1 |
| | | | | | 32 | neuromedin- | | |
| | | | | | | B/ranatensin | | |
| 17 | P07492 | 1 | GNHWAVGHLM | 10 | Neuromedin-C | Bombesin/ | | |
| | | | | | | neuromedin- | | |
| | | | | | | B/ranatensin | | |
| 18 | P01042 | 1 | RPPGFSPFR | 9 | Bradykinin | Bradykinin | | |
| 19 | P01042 | 1 | KRPPGFSPFR | 10 | Lysyl-bradykinin | Bradykinin | | |
| 20 | P01042 | 1 | ISLMKRPPGFSPFR | 14 | T-kinin | Bradykinin | | |
| 21 | P01258 | 1 | CGNLSTCMLGTYTQDFNKFHTFPQTAIGVGAP | 32 | Calcitonin | Calcitonin | T6, T11, T13, T21, T25 | 5 |
| 22 | P06881 | 1 | ACDTATCVTHRLAGLLSRSGGVVKNNFVPTNVGSK | 37 | Calcitonin gene- | Calcitonin | T9, S17 | 2 |
| | | | AF | | related peptide 1 | | | |
| 23 | P10092 | 1 | ACNTATCVTHRLAGLLSRSGGMVKSNFVPTNVGSK | 37 | Calcitonin gene- | Calcitonin | T9, S17, S25, T30, S34 | 5 |
| | | | AF | | related peptide 2 | | | |
| 24 | P10997 | 1 | KCNTATCATQRLANFLVHSSNNFGAILSSTNVGSNT | 37 | Islet amyloid | Calcitonin | T5, T9, T30 | 3 |
| | | | Y | | polypeptide | | | |
| 25 | P01258 | 1 | DMSSDLERDHRPHVSMPQNAN | 21 | Katacalcin | Calcitonin | S15 | 1 |
| 26 | Q16568 | 1 | QEDAELQPRALDIYSAVDDASHEKELIEALQEVLKK | 39 | CART(1-39) | CART | | |
| | | | LKS | | | | | |
| 27 | Q16568 | 1 | VPIYEKKYGQVPMCDAGEQCAVRKGARIGKLCDCP | 48 | CART(42-89) | CART | | |
| | | | RGTSCNSFLLKCL | | | | | |
| 28 | Q16568 | 1 | QEDAELQPRALDIYSAVDDASHEKELIEALQEVLKK | 89 | Cocaine- and | CART | | |
| | | | LKSKRVPIYEKKYGQVPMCDAGEQCAVRKGARIGK | | amphetamine- | | | |
| | | | LCDCPRGTSCNSFLLKCL | | regulated | | | |
| 29 | P23435 | 1 | GSAKVAFSAIRSTNH | 15 | [des-Ser1]- | Cerebellins | | |
| | | | | | cerebellin | | | |
| 30 | P23435 | 1 | SGSAKVAFSAIRSTNH | 16 | Cerebellin | Cerebellins | | |
| 31 | P23435 | 1 | QNETEPIVLEGKCLVVCDSNPTSDPTGTALGISVRSG | 172 | Cerebellin-1 | Cerebellins | | |
| | | | SAKVAFSAIRSTNHEPSEMSNRTMIIYFDQVLVNIGN | | | | | |
| | | | NFDSERSTFIAPRKGIYSFNFHVVKVYNRQTIQVSLM | | | | | |
| | | | LNGWPVISAFAGDQDVTREAASNGVLIQMEKGDRA | | | | | |
| | | | YLKLERGNLMGGWKYSTFSGFLVFPL | | | | | |
| 32 | Q8IUK8 | 1 | QNDTEPIVLEGKCLVVCDSSPSADGAVTSSLGISVRS | 173 | Cerebellin-2 | Cerebellins | | |
| | | | GSAKVAFSATRSTNHEPSEMSNRTMTIYFDQVLVNI | | | | | |
| | | | GNHFDLASSIFVAPRKGIYSFSFHVVKVYNRQTIQVS | | | | | |
| | | | LMQNGYPVISAFAGDQDVTREAASNGVLLLMERED | | | | | |
| | | | KVHLKLERGNLMGGWKYSTFSGFLVFPL | | | | | |
| 33 | Q6UW01 | 1 | QEGSEPVLLEGECLVVCEPGRAAAGGPGGAALGEA | 173 | Cerebellin-3 | Cerebellins | | |
| | | | PPGRVAFAAVRSHHHEPAGETGNGTSGAIYFDQVL | | | | | |
| | | | VNEGGGFDRASGSFVAPVRGVYSFRFHVVKVYNRQ | | | | | |
| | | | TVQVSLMLNTWPVISAFANDPDVTREAATSSVLLPL | | | | | |
| | | | DPGDRVSLRLRRGNLLGGWKYSSFSGFLIFPL | | | | | |
| 34 | Q9NTU7 | 1 | QNDTEPIVLEGKCLVVCDSNPATDSKGSSSSPLGISV | 174 | Cerebellin-4 | Cerebellins | | |
| | | | RAANSKVAFSAVRSTNHEPSEMSNKTRIIYFDQILVN | | | | | |
| | | | VGNFFTLESVFVAPRKGIYSFSFHVIKVYQSQTIQVN | | | | | |
| | | | LMLNGKPVISAFAGDKDVTREAATNGVLLYLDKED | | | | | |
| | | | KVYLKLEKGNLVGGWQYSTFSGFLVFPL | | | | | |
| 35 | P10645 | 1 | AYGFRGPGPQL | 11 | AL-11 | Chromogranin/ | | |
| | | | | | | secretogranin | | |
| 36 | P05060 | 1 | SAEFPDFYDSEEPVSTHQEAENEKDRADQTVLTEDE | 57 | CCB peptide | Chromogranin/ | S15 | 1 |
| | | | KKELENLAAMDLELQKIAEKF | | | secretogranin | | |
| 37 | P10645 | 1 | LPVNSPMNKGDTEVMKCIVEVISDTLSKPSPMPVSQ | 439 | Chromogranin-A | Chromogranin/ | S27, S30, S36, S80, E104, | 39 |
| | | | ECFETLRGDERILSILRHQNLLKELQDLALQGAKER | | | secretogranin | S108, A127, T128, A135, L136, | |
| | | | AHQQKKHSGFEDELSEVLENQSSQAELKEAVEEPSS | | | | S143, A151, N164, T165, P167, | |
| | | | KDVMEKREDSKEAEKSGEATDGARPQALPEPMQES | | | | A169, S170, S173, E182, L188, | |
| | | | KAEGNNQAPGEEEEEEEEATNTHPPASLPSQKYPGP | | | | S189, V193, S200, T233, L236, | |
| | | | QAEGDSEGLSQGLVDREKGLSAEPGWQAKREEEEE | | | | N237, P238, S241, Y244, P265, | |
| | | | EEEEAEAGEEAVPEEEGPTVVLNPHPSLGYKEIRKG | | | | E268, P273, S282, M291, V294, | |
| | | | ESRSEALAVDGAGKPGAEEAQDPEGKGEQEHSQQK | | | | T335, A360, S380, G395 | |
| | | | EEEEEMAVVPQGLFRGGKSGELEQEEERLSKEWEDS | | | | | |
| | | | KRWSKMDQLAKELTAEKRLEGQEEEEDNRDSSMK | | | | | |
| | | | LSFRARAYGFRGPGPQLRRGWRPSSREDSLEAGLPL | | | | | |
| | | | QVRGYPEEKKEEEGSANRRPEDQELESLSAIEAELE | | | | | |
| | | | KVAHQLQALRRG | | | | | |
| 38 | P10645 | 1 | EDSKEAEKSGEATDGARPQALPEPMQESKAEGNNQ | 92 | EA-92 | Chromogranin/ | A12, T13, A20, L21, S28, A36, | 17 |
| | | | APGEEEEEEEEATNTHPPASLPSQKYPGPQAEGDSEG | | | secretogranin | N49, T50, P52, A54, S55, S58, | |
| | | | LSQGLVDREKGLSAEPGWQA | | | | E67, L73, S74, V78, S85 | |
| 39 | P10645 | 1 | EEEGSANRRPEDQELESLSAIEAELEKVAHQLQALR | 37 | ER-37 | Chromogranin/ | S17 or S19 (Ambiguous) | 1 |
| | | | R | | | secretogranin | | |
| 40 | P10645 | 1 | EEEEEEEEEAEAGEEAVPEEEGPTVVLNPHPSL | 33 | ES-43 | Chromogranin/ | T24, L27, N28, P29, S32 | 5 |
| | | | | | | secretogranin | | |
| 41 | P05060 | 1 | FLGEGHHRVQENQMDKARRHPQGAWKELDRNYLN | 74 | GAWK peptide | Chromogranin/ | H7, N12, Y35, T72, A73 | 5 |
| | | | YGEEGAPGKWQQQGDLQDTKENREEARFQDKQYS | | | secretogranin | | |
| | | | SHHTAE | | | | | |
| 42 | P10645 | 1 | GYPEEKKEEEGSANRRPEDQELESLSAIEAELEKVA | 44 | GR-44 | Chromogranin/ | G1 | 1 |
| | | | HQLQALRR | | | secretogranin | | |
| 43 | P10645 | 1 | GWRPSSREDSLEAGLPLQV | 19 | GV-19 | Chromogranin/ | S6 | 1 |
| | | | | | | secretogranin | | |
| 44 | P10645 | 1 | LEGQEEEEDNRDSSMKLSF | 19 | LF-19 | Chromogranin/ | | |
| | | | | | | secretogranin | | |
| 45 | P10645 | 1 | SEALAVDGAGKPGAEEAQDPEGKGEQEHSQQKEEE | 48 | Pancreastatin | Chromogranin/ | P12, E15, P20, S29, M38, V41 | 6 |
| | | | EEMAVVPQGLFRG | | | secretogranin | | |
| 46 | P05060 | 1 | MPVDNRNHNEGMVTRCIIEVLSNALSKSSAPPITPEC | 657 | Secretogranin-1 | Chromogranin/ | T14, S26, S28, S29, P32, T59, | 40 |
| | | | RQVLKTSRKDVKDKETFENENTKFEVRLLRDPADA | | | secretogranin | S73, A75, S80, A84, E89, T96, | |
| | | | SEAHESSSRGEAGAPGEEDIQGPTKADTEKWAEGGG | | | | S120, S124, S129, T174, N176, | |
| | | | HSRERADEPQWSLYPSDSQVSEEVKTRHSEKSQRED | | | | S186, S273, S274, S278, P287, | |
| | | | EEEEEGENYQKGERGEDSSEEKHLEEPGETQNAFLN | | | | S297, M298, S300, D306, T310, | |
| | | | ERKQASAIKKEELVARSETHAAGHSQEKTHSREKSS | | | | Y328, P329, G330, K376, | |
| | | | QESGEETGSQENHPQESKGQPRSQEESEEGEEDATS | | | | M377, H426, Y454, T491, | |
| | | | EVDKRRTRPRHHHGRSRPDRSSQGGSLPSEEKGHPQ | | | | A492, T536, S557, N568, S611 | |
| | | | EESEESNVSMASLGEKRDHHSTHYRASEEEPEYGEE | | | | | |
| | | | IKGYPGVQAPEDLEWERYRGRGSEEYRAPRPQSEES | | | | | |
| | | | WDEEDKRNYPSLELDKMAHGYGEESEEERGLEPGK | | | | | |
| | | | GRHHRGRGGEPRAYFMSDTREEKRFLGEGHHRVQE | | | | | |
| | | | NQMDKARRHPQGAWKELDRNYLNYGEEGAPGKW | | | | | |
| | | | QQQGDLQDTKENREEARFQDKQYSSHHTAEKRKRL | | | | | |
| | | | GELFNPYYDPLQWKSSHFERRDNMNDNFLEGEEEN | | | | | |
| | | | ELTLNEKNFFPEYNYDWWEKKPFSEDVNWGYEKR | | | | | |
| | | | NLARVPKLDLKRQYDRVAQLDQLLHYRKKSAEFPD | | | | | |
| | | | FYDSEEPVSTHQEAENEKDRADQTVLTEDEKKELE | | | | | |
| | | | NLAAMDLELQKIAEKFSQRG | | | | | |
| 47 | P13521 | 1 | QRNQLLQKEPDLRLENVQKFPSPEMIRALEYIENLR | 587 | Secretogranin-2 | Chromogranin/ | S22, S45, A106, Y160, T161, | 16 |
| | | | QQAHKEESSPDYNPYQGVSVPLQQKENGDESHLPE | | | secretogranin | S164, T167, S229, T231, S348, | |
| | | | RDSLSEEDWMRIILEALRQAENEPQSAPKENKPYAL | | | | T420, N459, G499, S526, S536, | |
| | | | NSEKNFPMDMSDDYETQQWPERKLKHMQFPPMYE | | | | P543 | |
| | | | ENSRDNPFKRTNEIVEEQYTPQSLATLESVFQELGK | | | | | |
| | | | LTGPNNQKRERMDEEQKLYTDDEDDIYKANNIAYE | | | | | |
| | | | DVVGGEDWNPVEEKIESQTQEEVRDSKENIEKNEQI | | | | | |
| | | | NDEMKRSGQLGIQEEDLRKESKDQLSDDVSKVIAY | | | | | |
| | | | LKRLVNAAGSGRLQNGQNGERATRLFEKPLDSQSIY | | | | | |
| | | | QLIEISRNLQIPPEDLIEMLKTGEKPNGSVEPERELDL | | | | | |
| | | | PVDLDDISEADLDHPDLFQNRMLSKSGYPKTPGRAG | | | | | |
| | | | TEALPDGLSVEDILNLLGMESAANQKTSYFPNPYNQ | | | | | |
| | | | EKVLPRLPYGAGRSRSNQLPKAAWIPHVENRQMAY | | | | | |
| | | | ENLNDKDQELGEYLARMLVKYPEIINSNQVKRVPG | | | | | |
| | | | QGSSEDDLQEEEQIEQAIKEHLNQGSSQETDKLAPV | | | | | |
| | | | SKRFPVGPPKNDDTPNRQYWDEDLLMKVLEYLNQ | | | | | |
| | | | EKAEKGREHIAKRAMENM | | | | | |
| 48 | Q8WXD2 | 1 | FPKPGGSQDKSLHNRELSAERPLNEQIAEAEEDKIKK | 449 | Secretogranin-3 | Chromogranin/ | P41, T63, S103, T104, T125, | 15 |
| | | | TYPPENKPGQSNYSFVDNLNLLKAITEKEKIEKERQ | | | secretogranin | A138, D192, P193, T197, S198, | |
| | | | SIRSSPLDNKLNVEDVDSTKNRKLIDDYDSTKSGLD | | | | T200, T212, T217, A222, S340 | |
| | | | HKFQDDPDGLHQLDGTPLTAEDIVHKIAARIYEEND | | | | | |
| | | | RAVFDKIVSKLLNLGLITESQAHTLEDEVAEVLQKLI | | | | | |
| | | | SKEANNYEEDPNKPTSWTENQAGKIPEKVTPMAAI | | | | | |
| | | | QDGLAKGENDETVSNTLTLTNGLERRTKTYSEDNFE | | | | | |
| | | | ELQYFPNFYALLKSIDSEKEAKEKETLITIMKTLIDFV | | | | | |
| | | | KMMVKYGTISPEEGVSYLENLDEMIALQTKNKLEK | | | | | |
| | | | NATDNISKLFPAPSEKSHEETDSTKEEAAKMEKEYG | | | | | |
| | | | SLKDSTKDDNSNPGGKTDEPKGKTEAYLEAIRKNIE | | | | | |
| | | | WLKKHDKKGNKEDYDLSKMRDFINKQADAYVEK | | | | | |
| | | | GILDKEEAEAIKRIYSSL | | | | | |
| 49 | P13521 | 1 | TNEIVEEQYTPQSLATLESVFQELGKLTGPNNQ | 33 | Secretoneurin | Chromogranin/ | Y9, T10, S13, T16, | 4 |
| | | | | | | secretogranin | | |
| 50 | P10645 | 1 | SGELEQEEERLSKEWEDS | 18 | SS-18 | Chromogranin/ | | |
| | | | | | | secretogranin | | |
| 51 | P10645 | 1 | LPVNSPMNKGDTEVMKCIVEVISDTLSKPSPMPVSQ | 76 | Vasostatin-1 | Chromogranin/ | S27, S30, S36 | 3 |
| | | | ECFETLRGDERILSILRHQNLLKELQDLALQGAKER | | | secretogranin | | |
| | | | AHQQ | | | | | |
| 52 | P10645 | 1 | LPVNSPMNKGDTEVMKCIVEVISDTLSKPSPMPVSQ | 113 | Vasostatin-2 | Chromogranin/ | S27, S30, S36, S80, E104, | 6 |
| | | | ECFETLRGDERILSILRHQNLLKELQDLALQGAKER | | | secretogranin | S108 | |
| | | | AHQQKKHSGFEDELSEVLENQSSQAELKEAVEEPSS | | | | | |
| | | | KDVME | | | | | |
| 53 | P10645 | 1 | WSKMDQLA | 8 | WA-8 | Chromogranin/ | | |
| | | | | | | secretogranin | | |
| 54 | P10645 | 1 | WSKMDQLAKELTAE | 14 | WE-14 | Chromogranin/ | T12 | 1 |
| | | | | | | secretogranin | | |
| 55 | P01042 | 1 | QESQSEEIDCNDKDLFKAVDAALKKYNSQNQSNNQ | 626 | Kininogen-1 | Cystatin | T119, T133, T243, T255, E381, | 15 |
| | | | FVLYRIFLATKTVGSDTFYSFKYEIKEGDCPVQSGKT | | | | T382, T383, S385, T389, S390, | |
| | | | WQDCEYKDAAKAATGECTATVGKRSSTKFSVATQ | | | | T528, S586, T592, S593, T610 | |
| | | | TCQITPAEGPVVTAQYDCLGCVHPISTQSPDLEPILR | | | | | |
| | | | HGIQYENNNTQHSSLEMLNEVKRAQRQVVAGLNFR | | | | | |
| | | | ITYSIVQTNCSKENFLFLTPDCKSLWNGDTGECTDN | | | | | |
| | | | AYIDIQLRIASFSQNCDIYPGKDEVQPPTKICVGCPR | | | | | |
| | | | DIPTNSPELEETLTHTITKLNAENNATFYFKIDNVKK | | | | | |
| | | | ARVQVVAGKKYFIDFVARETTCSKESNEELTESCET | | | | | |
| | | | KKLGQSLDCNAEVYVVPWEKKIYPTVNCQPLGMIS | | | | | |
| | | | LMKRPPGESPERSSRIGEIKEETTVSPPHTSMAPAQD | | | | | |
| | | | EERDSGKEQGHTRRHDWGHEKQRKHNLGHGHKHE | | | | | |
| | | | RDQGHGHQRGHGLGHGHEQQHGLGHGHKFKLDD | | | | | |
| | | | DLEHQGGHVLDHGHKHKHGHGHGKHKNKGKKNG | | | | | |
| | | | KHNGWKFEHLASSSEDSTTPSAQTQEKFEGPTPIPSL | | | | | |
| | | | AKPGVTVTFSDFQDSDLIATMMPPISPAPIQSDDDWI | | | | | |
| | | | PDIQIDPNGLSENPISDEPDTTSPKCPGRPWKSVSEIN | | | | | |
| | | | PTTQMKESYYFDLTDGLS | | | | | |
| 56 | P01042 | 1 | QESQSEEIDCNDKDLFKAVDAALKKYNSQNQSNNQ | 362 | Kininogen-1 | Cystatin | T119, T133, T243, T255 | 4 |
| | | | FVLYRIFLATKTVGSDTFYSFKYEIKEGDCPVQSGKT | | heavy chain | | | |
| | | | WQDCEYKDAAKAATGECTATVGKRSSTKFSVATQ | | | | | |
| | | | TCQITPAEGPVVTAQYDCLGCVHPISTQSPDLEPILR | | | | | |
| | | | HGIQYENNNTQHSSLEMLNEVKRAQRQVVAGLNFR | | | | | |
| | | | ITYSIVQTNCSKENFLFLTPDCKSLWNGDTGECTDN | | | | | |
| | | | AYIDIQLRIASFSQNCDIYPGKDEVQPPTKICVGCPR | | | | | |
| | | | DIPTNSPELEETLTHTITKLNAENNATFYFKIDNVKK | | | | | |
| | | | ARVQVVAGKKYFIDFVARETTCSKESNEELTESCET | | | | | |
| | | | KKLGQSLDCNAEVYVVPWEKKIYPTVNCQPLGMIS | | | | | |
| | | | LMK | | | | | |
| 57 | P01042 | 1 | SSRIGEIKEETTVSPPHTSMAPAQDEERDSGKEQGH | 255 | Kininogen-1 | Cystatin | E10, T11, T12, S14, T18, S19, | 11 |
| | | | TRRHDWGHEKQRKHNLGHGHKHERDQGHGHQRG | | light chain | | T157, S215, T221, S222, T239 | |
| | | | HGLGHGHEQQHGLGHGHKFKLDDDLEHQGGHVLD | | | | | |
| | | | HGHKHKHGHGHGKHKNKGKKNGKHNGWKFEHL | | | | | |
| | | | ASSSEDSTTPSAQTQEKFEGPTPIPSLAKPGVTVITS | | | | | |
| | | | DFQDSDLIATMMPPISPAPIQSDDDWIPDIQIDPNGLS | | | | | |
| | | | FNPISDEPDTTSPKCPGRPWKSVSEINPTTQMKESYY | | | | | |
| | | | FDLTDGLS | | | | | |
| 58 | P01042 | 1 | WGHE | 4 | Low molecular | Cystatin | | |
| | | | | | weight growth- | | | |
| | | | | | promoting factor | | | |
| 59 | P05305 | 1 | CSCSSLMDKECVYFCHLDIIWVNTPEHVVPYGLGSP | 38 | Big endothelin-1 | Endothelin/ | | |
| | | | RS | | | sarafotoxin | | |
| 60 | P05305 | 1 | CSCSSLMDKECVYFCHLDIIW | 21 | Endothelin-1 | Endothelin/ | | |
| | | | | | | sarafotoxin | | |
| 61 | P20800 | 1 | CSCSSWLDKECVYFCHLDIIVV | 21 | Endothelin-2 | Endothelin/ | | |
| | | | | | | sarafotoxin | | |
| 62 | P14138 | 1 | CTCFTYKDKECVYYCHLDIIW | 21 | Endothelin-3 | Endothelin/ | | |
| | | | | | | sarafotoxin | | |
| 63 | O15130 | 1 | AGEGLNSQFVVSLAAPQRF | 18 | Neuropeptide AF | FMRFamide related | | |
| | | | | | | peptide | | |
| 64 | O15130 | 1 | FLFQPQRF | 8 | Neuropeptide FF | FMRFamide related | | |
| | | | | | | peptide | | |
| 65 | Q9HCQ7 | 1 | SLNFEELKDWGPKNVIKMSTPAVNKMPHSFANLPL | 37 | Neuropeptide | FMRFamide related | | |
| | | | RF | | NPSF (Potential) | peptide | | |
| 66 | Q9HCQ7 | 1 | VPNLPQRF | 8 | Neuropeptide | FMRFamide related | | |
| | | | | | NPVF | peptide | | |
| 67 | Q9HCQ7 | 1 | MPHSFANLPLRF | 12 | Neuropeptide | FMRFamide related | | |
| | | | | | RFRP-1 | peptide | | |
| 68 | Q9HCQ7 | 1 | SAGATANLPLRS | 12 | Neuropeptide | FMRFamide related | | |
| | | | | | RFRP-2 | peptide | | |
| | | | | | (Potential) | | | |
| 69 | O15130 | 1 | SQAFLFQPQRF | 11 | Neuropeptide SF | FMRFamide related | | |
| | | | | | | peptide | | |
| 70 | P81277 | 1 | TPDINPAWYASRGIRPVGRF | 20 | Prolactin- | FMRFamide related | | |
| | | | | | releasing peptide | peptide | | |
| | | | | | PrRP20 | | | |
| 71 | P81277 | 1 | SRTHRHSMEIRTPDINPAWYASRGIRPVGRF | 31 | Prolactin- | FMRFamide related | | |
| | | | | | releasing peptide | peptide | | |
| | | | | | PrRP31 | | | |
| 72 | P22466 | 1 | GWTLNSAGYLLGPHAVGNHRSFSDKNGLTS | 30 | Galanin | Galanin | T3, S23 | 2 |
| 73 | P22466 | 1 | ELRPEDDMKPGSFDRSIPENNIMRTIIEFLSFLHLKEA | 59 | Galanin | Galanin | A49 | 1 |
| | | | GALDRLLDLPAAASSEDIERS | | message- | | | |
| | | | | | associated | | | |
| | | | | | peptide | | | |
| 74 | Q9UBC7 | 1 | APAHRGRGGWTLNSAGYLLGPVLHLPQMGDQDGK | 60 | Galanin-like | Galanin | | |
| | | | RETALEILDLWKAIDGLPYSHPPQPS | | peptide | | | |
| 75 | P01350 | 1 | QLGPQGPPHLVADPSKKQGPWLEEEEEAYGWMDF | 34 | Big gastrin | Gastrin/ | | |
| | | | | | | cholecystokinin | | |
| 76 | P06307 | 1 | QPVPPADPAGSGLQRAEEAPRRQLRVSQRTDGESRA | 95 | Cholecystokinin | Gastrin/ | A8, T30, N65 | 3 |
| | | | HLGALLARYIQQARKAPSGRMSIVKNLQNLDPSHRI | | | cholecystokinin | | |
| | | | SDRDYMGWMDFGRRSAEEYEYPS | | | | | |
| 77 | P06307 | 1 | ISDRDYMGWMDF | 12 | Cholecystokinin- | Gastrin/ | | |
| | | | | | 12 | cholecystokinin | | |
| 78 | P06307 | 1 | LDPSHRISDRDYMGWMDF | 18 | Cholecystokinin- | Gastrin/ | | |
| | | | | | 18 (By | cholecystokinin | | |
| | | | | | similarity) | | | |
| 79 | P06307 | 1 | IVKNLQNLDPSHRISDRDYMGWMDF | 25 | Cholecystokinin- | Gastrin/ | N7 | 1 |
| | | | | | 25 (By | cholecystokinin | | |
| | | | | | similarity) | | | |
| 80 | P06307 | 1 | KAPSGRMSIVKNLQNLDPSHRISDRDYMGWMDF | 33 | Cholecystokinin- | Gastrin/ | N15 | 1 |
| | | | | | 33 | cholecystokinin | | |
| 81 | P06307 | 1 | YIQQARKAPSGRMSIVKNLQNLDPSHRISDRDYMG | 39 | Cholecystokinin- | Gastrin/ | N21 | 1 |
| | | | WMDF | | 39 | cholecystokinin | | |
| 82 | P06307 | 1 | GWMDF | 5 | Cholecystokinin- | Gastrin/ | | |
| | | | | | 5 (By similarity) | cholecystokinin | | |
| 83 | P06307 | 1 | VSQRTDGESRAHLGALLARYIQQARKAPSGRMSIV | 58 | Cholecystokinin- | Gastrin/ | T5, N40 | 2 |
| | | | KNLQNLDPSHRISDRDYMGWMDF | | 58 | cholecystokinin | | |
| 84 | P06307 | 1 | VSQRTDGESRAHLGALLARYIQQARKAPSGRMSIV | 49 | Cholecystokinin- | Gastrin/ | T5, N40 | 2 |
| | | | KNLQNLDPSHRISD | | 58 | cholecystokinin | | |
| | | | | | desnonopeptide | | | |
| | | | | | (By similarity) | | | |
| 85 | P06307 | 1 | YMGWMDF | 7 | Cholecystokinin- | Gastrin/ | | |
| | | | | | 7 (By similarity) | cholecystokinin | | |
| 86 | P06307 | 1 | DYMGWMDF | 8 | Cholecystokinin- | Gastrin/ | | |
| | | | | | 8 | cholecystokinin | | |
| 87 | P01350 | 1 | QGPWLEEEEEAYGWMDF | 17 | Gastrin | Gastrin/ | | |
| | | | | | | cholecystokinin | | |
| 88 | P01350 | 1 | WLEEEEEAYGWMDF | 14 | Gastrin-14 | Gastrin/ | | |
| | | | | | | cholecystokinin | | |
| 89 | P01350 | 1 | DLELPWLEQQGPASHHRRQLGPQGPPHLVADPSKK | 52 | Gastrin-52 | Gastrin/ | | |
| | | | QGPWLEEEEEAYGWMDF | | | cholecystokinin | | |
| 90 | P01350 | 1 | YGWMDF | 6 | Gastrin-6 | Gastrin/ | | |
| | | | | | | cholecystokinin | | |
| 91 | P01350 | 1 | SWKPRSQQPDAPLGTGANRDLELPWLEQQGPASHH | 71 | Gastrin-71 | Gastrin/ | L12 or G13 (Ambiguous) | 1 |
| | | | RRQLGPQGPPHLVADPSKKQGPWLEEEEEAYGWM | | | cholecystokinin | | |
| | | | DF | | | | | |
| 92 | P09681 | 1 | YAEGTFISDYSIAMDKIHQQDFVNWLLAQKGKKND | 42 | Gastric inhibitory | Glucagon | | |
| | | | WKHNITQ | | polypeptide | | | |
| 93 | P01275 | 1 | RSLQDTEEKSRSFSASQADPLSDPDQMNEDKRHSQG | 69 | Glicentin (By | Glucagon | S12, T39 | 2 |
| | | | TFTSDYSKYLDSRRAQDFVQWLMNTKRNRNNIA | | similarity) | | | |
| 94 | P01275 | 1 | RSLQDTEEKSRSFSASQADPLSDPDQMNED | 30 | Glicentin-related | Glucagon | S12 | 1 |
| | | | | | polypeptide (By | | | |
| | | | | | similarity) | | | |
| 95 | P01275 | 1 | HSQGTFTSDYSKYLDSRRAQDFVQWLMNT | 29 | Glucagon | Glucagon | T7 | 1 |
| 96 | P01275 | 1 | HDEFERHAEGTFTSDVSSYLEGQAAKEFIAWLVKG | 37 | Glucagon-like | Glucagon | T11 or T13 or S14 | 1 |
| | | | RG | | peptide 1 | | (Ambiguous) | |
| 97 | P01275 | 1 | HAEGTFTSDVSSYLEGQAAKEFIAWLVKGR | 31 | Glucagon-like | Glucagon | T5 or T7 or S8 (Ambiguous) | 1 |
| | | | | | peptide 1(7-36) | | | |
| 98 | P01275 | 1 | HAEGTFTSDVSSYLEGQAAKEFIAWLVKGRG | 30 | Glucagon-like | Glucagon | T5 or T7 or S8 (Ambiguous) | 1 |
| | | | | | peptide 1(7-37) | | | |
| 99 | P01275 | 1 | HADGSFSDEMNTILDNLAARDFINWLIQTKITD | 33 | Glucagon-like | Glucagon | | |
| | | | | | peptide 2 (By | | | |
| | | | | | similarity) | | | |
| 100 | P01282 | 1 | HADGVFTSDFSKLLGQLSAKKYLESLM | 27 | Intestinal peptide | Glucagon | T7, S8 | 2 |
| | | | | | PHM-27 | | | |
| 101 | P01282 | 1 | HADGVFTSDFSKLLGQLSAKKYLESLMGKRVSSNIS | 42 | Intestinal peptide | Glucagon | T7, S8 | 2 |
| | | | EDPVPV | | PHV-42 | | | |
| 102 | P01275 | 1 | HSQGTFTSDYSKYLDSRRAQDFVQWLMNTKRNRN | 37 | Oxyntomodulin | Glucagon | T7 | 1 |
| | | | NIA | | (By similarity) | | | |
| 103 | P18509 | 1 | DVAHGILNEAYRKVLDQLSAGKHLQSLVARGVGGS | 48 | PACAP-related | Glucagon | | |
| | | | LGGGAGDDAEPLS | | peptide | | | |
| 104 | P18509 | 1 | HSDGIFTDSYSRYRKQMAVKKYLAAVL | 27 | Pituitary | Glucagon | | |
| | | | | | adenylate | | | |
| | | | | | cyclase- | | | |
| | | | | | activating | | | |
| | | | | | polypeptide 27 | | | |
| 105 | P18509 | 1 | HSDGIFTDSYSRYRKQMAVKKYLAAVLGKRYKQR | 38 | Pituitary | Glucagon | | |
| | | | VKNK | | adenylate | | | |
| | | | | | cyclase- | | | |
| | | | | | activating | | | |
| | | | | | polypeptide 38 | | | |
| 106 | P09683 | 1 | HSDGTFTSELSRLREGARLQRLLQGLV | 27 | Secretin | Glucagon | T7, S8, S11 | 3 |
| 107 | P01286 | 1 | YADAIFTNSYRKVLGQLSARKLLQDIMSRQQGESN | 44 | Somatoliberin | Glucagon | | |
| | | | QERGARARL | | | | | |
| 108 | P01282 | 1 | HSDAVFTDNYTRLRKQMAVKKYLNSILN | 28 | Vasoactive | Glucagon | T7 | 1 |
| | | | | | intestinal | | | |
| | | | | | peptide | | | |
| 109 | P01148 | 1 | DAENLIDSFQEIVKEVGQLAETQRFECTTHQPRSPLR | 56 | GnRH-associated | GnRH | T28 | 1 |
| | | | DLKGALESLIEEETGQKKI | | peptide 1 | | | |
| 110 | O43555 | 1 | ALSSAQDPQNALRPPGRALDTAAGSPVQTAHGLPS | 84 | GnRH-associated | GnRH | | |
| | | | DALAPLDDSMPWEGRTTAQWSLHRKRHLARTLLT | | peptide 2 | | | |
| | | | AAREPRPAPPSSNKV | | | | | |
| 111 | P01148 | 1 | QHWSYGLRPG | 10 | Gonadoliberin-1 | GnRH | | |
| 112 | O43555 | 1 | QHWSHGWYPG | 10 | Gonadoliberin-2 | GnRH | | |
| 113 | P01148 | 1 | QHWSYGLRPGGKRDAENLIDSFQEIVKEVGQLAET | 69 | Progonadoliberin- | GnRH | T41 | 1 |
| | | | QRFECTTHQPRSPLRDLKGALESLIEEETGQKKI | | 1 | | | |
| 114 | O43555 | 1 | QHWSHGWYPGGKRALSSAQDPQNALRPPGRALDT | 97 | Progonadoliberin- | GnRH | | |
| | | | AAGSPVQTAHGLPSDALAPLDDSMPWEGRTTAQW | | 2 | | | |
| | | | SLHRKRHLARTLLTAAREPRPAPPSSNKV | | | | | |
| 115 | P01308 | 1 | GIVEQCCTSICSLYQLENYCN | 21 | Insulin A chain | Insulin | | |
| 116 | P01308 | 1 | FVNQHLCGSHLVEALYLVCGERGFFYTPKT | 30 | Insulin B chain | Insulin | T27 (Ambiguous) | 1 |
| 117 | P05019 | 1 | GPETLCGAELVDALQFVCGDRGFYFNKPTGYGSSSR | 70 | Insulin-like | Insulin | | |
| | | | RAPQTGIVDECCFRSCDLRRLEMYCAPLKPAKSA | | growth factor I | | | |
| 118 | P01344 | 1 | AYRPSETLCGGELVDTLQFVCGDRGFYFSRPASRVS | 67 | Insulin-like | Insulin | S50, T62 | 2 |
| | | | RRSRGIVEECCFRSCDLALLETYCATPAKSE | | growth factor II | | | |
| 119 | P01344 | 1 | YRPSETLCGGELVDTLQFVCGDRGFYFSRPASRVSR | 66 | Insulin-like | Insulin | S49, T61 | 2 |
| | | | RSRGIVEECCFRSCDLALLETYCATPAKSE | | growth factor II | | | |
| | | | | | Ala-25 Del | | | |
| 120 | P01344 | 1 | DVSTPPTVLPDNFPRYPVGKFFQYDTWKQSTQRL | 34 | Preptin | Insulin | S3, T4, T7 | 3 |
| 121 | P04090 | 1 | QLYSALANKCCHVGCTKRSLARFC | 24 | Relaxin A chain | Insulin | | |
| 122 | P04808 | 1 | PYVALFEKCCLIGCTKRSLAKYC | 23 | Relaxin A chain | Insulin | | |
| | | | | | (By similarity) | | | |
| 123 | P04090 | 1 | DSWMEEVIKLCGRELVRAQIAICGMSTWS | 29 | Relaxin B chain | Insulin | | |
| 124 | P04808 | 1 | VAAKWKDDVIKLCGRELVRAQIAICGMSTWS | 31 | Relaxin B chain | Insulin | | |
| | | | | | (By similarity) | | | |
| 125 | Q8WXF3 | 1 | DVLAGLSSSCCKWGCSKSEISSLC | 24 | Relaxin-3 A | Insulin | | |
| | | | | | chain (By | | | |
| | | | | | similarity) | | | |
| 126 | Q8WXF3 | 1 | RAAPYGVRLCGREFIRAVIFTCGGSRW | 27 | Relaxin-3 B | Insulin | | |
| | | | | | chain (By | | | |
| | | | | | similarity) | | | |
| 127 | Q15726 | 1 | YNWNSFGLRF | 10 | Kisspeptin-10 | KISS1 | | |
| 128 | Q15726 | 1 | LPNYNWNSFGLRF | 13 | Kisspeptin-13 | KISS1 | | |
| 129 | Q15726 | 1 | DLPNYNWNSFGLRF | 14 | Kisspeptin-14 | KISS1 | | |
| 130 | Q15726 | 1 | EPLEKVASVGNSRPTGQQLESLGLLAPGEQSLPCIL | 119 | Metastasis- | KISS1 | A69 | 1 |
| | | | RKPAATARLSRRGTSLSPPPESSGSPQQPGLSAPHSR | | suppressor KiSS- | | | |
| | | | QIPAPQGAVLVQREKDLPNYNVVNSFGLRFGKREAA | | 1 | | | |
| | | | PGNHGRSAGRG | | | | | |
| 131 | Q15726 | 1 | GTSLSPPPESSGSPQQPGLSAPHSRQIPAPQGAVLVQ | 54 | Metastin | KISS1 | A21 | 1 |
| | | | REKDLPNYNWNSFGLRF | | | | | |
| 132 | P41159 | 1 | VPIQKVQDDTKTLIKTIVTRINDISHTQSVSSKQKVT | 146 | Leptin | Leptin | | |
| | | | GLDFIPGLHPILTLSKMDQTLAVYQQILTSMPSRNVI | | | | | |
| | | | QISNDLENLRDLLHVLAFSKSCHLPWASGLETLDSL | | | | | |
| | | | GGVLEASGYSTEVVALSRLQGSLQDMLWQLDLSPG | | | | | |
| | | | C | | | | | |
| 133 | P20382 | 1 | DFDMLRCMLGRVYRPCWQV | 19 | Melanin- | Melanin- | | |
| | | | | | concentrating | concentrating | | |
| | | | | | hormone | hormone | | |
| 134 | P20382 | 1 | EIGDEENSAKFPI | 13 | Neuropeptide- | Melanin- | | |
| | | | | | glutamic acid- | concentrating | | |
| | | | | | isoleucine | hormone | | |
| 135 | P20382 | 1 | GSVAFPAENGVQNTESTQE | 19 | Neuropeptide- | Melanin- | T14, T16, T17 | 3 |
| | | | | | glycine-glutamic | concentrating | | |
| | | | | | acid (Potential) | hormone | | |
| 136 | P20382 | 1 | ILLSASKSIRNLDDDMVFNTFRLGKGFQKEDTAEKS | 144 | Pro-MCH | Melanin- | S36, S41, N72, T102, T104, | 6 |
| | | | VIAPSLEQYKNDESSFMNEEENKVSKNTGSKHNFLN | | | concentrating | T105 | |
| | | | HGLPLNLAIKPYLALKGSVAFPAENGVQNTESTQEK | | | hormone | | |
| | | | REIGDEENSAKFPIGRRDFDMLRCMLGRVYRPCWQ | | | | | |
| | | | V | | | | | |
| 137 | Q9UBU3 | 1 | GSSFLSPEHQRVQQRKESKKPPAKLQP | 27 | Ghrelins-27 | Motilin | | |
| 138 | Q9UBU3 | 1 | GSSFLSPEHQRVQQRKESKKPPAKLQPR | 28 | Ghrelins-28 | Motilin | | |
| 139 | P12872 | 1 | FVPIFTYGELQRMQEKERNKGQ | 22 | Motilin | Motilin | | |
| 140 | P12872 | 1 | SLSVWQRSGEEGPVDPAEPIREEENEMIKLTAPLEIG | 66 | Motilin- | Motilin | | |
| | | | MRMNSRQLEKYPATLEGLLSEMLPQHAAK | | associated | | | |
| | | | | | peptide | | | |
| 141 | Q9UBU3 | 1 | FNAPFDVGIKLSGVQYQQHSQAL | 23 | Obestatin | Motilin | | |
| 142 | P12872 | 1 | FVPIFTYGELQRMQEKERNKGQKKSLSVWQRSGEE | 90 | Promotilin | Motilin | | |
| | | | GPVDPAEPIREEENEMIKLTAPLEIGMRMNSRQLEK | | | | | |
| | | | YPATLEGLLSEMLPQHAAK | | | | | |
| 143 | Q15848 | 1 | ETTTQGPGVLLPLPKGACTGWMAGIPGHPGHNGAP | 226 | Adiponectin | NA | T4 | 1 |
| | | | GRDGRDGTPGEKGEKGDPGLIGPKGDIGETGVPGAE | | | | | |
| | | | GPRGFPGIQGRKGEPGEGAYVYRSAFSVGLETYVTI | | | | | |
| | | | PNMPIRETKIFYNQQNHYDGSTGKEHCNIPGLYYFA | | | | | |
| | | | YHITVYMKDVKVSLFKKDKAMLFTYDQYQENNVD | | | | | |
| | | | QASGSVLLHLEVGDQVWLQVYGEGERNGLYADND | | | | | |
| | | | NDSTFTGFLLYHDTN | | | | | |
| 144 | O00253 | 1 | AQMGLAPMEGIRRPDQALLPELPGLGLRAPLKKTT | 111 | Agouti-related | NA | T35 | 1 |
| | | | AEQAEEDLLQEAQALAEVLDLQDREPRSSRRCVRL | | protein | | | |
| | | | HESCLGQQVPCCDPCATCYCRFFNAFCYCRKLGTA | | | | | |
| | | | MNPCSRT | | | | | |
| 145 | Q9BZL1 | 1 | MIEVVCNDRLGKKVRVKCNTDDTIGDLKKLIAAQT | 73 | Ubiquitin-like | NA | | |
| | | | GTRWNKIVLKKWYTIFKDHVSLGDYEIHDGMNLEL | | protein 5 | | | |
| | | | YYQ | | | | | |
| 146 | P43490 | 1 | MNPAAEAEFNILLATDSYKVTHYKQYPPNTSKVYS | 491 | Nicotinamide | NAPRTase | | |
| | | | YFECREKKTENSKLRKVKYEETVEYGLQYILNKYL | | phosphoribosyltr | | | |
| | | | KGKVVTKEKIQEAKDVYKEHFQDDVFNEKGWNYI | | ansferase | | | |
| | | | LEKYDGHLPIEIKAVPEGFVIPRGNVLFTVENTDPEC | | | | | |
| | | | YWLTNWIETILVQSWYPITVATNSREQKKILAKYLL | | | | | |
| | | | ETSGNLDGLEYKLHDFGYRGVSSQETAGIGASAHL | | | | | |
| | | | VNFKGTDTVAGLALIKKYYGTKDPVPGYSVPAAEH | | | | | |
| | | | STITAWGKDHEKDAFEHIVTQFSSVPVSVVSDSYDI | | | | | |
| | | | YNACEKIWGEDLRHLIVSRSTQAPLIIRPDSGNPLDT | | | | | |
| | | | VLKVLEILGKKFPVTENSKGYKLLPPYLRVIQGDGV | | | | | |
| | | | DINTLQEIVEGMKQKMVVSIENIAFGSGGGLLQKLTR | | | | | |
| | | | DLLNCSEKCSYVVTNGLGINVEKDPVADPNKRSKK | | | | | |
| | | | GRLSLHRTPAGNFVTLEEGKGDLEEYGQDLLHTVF | | | | | |
| | | | KNGKVTKSYSFDEIRKNAQLNIELEAAHH | | | | | |
| 147 | P01160 | 1 | SLRRSSCFGGRMDRIGAQSGLGCNSFRY | 28 | Atrial natriuretic | Natriuretic peptide | S19, S25 | 2 |
| | | | | | factor | | | |
| 148 | P16860 | 1 | SPKMVQGSGCFGRKMDRISSSSGLGCKV | 28 | BNP(1-28) | Natriuretic peptide | | |
| 149 | P16860 | 1 | SPKMVQGSGCFGRKMDRISSSSGLGCKVL | 29 | BNP(1-29) | Natriuretic peptide | | |
| 150 | P16860 | 1 | SPKMVQGSGCFGRKMDRISSSSGLGCKVLR | 30 | BNP(1-30) | Natriuretic peptide | | |
| 151 | P16860 | 1 | PKMVQGSGCFGRKMDRISSSSGLGCKVLRR | 30 | BNP(2-31) | Natriuretic peptide | | |
| 152 | P16860 | 1 | KMVQGSGCFGRKMDRISSSSGLGCKVL | 27 | BNP(3-29) | Natriuretic peptide | | |
| 153 | P16860 | 1 | KMVQGSGCFGRKMDRISSSSGLGCKVLR | 28 | BNP(3-30) | Natriuretic peptide | | |
| 154 | P16860 | 1 | KMVQGSGCFGRKMDRISSSSGLGCKVLRRH | 30 | BNP(3-32) | Natriuretic peptide | | |
| 155 | P16860 | 1 | MVQGSGCFGRKMDRISSSSGLGCK | 24 | BNP(4-27) | Natriuretic peptide | | |
| 156 | P16860 | 1 | MVQGSGCFGRKMDRISSSSGLGCKVL | 26 | BNP(4-29) | Natriuretic peptide | | |
| 157 | P16860 | 1 | MVQGSGCFGRKMDRISSSSGLGCKVLR | 27 | BNP(4-30) | Natriuretic peptide | | |
| 158 | P16860 | 1 | MVQGSGCFGRKMDRISSSSGLGCKVLRR | 28 | BNP(4-31) | Natriuretic peptide | | |
| 159 | P16860 | 1 | MVQGSGCFGRKMDRISSSSGLGCKVLRRH | 29 | BNP(4-32) | Natriuretic peptide | | |
| 160 | P16860 | 1 | VQGSGCFGRKMDRISSSSGLGCKVL | 25 | BNP(5-29) | Natriuretic peptide | | |
| 161 | P16860 | 1 | VQGSGCFGRKMDRISSSSGLGCKVLRR | 27 | BNP(5-31) | Natriuretic peptide | | |
| 162 | P16860 | 1 | VQGSGCFGRKMDRISSSSGLGCKVLRRH | 28 | BNP(5-32) | Natriuretic peptide | | |
| 163 | P16860 | 1 | SPKIVIVQGSGCFGRKMDRISSSSGLGCKVLRRH | 32 | Brain natriuretic | Natriuretic peptide | | |
| | | | | | peptide 32 | | | |
| 164 | P01160 | 1 | NPMYNAVSNADLMDFKNLLDHLEEKMPLED | 30 | Cardiodilatin- | Natriuretic peptide | Y4, A6, S8 (Ambiguous) | 1 |
| | | | | | related peptide | | | |
| 165 | P23582 | 1 | GLSKGCFGLKLDRIGSMSGLGC | 22 | CNP-22 | Natriuretic peptide | | |
| 166 | P23582 | 1 | YKGANKKGLSKGCFGLKLDRIGSMSGLGC | 29 | CNP-29 | Natriuretic peptide | | |
| 167 | P23582 | 1 | DLRVDTKSRAAWARLLQEHPNARKYKGANKKGLS | 53 | CNP-53 | Natriuretic peptide | | |
| | | | KGCFGLKLDRIGSMSGLGC | | | | | |
| 168 | P16860 | 1 | HPLGSPGSASDLETSGLQEQRNHLQGKLSELQVEQT | 108 | Natriuretic | Natriuretic peptide | T48, E59 | 2 |
| | | | SLEPLQESPRPTGVWKSREVATEGIRGHRKMVLYTL | | peptides B | | | |
| | | | RAPRSPKMVQGSGCFGRKMDRISSSSGLGCKVLRR | | | | | |
| | | | H | | | | | |
| 169 | P58417 | 1 | ANLTNGGKSELLKSGSSKSTLKHIWTESSKDLSISRL | 250 | Neurexophilin-1 | Neurexophilin | | |
| | | | LSQTFRGKENDTDLDLRYDTPEPYSEQDLWDWLRN | | | | | |
| | | | STDLQEPRPRAKRRPIVKTGKFKMFGWGDFHSNIK | | | | | |
| | | | TVKLNLLITGKIVDHGNGTFSVYFRHNSTGQGNVSV | | | | | |
| | | | SLVPPTKIVEFDLAQQTVIDAKDSKSFNCRIEYEKVD | | | | | |
| | | | KATKNTLCNYDPSKTCYQEQTQSHVSWLCSKPFKV | | | | | |
| | | | ICIYISFYSTDYKLVQKVCPDYNYHSDTPYFPSG | | | | | |
| 170 | O95156 | 1 | KEVVHATEGLDWEDKDAPGTLVGNVVHSRIISPLR | 242 | Neurexophilin-2 | Neurexophilin | T7 | 1 |
| | | | LFVKQSPVPKPGPMAYADSMENFWDWLANITEIQE | | | | | |
| | | | PLARTKRRPIVKTGKFKKMFGWGDFHSNIKTVKLN | | | | | |
| | | | LLITGKIVDHGNGTFSVYFRHNSTGLGNVSVSLVPPS | | | | | |
| | | | KVVEFEVSPQSTLETKESKSFNCRIEYEKTDRAKKT | | | | | |
| | | | ALCNFDPSKICYQEQTQSHVSWLCSKPFKVICIYIAF | | | | | |
| | | | YSVDYKLVQKVCPDYNYHSETPYLSSG | | | | | |
| 171 | O95157 | 1 | QDDGPPGSEDPERDDHEGQPRPRVPRKRGHISPKSR | 230 | Neurexophilin-3 | Neurexophilin | | |
| | | | PMANSTLLGLLAPPGEAWGILGQPPNRPNHSPPPSA | | | | | |
| | | | KVKKIFGWGDFYSNIKTVALNLLVTGKIVDHGNGT | | | | | |
| | | | FSVHFQHNATGQGNISISLVPPSKAVEFHQEQQIFIEA | | | | | |
| | | | KASKIFNCRMEWEKVERGRRTSLCTHDPAKICSRDH | | | | | |
| | | | AQSSATWSCSQPFKVVCVYIAFYSTDYRLVQKVCP | | | | | |
| | | | DYNYHSDTPYYPSG | | | | | |
| 172 | O95158 | 1 | QIPESGRPQYLGLRPAAAGAGAPGQQLPEPRSSDGL | 285 | Neurexophilin-4 | Neurexophilin | | |
| | | | GVGRAWSWAWPTNHTGALARAGAAGALPAQRTK | | | | | |
| | | | RKPSIKAARAKKIFGWGDFYFRVHTLKFSLLVTGKI | | | | | |
| | | | VDHVNGTFSVYFRHNSSSLGNLSVSIVPPSKRVEFG | | | | | |
| | | | GVWLPGPVPHPLQSTLALEGVLPGLGPPLGMAAAA | | | | | |
| | | | AGPGLGGSLGGALAGPLGGALGVPGAKESRAFNCH | | | | | |
| | | | VEYEKTNRARKHRPCLYDPSQVCFTEHTQSQAAWL | | | | | |
| | | | CAKPFKVICIFVSFLSFDYKLVQKVCPDYNFQSEHPY | | | | | |
| | | | FG | | | | | |
| 173 | Q5H8A3 | 1 | ILQRGSGTAAVDFTKKDHTATWGRPFFLFRPRN | 33 | Neuromedin-S | Neuromedins | | |
| 174 | P48645 | 1 | FRVDEEFQSPFASQSRGYFLFRPRN | 25 | Neuromedin-U- | Neuromedins | | |
| | | | | | 25 | | | |
| 175 | Q8NG41 | 1 | WYKPAAGHSSYSVGRAAGLLSGLR | 24 | Neuropeptide B- | Neuromedins | | |
| | | | | | 23 | | | |
| 176 | Q8NG41 | 1 | WYKPAAGHSSYSVGRAAGLLSGLRRSPYA | 29 | Neuropeptide B- | Neuromedins | | |
| | | | | | 29 | | | |
| 177 | POC0P6 | 1 | SFRNGVGTGMKKTSFQRAKS | 20 | Neuropeptide S | Neuromedins | | |
| 178 | Q8N729 | 1 | WYKHVASPRYHTVGRAAGLLMGL | 23 | Neuropeptide W- | Neuromedins | | |
| | | | | | 23 | | | |
| 179 | Q8N729 | 1 | WYKHVASPRYHTVGRAAGLLMGLRRSPYLW | 30 | Neuropeptide W- | Neuromedins | | |
| | | | | | 30 | | | |
| 180 | P30990 | 1 | SDSEEEMKALEADFLTNMHTSKISKAHVPSWKMTL | 125 | Large | Neurotensin | | |
| | | | LNVCSLVNNLNSPAEETGEVHEEELVARRKLPTALD | | neuromedin N | | | |
| | | | GFSLEAMLTIYQLHKICHSRAFQHWELIQEDILDTGN | | | | | |
| | | | DKNGKEEVIKRKIPYIL | | | | | |
| 181 | P30990 | 1 | IPYIL | 5 | Neuromedin N | Neurotensin | | |
| 182 | P30990 | 1 | QLYENKPRRPYIL | 13 | Neurotensin | Neurotensin | | |
| 183 | P30990 | 1 | DSYYY | 5 | Tail peptide | Neurotensin | | |
| | | | | | (Potential) | | | |
| 184 | P01303 | 1 | SSPETLISDLLMRESTENVPRTRLEDPAMW | 30 | C-flanking | NPY | S1, S2, T16, T21, A28 | 3 |
| | | | | | peptide of NPY | | | |
| 185 | P01303 | 1 | YPSKPDNPGEDAPAEDMARYYSALRHYINLITRQRY | 36 | Neuropeptide Y | NPY | S3, T32 | 2 |
| 186 | P01298 | 1 | APLEPVYPGDNATPEQMAQYAADLRRYINMLTRPR | 36 | Pancreatic | NPY | T32 | 1 |
| | | | Y | | hormone | | | |
| 187 | P01298 | 1 | HKEDTLAFSEWGSPHAAVPR | 20 | Pancreatic | NPY | | |
| | | | | | icosapeptide | | | |
| 188 | P10082 | 1 | YPIKPEAPREDASPEELNRYYASLRHYLNLVTRQRY | 36 | Peptide YY | NPY | T32 | 1 |
| 189 | P10082 | 1 | IKPEAPREDASPEELNRYYASLRHYLNLVTRQRY | 34 | Peptide YY (3- | NPY | T30 | 1 |
| | | | | | 36) | | | |
| 190 | P80303 | 1 | VPIDIDKTKVQNIHPVESAKIEPPDTGLYYDEYLKQV | 82 | Nesfatin-1 | Nucleobindin | T8, I13, S18, T26, Y29, Y30, | 8 |
| | | | IDVLETDKHFREKLQKADIEEIKSGRLSKELDLVSHH | | | | T43, S72 | |
| | | | VRTKLDEL | | | | | |
| 191 | Q02818 | 1 | VPLERGAPNKEETPATESPDTGLYYHRYLQEVIDVL | 435 | Nucleobindin-1 | Nucleobindin | P8, T13, T16, S18, T21, Y24, | 22 |
| | | | ETDGHFREKLQAANAEDIKSGKLSRELDFVSHHVRT | | | | S67, T122, T136, S198, S294, | |
| | | | KLDELKRQEVSRLRMLLKAKMDAEQDPNVQVDHL | | | | S295, T309, H313, S343, T346, | |
| | | | NLLKQFEHLDPQNQHTFEARDLELLIQTATRDLAQY | | | | S352, Q381, A388, T400, L426, | |
| | | | DAAHHEEFKRYEMLKEHERRRYLESLGEEQRKEAE | | | | x433(deleterious mutation) | |
| | | | RKLEEQQRRHREHPKVNVPGSQAQLKEVWEELDGL | | | | | |
| | | | DPNRFNPKTFFILHDINSDGVLDEQELEALFTKELEK | | | | | |
| | | | VYDPKNEEDDMREMEEERLRMREHVMKNVDTNQ | | | | | |
| | | | DRLVTLEEFLASTQRKEFGDTGEGWETVEMHPAYT | | | | | |
| | | | EEELRRFEEELAAREAELNAKAQRLSQETEALGRSQ | | | | | |
| | | | GRLEAQKRELQQAVLHMEQRKQQQQQQQGHKAP | | | | | |
| | | | AAHPEGQLKFHPDTDDVPVPAPAGDQKEVDTSEKK | | | | | |
| | | | LLERLPEVEVPQHL | | | | | |
| 192 | P80303 | 1 | VPIDIDKTKVQNIHPVESAKIEPPDTGLYYDEYLKQV | 396 | Nucleobindin-2 | Nucleobindin | T8, I13, S18, T26, Y29, Y30, | 16 |
| | | | IDVLETDKHFREKLQKADIEEIKSGRLSKELDLVSHH | | | | T43, S72, S128, T139, T148, | |
| | | | VRTKLDELKRQEVGRLRMLIKAKLDSLQDIGMDHQ | | | | A331, Y365, P382, S384, L390 | |
| | | | ALLKQFDHLNHLNPDKEESTDLDMLIKAATSDLEH | | | | | |
| | | | YDKTRHEEFKKYEMMKEHERREYLKTLNEEKRKE | | | | | |
| | | | EESKILEMKKKHENHPKVNHPGSKDQLKEVWEET | | | | | |
| | | | DGLDPNDFDPKTFFKLHDVNSDGFLDEQELEALFTK | | | | | |
| | | | ELEKVYDPKNEEDDMVEMEEERLRMREHVMSEVD | | | | | |
| | | | TNKDRLVTLEEFLKATEKKEFLEPDSWETLDQQQFF | | | | | |
| | | | TEEELKEYENHALQENELKKKADELQKQKEELQRQ | | | | | |
| | | | HDQLEAQKLEYHQVIQQMEQKKLQQGIPPSGPAGE | | | | | |
| | | | LKFEPHI | | | | | |
| 193 | P01213 | 1 | YGGFLRKYPK | 10 | Alpha- | Opioid | | |
| | | | | | neoendorphin | | | |
| 194 | P01213 | 1 | YGGFLRKYP | 9 | Beta- | Opioid | | |
| | | | | | neoendorphin | | | |
| 195 | P01213 | 1 | YGGFLRRIRPKLKWDNQKRYGGFLRRQFKVVT | 32 | Big dynorphin | Opioid | | |
| 196 | P01213 | 1 | YGGFLRRIRPKLK | 13 | Dynorphin A(1- | Opioid | | |
| | | | | | 13) (By | | | |
| | | | | | similarity) | | | |
| 197 | P01213 | 1 | YGGFLRRIRPKLKWDNQ | 17 | Dynorphin A(1- | Opioid | | |
| | | | | | 17) | | | |
| 198 | P01213 | 1 | YGGFLRRI | 8 | Dynorphin A(1- | Opioid | | |
| | | | | | 8) (By similarity) | | | |
| 199 | P01213 | 1 | YGGFL | 5 | Leu-enkephalin | Opioid | | |
| 200 | P01213 | 1 | YGGFLRRQFKVVTRSQEDPNAYSGELFDA | 29 | Leumorphin | Opioid | | |
| 201 | P01210 | 1 | YGGFM | 5 | Met-enkephalin | Opioid | | |
| 202 | P01210 | 1 | YGGFMRGL | 8 | Met-enkephalin- | Opioid | | |
| | | | | | Arg-Gly-Leu | | | |
| 203 | P01210 | 1 | YGGFMRF | 7 | Met-enkephalin- | Opioid | | |
| | | | | | Arg-Phe | | | |
| 204 | Q13519 | 1 | MPRVRSLFQEQEEPEPGMEEAGEMEQKQLQ | 30 | Neuropeptide 1 | Opioid | P14 | 1 |
| | | | | | (Probable) | | | |
| 205 | Q13519 | 1 | FSEFMRQYLVLSMQSSQ | 17 | Neuropeptide 2 | Opioid | | |
| | | | | | (Probable) | | | |
| 206 | Q13519 | 1 | FGGFTGARKSARKLANQ | 17 | Nociceptin | Opioid | | |
| 207 | P01210 | 1 | DAEEDDSLANSSDLLKELLETGDNRERSHHQDGSD | 41 | PENK(143-183) | Opioid | | |
| | | | NEEEVS | | (By similarity) | | | |
| 208 | P01210 | 1 | FAEALPSDEEGESYSKEVPEME | 22 | PENK(237-258) | Opioid | | |
| | | | | | (By similarity) | | | |
| 209 | P01213 | 1 | YGGFLRRQFKVVT | 13 | Rimorphin | Opioid | | |
| 210 | P01210 | 1 | MDELYPMEPEEEANGSEILA | 20 | rimorphin | Opioid | | |
| 211 | P01210 | 1 | ECSQDCATCSYRLVRPADINFLACVMECEGKLPSLK | 73 | Synenkephalin | Opioid | | |
| | | | IWETCKELLQLSKPELPQDGTSTLRENSKPEESHLLA | | | | | |
| 212 | O43612 | 1 | QPLPDCCRQKTCSCRLYELLHGAGNHAAGILTL | 33 | Orexin-A | Orexin | | |
| 213 | O43612 | 1 | RSGPPGLQGRLQRLLQASGNHAAGILTM | 28 | Orexin-B | Orexin | | |
| 214 | P12272 | 1 | TRSAWLDSGVTGSGLEGDHLSDTSTTSLELDSR | 33 | Osteostatin | Parathyroid hormone | | |
| 215 | P12272 | 1 | AVSEHQLLHDKGKSIQDLRRREFLHHLIAEIHTAEIR | 141 | Parathyroid | Parathyroid hormone | T39 | 1 |
| | | | ATSEVSPNSKPSPNTKNHPVREGSDDEGRYLTQETN | | hormone-related | | | |
| | | | KVETYKEQPLKTPGKKKKGKPGKRKEQEKKKRRTR | | protein | | | |
| | | | SAWLDSGVTGSGLEGDHLSDTSTTSLELDSRRH | | | | | |
| 216 | P12272 | 1 | AVSEHQLLHDKGKSIQDLRRREFLHHLIAEIHTAEI | 36 | PTHrP[1-36] | Parathyroid hormone | | |
| 217 | P12272 | 1 | ATSEVSPNSKPSPNTKNHPVRFGSDDEGRYLTQETN | 57 | PTHrP[38-94] | Parathyroid hormone | T2 | 1 |
| | | | KVETYKEQPLKTPGKKKKGKP | | | | | |
| 218 | Q96A98 | 1 | SLALADDAAFRERARLLAALERRHWLNSYMHKLL | 39 | Tuberoinfundibular | Parathyroid hormone | | |
| | | | VLDAP | | peptide of | | | |
| | | | | | 39residues | | | |
| 219 | P01189 | 1 | YGGFMTSEKSQTPLVTLEKNAIIKNAYKKGE | 31 | Beta-endorphin | POMC | S10, T12, T16 | 3 |
| 220 | P01189 | 1 | SYSMEHFRWGKPVGKKRRPVKVYPNGAEDESAEA | 39 | Corticotropin | POMC | | |
| | | | FPLEF | | | | | |
| 221 | P01189 | 1 | PVKVYPNGAEDESAEAFPLEF | 21 | Corticotropin- | POMC | | |
| | | | | | like intermediary | | | |
| | | | | | peptide | | | |
| 222 | P01189 | 1 | ELTGQRLREGDGPDGPADDGAGAQADLEHSLLVAA | 89 | Lipotropin beta | POMC | S68, T70, T74 | 3 |
| | | | EKKDEGPYRMEHFRWGSPPKDKRYGGFMTSEKSQ | | | | | |
| | | | TPLVTLFKNAIIKNAYKKGE | | | | | |
| 223 | P01189 | 1 | ELTGQRLREGDGPDGPADDGAGAQADLEHSLLVAA | 56 | Lipotropin | POMC | | |
| | | | EKKDEGPYRMEHFRWGSPPKD | | gamma | | | |
| 224 | P01189 | 1 | SYSMEHFRWGKPV | 13 | Melanotropin | POMC | | |
| | | | | | alpha | | | |
| 225 | P01189 | 1 | DEGPYRMEHFRWGSPPKD | 18 | Melanotropin | POMC | | |
| | | | | | beta | | | |
| 226 | P01189 | 1 | YVMGHFRWDRF | 11 | Melanotropin | POMC | | |
| | | | | | gamma | | | |
| 227 | P01189 | 1 | WCLESSQCQDLTTESNLLECIRACKPDLSAETPMFP | 76 | NPP | POMC | T45 | 1 |
| | | | GNGDEQPLTENPRKYVMGHFRWDRFGRRNSSSSGS | | | | | |
| | | | SGAGQ | | | | | |
| 228 | P01189 | 1 | EDVSAGEDCGPLPEGGPEPRSDGAKPGPRE | 30 | Potential peptide | POMC | | |
| 229 | Q9UHG2 | 1 | LETPAPQVPARRLLPP | 16 | Big LEN (By | ProSAAS | T3, P4, A5 | 2 |
| | | | | | similarity) | | | |
| 230 | Q9UHG2 | 1 | AADHDVGSELPPEGVLGALLRVKRLETPAPQVPAR | 40 | Big PEN-LEN | ProSAAS | T27, P28, A29 | 2 |
| | | | RLLPP | | (By similarity) | | | |
| 231 | Q9UHG2 | 1 | ARPVKEPRGLSAASPPLAETGAPRRF | 26 | Big SAAS (By | ProSAAS | G9, S14, T20, G21, A22 | 5 |
| | | | | | similarity) | | | |
| 232 | Q9UHG2 | 1 | ARPVKEP | 7 | KEP (By | ProSAAS | | |
| | | | | | similarity) | | | |
| 233 | Q9UHG2 | 1 | LETPAPQVPA | 10 | Little LEN (By | ProSAAS | T3, P4, A5 | 2 |
| | | | | | similarity) | | | |
| 234 | Q9UHG2 | 1 | GLSAASPPLAETGAPRRF | 18 | Little SAAS (By | ProSAAS | G1, S6, T12, G13, A14 | 5 |
| | | | | | similarity) | | | |
| 235 | Q9UHG2 | 1 | AADHDVGSELPPEGVLGALLRV | 22 | PEN (By | ProSAAS | | |
| | | | | | similarity) | | | |
| 236 | Q9UHG2 | 1 | ARPVKEPRGLSAASPPLAETGAPRRFRRSVPRGEAA | 227 | ProSAAS | ProSAAS | G9, S14, T20, G21, A22, A82, | 13 |
| | | | GAVQELARALAHLLEAERQERARAEAQEAEDQQA | | | | N85, S86, A143, S173, A174, | |
| | | | RVLAQLLRVWGAPRNSDPALGLDDDPDAPAAQLAR | | | | T214, P215, A216 | |
| | | | ALLRARLDPAALAAQLVPAPVPAAALRPRPPVYDD | | | | | |
| | | | GPAGPDAEEAGDETPDVDPELLRYLLGRILAGSADS | | | | | |
| | | | EGVAAPRRLRRAADHDVGSELPPEGVLGALLRVKR | | | | | |
| | | | LETPAPQVPARRLLPP | | | | | |
| 237 | Q9HD89 | 1 | KTLCSMEEAINERIQEVAGSLIFRAISSIGLECQSVTS | 90 | Resistin | Resistin/FIZZ | | |
| | | | RGDLATCPRGFAVTGCTCGSACGSWDVRAETTCHC | | | | | |
| | | | QCAGMDWTGARCCRVQP | | | | | |
| 238 | Q9BQ08 | 1 | QCSLDSVMDKKIKDVLNSLEYSPSPISKKLSCASVKS | 88 | Resistin-like beta | Resistin/FIZZ | | |
| | | | QGRPSSCPAGMAVTGCACGYGCGSWDVQLETTCH | | | | | |
| | | | CQCSVVDWTTARCCHLT | | | | | |
| 239 | P83859 | 1 | QDEGSEATGFLPAAGEKTSGPLGNLAEELNGYSRK | 43 | QRF-amide | RFamide neuropeptide | | |
| | | | KGGFSFRF | | | | | |
| 240 | P06850 | 1 | SEEPPISLDLTFHLLREVLEMARAEQLAQQAHSNRK | 41 | Corticoliberin | Sauvagine/ | | |
| | | | LMEII | | | corticotropin- | | |
| | | | | | | releasing | | |
| | | | | | | factor/urotensin I | | |
| 241 | P55089 | 1 | DNPSLSIDLTFHLLRTLLELARTQSQRERAEQNRIIFD | 40 | Urocortin | Sauvagine/ | | |
| | | | SV | | | corticotropin- | | |
| | | | | | | releasing | | |
| | | | | | | factor/urotensin I | | |
| 242 | Q96RP3 | 1 | IVLSLDVPIGLLQILLEQARARAAREQATTNARILAR | 41 | Urocortin-2 | Sauvagine/ | | |
| | | | VGHC | | | corticotropin- | | |
| | | | | | | releasing | | |
| | | | | | | factor/urotensin I | | |
| 243 | Q969E3 | 1 | FTLSLDVPTNIMNLLFNIAKAKNLRAQAAANAHLM | 38 | Urocortin-3 | Sauvagine/ | | |
| | | | AQI | | | corticotropin- | | |
| | | | | | | releasing | | |
| | | | | | | factor/urotensin I | | |
| 244 | P01019 | 1 | DRVY | 4 | Angiotensin 1-4 | Serpin | | |
| 245 | P01019 | 1 | DRVYI | 5 | Angiotensin 1-5 | Serpin | | |
| 246 | P01019 | 1 | DRVYIHP | 7 | Angiotensin 1-7 | Serpin | | |
| 247 | P01019 | 1 | DRVYIHPFH | 9 | Angiotensin 1-9 | Serpin | | |
| 248 | P01019 | 1 | DRVYIHPFHL | 10 | Angiotensin-1 | Serpin | | |
| 249 | P01019 | 1 | DRVYIHPF | 8 | Angiotensin-2 | Serpin | | |
| 250 | P01019 | 1 | RVYIHPF | 7 | Angiotensin-3 | Serpin | | |
| 251 | P01019 | 1 | VYIHPF | 6 | Angiotensin-4 | Serpin | | |
| 252 | P01019 | 1 | DRVYIHPFHLVIHNESTCEQLAKANAGKPKDPTFIPA | 452 | Angiotensinogen | Serpin | T407 or S409 or T410 or T422 | 1 |
| | | | PIQAKTSPVDEKALQDQLVLVAAKLDFEDKLRAAM | | | | (Ambiguous) | |
| | | | VGMLANFLGFRIYGMHSELWGVVHGATVLSPTAVF | | | | | |
| | | | GTLASLYLGALDHTADRLQAILGVPWKDKNCTSRL | | | | | |
| | | | DAHKVLSALQAVQGLLVAQGRADSQAQLLLSTVV | | | | | |
| | | | GVFTAPGLHLKQPFVQGLALYTPVVLPRSLDFTELD | | | | | |
| | | | VAAEKIDRFMQAVTGWKTGCSLMGASVDSTLAFN | | | | | |
| | | | TYVHFQGKMKGFSLLAEPQEFWVDNSTSVSVPMLS | | | | | |
| | | | GMGTFQHWSDIQDNFSVTQVPFTESACLLLIQPHYA | | | | | |
| | | | SDLDKVEGLTFQQNSLNWMKKLSPRTIHLTMPQLV | | | | | |
| | | | LQGSYDLQDLLAQAELPAILHFELNLQKLSNDRIRV | | | | | |
| | | | GEVLNSIFFELEADEREPTESTQQLNKPEVLEVTLNR | | | | | |
| | | | PFLFAVYDQSATALHFLGRVANPLSTA | | | | | |
| 253 | P08185 | 1 | MDPNAAYVNMSNHHRGLASANVDFAFSLYKHLVA | 383 | Corticosteroid- | Serpin | | |
| | | | LSPKKNIFISPVSISMALAMLSLGTCGHTRAQLLQGL | | binding globulin | | | |
| | | | GFNLTERSETEIHQGFQHLHQLFAKSDTSLEMTMGN | | | | | |
| | | | ALFLDGSLELLESFSADIKHYYESEVLAMNFQDWAT | | | | | |
| | | | ASRQINSYVKNKTQGKIVDLFSGLDSPAILVLVNYIF | | | | | |
| | | | FKGTWTQPFDLASTREENFYVDETTVVKVPMMLQS | | | | | |
| | | | STISYLHDSELPCQLVQMNYVGNGTVFFILPDKGKM | | | | | |
| | | | NTVIAALSRDTINRWSAGLTSSQVDLYIPKVTISGVY | | | | | |
| | | | DLGDVLEEMGIADLFTNQANFSRITQDAQLKSSKVV | | | | | |
| | | | HKAVLQLNEEGVDTAGSTGVTLNLTSKPIILRFNQPF | | | | | |
| | | | IIMIFDHFTWSSLFLARVMNPV | | | | | |
| 254 | Q86U17 | 1 | QPLLAHGDKSLQGPQPPRHQLSEPAPAYHRITPTITN | 403 | Serpin A11 | Serpin | | |
| | | | FALRLYKELAADAPGNIFFSPVSISTTLALLSLGAQA | | | | | |
| | | | NTSALILEGLGFNLTETPEADIHQGFRSLLHTLALPSP | | | | | |
| | | | KLELKVGNSLFLDKRLKPRQHYLDSIKELYGAFAFS | | | | | |
| | | | ANFTDSVTTGRQINDYLRRQTYGQVVDCLPEFSQDT | | | | | |
| | | | FMVLANYIFFKAKWKHPFSRYQTQKQESFFVDERTS | | | | | |
| | | | LQVPMMHQKEMHRFLYDQDLACTVLQIEYRGNAL | | | | | |
| | | | ALLVLPDPGKMKQVEAALQPQTLRKWGQLLLPSLL | | | | | |
| | | | DLHLPRFSISGTYNLEDILPQIGLTNILNLEADFSGVT | | | | | |
| | | | GQLNKTISKVSHKAMVDMSEKGTEAGAASGLLSQP | | | | | |
| | | | PSLNTMSDPHAHFNRPFLLLLWEVTTQSLLFLGKVV | | | | | |
| | | | NPVAG | | | | | |
| 255 | Q8IW75 | 1 | LKPSFSPRNYKALSEVQGWKQRMAAKELARQNMD | 394 | Serpin A12 | Serpin | | |
| | | | LGFKLLKKLAFYNPGRNIFLSPLSISTAFSMLCLGAQ | | | | | |
| | | | DSTLDEIKQGFNFRKMPEKDLHEGFHYIIHELTQKT | | | | | |
| | | | QDLKLSIGNTLFIDQRLQPQRKFLEDAKNFYSAETIL | | | | | |
| | | | TNFQNLEMAQKQINDFISQKTHGKINNLIENIDPGTV | | | | | |
| | | | MLLANYIFFRARWKHEFDPNVTKEEDFFLEKNSSVK | | | | | |
| | | | VPMMFRSGIYQVGYDDKLSCTILEIPYQKNITAIFILP | | | | | |
| | | | DEGKLKHLEKGLQVDTFSRWKTLLSRRVVDVSVPR | | | | | |
| | | | LHMTGTFDLKKTLSYIGVSKIFEEHGDLTKIAPHRSL | | | | | |
| | | | KVGEAVHKAELKMDERGTEGAAGTGAQTLPMETP | | | | | |
| | | | LVVKIDKPYLLLIYSEKIPSVLFLGKIVNPIGK | | | | | |
| 256 | O75830 | 1 | SRCSAQKNTEFAVDLYQEVSLSHKDNIIFSPLGITLV | 387 | Serpin 12 | Serpin | | |
| | | | LEMVQLGAKGKAQQQIRQTLKQQETSAGEEFFVLK | | | | | |
| | | | SFFSAISEKKQEFTFNLANALYLQEGFTVKEQYLHG | | | | | |
| | | | NKEFFQSAIKLVDFQDAKACAEMISTWVERKTDGKI | | | | | |
| | | | KDMFSGEEFGPLTRLVLVNAIYFKGDWKQKFRKED | | | | | |
| | | | TQLINFTKKNGSTVKIPMMKALLRTKYGYFSESSLN | | | | | |
| | | | YQVLELSYKGDEFSLIIILPAEGMDIEEVEKLITAQQI | | | | | |
| | | | LKWLSEMQEEEVEISLPREKVEQKVDEKDVLYSLNI | | | | | |
| | | | TEIFSGGCDLSGITDSSEVYVSQVTQKVFFEINEDGS | | | | | |
| | | | EAATSTGIHIPVIMSLAQSQFIANHPFLFIMKHNPTES | | | | | |
| | | | ILFMGRVTNPDTQEIKGRDLDSL | | | | | |
| 257 | O00230 | 1 | DRMPCRNFFWKTFSSCK | 17 | Cortistatin-17 | Somastostatin | | |
| 258 | O00230 | 1 | QEGAPPQQSARRDRMPCRNFFWKITSSCK | 29 | Cortistatin-29 | Somastostatin | | |
| | | | | | (Potential) | | | |
| 259 | P61278 | 1 | AGCKNFFWKTFTSC | 14 | Somatostatin-14 | Somastostatin | | |
| 260 | P61278 | 1 | SANSNPAMAPRERKAGCKNFFWKTFTSC | 28 | Somatostatin-28 | Somastostatin | S1 or S4 (Ambiguous) | 1 |
| 261 | P01236 | 1 | LPICPGGAARCQVTLRDLFDRAVVLSHYIHNLSSEM | 199 | Prolactin | Somatotropin/ | | |
| | | | FSEFDKRYTHGRGFITKAINSCHTSSLATPEDKEQAQ | | | prolactin | | |
| | | | QMNQKDFLSLIVSILRSWNEPLYHLVTEVRGMQEAP | | | | | |
| | | | EAILSKAVEIEEQTKRLLEGMELIVSQVHPETKENEI | | | | | |
| | | | YPVWSGLPSLQMADEESRLSAYYNLLHCLRRDSHK | | | | | |
| | | | IDNYLKLLKCRIIHNNNC | | | | | |
| 262 | P20366 | 1 | ALNSVAYERSAMQNYE | 15 | C-terminal- | Tachykinin | A1, S4 | 2 |
| | | | | | flanking peptide | | | |
| 263 | P20366 | 1 | HKTDSFVGLM | 10 | Neurokinin A | Tachykinin | | |
| 264 | Q9UHF0 | 1 | DMHDFFVGLM | 10 | Neurokinin-B | Tachykinin | | |
| 265 | P20366 | 1 | DADSSIEKQVALLKALYGHGQISHKRHKTDSFVGL | 36 | Neuropeptide K | Tachykinin | | |
| | | | M | | | | | |
| 266 | P20366 | 1 | RPKPQQFFGLM | 11 | Substance P | Tachykinin | | |
| 267 | P20396 | 1 | QPEAAQQEAVTAAEHPGLDDFLRQVERLLFLRENIQ | 218 | Pro-thyrotropin- | TRH | I50 | 1 |
| | | | RLQGDQGEHSASQIFQSDWLSKRQHPGKREEEEEEG | | | releasing | | |
| | | | VEEEEEEEGGAVGPHKRQHPGRREDEASWSVDVTQ | | | hormone | | |
| | | | HKRQHPGRRSPWLAYAVPKRQHPGRRLADPKAQR | | | | | |
| | | | SWEEEEEEEEREEDLMPEKRQHPGKRALGGPCGPQ | | | | | |
| | | | GAYGQAGLLLGLLDDLSRSQGAEEKRQHPGRRAA | | | | | |
| | | | WVREPLEE | | | | | |
| 268 | P20396 | 1 | QHP | 3 | Thyrotropin- | TRH | | |
| | | | | | releasing | | | |
| | | | | | hormone | | | |
| 269 | O95399 | 1 | ETPDCFWKYCV | 11 | Urotensin-2 | Urotensin-2 | | |
| 270 | Q76510 | 1 | ACFWKYCV | 8 | Urotensin-2B | Urotensin-2 | | |
| 271 | P01185 | 1 | CYFQNCPRG | 9 | Arg-vasopressin | Vasopressin/oxytocin | | |
| 272 | P01185 | 1 | ASDRSNATQLDGPAGALLLRLVQLAGAPEPFLPAQP | 39 | Copeptin | Vasopressin/oxytocin | A27 | 1 |
| | | | DAY | | | | | |
| 273 | P01178 | 1 | AAPDLDVRKCLPCGPGGKGRCFGPNICCAEELGCFV | 94 | Neurophysin 1 | Vasopressin/oxytocin | | |
| | | | GTAEALRCQEENYLPSPCQSGQKACGSGGRCAVLG | | | | | |
| | | | LCCSPDGCHADPACDAEATFSQR | | | | | |
| 274 | P01185 | 1 | AMSDLELRQCLPCGPGGKGRCFGPSICCADELGCFV | 93 | Neurophysin 2 | Vasopressin/oxytocin | | |
| | | | GTAEALRCQEENYLPSPCQSGQKACGSGGRCAAFG | | | | | |
| | | | VCCNDESCVTEPECREGFFIRRA | | | | | |
| 275 | P01178 | 1 | CYIQNCPLG | 9 | Oxytocin | Vasopressin/oxytocin | | |
| 276 | O15240 | 1 | TLQPPSALRRRHYHHALPPSRHYP | 24 | Antimicrobial | VGF | | |
| | | | | | peptide | | | |
| | | | | | VGF[554-577] | | | |
| 277 | O15240 | 1 | RPESALLGGSEAGERLLQQGLAQVEA | 26 | Neuroendocrine | VGF | | |
| | | | | | regulatory | | | |
| | | | | | peptide-1 | | | |
| 278 | O15240 | 1 | QAEATRQAAAQEERLADLASDLLLQYLLQGGARQR | 38 | Neuroendocrine | VGF | | |
| | | | GLG | | regulatory | | | |
| | | | | | peptide-2 | | | |
| 279 | O15240 | 1 | APPGRPEAQPPPLSSEHKEPVAGDAVPGPKDGSAPE | 591 | Neurosecretory | VGF | P1, S12, P202, S205, | 5 |
| | | | VRGARNSEPQDEGELFQGVDPRALAAVLLQALDRP | | protein VGF | | T477 | |
| | | | ASPPAPSGSQQGPEEEAAEALLTETVRSQTHSLPAPE | | | | | |
| | | | SPEPAAPPRPQTPENGPEASDPSEELEALASLLQELR | | | | | |
| | | | DFSPSSAKRQQETAAAETETRTHTLTRVNLESPGPE | | | | | |
| | | | RVWRASWGEFQARVPERAPLPPPAPSQFQARMPDS | | | | | |
| | | | GPLPETHKFGEGVSSPKTHLGEALAPLSKAYQGVAA | | | | | |
| | | | PFPKARRPESALLGGSEAGERLLQQGLAQVEAGRR | | | | | |
| | | | QAEATRQAAAQEERLADLASDLLLQYLLQGGARQR | | | | | |
| | | | GLGGRGLQEAAEERESAREEEEAEQERRGGEERVG | | | | | |
| | | | EEDEEAAEAEAEAEEAERARQNALLFAEEEDGEAG | | | | | |
| | | | AEDKRSQEETPGHRRKEAEGTEEGGEEEDDEEMDP | | | | | |
| | | | QTIDSLIELSTKLHLPADDVVSIIEEVEEKRKRKKNA | | | | | |
| | | | PPEPVPPPRAAPAPTHVRSPQPPPPAPAPARDELPDW | | | | | |
| | | | NEVLPPWDREEDEVYPPGPYHPFPNYIRPRTLQPPSA | | | | | |
| | | | LRRRHYHHALPPSRHYPGREAQARRAQEEAEAEER | | | | | |
| | | | RLQEQEELENYIEHVLLRRP | | | | | |

### EMBODIMENTS

1. An isolated peptide hormone, such as recombinant, such as a neuropeptide comprising one or more O-linked glycan at a predetermined specific site, such as in the receptor-binding region.

2. The peptide hormone according to embodiment 1, wherein the one or more O-glycan structures include a glycan structure selected from a core1, core2, core3, or core4 structure with sialic acid capping, such as a structure as illustrated in FIG. 1.

3. The peptide hormone according to embodiments 1 or 2, wherein the one or more O-glycan structures include a Tn (GalNAc) structure.

4. The peptide hormone according to any one of embodiments 1-3, wherein the one or more O-glycan structures include Tn (GalNAc) structure with one sialic acid capping (alpha2-6).

5. The peptide hormone according to any one of embodiments 1-4, wherein the one or more O-glycan structures include the core1 structures with one sialic acid capping (alpha2-6).

6. The peptide hormone according to any one of embodiments 1-5, wherein the one or more O-glycan structures include the core1 structures with one sialic acid capping (alpha 2-3)

7. The peptide hormone according to any one of embodiments 1-6, wherein the one or more O-glycan structures include the core1 structures with two sialic acids capping (alpha 2-3 and alpha 2-6).

8. The peptide hormone according to any one of embodiments 1-7, which peptide hormone has improved, such as increased stability and/or circulatory half-life and/or other pharmacokinetic properties, such as improved stability in in vitro assays, plasma and/or bodyfluids.

9. The peptide hormone according to any one of embodiments 1-8, which peptide hormone has lower bioactivity in receptor signalling, such as decreased receptor stimulation in in vitro cell assays and/or in man.

10. The peptide hormone according to any one of embodiments 1-9, which peptide hormone exhibits improved receptor stimulation in in vitro cell assays and/or in man.

11. The peptide hormone according to any one of embodiments 1-10, which peptide hormone exhibits altered blood-brain barrier uptake in animals or in man, such as increased blood-brain barrier uptake in animals or in man, or decreased blood-brain barrier uptake in animals or in human.

12. The peptide hormone according to any one of embodiments 1-11, which peptide hormone exhibits receptor sub-type selectivity switch.

13. The peptide hormone according to any one of embodiments 1-12, which peptide hormone is specific to one or more tissue in human, such as specific to tissue of the nervous system.

14. The peptide hormone according to any one of embodiments 1-13, which peptide hormone is selected from any one of tables 4, 5, or 6, such as selected from the list consisting of a peptide of the Neuropeptide Y family, such as NPY, PPY and PYY; a peptide of the Glucagon/Secretin family, such as GIP, Glucagon, GLP-1, GLP-2, PACAP, Secretin, PHM-27/PHV-42, Somatoliberin, and VIP; a peptide of the Natriuretic peptide family, such as ANP, BNP and CNP, a peptide of the calcitonin family, such as calcitonin, and amylin.

15. The peptide hormone according to any one of embodiments 1-14, which peptide hormone is not found in nature.

16. The peptide hormone according to any one of embodiments 1-15, which peptide hormone is a truncated version or a variant as compared to the corresponding wild-type peptide hormone found in nature.

17. The peptide hormone according to any one of embodiments 1-16, which peptide hormone is selected from any one of table 6 comprising one or more O-linked glycan at a site as indicated in table 6, such as at a bold underlined position and/or an italic underlined position.

18. The peptide hormone according to any one of embodiments 1-17, which peptide hormone is selected from any one of table 6 comprising at least, not more than, or the exact number of O-linked glycan sites as indicated in table 6.

19. The peptide hormone according to any one of embodiments 1-16, which peptide hormone is selected from any one of table 5.

20. A host cell comprising two or more glycosyltransferase genes that have been inactivated such that

(a) Homogenous Tn (GalNAc) glycosylation is obtained by inactivation and/or downregulation of one or more genes selected from COSMC and C1GALT1;
(b) Homogenous T (Gal/GalNAc) glycosylation is obtained by inactivation and/or downregulation of one or more genes selected from GCNT1, GCNT3, GCNT4, B3GNT6; and
(c) Homogenous ST or STn glycosylation is obtained by inactivation and/or downregulation of one or more genes selected from ST6GALNAC1-6, ST3GAL1, GCNT3, GCNT4, B3GNT6.

21. The host cell according to embodiment 20, further comprising a gene encoding an exogenous peptide hormone, such as a peptide hormone as defined in any one of embodiments 14-19.

22. A method for producing an isolated peptide hormone comprising one or more O-linked glycan at a predetermined specific site, such as in the receptor-binding region, the method comprising;
a) inactivation and/or downregulation of one or more glycosyltransferases, and/or knock in of one or more glycosyltransferases, or any combination hereof in a host cell, and
b) expression of said peptide hormone in said host cell.

23. The method according to embodiment 22, wherein one or more genes selected from COSMC, C1GALT1, GCNT1, GCNT3, GCNT4, B3GNT6, ST6GALNAC1-6, ST3GAL1 has been inactivated and/or downregulated.

24. The method according to embodiments 22 or 23, wherein said peptide hormone produced is as defined in any one of embodiments 1-19.

25. The method according to any one of embodiments 22-24, wherein said host cell is as defined in any of embodiments 20-21.

26. A method for the production of recombinant glycosylated peptide hormones that do not have specific types of glycosylation, the method comprising the step of inactivating two or more glycogenes to block and/or truncate one or more glycosylation pathways.

27. A method for the production of an isolated peptide hormone, such as a neuropeptide comprising one or more O-linked glycan at a predetermined specific site, such as in the receptor-binding region, said method comprising
a) providing a non-O-glycosylated peptide hormone; and
b) treating said non-O-glycosylated synthetic peptide hormone with one or more recombinant purified glycosyl transferase, such as a GalNAc-transferase, such as GalNAc-T1, T2, T3, T4, T5, T6, T7, T10, T11, T12, T13, T14, and/or T16, and/or a Galactosyl-transferases (C1GalT1) and/or a sialyl-transferases, such as ST6GalNAc1 and/or ST3Gall under conditions to add one or more specific O-linked glycan to said peptide hormone.

28. The method according to embodiment 27, wherein said non-O-glycosylated peptide hormone is provided as a chemically produced peptide hormone produced using solid phase peptide synthesis Fmoc SPPS.

29. The method according to embodiment 27, wherein said non-O-glycosylated peptide hormone is provided as a recombinantly produced peptide hormone, such as produced in a production cell line.

30. The method according to any one of embodiments 27-29, wherein said peptide hormone produced is as defined in any one of embodiments 1-19.

31. A method for the production of an isolated peptide hormone, such as a neuropeptide comprising one or more O-linked glycan at a predetermined specific site, such as in the receptor-binding region, said method comprising the building of said peptide hormone using solid phase peptide synthesis Fmoc SPPS including the use of glycosylated amino acids building blocks at said predetermined specific site(s).

32. The method according to embodiment 31, wherein the peptide hormone produced is as defined in any one of embodiments 1-19.

### ADDITIONAL EMBODIMENTS

a. An isolated peptide hormone, such as recombinant, such as a neuropeptide comprising one or more O-linked glycan at a predetermined specific site, such as in the receptor-binding region.

b. The peptide hormone according to embodiment a), wherein the one or more O-glycan structures include a glycan structure selected from a core1, core2, core3, or core4 structure with sialic acid capping, such as a structure as illustrated in FIG. 1.

c. The peptide hormone according to embodiments a) or b), wherein the one or more O-glycan structures include a Tn (GalNAc) structure.

d. The peptide hormone according to any one of embodiments a) to c), wherein the one or more O-glycan structures include Tn (GalNAc) structure with one sialic acid capping (alpha2-6).

e. The peptide hormone according to any one of embodiments a)-d), wherein the one or more O-glycan structures include the core1 structures with one sialic acid capping (alpha2-6).

f. The peptide hormone according to any one of embodiments a)-e), wherein the one or more O-glycan structures include the core1 structures with one sialic acid capping (alpha 2-3)

g. The peptide hormone according to any one of embodiments a)-f), wherein the one or more O-glycan structures include the core1 structures with two sialic acids capping (alpha 2-3 and alpha 2-6).

h. The peptide hormone according to any one of embodiments a)-g), which peptide hormone has improved, such as increased stability and/or circulatory half-life and/or other pharmacokinetic properties, such as improved stability in in vitro assays, plasma and/or bodyfluids.

i. The peptide hormone according to any one of embodiments a)-h), which peptide hormone has lower bioactivity in receptor signalling, such as decreased receptor stimulation in in vitro cell assays and/or in man.

j. The peptide hormone according to any one of embodiments a)-i), which peptide hormone exhibits improved receptor stimulation in in vitro cell assays and/or in man.

k. The peptide hormone according to any one of embodiments a)-j), which peptide hormone exhibits altered blood-brain barrier uptake in animals or in man, such as increased blood-brain barrier uptake in animals or in man, or decreased blood-brain barrier uptake in animals or in human.

l. The peptide hormone according to any one of embodiments a)-k), which peptide hormone exhibits receptor sub-type selectivity switch.

m. The peptide hormone according to any one of embodiments a)-l), which peptide hormone is selected from any one of tables 4, 5, or 6, such as selected from the list consisting of a peptide of the Neuropeptide Y family, such as NPY, PPY and PYY; a peptide of the Glucagon/Secretin family, such as GIP, Glucagon, GLP-1, GLP-2, PACAP, Secretin, PHM-27/PHV-42, Somatoliberin, and VIP; a peptide of the Natriuretic peptide family, such as ANP, BNP and CNP, a peptide of the calcitonin family, such as calcitonin, and amylin.

n. The peptide hormone according to any one of embodiments a)-m), which peptide hormone is not found in nature, such as a truncated version or a variant as compared to the corresponding wild-type peptide hormone found in nature.

o. The peptide hormone according to any one of embodiments a)-n), which peptide hormone is selected from any one of table 6 comprising one or more O-linked glycan at a site as indicated in table 6, such as at a bold underlined position and/or an italic underlined position.

### LIST OF REFERENCES

1 Seidah, N. G. What lies ahead for the proprotein convertases? Ann N Y Acad Sci 1220, 149-161, doi: 10.1111/j. 1749-6632.2010.05883.x (2011).
2 Milgram, S. L., Mains, R. E. & Eipper, B. A. COOH-terminal signals mediate the trafficking of a peptide processing enzyme in endocrine cells. J Cell Biol 121, 23-36 (1993).
3 Bundgaard, J. R., Vuust, J. & Rehfeld, J. F. Tyrosine O-sulfation promotes proteolytic processing of progastrin. The EMBO journal 14, 3073-3079 (1995).
4 Drazic, A., Myklebust, L. M., Ree, R. & Arnesen, T. The world of protein acetylation. Biochim Biophys Acta 1864, 1372-1401, doi:10.1016/j.bbapap.2016.06.007 (2016).
5 Secher, A. et at. Analytic framework for peptidomics applied to large-scale neuropeptide identification. Nature communications 7, 11436, doi:10.1038/ncomms11436 (2016).
6 Mentlein, R. Dipeptidyl-peptidase IV (CD26)—role in the inactivation of regulatory peptides. Regulatory peptides 85, 9-24 (1999).
7 Potter, L. R. Natriuretic peptide metabolism, clearance and degradation. Febs J 278, 1808-1817, doi:10.1111/j.1742-4658.2011.08082.x (2011).
8 Deacon, C. F., Nauck, M. A., Meier, J., Hucking, K. & Holst, J. J. Degradation of endogenous and exogenous gastric inhibitory polypeptide in healthy and in type 2 diabetic subjects as revealed using a new assay for the intact peptide. J Clin Endocrinol Metab 85, 3575-3581, doi: 10.1210/jcem.85.10.6855 (2000).
9 Halim, A., Ruetschi, U., Larson, G. & Nilsson, J. LC-MS/MS characterization of O-glycosylation sites and glycan structures of human cerebrospinal fluid glycoproteins. Journal of proteome research 12, 573-584, doi:10.1021/pr300963 h (2013).
10 Halim, A., Nilsson, J., Ruetschi, U., Hesse, C. & Larson, G. Human urinary glycoproteomics; attachment site specific analysis of N- and O-linked glycosylations by CID and ECD. Molecular & cellular proteomics: MCP 11, Mill 013649, doi: 10.1074/mcp.M 111.013649 (2012).
11 Khetarpal, S. A. et at Loss of Function of GALNT2 Lowers High-Density Lipoproteins in Humans, Nonhuman Primates, and Rodents. Cell Metab 24, 234-245, doi: 10.1016/j.cmet.2016.07.012 (2016).
12 King, S. L. et at Characterizing the O-glycosylation landscape of human plasma, platelets, and endothelial cells. Blood Advances 1, 429-442, doi: 10.1182/bloodadvances. 2016002121 (2017).
13 Yu, Q. et at Targeted Mass Spectrometry Approach Enabled Discovery of O-Glycosylated Insulin and Related Signaling Peptides in Mouse and Human Pancreatic Islets. Anal Chem, doi:10.1021/acs.analchem.7b01926 (2017).
14 Cao, F. et at Detection of Biosynthetic Precursors, Discovery of Glycosylated Forms, and Homeostasis of Calcitonin in Human Cancer Cells. Anal Chem 89, 6992-6999, doi: 10.1021/acs.analchem.7b00457 (2017).
15 Bennett, E. P. et at Control of mucin-type O-glycosylation: A classification of the polypeptide GalNAc-transferase gene family. Glycobiology 22, 736-756, doi:Doi 10.1093/Glycob/Cwr182 (2012).
16 Topaz, O. et at Mutations in GALNT3, encoding a protein involved in O-linked glycosylation, cause familial tumoral calcinosis. Nat Genet 36, 579-581, doi:10.1038/ng1358 (2004).
17 Kato, K. et at Polypeptide GalNAc-transferase T3 and familial tumoral calcinosis. Secretion of fibroblast growth factor 23 requires O-glycosylation. J Biol Chem 281, 18370-18377, doi:M602469200 [pii]10.1074/jbe.M602469200 (2006).
18 Schjoldager, K. T. et at Probing isoform-specific functions of polypeptide GalNAc-transferases using zinc finger nuclease glycoengineered SimpleCells. Proc Natl Acad Sci U SA 109, 9893-9898, doi: 10.1073/pnas. 1203563109 (2012).
19 Chretien, M. & Mbikay, M. 60 YEARS OF POMC: From the prohormone theory to pro-opiomelanocortin and to proprotein convertases (PCSK1 to PCSK9). J Mol Endocrinol 56, T49-62, doi: 10.1530/JME-15-0261 (2016).
20 Walsh, G. & Jefferis, R. Post-translational modifications in the context of therapeutic proteins. Nature biotechnology 24, 1241-1252, doi:10.1038/nbt1252 (2006).
21 Tenno, M. et al. Initiation of protein O glycosylation by the polypeptide GalNAcT-1 in vascular biology and humoral immunity. Mol Cell Biol 27, 8783-8796, doi:10.1128/MCB.01204-07 (2007).
22 Tian, E. & Ten Hagen, K. G. A UDP-GalNAc:polypeptide N-acetylgalactosaminyltransferase is required for epithelial tube formation. J Biol Chem 282, 606-614, doi: 10.1074/jbe.M606268200 (2007).
23 Freire-de-Lima, L. et al. Involvement of O-glycosylation defining oncofetal fibronectin in epithelial-mesenchymal transition process. Proc Natl Acad Sci USA 108, 17690-17695, doi: 10.1073/pnas. 1115191108 (2011).
24 Schjoldager, K. T. & Clausen, H. Site-specific protein O-glycosylation modulates proprotein processing—deciphering specific functions of the large polypeptide GalNAc-transferase gene family. Biochim Biophys Acta 1820, 2079-2094, doi:10.1016/j.bbagen.2012.09.014 (2012).
25 Tagliabracci, V. S. et al. Dynamic regulation of FGF23 by Fam20C phosphorylation, GalNAc-T3 glycosylation, and furin proteolysis. Proc Natl Acad Sci USA 111, 5520-5525, doi: 10.1073/pnas. 1402218111 (2014).
26 Goth, C. K. et at. A systematic study of modulation of ADAM-mediated ectodomain shedding by site-specific O-glycosylation. Proc Natl Acad Sci USA 112, 14623-14628, doi: 10.1073/pnas. 1511175112 (2015).
27 Norden, R. et al. O-linked glycosylation of the mucin domain of the herpes simplex virus type 1-specific glycoprotein gC-1 is temporally regulated in a seed-and-spread manner. The Journal of biological chemistry 290, 5078-5091, doi:10.1074/jbc.M114.616409 (2015).
28 Schjoldager, K. T. et al. Deconstruction of O-glycosylation—GalNAc-T isoforms direct distinct subsets of the O-glycoproteome. EMBO Rep 16, 1713-1722, doi: 10.15252/embr.201540796 (2015).
29 Tian, E. et al. Galnt1 is required for normal heart valve development and cardiac function. PLoS One 10, e0115861, doi:10.1371/journal.pone.0115861 (2015).
30 Akasaka-Manya, K. et al. Excess APP O-glycosylation by GalNAc-T6 decreases Abeta production. Journal of biochemistry 161, 99-111, doi: 10.1093/jb/mvw056 (2017).
31 Lin, J. et al. GALNT6 Stabilizes GRP78 Protein by O-glycosylation and Enhances its Activity to Suppress Apoptosis Under Stress Condition. Neoplasia (New York, N.Y. 19, 43-53, doi: 10.1016/j.neo.2016.11.007 (2017).
32 Goth, C. K. et al. Site-specific O-glycosylation by Polypeptide GalNAc-transferase T2 Co-regulates Beta1-adrenergic Receptor N-terminal Cleavage. J Biol Chem, doi: 10.1074/j be. Ml 16.730614 (2017).
33 Schjoldager, K. T. B. G. et al. A Systematic Study of Site-specific GalNAc-type O-Glycosylation Modulating Proprotein Convertase Processing. Journal of Biological Chemistry 286, 40122-40132, doi:10.1074/jbc.M111.287912 (2011).
34 Schjoldager, K. T. B. G. et al. O-Glycosylation Modulates Proprotein Convertase Activation of Angiopoietin-like Protein 3 POSSIBLE ROLE OF POLYPEPTIDE GalNAc-TRANSFERASE-2 IN REGULATION OF CONCENTRATIONS OF PLASMA LIPIDS. Journal of Biological Chemistry 285, 36293-36303, doi:10.1074/jbc.M110.156950 (2010).
35 Pedersen, N. B. et al. Low density lipoprotein receptor class a repeats are o-glycosylated in linker regions. J Biol Chem 289, 17312-17324, doi:10.1074/jbc.M113.545053 (2014).
36 Wang, S. et al. Site-specific O-glycosylation of members of the low-density lipoprotein receptor superfamily enhances ligand interactions. J Biol Chem, doi: 10.1074/j be. Ml 17.817981 (2018).
37 Pedragosa-Badia, X., Stichel, J. & Beck-Sickinger, A. G. Neuropeptide Y receptors: how to get subtype selectivity. Front Endocrinol (Lausanne) 4, 5, doi: 10.3389/fendo.2013.00005 (2013).
38 Tan, T. et al. The effect of a subcutaneous infusion of GLP-1, OXM and PYY on Energy intake and Expenditure in Obese volunteers. J Clin Endocrinol Metab, doi:10.1210/jc.2017-00469 (2017).
39 van Deursen, V. M. et al. Nesiritide, renal function, and associated outcomes during hospitalization for acute decompensated heart failure: results from the Acute Study of Clinical Effectiveness of Nesiritide and Decompensated Heart Failure (ASCEND-HF). Circulation 130, 958-965, doi:10.1161/CIRCULATIONAHA.113.003046 (2014).
40 Boerrigter, G. & Burnett, J. C., Jr. Natriuretic peptides renal protective after all? J Am Coll Cardiol 58, 904-906, doi:10.1016/j.jacc.2010.12.053 (2011).
41 Hata, N. et al. Effects of carperitide on the long-term prognosis of patients with acute decompensated chronic heart failure: the PROTECT multicenter randomized controlled study. Circ J 72, 1787-1793 (2008).
42 Fosgerau, K. & Hoffmann, T. Peptide therapeutics: current status and future directions. Drug Discov Today 20, 122-128, doi:10.1016/j.drudis.2014.10.003 (2015).
43 Yi, J., Warunek, D. & Craft, D. Degradation and Stabilization of Peptide Hormones in Human Blood Specimens. PLoS One 10, e0134427, doi: 10.1371/journal.pone.0134427 (2015).
44 Chapter, M. C. et at. Chemical modification of class II G protein-coupled receptor ligands: frontiers in the development of peptide analogs as neuroendocrine pharmacological therapies. Pharmacol Ther 125, 39-54, doi:10.1016/j.pharmthera.2009.07.006 (2010).
45 DeFrees, S. et al. GlycoPEGylation of recombinant therapeutic proteins produced in Escherichia coli. Glycoblology 16, 833-843, doi:10.1093/glycob/cwl004 (2006).
46 Meurer, J. A., Colca, J. R., Burton, P. S. & Elhammer, A. P. Properties of native and in vitro glycosylated forms of the glucagon-like peptide-1 receptor antagonist exendin(9-39). Metabolism: clinical and experimental 48, 716-724 (1999).
47 Egleton, R. D. et al. Improved blood-brain barrier penetration and enhanced analgesia of an opioid peptide by glycosylation. J Pharmacol Exp Ther 299, 967-972 (2001).
48 Kihlberg, J. et al. Glycosylated peptide hormones: pharmacological properties and conformational studies of analogues of [1-desamino,8-D-arginine]vasopressin. J Med Chem 38, 161-169 (1995).
49 Ueda, T. et al. Chemoenzymatic synthesis of glycosylated glucagon-like peptide 1: effect of glycosylation on proteolytic resistance and in vivo blood glucose-lowering activity. Journal of the American Chemical Society 131, 6237-6245, doi:10.1021/ja900261 g (2009).
50 Pedersen, S. L., Steentoft, C., Vrang, N. & Jensen, K. J. Glyco-scan: varying glycosylation in the sequence of the peptide hormone PYY3-36 and its effect on receptor selectivity. Chembiochem 11, 366-374, doi:10.1002/cbic.200900661 (2010).
51 Sato, M. et al. Glycoinsulins: dendritic sialyloligosaccharide-displaying insulins showing a prolonged blood-sugar-lowering activity. Journal of the American Chemical Society 126, 14013-14022, doi: 10.1021/ja046426l (2004).
52 Dangoor, D. et al. Novel glycosylated VIP analogs: synthesis, biological activity, and metabolic stability. J Pept Sci 14, 321-328, doi: 10.1002/psc.932 (2008).
53 Semenov, A. G. et al. Processing of pro-brain natriuretic peptide is suppressed by O-glycosylation in the region close to the cleavage site. Clin Chem 55, 489-498, doi: 10.1373/clinchem.2008.113373 (2009).
54 Duguay, S. J. et al. Post-translational processing of the insulin-like growth factor-2 precursor. Analysis of O-glycosylation and endoproteolysis. The Journal of biological chemistry 273, 18443-18451 (1998).
55 Birch, N. P., Estivariz, F. E., Bennett, H. P. & Loh, Y. P. Differential glycosylation of N-POMC1-77 regulates the production of gamma 3-MSH by purified pro-opiomelanocortin converting enzyme. A possible mechanism for tissue-specific processing. FEBS Lett 290, 191-194 (1991).
56 Patzelt, C. & Weber, B. Early O-glycosidic glycosylation of proglucagon in pancreatic islets: an unusual type of prohormonal modification. The EMBO journal 5, 2103-2108 (1986).
57 Luckenbill, K. N. et al. Cross-reactivity of BNP, NT-proBNP, and proBNP in commercial BNP and NT-proBNP assays: preliminary observations from the IFCC Committee for Standardization of Markers of Cardiac Damage. Clin Chem 54, 619-621, doi: 10.1373/clinchem.2007.097998 (2008).
58 Stovold, R. et al. Biomarkers for small cell lung cancer: neuroendocrine, epithelial and circulating tumour cells. Lung Cancer 76, 263-268, doi:10.1016/j.lungcan.2011.11.015 (2012).
59 Wang, J. et al. Identification of kininogen-1 as a serum biomarker for the early detection of advanced colorectal adenoma and colorectal cancer. PLoS One 8, e70519, doi:10.1371/journal.pone.0070519 (2013).
60 Goetze, J. P., Alehagen, U., Flyvbjerg, A. & Rehfeld, J. F. Chromogranin A as a biomarker in cardiovascular disease. Biomark Med 8, 133-140, doi:10.2217/bmm.13.102 (2014).
61 Nielsen, S. J., Iversen, P., Rehfeld, J. F., Jensen, H. L. & Goetze, J. P. C-type natriuretic peptide in prostate cancer. APMIS 117, 60-67, doi: 10.1111/j. 1600-0463.2008.00016.x 60 (2009).
62 Ju, T., Aryal, R. P., Stowell, C. J. & Cummings, R. D. Regulation of protein O-glycosylation by the endoplasmic reticulum-localized molecular chaperone Cosmc. J Cell Biol 182, 531-542, doi:10.1083/jcb.200711151 (2008).
63 Steentoft, C. et al. Mining the O-glycoproteome using zinc-finger nuclease-glycoengineered SimpleCell lines. Nat Methods 8, 977-982, doi:10.1038/nmeth.1731 (2011).
64 Steentoft, C. et al. Precision genome editing: A small revolution for glycobiology. Glycobiology 24, 663-680, doi: 10.1093/glycob/cwu046 (2014).
65 Yamane-Ohnuki, N. et at Establishment of FUT8 knockout Chinese hamster ovary cells: an ideal host cell line for producing completely defucosylated antibodies with enhanced antibody-dependent cellular cytotoxicity. Biotechnol Bioeng 87, 614-622, doi: 10.1002/bit. 20151 (2004).
66 Malphettes, L. et at Highly efficient deletion of FUT8 in CHO cell lines using zinc-finger nucleases yields cells that produce completely nonfucosylated antibodies. Biotechnol Bioeng 106, 774-783, doi: 10.1002/bit.22751 (2010).
67 Ronda, C. et at Accelerating genome editing in CHO cells using CRISPR Cas9 and CRISPy, a web-based target finding tool. Biotechnol Bioeng 111, 1604-1616, doi: 10.1002/bit.25233 (2014).
68 El Mai, N., Donadio-Andrei, S., Iss, C., Calabro, V. & Ronin, C. Engineering a human-like glycosylation to produce therapeutic glycoproteins based on 6-linked sialylation in CHO cells. Methods in molecular biology 988, 19-29, doi:10.1007/978-1-62703-327-5_2 (2013).
69 Sewell, R. et at The ST6GalNAc-I sialyltransferase localizes throughout the Golgi and is responsible for the synthesis of the tumor-associated sialyl-Tn O-glycan in human breast cancer. The Journal of biological chemistry 281, 3586-3594, doi: 10.1074/jbc.M511826200 (2006).
70 Torang, S. et at In vivo and in vitro degradation of peptide YY3-36 to inactive peptide YY3-34 in humans. Am J Physiol Regul Integr Comp Physiol 310, R866-874, doi: 10.1152/ajpregu.00394.2015 (2016).
71 Steentoft, C. et at Precision mapping of the human O-GalNAc glycoproteome through SimpleCell technology. The EMBO journal 32, 1478-1488, doi:10.1038/emboj.2013.79 (2013).
72 Levery, S. B. et at Advances in mass spectrometry driven O-glycoproteomics. Biochim Biophys Acta, doi:10.1016/j.bbagen.2014.09.026 (2014).
73 Schietinger, A. et at A mutant chaperone converts a wild-type protein into a tumor-specific antigen. Science 314, 304-308, doi:10.1126/science.1129200 (2006).
74 Yabu, M., Korekane, H. & Miyamoto, Y. Precise structural analysis of O-linked oligosaccharides in human serum. Glycobiology 24, 542-553, doi:10.1093/glycob/cwu022 (2014).
75 Locke, A. E. et at Genetic studies of body mass index yield new insights for obesity biology. Nature 518, 197-206, doi:10.1038/nature14177 (2015).
76 Zougman, A. et at Integrated analysis of the cerebrospinal fluid peptidome and proteome. Journal of proteome research 7, 386-399, doi:10.1021/pr070501k (2008).
77 Lottspeich, F., Kellermann, J., Henschen, A., Foertsch, B. & Muller-Esterl, W. The amino acid sequence of the light chain of human high-molecular-mass kininogen. European journal of biochemistry/FEBS 152, 307-314 (1985).
78 Makimattila, S., Fineman, M. S. & Yki-Jarvinen, H. Deficiency of total and nonglycosylated amylin in plasma characterizes subjects with impaired glucose tolerance and type 2 diabetes. J Clin Endocrinol Metab 85, 2822-2827, doi: 10.1210/jcem.85.8.6721 (2000).
79 Schellenberger, U. et at The precursor to B-type natriuretic peptide is an O-linked glycoprotein. Arch Biochem Biophys 451, 160-166, doi:10.1016/j.abb.2006.03.028 (2006).
80 Pal, K., Melcher, K. & Xu, H. E. Structure and mechanism for recognition of peptide hormones by Class B G-protein-coupled receptors. Acta Pharmacol Sin 33, 300-311, 55 doi:10.1038/aps.2011.170 (2012).
81 Naot, D. & Cornish, J. The role of peptides and receptors of the calcitonin family in the regulation of bone metabolism. Bone 43, 813-818, doi: 10.1016/j.bone.2008.07.003 (2008).
82 Muff, R., Born, W. & Fischer, J. A. Calcitonin, calcitonin gene-related peptide, adrenomedullin and amylin: homologous peptides, separate receptors and overlapping biological actions. European journal of endocrinology/European Federation of Endocrine Societies 133, 17-20 (1995).
83 Tagashira, M., Iijima, H. & Toma, K. An NMR study of O-glycosylation induced structural changes in the alpha-helix of calcitonin. Glycoconj J 19, 43-52 (2002).
84 Fisone, G. et al. N-terminal galanin-(1-16) fragment is an agonist at the hippocampal galanin receptor. Proc Natl Acad Sci USA 86, 9588-9591 (1989).
85 Cabrele, C. & Beck-Sickinger, A. G. Molecular characterization of the ligand-receptor interaction of the neuropeptide Y family. J Pept Sci 6, 97-122, doi: 10.1002/(SICI)1099-1387(200003)6:3<97::AID-PSC236>3.0.CO;2-E (2000).
86 Nicole, P. et al. Identification of key residues for interaction of vasoactive intestinal peptide with human VPAC1 and VPAC2 receptors and development of a highly selective VPAC1 receptor agonist. Alanine scanning and molecular modeling of the peptide. J Biol Chem 275, 24003-24012, doi: 10.1074/jbc.M002325200 (2000).
87 Cerda-Reverter, J. M. & Larhammar, D. Neuropeptide Y family of peptides: structure, anatomical expression, function, and molecular evolution. Biochem Cell Biol 78, 371-392 (2000).
88 Scarborough, R. M. et al. D-amino acid-substituted atrial natriuretic peptide analogs reveal novel receptor recognition requirements. J Biol Chem 263, 16818-16822 (1988).
89 Campbell, D. J. Long-term neprilysin inhibition—implications for ARNIs. Nat Rev Cardiol 14, 171-186, doi:10.1038/nrcardio.2016.200 (2017).
90 Lambeir, A. M., Durinx, C., Scharpe, S. & De Meester, I. Dipeptidyl-peptidase IV from bench to bedside: an update on structural properties, functions, and clinical aspects of the enzyme DPP IV. Crit Rev Clin Lab Sci 40, 209-294, doi:10.1080/713609354 (2003).
91 Nauck, M. Incretin therapies: highlighting common features and differences in the modes of action of glucagon-like peptide-1 receptor agonists and dipeptidyl peptidase-4 inhibitors. Diabetes Obes Metab 18, 203-216, doi: 10.1111/dom. 12591 (2016).
92 Deacon, C. F. et al. Dipeptidyl peptidase IV resistant analogues of glucagon-like peptide-1 which have extended metabolic stability and improved biological activity. Diabetologia 41, 271-278, doi: 10.1007/s001250050903 (1998).
93 Plamboeck, A., Holst, J. J., Carr, R. D. & Deacon, C. F. Neutral endopeptidase 24.11 and dipeptidyl peptidase IV are both mediators of the degradation of glucagon-like peptide 1 in the anaesthetised pig. Diabetologia 48, 1882-1890, doi:10.1007/s00125-005-1847-7 (2005).
94 Hupe-Sodmann, K. et al. Characterisation of the processing by human neutral endopeptidase 24.11 of GLP-1(7-36) amide and comparison of the substrate specificity of the enzyme for other glucagon-like peptides. Regulatory peptides 58, 149-156 (1995).
95 Torang, S., Veedfald, S., Rosenkilde, M. M., Hartmann, B. & Holst, J. J. The anorexic hormone Peptide YY3-36 is rapidly metabolized to inactive Peptide YY3-34 in vivo. Physiol Rep 3, doi: 10.14814/phy2.12455 (2015).

1. A formulation comprising at least one molecule of a peptide hormone species exhibiting a specific glycosylation pattern of one or more O-linked glycan(s) at a predetermined specific site of said peptide hormone species, wherein specific, defined glycosylation pattern means that the each molecule of said peptide hormone in said pharmaceutical formulation displays structural homogeneity with respect to the site of glycan attachment and/or with respect to the glycan attachment.

2. A formulation comprising at least one molecule of a peptide hormone species according to claim 1, wherein said peptide hormone species is selected from the group of sequences comprising SEQ ID NOs: 1, 2, 3, 5, 6, 7, 14, 15, 16, 21, 22, 23, 24, 25, 36, 37, 38, 39, 40, 41, 42, 43, 45, 46, 47, 48, 49, 51, 52, 54, 55, 56, 57, 72, 73, 74, 76, 79, 8, 81, 83, 84, 91, 92, 93, 94, 95, 96, 97, 98, 99, 100, 101, 102, 106, 107, 108, 109, 113, 116, 117, 118, 119, 120, 130, 131, 135, 136, 143, 144, 147, 163, 164, 167, 168, 170, 184, 185, 186, 188, 189, 190, 191, 192, 204, 215, 217, 219, 222, 227, 229, 230, 231, 233, 234, 236, 252, 260, 262, 267, 272, and/or 279.

3. A formulation comprising at least one molecule of a peptide hormone species according to claim 1, wherein said peptide hormone species is selected from the group of sequences comprising SEQ ID NOs: 6, 7, 21, 22, 23, 24, 72, 74, 92, 95, 97, 99, 106, 107, 108, 116, 117, 118, 147, 163, 167, 185, 186, and/or 188.

4. A formulation comprising at least one molecule of a peptide hormone species according to claim 1, wherein said peptide hormone species is selected from the group of sequences comprising SEQ ID NOs: 72, 95, 97, 106, 108, 147, 185, and/or 188.

5. A formulation comprising at least one molecule of a peptide hormone species according to claim 1, wherein said peptide hormone species is SEQ ID NO: 147.

6. A formulation comprising at least one molecule of a peptide hormone species according to claim 1, wherein said predetermined specific site of said peptide hormone is within the receptor-binding region.

7. A formulation comprising at least one molecule of a peptide hormone species according to claim 1, wherein said peptide hormone exhibiting an specific glycosylation pattern of one or more O-linked glycan(s) at a predetermined specific site of said peptide hormone is obtainable by recombinant production using a host cell in which one or more glycosyltransferase gene(s) is/are inactivated or downregulated by inactivation and/or downregulation of one or more gene(s) selected from COSMC and C1GALT1; and/or by inactivation and/or downregulation of one or more gene(s) selected from GCNT3, GCNT4, B3GNT6, and/or by inactivation and/or downregulation and/or upregulation/activation of one or more gene(s) selected from ST6GALNAC1-6, ST3GAL1, GCNT3, GCNT4, and/or B3GNT6.

8. A formulation comprising at least one molecule of a peptide hormone species according to claim 1, wherein said peptide hormone species exhibits a specific glycosylation pattern of one or more O-linked glycan(s) at a predetermined specific site of said peptide hormone and, wherein the one or more O-glycan structures include a glycan structure selected from a core1, core2, core3, or core4 structure with optional elongation and sialic acid capping, wherein optionally the first monosaccharide attached in the synthesis of O-linked glycans is N-acetyl-galactosamine and wherein a core1 structure may be obtained by the addition of galactose, and wherein a core2 structure may be obtained by the addition of N-acetyl-glucosamine to the N-acetyl-galactosamine of the core1 structure and wherein the core3 structures may be obtained by the addition of a single N-acetyl-glucosamine to the first monosaccharide N-acetyl-galactosamin and core4 structures may be obtained by the addition of a second N-acetyl-glucosamine to the core3 structure.

9. A formulation comprising at least one molecule of a peptide hormone species according to claim 1, wherein the one or more O-glycan structures include a Tn (GalNAc) structure.

10. A formulation comprising at least one molecule of a peptide hormone species according to claim 1, wherein the one or more O-glycan structures include Tn (GalNAc) structure with one sialic acid capping (alpha2-6).

11. A formulation comprising at least one molecule of a peptide hormone species according to claim 1, wherein the one or more O-glycan structures include the core1 structures with one sialic acid capping (alpha2-6).

12. A formulation comprising at least one molecule of a peptide hormone species according to claim 1, wherein the one or more O-glycan structures include the core1 structures with one sialic acid capping (alpha 2-3)

13. A formulation comprising at least one molecule of a peptide hormone species according claim 1, wherein the one or more O-glycan structures include the core1 structures with two sialic acids capping (alpha 2-3 and alpha 2-6).

14. A formulation according to claim 1, wherein said formulation is a pharmaceutical formulation.

15. A formulation comprising at least one molecule of a peptide hormone species as defined in claim 1, wherein said peptide hormone species comprises one or more O-linked glycan at a site indicated in and one of Tables 6A to 6E, particularly, wherein the site is a site in a human peptide hormone, more particularly, wherein the site is a conserved site in a human peptide hormone.

16. A formulation comprising at least one molecule of a peptide hormone species as defined in claim 15, wherein said peptide hormone species comprises one or more O-linked glycan at a site indicated in Table 6A, particularly, wherein the site is a site in a human peptide hormone, more particularly, wherein the site is a conserved site in a human peptide hormone.

17. A formulation comprising at least one molecule of a peptide hormone species as defined in claim 15, wherein said peptide hormone species comprises one or more O-linked glycan at a site of a peptide hormone indicated in Table 6B, particularly, wherein the site is a site in a human peptide hormone, more particularly, wherein the site is a conserved site in a human peptide hormone.

18. A formulation comprising at least one molecule of a peptide hormone species as defined in claim 15, wherein said peptide hormone species comprises one or more O-linked glycan at a site of a peptide hormone indicated in Table 6C, particularly, wherein the site is a site in a human peptide hormone, more particularly, wherein the site is a conserved site in a human peptide hormone.

19. A formulation comprising at least one molecule of a peptide hormone species as defined in claim 15, wherein said peptide hormone species comprises one or more O-linked glycan at a site of a peptide hormone indicated in Table 6C, particularly, wherein the site is a site in a human peptide hormone, more particularly, wherein the site is a conserved site in a human peptide hormone.

20. A formulation comprising at least one molecule of a peptide hormone species as defined in claim 15, wherein said peptide hormone species comprises one or more O-linked glycan at a site of a peptide hormone indicated in Table 6D, particularly, wherein the site is a site in a human peptide hormone, more particularly, wherein the site is a conserved site in a human peptide hormone.

21. A modified peptide hormone comprising one or more O-linked glycan at a predetermined specific site selected from the group comprising the peptide hormones indicated in 6A, particularly, wherein the site is a site in a human peptide hormone, more particularly, wherein the site is a conserved site in a human peptide hormone, and wherein said peptide hormone is selected from the group comprising SEQ ID Nos: 1, 2, 3, 5, 6, 7, 14, 15, 16, 21, 22, 23, 24, 25, 36, 37, 38, 39, 40, 41, 42, 43, 45, 46, 47, 48, 49, 51, 52, 54, 55, 56, 57, 72, 73, 74, 76, 79, 8, 81, 83, 84, 91, 92, 93, 94, 95, 96, 97, 98, 99, 100, 101, 102, 106, 107, 108, 109, 113, 116, 117, 118, 119, 120, 130, 131, 135, 136, 143, 144, 147, 163, 164, 167, 168, 170, 184, 185, 186, 188, 189, 190, 191, 192, 204, 215, 217, 219, 222, 227, 229, 230, 231, 233, 234, 236, 252, 260, 262, 267, 272, and/or 279.

22. The modified peptide hormone comprising one or more O-linked glycan at a predetermined specific site selected from the group comprising the peptide hormones indicated in 6B, particularly, wherein the site is a site in a human peptide hormone, more particularly, wherein the site is a conserved site in a human peptide hormone, and wherein said peptide hormone is selected from the group comprising SEQ ID Nos: 6, 7, 21, 22, 23, 24, 72, 74, 92, 95, 97, 99, 106, 107, 108, 116, 117, 118, 147, 163, 167, 185, 186, and/or 188.

23. The modified peptide hormone comprising one or more O-linked glycan at a predetermined specific site selected from the group comprising the peptide hormones indicated in 6C, particularly, wherein the site is a site in a human peptide hormone, more particularly, wherein the site is a conserved site in a human peptide hormone, and wherein said peptide hormone is selected from the group comprising SEQ ID Nos: 72, 95, 97, 106, 108, 147, 185, and/or 188.

24. The modified peptide hormone comprising one or more O-linked glycan at a predetermined specific site selected from the group comprising the peptide hormones indicated in 6D, particularly, wherein the site is a site in a human peptide hormone, more particularly, wherein the site is a conserved site in a human peptide hormone, and wherein said peptide hormone is depicted in SEQ ID NO: 147.

25. The modified peptide hormone according to claim 21, wherein the one or more O-glycan structures include a glycan structure selected from a core1, core2, core3, or core4 structure with sialic acid capping, wherein the one or more O-glycan structures include a glycan structure selected from a core1, core2, core3, or core4 structure with optional elongation and sialic acid capping, wherein optionally the first monosaccharide attached in the synthesis of O-linked glycans is N-acetyl-galactosamine and wherein a core1 structure may be obtained by the addition of galactose, and wherein a core2 structure may be obtained by the addition of N-acetyl-glucosamine to the N-acetyl-galactosamine of the core1 structure and wherein the core3 structures may be obtained by the addition of a single N-acetyl-glucosamine to the first monosaccharide N-acetyl-galactosamin and core4 structures may be obtained by the addition of a second N-acetyl-glucosamine to the core3 structure.

26. The modified peptide hormone according to claim 21, wherein the one or more O-glycan structures include a Tn (GalNAc) structure.

27. The modified peptide hormone according to claim 21, wherein the one or more O-glycan structures include Tn (GalNAc) structure with one sialic acid capping (alpha2-6).

28. The modified peptide hormone according to claim 21, wherein the one or more O-glycan structures include the core1 structures with one sialic acid capping (alpha2-6).

29. The modified peptide hormone according to claim 21, wherein the one or more O-glycan structures include the core1 structures with one sialic acid capping (alpha 2-3)

30. The modified peptide hormone according to claim 21, wherein the one or more O-glycan structures include the core1 structures with two sialic acids capping (alpha 2-3 and alpha 2-6).
